(19) United States
(12) Patent Application Publication
(10) Pub. No.: US 2003/0162788 A1
(43) Pub. Date: Aug. 28, 2003

(54) Combination of MTP inhibitors or apoB-secretion inhibitors with fibrates for use as pharmaceuticals

(75) Inventors: Leo Thomas, Biberach (DE); Michael Mark, Biberach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma KG, Ingelheim (DE)

(21) Appl. No.: 10/339,088

(22) Filed: Jan. 9, 2003

Related U.S. Application Data
(60) Provisional application No. 60/353,397, filed on Feb. 1, 2002.
(60) Provisional application No. 60/435,386, filed on Dec. 20, 2002.

(30) Foreign Application Priority Data
Jan. 10, 2002 (DE) 10200633.4
Dec. 2, 2002 (DE) 10256184.2

(51) Int. Cl. A61K 31/50; A61K 31/501; A61K 31/497; A61K 31/505; A61K 31/4439

(52) U.S. Cl. 514/252.05; 514/255.05; 514/256; 514/318; 514/341; 514/342; 514/343

(57) ABSTRACT

The invention relates to the use of fibrates for lowering the liver toxicity of MTP inhibitors as well as pharmaceutical compositions containing an MTP inhibitor and a fibrate.

Benefit of U.S. Provisional Application Serial No. 60/353,397 filed on Feb. 1, 2002 and U.S. Provisional Application Serial No. 60/435,386 filed on Dec. 20, 2002 is hereby claimed, and said applications are herein incorporated by reference.

The invention relates to the use of a combination of inhibitors of Microsomal Triglyceride Transfer Protein (MTP) with fibrates for treating hyperlipidaemia, dyslipidaemia, atherosclerosis, diabetes mellitus, obesity and pancreatitis with the purpose of reducing the mechanism-induced side effects of an MTP inhibitor in the liver by combination with a fibrate and thereby at least maintaining the activity of the MTP inhibitor, pharmaceutical compositions containing this combination and the preparation thereof. MTP inhibitors lower the lipid concentration in the blood by inhibiting the secretion of apolipoprotein B (apoB)-containing lipoproteins in the liver and intestines. This leads to an accumulation of lipids (steatosis) in the target organs which can lead to cell damage in the liver in particular. The cell damage can be detected in positive liver function tests (e.g. an increase in transaminases).

Surprisingly, it has now been found that the steatosis caused by MTP inhibitors is reduced in combination with fibrates which stimulate metabolism of the fatty acids in the liver and that the liver function tests revert to normal. As a result, on the one hand the MTP inhibitors have a positive therapeutic effect but at the same time the mechanism-induced toxicity is prevented. Moreover, the combination with fibrate can also potentiate the positive lipid-modulating activity of the MTP inhibitor (synergistic effect). The invention relates to all MTP inhibitors. Similarly, all fibrates are included. The two active substances may be administered both simultaneously in a single pharmaceutical preparation or successively in two pharmaceutical preparations. They are preferably administered in a single preparation.

### BACKGROUND TO THE INVENTION

1. Inhibitors of the Microsomal Triglyceride Transfer Protein

Microsomal Triglyceride Transfer Protein (MTP) catalyses the transporting of lipids between phospholipid surfaces [Wetterau J R et al., Biochim Biophys Acta 1345, 136-150 (1997)]. The protein is found in the lumen of liver and intestinal microsomes. MTP is a heterodimer which consists of an MTP-specific large subunit (97 kD) and protein disulphide isomerase (PDI, 58 kD). PDI is a widely distributed protein of the endoplasmatic reticulum (ER) and an essential component for the structural and functional integrity of MTP. MTP is necessary for the intracellular production of apolipoprotein B (apoB)-containing plasma lipoproteins. Although the precise role of MTP in the composition of the lipoproteins is not known, it very probably transports lipids from the membrane of the ER to the lipoprotein particles forming in the lumen of the ER.

Apolipoprotein B is the main protein component of hepatic VLDL (very low density lipoproteins) and intestinal chylomicrons. Substances that inhibit MTP reduce the secretion of apoB-containing lipoproteins [Haghpassand M et al., J lipid Res 37, 1468-1480 (1996); Jamil H et al., Proc Natl Acad Sci USA 93, 11991-11995 (1996); Wetterau J R et al., Science 282, 751-754 (1998)]. Therefore, any inhibition of MTP lowers the plasma concentrations of cholesterol and triglycerides in apoB-containing lipoproteins. This has been demonstrated in hamsters and rabbits [Wetterau J R et al., Science 282, 751-754 (1998)], in heterozygotic MTP-deficient mice [Raabe M et al., Proc Natl Acad Sci USA 95, 8686-8691 (1998)] and in clinical trials in humans [Roevens P et al., Atherosclerosis 144, 38-39 (1999); Wilder D E, Drugs Affecting lipid Metabolism—XIVth International Symposium, New York, N.Y., USA, Sep. 9-12, 2001, Abstract; Farnier M, Drugs Affecting lipid Metabolism—XIVth International Symposium, New York, N.Y., USA, Sep. 9-12, 2001, Abstract].

ApoB-containing triglyceride-rich lipoproteins and the residues thereof enriched with cholesterol (e.g. LDL) are atherogenic and contribute to the morbidity and mortality of coronary heart disease. The correlation between the concentration of LDL-cholesterol (or total cholesterol as a closely related representative parameter) and clinical findings is generally recognised. Numerous intervention studies have shown a reduction in coronary events under lipid-reducing treatment. One advantage turned out to be secondary prevention in patients both with raised cholesterol levels (4S [Anonymous, Lancet 8934,1383-1389 (1994)], POSCH [Buchwald H et al., Archives of Internal Medicine 11, 1253-1261 (1998)], CDP [Canner P L et al., J. Am. Coll. Cardiol. 6, 1245-1255 (1986)]) and with normal to borderline cholesterol levels (LIPID [Anonymous, New England Journal of Medicine 19, 1349-1357 (1998)], CARE [Pfeffer M A et al., Journal of the American College of Cardiology 1, 125-130 (1999)], LRC-CPPT [Anonymous, Archives of Internal Medicine 7, 1399-1410 (1992)], Helsinki Heart Study [Frick M H et al., New England Journal of Medicine 20, 1237-1245 (1987)]), and also primary prevention in people with raised cholesterol levels (WOSCOPS [Shepherd J et al., New England Journal of Medicine 20, 1301-1307 (1995)]) and without raised cholesterol levels (AFCAPS [Downs J R et al., JAMA 20, 1615-1622 (1998)]).

In a recently completed meta-analysis of 17 prospective studies raised triglyceride levels were an independent risk factor for coronary heart disease [Austin M A et al., American Journal of Cardiology 4A, 7B-12B (1998)]. The ARIC study showed that raised postprandial triglyceride levels are an independent risk factor for atherosclerosis, even after taking into account the lipid levels found when fasting [Sharrett A R et al., Arterioscler Thromb Vasc Biol 15, 2122-2129 (1995)]. In the Guidelines of the National Cholesterol Education Program of the National Heart, Lung and Blood Institute of the USA (Adult Treatment Panel III, ATP III) raised triglyceride levels were regarded as an independent risk factor for atherosclerosis and coronary heart disease [JAMA 285, 2486-2497 (2001)]. Moreover, there are indications that other lipid parameters connected with apoB such as Lp(a) are risk factors for the development of atherosclerotic cardiovascular diseases [Ridker P M et al., JAMA 270, 2195-2199 (1993); Bostom A G et al., JAMA 276, 544-548 (1996)].

Substances that inhibit MTP in the liver or in the intestines are consequently useful for lowering the concentration of apoB-containing lipoproteins in the plasma. This includes the states of general and postprandial hypercholesterolaemia and hypertriglyceridaemia. The treatment of raised levels of Lp(a) is also included. Since apoB-containing lipoproteins contribute to the development of atherosclerosis, these substances are also useful for preventing and treating atherosclerotic diseases. They are also useful for treating dyslipidaemic states and complications in related diseases such as diabetes mellitus (type II diabetes), obesity and pancreatitis. The inhibition of the intestinal absorption of fats from the food by MTP inhibitors is useful for treating conditions such as obesity and diabetes mellitus in which an excessive fat intake contributes significantly to the development of the disease [Grundy S M, Am J Clin Nutr 57(suppl), 563S-572S (1998)].

2. Fibrates

Derivatives of fibric acid (fibrates) represent a category of lipid reducing substances which in particular lower triglycerides in the plasma and increase HDL cholesterol [Miller D B & Spence J D, Clin Pharmacokinet 34, 155-162 (1998)]. The effects on LDL cholesterol on the other hand are less marked and more variable. The VA-HIT study (Veterans Affairs Cooperative Studies Program High-Density lipoprotein cholesterol Intervention Trial) showed for the first time that increasing HDL cholesterol lowers morbidity and mortality [New England Journal of Medicine 431, 410-418 (1999)]. The category of fibrates on the market includes clofibrat [Kesaniemi Y A & Grundy S M, JAMA 251, 2241-2247 (1984)], bezafibrat [Goa K L et al., Drugs 52, 725-753 (1996)], ciprofibrat [Turpin G & Bruckert E, Atherosclerosis 124 Suppl, S83-S87 (1996)], fenofibrat [Balfour J A et al., Drugs 40, 260-290 (1990); Packard C J, Eur Heart J 19 Suppl A, A62-A65 (1998)] and gemfibrozil [Spencer C M & Barradell L B, Drugs 51, 982-1018 (1996)].

The clinical effects of the fibrates are produced by changes in the transcription of genes which play important parts in lipid metabolism. Changes in transcription are based on the activation of a transcription factor, peroxisome-proliferator-activated receptor alpha (PPARα). Peroxisome-proliferator-activated receptors (PPARs) belong to the family of the nuclear hormone receptors. PPARα, the first member of this family to be identified, is expressed mainly in tissues that have a high rate of β-oxidation (liver, kidney, heart, muscle). PPARα is activated by fatty acids in the food, by eicosanoids and pharmacologically by fibrates. In mechanistic terms fibrates are PPARα agonists [Gervois P et al., Clin Chem Lab Med 38, 3-11 (2000)]. PPARα mediates the lipid-modifying effects of the fibrates in the treatment of hypertriglyceridaemia and hypoalphalipoproteinaemia. PPARα is regarded as the chief regulator of intra- and extracellular lipid metabolism. After activation by fibrates PPARα down-regulates the expression of the apolipoprotein C-III gene and up-regulates the expression of the lipoprotein lipase gene, leading to potentiation of the VLDL catabolism. In addition, the activation of PPARα leads to the induction of the genes for apolipoprotein A-I and A-II, resulting in an increase in HDL cholesterol. PPARα activation also causes up-regulation of the genes for the cholesterol transporters ABCA-1 and SR-B1 and consequently an increase in the reverse transportation of cholesterol.

In connection with the present invention the role played by PPARα in intracellular lipid metabolism is particularly important [Everett L et al., Liver 20, 191-199 (2000)]. The activation of PPARα leads to an increase in the gene expression of enzymes which are needed for the β-oxidation of fatty acids. These include first of all enzymes of fatty acid activation (Acyl-CoA synthetase, fatty acid-binding proteins) and enzymes that mediate the entry of the fatty acids into mitochondria (carnitin-palmitoyl transferase I). In addition, enzymes of mitochondrial β-oxidation of fatty acids are induced (e.g. Acyl-CoA dehydrogenase, 3-ketoacyl-CoA thiolase). In rodents in particular, enzymes of the peroxisomal β-oxidation of fatty acids (e.g. Acyl-CoA oxidase) and microsomal c-oxidation of fatty acids (e.g. cytochrome P450 4A1 (lauryl ω-hydroxylase)) are up-regulated.

### DETAILED DESCRIPTION

MTP inhibitors lower the fasting concentration of cholesterol and triglycerides in the blood by inhibiting the secretion of lipoproteins in the liver (Very Low Density lipoproteins, VLDL). This results in an accumulation of the lipids in the hepatocytes (hepatic steatosis). As soon as a certain level of steatosis is reached, this causes damage to the liver cells. This cell damage can be detected by the release of intracellular enzymes which are then found in greater amounts in the blood. These enzymes which indicate hepatocellular damage include alanine-aminotransferase (ALT), aspartate-aminotransferase (AST) and glutamate dehydrogenase (GLDH). The cell damage caused by hepatic steatosis greatly restricts the use of effective MTP inhibitors.

The present invention shows a way of reducing the mechanism-induced side effects of an MTP inhibitor in the liver. When an MTP inhibitor is combined with a fibrate the β-oxidation of fatty acids in the liver is stimulated by the PPARα agonism of the fibrate. The fatty acids released from the accumulated triglycerides after hydrolysis can thus be broken down to a greater extent. The content of triglycerides and free fatty acids in the liver falls. The hepatic steatosis is thereby reduced to a level which is no longer harmful to the liver cells. This can be recognised by normal levels of hepatocellular enzymes in the blood. In this way, the effective lipid reduction caused by MTP inhibitors in the blood can be achieved without any toxic side effects in the liver.

According to another aspect of the invention the effects of MTP inhibitors and fibrates on lipids in the blood complement one another. The lowering of cholesterol and triglycerides can be potentiated by combining the two categories of active substance. In addition, increasing HDL cholesterol is a special property of fibrates. This makes it possible to combine the effect of MTP inhibitors on lowering triglycerides and atherogenic cholesterol in lipoproteins containing apolipoprotein B with the desired increase in HDL cholesterol by means of fibrates.

The invention relates generally to the combination of any desired MTP inhibitor with any desired fibrate in order to prevent the mechanism-induced liver toxicity of MTP inhibitors. At the same time the desired activity of the MTP inhibitor is increased.

According to the invention, MTP inhibitors of general formula I

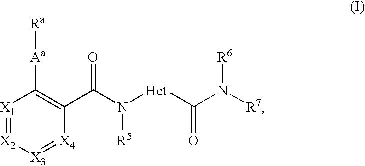

the tautomers, the diastereomers, the enantiomers, the mixtures and salts thereof, particularly the physiologically acceptable salts thereof, may be used.

In general formula I

X₁ denotes the group CR¹,

X₂ denotes the group CR²,

X₃ denotes the group CR³ and

X₄ denotes the group CR⁴ or

one or two of the groups X₁ to X₄ in each case denote a nitrogen atom and the remainder of the groups X₁ to X₄ denote three or two of the groups CR¹ to CR⁴,

while R¹, R², R³ and R⁴ in each case denote a hydrogen atom or

one or two of the groups R¹ to R⁴ independently of one another in each case denote a fluorine, chlorine or bromine atom, a C₁₋₃-alkyl group, a trifluoromethyl, hydroxy, C₁₋₃-alkoxy, trifluoromethoxy, amino, C₁₋₃-alkylamino or di-(C₁₋₃-alkyl)-amino group and the remainder of the groups R¹ to R⁴ in each case represent a hydrogen atom,

while R⁴ additionally together with R⁵ may assume the meaning of a —(CH₂)ₙ— bridge wherein n denotes the number 1, 2 or 3, and

A^{a} denotes a bond, an oxygen or sulphur atom, an —NH, —N(C₁₋₃-alkyl), sulphinyl, sulphonyl or carbonyl group,

one of the groups —CH₂—, —(CH₂)₂—, —CH═CH—, —C≡C—, —OCH₂—, —CH₂O—, —NH—CH₂—, —CH₂—NH—, —NH—CO—, —CO—NH—, —NH—SO₂— or —SO₂—NH—,

wherein a hydrogen atom bound to a carbon atom and/or a hydrogen atom bound to a nitrogen atom may be replaced in each case by a C₁₋₃-alkyl group and wherein a heteroatom of the group A^{a} is not linked to a nitrogen atom of a 5-membered heteroaryl group of the group R^{a},

R^{a} denotes a phenyl, 1-naphthyl or 2-naphthyl group,

a 5-membered heteroaryl group bound via a carbon or nitrogen atom, which contains

an imino group optionally substituted by a C₁₋₄-alkyl or C₁₋₄-alkylcarbonyl group, an oxygen or sulphur atom,

an imino group optionally substituted by a C₁₋₄-alkyl group or an oxygen or sulphur atom and additionally a nitrogen atom or

an imino group optionally substituted by a C₁₋₄-alkyl group and two nitrogen atoms or

an oxygen or sulphur atom and two nitrogen atoms,

a 6-membered heteroaryl group which contains one or two nitrogen atoms,

while a phenyl ring may be fused to the said 5- or 6-membered heteroaryl groups via two adjacent carbon atoms and the bicyclic heteroaryl groups thus formed may be bound via the heteroaromatic or carbocyclic moiety and

wherein the said phenyl and naphthyl groups as well as the mono- and bicyclic heteroaryl groups in the carbon skeleton may be monosubstituted by a fluorine, chlorine or bromine atom, by a C₁₋₄-alkyl group, by a C₃₋₇-cycloalkyl, trifluoromethyl, phenyl, hydroxy, C₁₋₃-alkoxy, trifluoromethoxy, amino, C₁₋₃-alkylamino, di-(C₁₋₃-alkyl)amino, acetylamino, N—(C₁₋₃-alkyl)-acetylamino, propionylamino, N—(C₁₋₃-alkyl)-propionylamino, acetyl, propionyl, C₁₋₃-alkoxycarbonyl, aminocarbonyl, C₁₋₃-alkylamino-carbonyl, di-(C₁₋₃-alkyl)amino-carbonyl or cyano group or, with the exception of 5-membered heteroaryl groups containing more than two heteroatoms, may also be disubstituted by the abovementioned substituents, while the substituents may be identical or different,

a C₃₋₇-cycloalkyl group, wherein

in each case the methylene group in the 4 position of a 6- or 7-membered cycloalkyl group may be replaced by an oxygen or sulphur atom, by a sulphinyl or sulphonyl group or by an imino group optionally substituted by a C₁₋₅-alkyl, phenyl, C₁₋₄-alkyl-carbonyl, C₁₋₄-alkoxy-carbonyl, C₁₋₃-alkyl-aminocarbonyl or di-(C₁₋₃-alkyl)-aminocarbonyl group,

a 4- to 7-membered cycloalkyleneimino group wherein

the cycloalkylene moiety may be fused to a phenyl ring or

one or two hydrogen atoms may be replaced in each case by a C₁₋₃-alkyl group and/or

in each case the methylene group in the 4 position of a 6- or 7-membered cycloalkyleneimino group may be substituted by a hydroxycarbonyl, C₁₋₃-alkoxycarbonyl, amino, C₁₋₃-alkylamino, di-(C₁₋₃-alkyl)amino, aminocarbonyl, C₁₋₃-alkylamino-carbonyl, di-(C₁₋₃-alkyl)-aminocarbonyl or phenyl-C₁₋₃-alkylamino group or

may be replaced by an oxygen or sulphur atom, by a sulphinyl or sulphonyl group or by an imino group optionally substituted by a C₁₋₅-alkyl, phenyl, C₁₋₄-alkyl-carbonyl, C₁₋₄-alkoxy-carbonyl, C₁₋₃-alkyl-aminocarbonyl or di-(C₁₋₃-alkyl)-aminocarbonyl group or

in a 5-, 6- or 7-membered cycloalkyleneimino group a —CH₂— group linked to the imino nitrogen atom may be replaced by a carbonyl group or

a —(CH₂)₂— group linked to the imino nitrogen atom may be replaced by a —CO—NR⁸— group or

a —(CH₂)₃— group linked to the imino nitrogen atom may be replaced by a —CO—NR⁸—CO— group,

while R⁸ denotes a hydrogen atom or a C₁₋₃-alkyl group,

R⁵ denotes a hydrogen atom or a C₁₋₅-alkyl group,

Het denotes a 5-membered heteroarylene group bound via two carbon atoms or, if Het denotes a double-bonded pyrrole group, it may also be bound via a carbon atom and the imino-nitrogen atom, the latter being linked to the adjacent carbonyl group in formula (I), which contains

an imino group substituted by the group R⁹, an oxygen or sulphur atom or

an imino group substituted by the group R⁹ or an oxygen or sulphur atom and additionally a nitrogen atom,

while R⁹ denotes a hydrogen atom, a C₁₋₅-alkyl group, a C₂₋₃-alkyl group terminally substituted by an amino, C₁₋₃-alkylamino, di-(C₁₋₃-alkyl)-amino or C₁₋₅-alkoxy-carbonyl-amino group, a carboxy-C₁₋₃-alkyl, C₁₋₃-alkoxycarbonyl-C₁₋₃-alkyl, phenyl, phenyl-C₁₋₃-alkyl, C₁₋₅-alkylcarbonyl or phenylcarbonyl group or R⁹ together with R⁶ denotes a —(CH₂)ₚ— bridge, wherein p denotes the number 2 or 3,

or an imino group optionally substituted by a C₁₋₃-alkyl group and two nitrogen atoms or

an oxygen or sulphur atom and two nitrogen atoms,

or a 6-membered heteroarylene group which contains one or two nitrogen atoms,

while the abovementioned heteroarylene groups in the carbon skeleton may be monosubstituted by a fluorine, chlorine or bromine atom, by a C₁₋₅-alkyl group, by a C₃₋₇-cycloalkyl, trifluoromethyl, hydroxy, C₁₋₃-alkoxy, trifluoromethoxy, amino, C₁₋₃-alkylamino, di-(C₁₋₃-alkyl)amino, acetylamino, N—(C₁₋₃-alkyl)-acetylamino, propionylamino, N—(C₁₋₃-alkyl)-propionylamino, acetyl, propionyl, benzoyl, C₁₋₃-alkoxy-carbonyl, aminocarbonyl, C₁₋₃-alkylamino-carbonyl- di-(C₁₋₃-alkyl)-amino-carbonyl or cyano group or, with the exception of 5-membered monocyclic heteroaryl groups containing more than one heteroatom, may also be disubstituted by the abovementioned substituents, while the substituents may be identical or different,

R⁶ denotes a hydrogen atom or a C₁₋₆-alkyl group,

R⁷ denotes a C₁₋₉-alkyl group,

a straight-chain or branched, mono-, di- or triunsaturated C₃₋₉-alkenyl or C₃₋₉-alkynyl group, while the multiple bonds are isolated from the nitrogen-carbon bond,

a straight-chain C₂₋₆-alkyl group which is terminally substituted by an amino, C₁₋₃-alkylamino or di-(C₁₋₃-alkyl)-amino group,

a C₁₋₆-alkyl group substituted by a C₃₋₇-cycloalkyl group, while

a hydrogen atom in the 3 position of the cyclopentyl group and in the 4 position of a 6- or 7-membered cycloalkyl group may be replaced in each case by a hydroxy, hydroxy-C₁₋₃-alkyl, C₁₋₅-alkoxy, C₁₋₅-alkoxy-C₁₋₃-alkyl, phenyl-C₁₋₃-alkoxy-C₁₋₃-alkyl, amino, C₁₋₅-alkylamino, di-(C₁₋₅-alkyl)amino, phenyl-C₁₋₃-alkylamino, C₁₋₅-alkyl-carbonylamino, benzoylamino, amino-C₁₋₃-alkyl, C₁₋₃-alkylamino-C₁₋₃-alkyl, di-(C₁₋₃-alkyl)amino-C₁₋₃-alkyl, phenyl-C₁₋₃-alkylamino-C₁₋₃-alkyl, C₁₋₃-alkyl-carbonylamino-C₁₋₃-alkyl, benzoylamino-C₁₋₃-alkyl, phenylamino-carbonyl, phenyl-C₁₋₃-alkylamino-carbonyl, carboxy or C₁₋₃-alkoxy-carbonyl group or

in each case the methylene group in the 4 position of a 6- or 7-membered cycloalkyl group may be replaced by an oxygen or sulphur atom or by an imino group optionally substituted by a C₁₋₆-alkyl, phenyl, C₁₋₆-alkyl-carbonyl, benzoyl,

phenyl-(C₁₋₃-alkyl)-carbonyl, C₁₋₆-alkyl-aminocarbonyl,

di-(C₁₋₅-alkyl)-aminocarbonyl, phenylaminocarbonyl,

N—(C₁₋₃-alkyl)-phenylaminocarbonyl, phenyl-C₁₋₃-alkylamino-carbonyl or

N—(C₁₋₃-alkyl)-phenyl-C₁₋₃-alkylamino-carbonyl group or

in a 5- or 6-membered cycloalkyl group one or two single bonds separated from each other by at least one bond and separated from position 1 may in each case be fused to a phenyl group, while in a bi- or tricyclic ring system thus formed the hydrogen atom bound to the saturated carbon atom in position 1 may be replaced by a C₁₋₅-alkylamino-carbonyl, di-(C₁₋₅-alkyl)amino-carbonyl, phenyl-C₁₋₃-alkylamino-carbonyl or C₁₋₅-alkoxy-carbonyl group, wherein terminal methyl groups in each case may be wholly or partially fluorinated,

a C₁₋₆-alkyl group optionally substituted by a C₃₋₇-cycloalkyl group, which is substituted

by a carboxy or C₁₋₃-alkoxycarbonyl group,

by a phenyl, 1-naphthyl or 2-naphthyl group,

by a 5-membered heteroaryl group bound via a carbon or nitrogen atom, which contains

an imino group optionally substituted by a C₁₋₃-alkyl, trifluoromethyl, phenyl, phenyl-C₁₋₃-alkyl, C₁₋₃-alkylcarbonyl, phenylcarbonyl or phenyl-C₁₋₃-alkylcarbonyl group, an oxygen or sulphur atom,

an imino group optionally substituted by a C₁₋₃-alkyl group or an oxygen or sulphur atom and additionally a nitrogen atom or

an imino group optionally substituted by a C₁₋₃-alkyl group and two nitrogen atoms or

an oxygen or sulphur atom and two nitrogen atoms,

by a 6-membered heteroaryl group, which contains one or two nitrogen atoms,

while a phenyl ring may be fused to the abovementioned 5- or 6-membered heteroaryl groups via two adjacent carbon atoms and the bicyclic heteroaryl groups thus formed may be bound via the heteroaromatic or carbocyclic moiety,

while the abovementioned phenyl and naphthyl groups as well as the mono- and bicyclic heteroaryl groups in the carbon skeleton may be monosubstituted by a fluorine, chlorine or bromine atom, by a C₁₋₅-alkyl, trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, amino-C₁₋₃-alkyl, C₁₋₃-alkylamino-C₁₋₃-alkyl, di-(C₁₋₃-alkyl)amino-C₁₋₃-alkyl, C₁₋₅-alkoxy-carbonylamino-C₁₋₃-alkyl, acetylamino, propionylamino, N—(C₁₋₃-alkyl)-benzoylamino, acetyl, propionyl, carboxy, C₁₋₃-alkoxy-carbonyl, C₁₋₃-alkoxy-carbonyl-C₁₋₃-alkyl, aminocarbonyl, C₁₋₃-alkylamino-carbonyl, di-(C₁₋₃-alkyl)amino-carbonyl, or cyano group or, with the exception of 5-membered heteroaryl groups containing more than two heteroatoms, may also be disubstituted by the abovementioned substituents, while the substituents may be identical or different,

a C₁₋₆-alkyl group substituted by a phenyl group and a carboxy, C₁₋₃-alkoxy-carbonyl, aminocarbonyl, C₁₋₃-alkyl-aminocarbonyl or di-(C₁₋₃-alkyl)-aminocarbonyl group,

a phenyl-C₂₋₅-alkenylene-CH₂, phenyl-C₂₋₅-alkynylene-CH₂, heteroaryl-C₂₋₅-alkenylene-CH₂ or heteroaryl-C₂₋₅-alkynylene-CH₂ group, wherein a hydrogen atom of the methylene group in position 1 may be replaced by a C₁₋₃-alkyl group and independently thereof the phenyl moiety as well as the heteroaryl moiety may be mono- or disubstituted by fluorine, chlorine or bromine atoms, by C₁₋₆-alkyl, C₃₋₇-cycloalkyl, trifluoromethyl, C₁₋₃-alkoxy, phenyl, heteroaryl or cyano groups, while the substituents may be identical or different and disubstitution by two aromatic groups is excluded,

while heteroaryl denotes a 5-membered heteroaryl group bound via a carbon or nitrogen atom, which contains

an imino group substituted optionally by a C₁₋₃-alkyl group, an oxygen or sulphur atom,

an imino group substituted optionally by a C₁₋₃-alkyl group or an oxygen or sulphur atom and additionally a nitrogen atom or

an imino group substituted optionally by a C₁₋₃-alkyl group and two nitrogen atoms or

an oxygen or sulphur atom and two nitrogen atoms,

or a 6-membered heteroaryl group, which contains one or two nitrogen atoms,

while a phenyl ring may be fused to the abovementioned 5- or 6-membered heteroaryl groups via two adjacent carbon atoms and the bicyclic heteroaryl groups thus formed may be bound via the heteroaromatic or carbocyclic moiety,

the group R^{b}-A^{b}-E^{b}-C₁₋₃-alkyl optionally substituted in the C₁₋₃-alkyl moiety by a C₁₋₄-alkyl or C₃₋₅-cycloalkyl group, wherein

R^{b} denotes a phenyl group optionally mono- or disubstituted by fluorine, chlorine, bromine or iodine atoms, by C₁₋₄-alkyl, C₂₋₄-alkenyl, C₂₋₄-alkynyl, C₃₋₇-cycloalkyl, trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, amino-C₁₋₃-alkyl, C₁₋₃-alkylamino-C₁₋₃-alkyl, di-(C₁₋₃-alkyl)amino-C₁₋₃-alkyl, acetylamino, propionylamino, acetyl, propionyl, carboxy, C₁₋₃-alkoxy-carbonyl, C₁₋₃-alkoxycarbonyl-C₁₋₃-alkyl, aminocarbonyl, C₁₋₃-alkylamino-carbonyl, di-(C₁₋₃-alkyl)amino-carbonyl or cyano groups, while the substituents may be identical or different,

a 5-membered heteroaryl group which

may be bound via a carbon atom or, if A^{b} denotes a bond, a —CH₂, —(CH₂)₂, sulphonyl or carbonyl group, may also be bound via a nitrogen atom and which contains

an imino group optionally substituted by a C₁₋₃-alkyl group, an oxygen or sulphur atom,

an imino group optionally substituted by a C₁₋₃-alkyl group or an oxygen or sulphur atom and additionally a nitrogen atom or

an imino group optionally substituted by a C₁₋₃-alkyl group and two nitrogen atoms or

an oxygen or sulphur atom and two nitrogen atoms,

a 6-membered heteroaryl group, which contains one or two nitrogen atoms,

while a phenyl ring may be fused to the abovementioned 5- or 6-membered heteroaryl groups via two adjacent carbon atoms and the bicyclic heteroaryl groups thus formed may be bound via the heteroaromatic or carbocyclic moiety, while the abovementioned mono- and bicyclic heteroaryl groups may be monosubstituted in the carbon skeleton by a fluorine, chlorine or bromine atom, by a C₁₋₄-alkyl, C₂₋₄-alkenyl, C₂₋₄-alkynyl, C₃₋₇-cycloalkyl, trifluoromethyl, phenyl, hydroxy, C₁₋₃-alkoxy, trifluoromethoxy, amino, C₁₋₃-alkylamino, di-(C₁₋₃-alkyl)-amino, acetylamino, propionylamino, acetyl, propionyl, C₁₋₃-alkoxy-carbonyl, aminocarbonyl, C₁₋₃-alkylamino-carbonyl, di-(C₁₋₃-alkyl)amino-carbonyl or cyano group or, with the exception of 5-membered heteroaryl groups containing more than two heteroatoms, may also be disubstituted by the abovementioned substituents, while the substituents may be identical or different,

a C₃₋₇-cycloalkyl group wherein

one or two hydrogen atoms in each case may be replaced by a C₁₋₃-alkyl group and/or

in each case the methylene group in the 4 position of a 6- or 7-membered cycloalkyl group may be replaced by an oxygen or sulphur atom, by a sulphinyl, sulphonyl or by an imino group optionally substituted by a C₁₋₃-alkyl, C₁₋₃-alkyl-carbonyl, C₁₋₃-alkoxy-carbonyl, C₁₋₃-alkyl-aminocarbonyl or di-(C₁₋₃-alkyl)-aminocarbonyl group or

the two hydrogen atoms of the methylene group in the 3-position of a cyclopentyl group or in 3- or 4-position of a cyclohexyl or cycloheptyl group may be replaced by an n-butylene, n-pentylene, n-hexylene, 1,2-ethylenedioxy or 1,3-propylenedioxy group and in the rings thus formed one or two hydrogen atoms may be replaced by C₁₋₃-alkyl groups,

a 4- to 7-membered cycloalkyleneimino group wherein

the cycloalkylene moiety may be fused to a phenyl ring or one or two hydrogen atoms in each case may be replaced by a C₁₋₃-alkyl group and/or

in each case the carbon atom in the 4 position of a 6- or 7-membered cycloalkyleneimino group may be substituted by a hydroxy-C₁₋₃-alkyl, C₁₋₆-alkoxy-C₁₋₃-alkyl, hydroxycarbonyl, C₁₋₆-alkoxycarbonyl, aminocarbonyl, C₁₋₃-alkylamino-carbonyl, di-(C₁₋₃-alkyl)-aminocarbonyl-, 4- to 7-membered cycloalkyleneimino, phenyl, 4-(C₁₋₃-alkyl)-1,2,4-triazol-3-yl, phenyl-C₁₋₃-alkylamino or N—(C₁₋₃-alkyl)-phenyl-C₁₋₃-alkylamino group or

may be replaced by an oxygen or sulphur atom, by a sulphinyl or sulphonyl group or by an imino group optionally substituted by a C₁₋₃-alkyl, phenyl, C₁₋₃-alkyl-carbonyl, benzoyl, phenyl-C₁₋₃-alkyl-carbonyl, C₁₋₃-alkyl-aminocarbonyl, di-(C₁₋₃-alkyl)-aminocarbonyl, phenylaminocarbonyl or N—(C_{1 3}-alkyl)-phenylaminocarbonyl group or

the two hydrogen atoms of the methylene group in the 3 position of a 5-membered cycloalkyleneimino group or in the 3 or 4 position of a 6- or 7-membered cycloalkyleneimino group may be replaced by an n-butylene, n-pentylene, n-hexylene, 1,2-ethylenedioxy or 1,3-propylenedioxy group and in the rings thus formed one or two hydrogen atoms may be replaced by C₁₋₃-alkyl groups or

in a 5-, 6- or 7-membered cycloalkyleneimino group a —CH₂— group linked to the imino nitrogen atom may be replaced by a carbonyl group or a —(CH₂)₂— group linked to the imino nitrogen atom may be replaced by a —CO—NR⁸— group or

a —(CH₂)₁₋₃— group linked to the imino nitrogen atom may be replaced by a —CO—NR⁸—CO— group,

while R⁸ denotes a hydrogen atom or a C₁₋₃-alkyl group,

A^{b} denotes a bond, an oxygen or sulphur atom, an —NH, —N(C₁₋₃-alkyl), sulphinyl, sulphonyl or a carbonyl group,

one of the groups —CH₂—, —(CH₂)₂—, —O—CH₂—, —CH₂—O—, NH—CH₂—, —CH₂—NH—, —NH—CO—, —CO—NH—, —NH—SO—₂, —SO₂—NH—, —CH═CH— or —C≡C—

wherein a hydrogen atom bound to a carbon atom and/or a hydrogen atom bound to a nitrogen atom may be replaced by a C₁₋₃-alkyl group in each case and a heteroatom of the group A^{b} is not linked to a nitrogen atom of a 5-membered heteroaryl group of the group R^{b},

E^{b} denotes a phenylene group optionally substituted by a fluorine, chlorine or bromine atom, by a C₁₋₄-alkyl group, by a trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, amino-C₁₋₃-alkyl, C₁₋₃-alkylamino-C₁₋₃-alkyl, di-(C₁₋₃-alkyl)amino-C₁₋₃-alkyl, acetylamino, propionylamino, acetyl, propionyl, carboxy, C₁₋₃-alkoxy-carbonyl, C₁₋₃-alkoxy-carbonyl-C₁₋₃-alkyl, aminocarbonyl, C₁₋₃-alkylamino-carbonyl, di-(C₁₋₃-alkyl)amino-carbonyl or cyano group,

the group R^{c}-A^{c}-E^{c}-C₁₋₃-alkyl optionally substituted in the C₁₋₃-alkyl moiety by a C₁₋₄-alkyl or C₃₋₅-cycloalkyl group wherein

R^{c} assumes the meanings given for R^{b} hereinbefore, while any reference to A^{b} must be replaced by a reference to A^{c},

A^{c} assumes the meanings given for A^{b} hereinbefore, while any reference to R^{b} must be replaced by a reference to R^{c},

E^{c} denotes a 5-membered heteroarylene group bound via two carbon atoms or via a carbon atom and an imino-nitrogen atom, while the imino-nitrogen atom of the heteroarylene group is not linked to a heteroatom of the group A^{c} and the heteroarylene group contains

an imino group optionally substituted by a C₁₋₃-alkyl group, an oxygen or sulphur atom,

an imino group optionally substituted by a C₁₋₃-alkyl group or an oxygen or sulphur atom and additionally a nitrogen atom or

an imino group optionally substituted by a C₁₋₃-alkyl group and two nitrogen atoms or

an oxygen or sulphur atom and two nitrogen atoms,

or a 6-membered heteroarylene group, which contains one or two nitrogen atoms,

while a phenyl ring may be fused to the abovementioned 5-membered heteroarylene groups containing one or two heteroatoms as well as to the abovementioned 6-membered heteroarylene groups via two adjacent carbon atoms and the bicyclic heteroarylene groups thus formed may be bound via the heteroaromatic and/or carbocyclic moiety,

and while the abovementioned mono- and bicyclic heteroarylene groups in the carbon skeleton may be substituted by a fluorine, chlorine or bromine atom, by a C₁₋₄-alkyl group, by a C₃₋₇-cycloalkyl, trifluoromethyl, hydroxy, C₁₋₃-alkoxy, trifluoromethoxy, amino, C₁₋₃-alkylamino, acetylamino, propionylamino, acetyl, propionyl, C₁₋₃-alkoxy-carbonyl, aminocarbonyl, C₁₋₃-alkylamino-carbonyl or cyano group,

or R⁶ and R⁷ together denote an n-alkylene bridge with 3 to 6 carbon atoms, wherein

one or two hydrogen atoms in each case may be replaced by a C₁₋₃-alkyl group and/or

a —CH₂—CH₂— group may be replaced by a 1,2-linked phenylene group which may be mono- or disubstituted by fluorine, chlorine or bromine atoms, by C₁₋₃-alkyl, trifluoromethyl, hydroxy, C₁₋₃-alkoxy, trifluoromethoxy, amino, C₁₋₃-alkylamino, di-(C₁₋₃-alkyl)amino, acetylamino, propionylamino, acetyl, propionyl, C₁₋₃-alkoxycarbonyl, aminocarbonyl, C₁₋₃-alkylamino-carbonyl, cyano, phenyloxy or phenyl-C₁₋₃-alkyl groups, while disubstitution with the last-named group is excluded,

while the abovementioned phenyloxy- and phenyl-C₁₋₃-alkyl group in the phenyl moiety may in turn be substituted by a fluorine, chlorine or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino or cyano group,

or in each case the carbon atom in the 3 position of a n-pentylene or n-hexylene group may be monosubstituted by a C₁₋₃-alkyl group terminally substituted by a phenyl, cyano, hydroxy, C₁₋₃-alkoxy, amino, C₁₋₃-alkylamino, di-(C₁₋₃-alkyl)-amino or a 5- to 7-membered cycloalkyleneimino group, by a carboxy, C₁₋₃-alkoxycarbonyl, amino-C₁₋₃-alkyl, C₁₋₃-alkylamino-C₁₋₃-alkyl, N—C₁₋₃-alkyl-N—(C₁₋₃-alkyl-carbonyl)-amino-C₁₋₃-alkyl, di-(C₁₋₃-alkyl)-amino-C₁₋₃-alkyl, aminocarbonyl, C₁₋₃-alkylamino-carbonyl or di-(C₁₋₃-alkyl)-aminocarbonyl group or may be disubstituted by a phenyl group and a cyano, hydroxy or C₁₋₃-alkoxy group or

the methylene group in the 3 position of a n-pentylene or n-hexylene group may be replaced by an oxygen or sulphur atom, by a sulphinyl or sulphonyl group or by an imino group optionally substituted by a C₁₋₃-alkyl, phenyl-C₁₋₃-alkyl, C₁₋₃-alkyl-carbonyl, benzoyl, C₁₋₃-alkyl-aminocarbonyl, di-(C₁₋₃-alkyl)-aminocarbonyl, phenylaminocarbonyl or N—(C₁₋₃-alkyl)-phenylaminocarbonyl group or

a methylene group in position 1 of an n-butylene, n-pentylene or n-hexylene group may be replaced by a carbonyl group,

while the phenyl groups mentioned as being unsubstituted or monosubstituted in the definition of the abovementioned groups as well as aromatic or heteroaromatic parts of molecules may, unless otherwise stated, optionally additionally be substituted in the carbon skeleton by fluorine, chlorine or bromine atoms, by C₁₋₃-alkyl groups, by trifluoromethyl, hydroxy, C₁₋₃-alkoxy, trifluoromethoxy, amino, C₁₋₃-alkylamino, acetylamino, acetyl, C₁₋₃-alkoxycarbonyl, aminocarbonyl, C₁₋₃-alkylamino-carbonyl or cyano groups, while the substituents may be identical or different and the resulting aromatic groups and parts of molecules may be at most disubstituted,

the hydrogen atoms in the C₁₋₃-alkyl and alkoxy groups mentioned in the definition of the above groups may be wholly or partially replaced by fluorine atoms and

the alkyl and alkoxy groups mentioned in the definition of the above groups or in the alkyl moieties contained in the groups of formula I defined above with more than two carbon atoms may be straight-chain or branched, unless otherwise specified.

The carboxy groups mentioned in the definition of the abovementioned groups may be replaced by a group which can be converted into a carboxy group in vivo or by a group which is negatively charged under physiological conditions,

and furthermore the amino and imino groups mentioned in the definition of the abovementioned groups may be substituted by a group which can be cleaved in vivo. Such groups are described for example in WO 98/46576 and by N. M. Nielsen et al. in International Journal of Pharmaceutics 39, 75-85 (1987).

By a group which can be converted in vivo into a carboxy group is meant, for example, a hydroxymethyl group, a carboxy group esterified with an alcohol wherein the alcohol moiety is preferably a C₁₋₆-alkanol, a phenyl-C₁₋₃-alkanol, a C₃₋₉-cycloalkanol, while a C₅₋₈-cycloalkanol may additionally be substituted by one or two C₁₋₃-alkyl groups, a C₅₋₈-cycloalkanol wherein a methylene group in the 3 or 4 position is replaced by an oxygen atom or by an imino group optionally substituted by a C₁₋₃-alkyl, phenyl-C₁₋₃-alkyl, phenyl-C₁₋₃-alkoxycarbonyl or C₂₋₆-alkanoyl group and the cycloalkanol moiety may additionally be substituted by one or two C₁₋₃-alkyl groups, a C₄₋₇-cycloalkenol, a C₃₋₅-alkenol, a phenyl-C₃₋₅-alkenol, a C₃₋₅-alkynol or phenyl-C₃₋₅-alkynol with the proviso that no bonds to the oxygen atom start from a carbon atom which carries a double or triple bond, a C₃₋₈-cycloalkyl-C₁₋₃-alkanol, a bicycloalkanol with a total of 8 to 10 carbon atoms which may additionally be substituted in the bicycloalkyl moiety by one or two C₁₋₃-alkyl groups, a 1,3-dihydro-3-oxo-1-isobenzofuranol or an alcohol of formula

Rₚ—CO—O—(R_{q}CRᵣ)—OH,

wherein

Rₚ denotes a C₁₋₈-alkyl, C₅₋₇-cycloalkyl, C₁₋₈-alkyloxy, C₅₋₇-cycloalkyloxy, phenyl or phenyl-C₁₋₃-alkyl group,

R_{q} denotes a hydrogen atom, a C₁₋₃-alkyl, C₅₋₇-cycloalkyl or phenyl group and

Rᵣ denotes a hydrogen atom or a C₁₋₃-alkyl group,

by a group which is negatively charged under physiological conditions is meant, for example, a tetrazol-5-yl, phenylcarbonylaminocarbonyl, trifluoromethylcarbonylaminocarbonyl, C₁₋₆-alkylsulphonylamino, phenylsulphonylamino, benzylsulphonylamino, trifluoromethylsulphonylamino, C₁₋₆-alkylsulphonylaminocarbonyl, phenylsulphonylaminocarbonyl, benzylsulphonylaminocarbonyl or perfluoro-C₁₋₆-alkylsulphonylaminocarbonyl group

and by a group which can be cleaved in vivo from an imino or amino group is meant, for example, a hydroxy group, an acyl group such as a phenylcarbonyl group optionally mono- or disubstituted by fluorine, chlorine, bromine or iodine atoms, by C₁₋₃-alkyl or C₁₋₃-alkoxy groups, while the substituents may be identical or different, a pyridinoyl group or a C₁₋₁₆-alkanoyl group such as the formyl, acetyl, propionyl, butanoyl, pentanoyl or hexanoyl group, a 3,3,3-trichloropropionyl or allyloxycarbonyl group, a C₁₋₁₆-alkoxycarbonyl or C₁₋₁₆-alkylcarbonyloxy group, wherein hydrogen atoms may be wholly or partially replaced by fluorine or chlorine atoms such as the methoxycarbonyl, ethoxycarbonyl, propoxycarbonyl, isopropoxycarbonyl, butoxycarbonyl, tert.butoxycarbonyl, pentoxycarbonyl, hexoxycarbonyl, octyloxycarbonyl, nonyloxycarbonyl, decyloxycarbonyl, undecyloxycarbonyl, dodecyloxycarbonyl, hexadecyloxycarbonyl, methylcarbonyloxy, ethylcarbonyloxy, 2,2,2-trichloroethylcarbonyloxy, propylcarbonyloxy, isopropylcarbonyloxy, butylcarbonyloxy, tert.butylcarbonyloxy, pentylcarbonyloxy, hexylcarbonyloxy, octylcarbonyloxy, nonylcarbonyloxy, decylcarbonyloxy, undecylcarbonyloxy, dodecylcarbonyloxy or hexadecylcarbonyloxy group, a phenyl-C₁₋₆-alkoxycarbonyl group such as the benzyloxycarbonyl, phenylethoxycarbonyl or phenylpropoxycarbonyl group, a 3-amino-propionyl group wherein the amino group may be mono- or disubstituted by C₁₋₆-alkyl or C₃₋₇-cycloalkyl groups and the substituents may be identical or different, a C₁₋₃-alkylsulphonyl-C₂₋₄-alkoxycarbonyl, C₁₋₃-alkoxy-C₂₋₄-alkoxy-C₂₋₄-alkoxycarbonyl, Rₚ—CO—O—(R_{q}CRᵣ)—O—CO, C₁₋₆-alkyl-CO—NH—(RₛCRₜ)—O—CO— or C₁₋₆-alkyl-CO—O—(RₛCRₜ)—(RₛCRₜ)—O—CO— group, wherein Rₚ to Rᵣ are as hereinbefore defined,

Rₛ and Rₜ, which may be identical or different, denote hydrogen atoms or C₁₋₃-alkyl groups.

Preferred compounds of the above general formula I are those wherein

X₁ to X₄ are as hereinbefore defined,

A^{a} denotes a bond, an oxygen atom, a —NH, —N(C₁₋₃-alkyl), sulphonyl or carbonyl group,

one of the groups —CH₂—, —(CH₂)₂—, —NH—CH₂—, —CH₂—NH—, —NH—CO—, —CO—NH—, —NH—SO₂— or —SO₂—NH—,

wherein a hydrogen atom bound to a carbon atom and/or a hydrogen atom bound to a nitrogen atom may be replaced in each case by a C₁₋₃-alkyl group and a heteroatom of group A^{a} is not linked to a nitrogen atom of a 5-membered heteroaryl group of the group R^{a},

R¹ denotes a phenyl group,

a 5-membered heteroaryl group bound via a carbon or nitrogen atom which contains

an imino group optionally substituted by a C₁₋₄-alkyl or C₁₋₄-alkylcarbonyl group, an oxygen or sulphur atom or

an imino group optionally substituted by a C₁₋₄-alkyl group or an oxygen or sulphur atom and additionally a nitrogen atom,

a 6-membered heteroaryl group, which contains one or two nitrogen atoms,

while the abovementioned phenyl and heteroaryl groups may be substituted in the carbon skeleton by a fluorine, chlorine or bromine atom, by a C₁₋₄-alkyl group, by a C₃₋₇-cycloalkyl, trifluoromethyl, phenyl, hydroxy, C₁₋₃-alkoxy, trifluoromethoxy, amino, C₁₋₃-alkylamino, di-(C₁₋₃-alkyl)amino, acetylamino, N—(C₁₋₃-alkyl)-acetylamino, acetyl or cyano group,

a C₃₋₇-cycloalkyl group, wherein

the methylene group in the 4 position of a 6-membered cycloalkyl group may be replaced by an oxygen or sulphur atom, by a sulphonyl group or by an imino group optionally substituted by a C₁₋₃-alkyl, phenyl, C₁₋₄-alkyl-carbonyl or C₁₋₄-alkoxy-carbonyl group,

a 4- to 7-membered cycloalkyleneimino group wherein

one or two hydrogen atoms in each case may be replaced by a C₁₋₃-alkyl group and/or

in each case the methylene group in the 4 position of a 6- or 7-membered cycloalkyleneimino group may be replaced by an oxygen or sulphur atom, by a sulphonyl group or by an imino group optionally substituted by a C₁₋₅-alkyl, phenyl, C₁₋₄-alkyl-carbonyl, C₁₋₄-alkoxy-carbonyl, C₁₋₃-alkyl-aminocarbonyl or di-(C₁₋₃-alkyl)-aminocarbonyl group or

in a 5-, 6- or 7-membered cycloalkyleneimino group a —CH₂— group linked to the imino nitrogen atom may be replaced by a carbonyl group or a —(CH₂)₂— group linked to the imino nitrogen atom may be replaced by a —CO—NR⁸— group or

a —(CH₂)₃— group linked to the imino nitrogen atom may be replaced by a —CO—NR⁸—CO— group,

while R⁸ denotes a hydrogen atom or a C₁₋₃-alkyl group,

R⁵ denotes a hydrogen atom or a C₁₋₃-alkyl group,

Het denotes a 5-membered heteroarylene group bound via two carbon atoms which contains

an imino group substituted by the group R⁹, an oxygen or sulphur atom or

an imino group substituted by the group R⁹ or an oxygen or sulphur atom and additionally a nitrogen atom,

while R⁹ denotes a hydrogen atom, a C₁₋₅-alkyl group, a —C₂₋₃-alkyl group terminally substituted by an amino, C₁₋₃-alkylamino, di-(C₁₋₃-alkyl)-amino or C₁₋₅-alkoxy-carbonyl-amino group, a carboxy-C₁₋₃-alkyl, C₁₋₃-alkoxycarbonyl-C₁₋₃-alkyl, phenyl, phenyl-C₁₋₃-alkyl, C₁₋₅-alkylcarbonyl or phenylcarbonyl group or R⁹ together with R⁶ denotes a —(CH₂)ₚ— bridge wherein p denotes the number 2 or 3,

or an imino group optionally substituted by a C₁₋₃-alkyl group and two nitrogen atoms or

an oxygen or sulphur atom and two nitrogen atoms,

or a 6-membered heteroarylene group, which contains one or two nitrogen atoms,

while the abovementioned heteroarylene groups in the carbon skeleton may be substituted by a fluorine, chlorine or bromine atom, by a C₁₋₃-alkyl group, by a cyclopropyl, trifluoromethyl, C₁₋₃-alkoxy, trifluoromethoxy, C₁₋₃-alkylamino, di-(C₁₋₃-alkyl)amino, acetylamino, N—(C₁₋₃-alkyl)-acetylamino, acetyl, C₁₋₃-alkylamino-carbonyl or di-(C₁₋₃-alkyl)amino-carbonyl group,

R⁶ denotes a hydrogen atom or a C₁₋₄-alkyl group,

R⁷ denotes a C₁₋₆-alkyl group,

a straight-chain C₂₋₆-alkyl group which is terminally substituted by an amino, C₁₋₃-alkylamino or di-(C₁₋₃-alkyl)-amino group,

a C₁₋₆-alkyl group substituted by an C₃₋₇-cycloalkyl group, while

a hydrogen atom in the 3 position of the cyclopentyl group and in the 4 position of a 6- or 7-membered cycloalkyl group may be replaced in each case by a C₁₋₅-alkoxy, phenyl-C₁₋₃-alkoxy-C₁₋₃-alkyl, phenyl-C₁₋₃-alkylamino, C₁₋₅-alkylcarbonylamino, benzoylamino, phenyl-C₁₋₃-alkylamino-C₁₋₃-alkyl, benzoylamino-C₁₋₃-alkyl, phenylamino-carbonyl, phenyl-C₁₋₃-alkylamino-carbonyl, carboxy or C₁₋₃-alkoxy-carbonyl group or

in each case the methylene group in the 4 position of a 6- or 7-membered cycloalkyl group may be replaced by an imino group optionally substituted by a phenyl, C₁₋₆-alkyl-carbonyl, benzoyl, phenyl-(C₁₋₃-alkyl)-carbonyl, phenylaminocarbonyl, N—(C₁₋₃-alkyl)-phenylaminocarbonyl, phenyl-C₁₋₃-alkylamino-carbonyl or N—(C₁₋₃-alkyl)-phenyl-C₁₋₃-alkylamino-carbonyl group or

in a 5- or 6-membered cycloalkyl group one or two single bonds separated by at least one bond from each other and from position 1 may each be fused to a phenyl group, while in a bi-or tricyclic ring system thus formed the hydrogen atom bound to the saturated carbon atom in position 1 may be replaced by a C₁₋₃-alkylamino-carbonyl, di-(C₁₋₃-alkyl)amino-carbonyl or C₁₋₅-alkoxy-carbonyl group, while terminal methyl groups in each case may be wholly or partly fluorinated,

a C₁₋₆-alkyl group optionally substituted by a C₃₋₇-cycloalkyl group which is substituted

by a carboxy or C₁₋₃-alkoxycarbonyl group,

by a phenyl, 1-naphthyl or 2-naphthyl group,

by a 5-membered heteroaryl group bound via a carbon or nitrogen atom which contains

an imino group optionally substituted by a C₁₋₃-alkyl or trifluoromethyl group, an oxygen or sulphur atom or

an imino group optionally substituted by a C₁₋₃-alkyl group or an oxygen or sulphur atom and additionally a nitrogen atom,

by a 6-membered heteroaryl group, which contains one or two nitrogen atoms,

while the abovementioned phenyl groups as well as the heteroaryl groups in the carbon skeleton may be monosubstituted by a fluorine, chlorine or bromine atom, by a C₁₋₃-alkyl, trifluoromethyl, C₁₋₃-alkoxy, fluoromethoxy, difluoromethoxy, trifluoromethoxy, C₁₋₃-alkylamino, di-(C₁₋₃-alkyl)amino, amino-C₁₋₃-alkyl, acetylamino, acetyl, C₁₋₃-alkoxy-carbonyl-C₁₋₃-alkyl, C₁₋₅-alkoxy-carbonylamino-C₁₋₃-alkyl, C₁₋₃-alkylamino-carbonyl or di-(C₁₋₃-alkyl)amino-carbonyl group or may also be disubstituted by the abovementioned substituents, while the substituents may be identical or different,

a C₁₋₆-alkyl group substituted by a phenyl group and a carboxy, C₁₋₃-alkoxy-carbonyl, aminocarbonyl, C₁₋₃-alkyl-aminocarbonyl or di-(C₁₋₃-alkyl)-aminocarbonyl group,

a phenyl-C₂₋₃-alkenylene-CH₂ or phenyl-C₂₋₃-alkynylene-CH₂ group, wherein a hydrogen atom of the methylene group in the 1 position may be replaced by a methyl group and independently thereof the phenyl moiety may be substituted by a fluorine, chlorine or bromine atom, by a C₁₋₄-alkyl, C₃₋₇-cycloalkyl, trifluoromethyl, C₁₋₃-alkoxy, phenyl, pyridyl, pyrimidinyl, pyrazinyl, thienyl, pyrrolyl, pyrazolyl or thiazolyl group,

the group R^{b}-A^{b}-E^{b}-C₁₋₃-alkyl optionally substituted by a methyl group in the C₁₋₃-alkyl moiety, wherein

R^{b} denotes a phenyl group optionally mono- or disubstituted by fluorine, chlorine or bromine atoms, by C₁₋₃-alkyl, cyclopropyl, trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, acetylamino, acetyl, carboxy, C₁₋₃-alkoxy-carbonyl, aminocarbonyl, C₁₋₃-alkylamino-carbonyl, di-(C₁₋₃-alkyl)amino-carbonyl or cyano groups, while the substituents may be identical or different,

a 5-membered heteroaryl group which

may be bound via a carbon atom or, if A^{b} denotes a bond, a —CH₂, —(CH₂)₂, sulphonyl or carbonyl group, may also be bound via a nitrogen atom and

contains an imino group optionally substituted by a C₁₋₃-alkyl group, an oxygen or sulphur atom,

an imino group optionally substituted by a C₁₋₃-alkyl group or an oxygen or sulphur atom and additionally a nitrogen atom or

an imino group optionally substituted by a C₁₋₃-alkyl group and two nitrogen atoms or

an oxygen or sulphur atom and two nitrogen atoms,

a 6-membered heteroaryl group, which contains one or two nitrogen atoms,

while the abovementioned heteroaryl groups in the carbon skeleton may be monosubstituted by a fluorine, chlorine or bromine atom, by a C₁₋₄-alkyl, C₃₋₇-cycloalkyl, trifluoromethyl, phenyl, hydroxy, C₁₋₃-alkoxy, trifluoromethoxy, amino, C₁₋₃-alkylamino, di-(C₁₋₃-alkyl)-amino, acetylamino, acetyl, C₁₋₃-alkoxy-carbonyl, aminocarbonyl, C₁₋₃-alkylamino-carbonyl or di-(C₁₋₃-alkyl)amino-carbonyl group or, with the exception of 5-membered heteroaryl groups containing more than two heteroatoms, may also be disubstituted by the abovementioned substituents, while the substituents may be identical or different,

a C₃₋₇-cycloalkyl group wherein

one or two hydrogen atoms in each case may be replaced by a C₁₋₃-alkyl group and/or

the methylene group in the 4 position of a cyclohexyl group may be replaced by an oxygen atom, by a sulphonyl group or by an imino group optionally substituted by a C₁₋₃-alkyl, C₁₋₃-alkyl-carbonyl, C₁₋₃-alkoxy-carbonyl, C₁₋₃-alkyl-aminocarbonyl or di-(C₁₋₃-alkyl)-aminocarbonyl group or

the two hydrogen atoms of the methylene group in the 3 position of a cyclopentyl group or in the 3- or 4-position of a cyclohexyl or cycloheptyl group may be replaced by an n-butylene, n-pentylene, n-hexylene, 1,2-ethylenedioxy or 1,3-propylenedioxy group,

a 4- to 7-membered cycloalkyleneimino group wherein

the cycloalkylene moiety may be fused to a phenyl ring or

one or two hydrogen atoms in each case may be replaced by a C₁₋₃-alkyl group and/or

in each case the carbon atom in the 4 position of a 6- or 7-membered cycloalkyleneimino group may be substituted by a 4- to 7-membered cycloalkyleneimino, phenyl or 4-(C₁₋₃-alkyl)-1,2,4-triazol-3-yl group or

may be replaced by an oxygen atom, by a sulphonyl group or by an imino group optionally substituted by a C₁₋₃-alkyl, C₁₋₃-alkyl-carbonyl, C₁₋₃-alkyl-aminocarbonyl or di-(C₁₋₃-alkyl)-aminocarbonyl group or

the two hydrogen atoms of the methylene group in the 3 position of a 5-membered cycloalkyleneimino group or in the 3 or 4 position of a 6- or 7-membered cycloalkyleneimino group may be replaced by an n-butylene, n-pentylene, n-hexylene, 1,2-ethylenedioxy or 1,3-propylenedioxy group or

in a 5-, 6- or 7-membered cycloalkyleneimino group a —CH₂— group linked to the imino nitrogen atom may be replaced by a carbonyl group

A^{b} denotes a bond, an oxygen atom, a —NH, —N(C₁₋₃-alkyl), sulphonyl or a carbonyl group,

one of the groups —CH₂—, —(CH₂)₂—, —C≡C—, —O—CH₂—, —CH₂—O—, NH—CH₂—, —CH₂—NH—, —NH—CO—, —CO—NH—, —NH—SO₂—, —SO₂—NH—,

wherein a hydrogen atom bound to a carbon atom and/or a hydrogen atom bound to a nitrogen atom may be replaced by a C₁₋₃-alkyl group in each case and a heteroatom of group A^{b} is not linked to a nitrogen atom of a 5-membered heteroaryl group of the group R^{b}, and

E^{b} denotes a phenylene group optionally substituted by a fluorine, chlorine or bromine atom, by a C₁₋₄-alkyl group, by a trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, acetylamino, acetyl, carboxy, C₁₋₃-alkoxy-carbonyl, C₁₋₃-alkoxy-carbonyl-C₁₋₃-alkyl, aminocarbonyl, C₁₋₃-alkylamino-carbonyl, di-(C₁₋₃-alkyl)amino-carbonyl or cyano group, or

the group R^{c}-A^{c}-E^{c}-C₁₋₃-alkyl, wherein

R^{c} has the meanings given for R^{b} hereinbefore, while any reference to A^{b} must be replaced by a reference to A^{c},

A^{c} denotes a bond, an oxygen atom, a —CH₂, —NH, —N(₁₋₃-alkyl), —NH—CO, —CO—NH or carbonyl group,

while a heteroatom of the group A^{c} is not linked to a nitrogen atom of a 5-membered heteroaryl group of the group R^{c}, and

E^{c} denotes a 5-membered heteroarylene group bound via two carbon atoms or via a carbon atom and an imino-nitrogen atom, while the imino-nitrogen atom of the heteroarylene group is not linked to a heteroatom of the group A^{c} and the heteroarylene group contains

an imino group optionally substituted by a C₁₋₃-alkyl group, an oxygen or sulphur atom,

an imino group optionally substituted by a C₁₋₃-alkyl group or an oxygen or sulphur atom and additionally a nitrogen atom or

an imino group optionally substituted by a C₁₋₃-alkyl group and two nitrogen atoms or

an oxygen or sulphur atom and two nitrogen atoms,

or a 6-membered heteroarylene group, which contains one or two nitrogen atoms,

while the abovementioned 5- and 6-membered heteroarylene groups in the carbon skeleton may be substituted by a fluorine, chlorine or bromine atom, by a C₁₋₄-alkyl group, by a C₃₋₇-cycloalkyl, trifluoromethyl, hydroxy, C₁₋₃-alkoxy, trifluoromethoxy, amino, C₁₋₃-alkylamino, acetylamino, acetyl, C₁₋₃-alkoxycarbonyl, aminocarbonyl, C₁₋₃-alkylamino-carbonyl or cyano group,

or R⁶ and R⁷ together denote an n-alkylene bridge with 4 or 5 carbon atoms wherein

a hydrogen atom may be replaced by a C₁₋₃-alkyl group and/or a —CH₂—CH₂ group may be replaced by a 1,2-linked phenylene group, which may be substituted by a fluorine, chlorine or bromine atom, by a C₁₋₃-alkyl, trifluoromethyl, hydroxy, C₁₋₃-alkoxy, trifluoromethoxy, amino, C₁₋₃-alkylamino, di-(C₁₋₃-alkyl)amino, acetylamino, acetyl, C₁₋₃-alkoxy-carbonyl, aminocarbonyl, C₁₋₃-alkylamino-carbonyl or cyano group or by a phenyloxy or phenyl-C₁₋₃-alkyl group optionally substituted in the phenyl moiety by a fluorine, chlorine or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino or cyano group,

or the carbon atom in the 3 position of an n-pentylene group may be monosubstituted by a C₁₋₃-alkyl group terminally substituted by an amino, C₁₋₃-alkylamino, di-(C₁₋₃-alkyl)-amino or a 5- to 7-membered cycloalkyleneimino group, by a phenyl, C₁₋₃-alkoxycarbonyl, aminocarbonyl, C₁₋₃-alkylamino-carbonyl or di-(C₁₋₃-alkyl)-aminocarbonyl group or may be disubstituted by a phenyl group and a cyano group or

the methylene group in the 3 position of an n-pentylene group may be replaced by an oxygen atom, by a sulphonyl group or by an imino group optionally substituted by a C₁₋₃-alkyl or C₁₋₃-alkyl-carbonyl group,

while the phenyl groups mentioned as being unsubstituted or monosubstituted in the definition of the abovementioned groups as well as aromatic or heteroaromatic parts of molecules may, unless otherwise stated, optionally additionally be substituted in the carbon skeleton by a fluorine, chlorine or bromine atom, by a C₁₋₃-alkyl group, by a trifluoromethyl, hydroxy, C₁₋₃-alkoxy, trifluoromethoxy, amino, C₁₋₃-alkylamino, acetylamino, acetyl, C₁₋₃-alkoxycarbonyl, aminocarbonyl, C₁₋₃-alkylamino-carbonyl or cyano group,

the alkyl and alkoxy groups mentioned in the definition of the above groups or in the alkyl moieties contained in the groups of formula I defined above with more than two carbon atoms may be straight-chain or branched, unless otherwise specified,

the carboxy groups mentioned in the definition of the abovementioned groups may be replaced by a group which can be converted into a carboxy group in vivo or by a group which is negatively charged under physiological conditions, and/or the amino and imino groups mentioned in the definition of the abovementioned groups may be substituted by a group which can be cleaved in vivo,

their tautomers, their diastereomers, their enantiomers, the mixtures and the salts thereof.

Particularly preferred compounds of the above general formula I are those wherein

X₁ denotes the group CR¹,

X₂ denotes the group CR²,

X₃ denotes the group CR³ and

X₄ denotes the group CR⁴ or

one of the groups X₁ to X₄ denotes a nitrogen atom and the remainder of the groups X₁ to X₄ denote three of the groups CR¹ to CR⁴,

while R¹, R², R³ and R⁴ in each case denote a hydrogen atom or

one or two of the groups R¹ to R⁴ independently of one another in each case denote a fluorine, chlorine or bromine atom, a C₁₋₃-alkyl group, a trifluoromethyl, amino, C₁₋₃-alkylamino or di-(C₁₋₃-alkyl)-amino group and the remainder of the groups R¹ to R⁴ in each case represent a hydrogen atom,

while R⁴ additionally together with R⁵ may assume the meaning of a —(CH₂)ₙ— bridge wherein n denotes the number 1, 2 or 3, and

A^{a} denotes a bond, an oxygen atom, a —CH₂—, —(CH₂)₂—, —NH—, —N(C₁₋₃-alkyl), sulphonyl or carbonyl group or an —NH—CH₂—, —NH—CO—, —NH—SO₂ group linked to the group R^{a} in formula (I) via the carbon or sulphur atom,

while a heteroatom of the group A^{a} is not linked to a nitrogen atom of a 5-membered heteroaryl group of the group R^{a},

R^{a} denotes a phenyl or pyridinyl group,

a pyrrolyl, furanyl, thienyl, pyrazolyl, imidazolyl, oxazolyl, isoxazolyl or thiazolyl group bound via a carbon or nitrogen atom,

while a nitrogen atom of the pyrrolyl, pyrazolyl and imidazolyl group may be substituted by a C₁₋₃-alkyl group and the phenyl group as well as the abovementioned heteroaromatic groups in the carbon skeleton may be substituted by a fluorine, chlorine or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino or cyano group,

a 5- to 7-membered cycloalkyleneimino group wherein

the methylene group in the 4 position of a 6-membered cycloalkyleneimino group may be substituted by a methyl group or replaced by an oxygen or sulphur atom or by an imino group optionally substituted by a C₁₋₃-alkyl group or

in a piperidino group a —CH₂— group linked to the imino nitrogen atom may be replaced by a carbonyl group or a —(CH₂)₂— group linked to the imino nitrogen atom may be replaced by a —CO—NR⁸— group or a —(CH₂)₁₋₃— group linked to the imino nitrogen atom may be replaced by a —CO—NR⁸—CO— group,

while R⁸ denotes a hydrogen atom or a C₁₋₃-alkyl group,

R⁵ denotes a hydrogen atom or a C₁₋₃-alkyl group,

Het denotes a 5-membered heteroarylene group bound via two carbon atoms which contains

an imino group substituted by the group R⁹, an oxygen or sulphur atom or

an imino group substituted by the group R⁹ or an oxygen or sulphur atom and additionally contains a nitrogen atom,

while R⁹ denotes a hydrogen atom, a C₁₋₃-alkyl group, a —C₂₋₃-alkyl group terminally substituted by an amino, C₁₋₃-alkylamino, di-(C₁₋₃-alkyl)-amino or C₁₋₄-alkoxy-carbonyl-amino group, a carboxy-C₁₋₃-alkyl, C₁₋₃-alkoxy-carbonyl-C₁₋₃-alkyl or C₁₋₃-alkylcarbonyl group or R⁹ together with R⁶ denotes a —(CH₂)ₚ— bridge wherein p is the number 2 or 3,

or a pyridinylene or pyrimidinylene group,

while the abovementioned heteroarylene groups in the carbon skeleton may be substituted by a fluorine, chlorine or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino or cyano group,

R⁶ denotes a hydrogen atom or a C₁₋₃-alkyl group,

R⁷ denotes a C₁₋₆-alkyl group,

a straight-chain C₂₋₆-alkyl group which is terminally substituted by an amino, C₁₋₃-alkylamino or di-(C₁₋₃-alkyl)-amino group,

a C₁₋₄-alkyl group terminally substituted by a C₃₋₇-cycloalkyl group, while

a hydrogen atom in the 4 position of a cyclohexyl group may be replaced by a C₁₋₅-alkoxy, C₁₋₃-alkoxy-C₁₋₃-alkyl, phenyl-C₁₋₃-alkoxy-methyl, phenyl-C₁₋₃-alkylamino, phenyl-C₁₋₂-alkyl-carbonylamino, benzoylamino, phenylaminocarbonyl, phenyl-C₁₋₃-alkyl-aminocarbonyl, carboxy or C₁₋₃-alkoxy-carbonyl group or

in a cyclopentyl group one or two single bonds separated from each other and from position 1 by at least one bond may each be fused to a phenyl group, while in a bi- or tricyclic ring system thus formed the hydrogen atom bound to the saturated carbon atom in the 1 position may be replaced by a C₁₋₃-alkylaminocarbonyl or di-(C₁₋₃-alkyl)amino-carbonyl group, wherein terminal methyl groups in each case may be wholly or partly fluorinated,

a C₁₋₆-alkyl group optionally substituted by a C₃₋₅-cycloalkyl group which is substituted

by a carboxy or C₁₋₃-alkoxycarbonyl group or

by a phenyl, 1-naphthyl, 2-naphthyl, pyridinyl, pyrimidinyl, pyrrolyl, furanyl, thienyl, pyrazolyl, imidazolyl, oxazolyl, isoxazolyl, thiazolyl or isothiazolyl group,

while a nitrogen atom of the pyrrolyl, pyrazolyl and imidazolyl group may be substituted by a C₁₋₃-alkyl or trifluoromethyl group and the phenyl group as well as the abovementioned heteroaromatic groups in the carbon skeleton may be substituted by a fluorine, chlorine or bromine atom, by a C₁₋₄-alkyl, trifluoromethyl, C₁₋₃-alkoxy, fluoromethoxy, difluoromethoxy, trifluoromethoxy,

C₁₋₄-alkoxy-carbonylamino-C₁₋₃-alkyl, amino, C₁₋₃-alkylamino, di-(C₁₋₃-alkyl)amino or cyano group,

a C_{1 6}-alkyl group substituted by a phenyl group and a carboxy or C₁₋₃-alkoxy-carbonyl group,

a phenyl-C₂₋₃-alkynylene-CH₂ group wherein a hydrogen atom of the methylene group in the 1 position may be replaced by a methyl group and independently thereof the phenyl moiety may be substituted by a fluorine, chlorine or bromine atom or by a C₁₋₄-alkyl, trifluoromethyl, C₁₋₃-alkoxy, phenyl or cyano group,

the group R^{b}-A^{b}-E^{b}-C₁₋₃-alkyl optionally substituted in the C₁₋₃-alkyl moiety by a methyl group, wherein

R^{b} denotes a phenyl group optionally substituted by a fluorine, chlorine or bromine atom, by a C₁₋₃-alkyl, trifluoromethyl, hydroxy, C₁₋₃-alkoxy, fluoromethoxy, difluoromethoxy, trifluoromethoxy, carboxy or C₁₋₃-alkoxycarbonyl group,

a 5-membered heteroaryl group which

may be bound via a carbon atom or, if A^{b} denotes a bond, may also be bound via a nitrogen atom and contains

an imino group optionally substituted by a C₁₋₃-alkyl group, an oxygen or sulphur atom,

an imino group optionally substituted by a C₁₋₃-alkyl group or an oxygen or sulphur atom and additionally a nitrogen atom or

an imino group optionally substituted by a C₁₋₃-alkyl group and two nitrogen atoms or

an oxygen or sulphur atom and two nitrogen atoms,

a 6-membered heteroaryl group, which contains one or two nitrogen atoms,

while the abovementioned heteroaryl groups may be monosubstituted in the carbon skeleton by a fluorine, chlorine or bromine atom, by a C₁₋₃-alkyl, trifluoromethyl, phenyl, C₁₋₃-alkoxy, trifluoromethoxy, amino, C₁₋₃-alkylamino, di-(C₁₋₃-alkyl)-amino or acetylamino group or, with the exception of 5-membered heteroaryl groups containing more than two heteroatoms, may also be disubstituted by a C₁₋₄-alkyl group and one substituent selected from fluorine, chlorine, bromine, C₁₋₃-alkyl, trifluoromethyl, phenyl, C₁₋₃-alkoxy and trifluoromethoxy,

a C₃₋₆-cycloalkyl group, wherein

the two hydrogen atoms of the methylene group in the 3 position of a cyclopentyl group or in the 3- or 4-position of a cyclohexyl group may be replaced by an n-butylene, n-pentylene or 1,2-ethylenedioxy group,

a 5- to 7-membered cycloalkyleneimino group wherein

the cycloalkylene moiety may be fused to a phenyl ring or

a hydrogen atom may be replaced by a C₁₋₃-alkyl group and/or in each case the carbon atom in the 4 position of a 6- or 7-membered cycloalkyleneimino group may be substituted by a 4- to 7-membered cycloalkyleneimino, phenyl or 4-(C₁₋₃-alkyl)-1,2,4-triazol-3-yl group or the two hydrogen atoms of the methylene group in the 3 position of a 5-membered cycloalkyleneimino group or in the 3 or 4 position of a 6- or 7-membered cycloalkyleneimino group may be replaced by an n-butylene, n-pentylene or 1,2-ethylenedioxy group,

A^{b} denotes a bond, an oxygen atom, a —CH₂—, —NH—, —O—CH₂—, carbonyl, —NH—CO— or —CO—NH— group,

wherein a hydrogen atom bound to a nitrogen atom may be replaced in each case by a C₁₋₃-alkyl group,

E^{b} denotes a phenylene group optionally substituted by a fluorine, chlorine or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino or C₁₋₃-alkoxy-carbonyl group, or

the group R^{c}-A^{c}-E^{c}-C₁₋₃-alkyl, wherein

R^{c} denotes a phenyl group optionally substituted by a fluorine, chlorine or bromine atom, by a C₁₋₃-alkyl, trifluoromethyl, C₁₋₃-alkoxy, trifluoromethoxy, carboxy or C₁₋₃-alkoxy-carbonyl group or

a 5- to 7-membered cycloalkyleneimino group wherein

the cycloalkylene moiety may be fused to a phenyl ring or

a hydrogen atom may be replaced by a C₁₋₃-alkyl group and/or

the two hydrogen atoms of the methylene group in the 3 position of a 5-membered cycloalkyleneimino group or in the 3 or 4 position of a 6- or 7-membered cycloalkyleneimino group may be replaced by an n-butylene, n-pentylene or 1,2-ethylenedioxy group,

A^{c} denotes a bond,

E^{c} denotes a 5-membered heteroarylene group bound via two carbon atoms which contains

an imino group optionally substituted by a C₁₋₃-alkyl group, an oxygen or sulphur atom,

an imino group optionally substituted by a C₁₋₃-alkyl group or an oxygen or sulphur atom and additionally a nitrogen atom or

an imino group optionally substituted by a C₁₋₃-alkyl group and two nitrogen atoms or

an oxygen or sulphur atom and two nitrogen atoms,

or a pyridinylene, pyridazinylene or pyrimidinylene group,

while the abovementioned 5- and 6-membered heteroarylene groups in the carbon skeleton may be substituted by a fluorine, chlorine or bromine atom, by a C₁₋₃-alkyl, trifluoromethyl, C₁₋₃-alkoxy, trifluoromethoxy, amino, C₁₋₃-alkylamino, acetylamino, C₁₋₃-alkoxy-carbonyl or cyano group,

or R⁶ and R⁷ together denote an n-alkylene bridge with 4 or 5 carbon atoms, wherein

a hydrogen atom may be replaced by a C₁₋₃-alkyl group and/or a —CH₂—CH₂— group may be replaced by a 1,2-linked phenylene group optionally substituted by a phenyloxy or benzyl group, while

the phenyloxy or benzyl group in the aromatic moiety and the phenylene group may be substituted independently of one another by a fluorine, chlorine or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino, C₁₋₃-alkoxy-carbonyl or cyano group,

or the carbon atom in the 3 position of an n-pentylene group may be monosubstituted by a C₁₋₃-alkyl group terminally substituted by an amino, C₁₋₃-alkylamino, di-(C₁₋₃-alkyl)-amino, acetylamino or N-(methyl)-acetylamino group or a 5- to 7-membered cycloalkyleneimino group or may be disubstituted by a phenyl group and a cyano group,

while the phenyl groups mentioned in the definition of the abovementioned groups may, unless otherwise stated, be substituted by a fluorine, chlorine or bromine atom, by a C₁₋₃-alkyl group, by a trifluoromethyl, C₁₋₃-alkoxy, trifluoromethoxy, phenyl, amino, C₁₋₃-alkylamino, acetylamino, C₁₋₃-alkoxycarbonyl or cyano group,

the alkyl and alkoxy groups mentioned in the definition of the above groups or in the alkyl moieties contained in the groups of formula I defined above with more than two carbon atoms may be straight-chain or branched, unless otherwise specified,

the carboxy groups mentioned in the definition of the abovementioned groups may be replaced by a group which can be converted into a carboxy group in vivo or by a group which is negatively charged under physiological conditions, and/or the amino and imino groups mentioned in the definition of the abovementioned groups may be substituted by a group which can be cleaved in vivo,

their tautomers, their diastereomers, their enantiomers, the mixtures and the salts thereof.

Most particularly preferred compounds of formula I are those wherein

X₁ denotes the group CR¹,

X₂ denotes the group CR²,

X₃ denotes the group CR³ and

X₄ denotes the group CR⁴ or

one of the groups X₁ to X₄ denotes a nitrogen atom and the remainder of the groups X₁ to X₄ denote three of the groups CR¹ to CR⁴,

while R¹, R², R³ and R⁴ in each case denote a hydrogen atom or

one or two of the groups R¹ to R⁴ independently of one another each denote a fluorine, chlorine or bromine atom, a C₁₋₃-alkyl group, a trifluoromethyl, amino, C₁₋₃-alkylamino or di-(C₁₋₃-alkyl)-amino group and the remainder of the groups R¹ to R⁴ each represent a hydrogen atom,

while R⁴ additionally together with R⁵ may assume the meaning of a —(CH₂)ₙ-bridge wherein n denotes the number 1, 2 or 3, and

A^{a} denotes a bond, an oxygen atom, a —CH₂—, —(CH₂)₂—, —NH—, —N(C₁₋₃-alkyl)-, sulphonyl or carbonyl group or a —NH—CH₂—, —NH—CO—, —NH—SO₂— group linked to the group R^{a} in formula (I) via the carbon or sulphur atom,

while a heteroatom of group A^{a} is not linked to a nitrogen atom of a 5-membered heteroaryl group of the group R^{a},

R^{a} denotes a phenyl or pyridinyl group,

a pyrrolyl, furanyl, thienyl, pyrazolyl, imidazolyl, oxazolyl, isoxazolyl or thiazolyl group bound via a carbon or nitrogen atom,

while a nitrogen atom of the pyrrolyl, pyrazolyl and imidazolyl group may be substituted by a C₁₋₃-alkyl group and the phenyl group as well as the abovementioned heteroaromatic groups in the carbon skeleton may be substituted by a fluorine, chlorine or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino or cyano group,

a 5- to 7-membered cycloalkyleneimino group wherein

the methylene group in the 4 position of a 6-membered cycloalkyleneimino group may be substituted by a methyl group or may be replaced by an oxygen or sulphur atom or by an imino group optionally substituted by a C₁₋₃-alkyl group or

in a piperidino group a —CH₂— group linked to the imino nitrogen atom may be replaced by a carbonyl group or a —(CH₂)₂— group linked to the imino nitrogen atom may be replaced by a —CO—NR⁸— group or a —(CH₂)₃— group linked to the imino nitrogen atom may be replaced by a —CO—NR⁸—CO— group,

while R⁸ denotes a hydrogen atom or a C₁₋₃-alkyl group,

R⁵ denotes a hydrogen atom or a C₁₋₃-alkyl group,

Het denotes a 2,4-linked pyrrolylene or imidazolylene group which are bound in each case via the 2 position to the adjacent carbonyl group of formula I and

are substituted at a nitrogen atom by a C₁₋₃-alkyl group and in the carbon skeleton may be substituted by a C₁₋₃-alkyl group or a trifluoromethyl group,

R⁶ denotes a hydrogen atom or a C₁₋₃-alkyl group,

R⁷ denotes a C₁₋₄-alkyl group terminally substituted by a C₃₋₇-cycloalkyl group, while

a hydrogen atom in the 4 position of a cyclohexyl group may be replaced by a C₁₋₅-alkoxy, C₁₋₃-alkoxy-C₁₋₃-alkyl, phenyl-C₁₋₃-alkoxy-methyl, phenyl-C₁₋₃-alkylamino, phenyl-C₁₋₂-alkyl-carbonylamino, benzoylamino, phenylaminocarbonyl, phenyl-C₁₋₃-alkyl-aminocarbonyl, carboxy or C₁₋₃-alkoxy-carbonyl group or

in a cyclopentyl group one or two single bonds separated from each other and from position 1 by at least one bond may each be fused to a phenyl group, while in a bi- or tricyclic ring system thus formed the hydrogen atom bound to the saturated carbon atom in the 1 position may be replaced by a C₁₋₃-alkylaminocarbonyl or di-(C₁₋₃-alkyl)amino-carbonyl group, while terminal methyl groups may each be wholly or partly fluorinated,

a C₁₋₆-alkyl group optionally substituted by a C₃₋₅-cycloalkyl group which is substituted

by a phenyl, 1-naphthyl, 2-naphthyl, pyridinyl, pyrimidinyl, pyrrolyl, furanyl, thienyl, pyrazolyl, imidazolyl, oxazolyl, isoxazolyl, thiazolyl or isothiazolyl group,

while a nitrogen atom of the pyrrolyl, pyrazolyl and imidazolyl group may be substituted by a C₁₋₃-alkyl or trifluoromethyl group and the phenyl group and the abovementioned heteroaromatic groups in the carbon skeleton may be substituted by a fluorine, chlorine or bromine atom, by a C₁₋₄-alkyl, trifluoromethyl, C₁₋₃-alkoxy, fluoromethoxy, difluoromethoxy, trifluoromethoxy, C₁₋₄-alkoxy-carbonylamino-C₁₋₃-alkyl, amino, C₁₋₃-alkylamino, di-(C₁₋₃-alkyl)amino or cyano group,

a C₁₋₆-alkyl group substituted by a phenyl group and a carboxy or C₁₋₃-alkoxy-carbonyl group,

a phenyl-C₂₋₃-alkynylene-CH₂ group wherein a hydrogen atom of the methylene group may be replaced in the 1 position by a methyl group and independently thereof the phenyl moiety may be substituted by a fluorine, chlorine or bromine atom, by a C₁₋₄-alkyl, trifluoromethyl, C₁₋₃-alkoxy, phenyl or cyano group,

the group R^{b}-A^{b}-E^{b}-C₁₋₃-alkyl optionally substituted in the C₁₋₃-alkyl moiety by a methyl group, wherein

R^{b} denotes a phenyl group optionally substituted by a fluorine, chlorine or bromine atom, by a C₁₋₃-alkyl, trifluoromethyl, hydroxy, C₁₋₃-alkoxy, fluoromethoxy, difluoromethoxy, trifluoromethoxy, carboxy or C₁₋₃-alkoxycarbonyl group,

a 5-membered heteroaryl group which

may be bound via a carbon atom or, if A^{b} denotes a bond, may also be bound via a nitrogen atom and contains

an imino group optionally substituted by a C₁₋₃-alkyl group, an oxygen or sulphur atom,

an imino group optionally substituted by a C₁₋₃-alkyl group or an oxygen or sulphur atom and additionally a nitrogen atom or

an imino group optionally substituted by a C₁₋₃-alkyl group and two nitrogen atoms or

an oxygen or sulphur atom and two nitrogen atoms,

a 6-membered heteroaryl group which contains one or two nitrogen atoms,

while the abovementioned heteroaryl groups in the carbon skeleton may be monosubstituted by a fluorine, chlorine or bromine atom, by a C₁₋₃-alkyl, trifluoromethyl, phenyl, C₁₋₃-alkoxy, trifluoromethoxy, amino, C₁₋₃-alkylamino, di-(C₁₋₃-alkyl)-amino or acetylamino group or, with the exception of 5-membered heteroaryl groups containing more than two heteroatoms, may also be disubstituted by a C₁₋₄-alkyl group and a substituent selected from fluorine, chlorine, bromine, C₁₋₃-alkyl, trifluoromethyl, phenyl, C₁₋₃-alkoxy and trifluoromethoxy,

a C₃₋₆-cycloalkyl group, while

the two hydrogen atoms of the methylene group in the 3 position of a cyclopentyl group or in the 3- or 4-position of a cyclohexyl group may be replaced by an n-butylene, n-pentylene or 1,2-ethylenedioxy group,

a 5- to 7-membered cycloalkyleneimino group wherein

the cycloalkylene moiety may be fused to a phenyl ring or

a hydrogen atom may be replaced by a C₁₋₃-alkyl group and/or

in each case the carbon atom in the 4 position of a 6- or 7-membered cycloalkyleneimino group may be substituted by a 4- to 7-membered cycloalkyleneimino, phenyl or 4-(C₁₋₃-alkyl)-1,2,4-triazol-3-yl group or

the two hydrogen atoms of the methylene group in the 3 position of a 5-membered cycloalkyleneimino group or in the 3 or 4 position of a 6- or 7-membered cycloalkyleneimino group may be replaced by an n-butylene, n-pentylene or 1,2-ethylenedioxy group,

A^{b} denotes a bond, an oxygen atom, a —CH₂—, —NH—, —O—CH₂—, carbonyl, —NH—CO— or —CO—NH— group,

wherein a hydrogen atom bound to a nitrogen atom may be replaced in each case by a C₁₋₃-alkyl group,

E^{b} denotes a phenylene group optionally substituted by a fluorine, chlorine or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino or C₁₋₃-alkoxy-carbonyl group, or

the group R^{c}-A^{c}-E^{c}-C₁₋₃-alkyl, wherein

R^{c} denotes a phenyl group optionally substituted by a fluorine, chlorine or bromine atom, by a C₁₋₃-alkyl, trifluoromethyl, C₁₋₃-alkoxy, trifluoromethoxy, carboxy or C₁₋₃-alkoxy-carbonyl group or

a 5- to 7-membered cycloalkyleneimino group wherein

the cycloalkylene moiety may be fused to a phenyl ring or

a hydrogen atom may be replaced by a C₁₋₃-alkyl group and/or

the two hydrogen atoms of the methylene group in the 3 position of a 5-membered cycloalkyleneimino group or in the 3 or 4 position of a 6- or 7-membered cycloalkyleneimino group may be replaced by an n-butylene, n-pentylene or 1,2-ethylenedioxy group,

A^{c} denotes a bond,

E^{c} denotes a 5-membered heteroarylene group bound via two carbon atoms which contains

an imino group optionally substituted by a C₁₋₃-alkyl group, an oxygen or sulphur atom,

an imino group optionally substituted by a C₁₋₃-alkyl group or an oxygen or sulphur atom and additionally a nitrogen atom or

an imino group optionally substituted by a C₁₋₃-alkyl group and two nitrogen atoms or

an oxygen or sulphur atom and two nitrogen atoms,

or a pyridinylene, pyridazinylene or pyrimidinylene group,

while the abovementioned 5- and 6-membered heteroarylene groups in the carbon skeleton may be substituted by a fluorine, chlorine or bromine atom, by a C₁₋₃-alkyl, trifluoromethyl, C₁₋₃-alkoxy, trifluoromethoxy, amino, C₁₋₃-alkylamino, acetylamino, C₁₋₃-alkoxy-carbonyl or cyano group,

or R⁶ and R⁷ together denote an n-alkylene bridge with 4 or 5 carbon atoms wherein

a hydrogen atom may be replaced by a C₁₋₃-alkyl group and/or

a —CH₂—CH₂— group may be replaced by a 1,2-linked phenylene group optionally substituted by a phenyloxy or benzyl group, while

the phenyloxy or benzyl group in the aromatic moiety and the phenylene group may be substituted independently of one another by a fluorine, chlorine or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino, C₁₋₃-alkoxy-carbonyl or cyano group,

or the carbon atom in the 3 position of an n-pentylene group may be monosubstituted by a C₁₋₃-alkyl group terminally substituted by an amino, C₁₋₃-alkylamino, di-(C₁₋₃-alkyl)-amino, acetylamino or N-(methyl)-acetylamino group or a 5- to 7-membered cycloalkyleneimino group or may be disubstituted by a phenyl group and a cyano group,

while the phenyl groups mentioned in the definition of the abovementioned groups may, unless otherwise stated, be substituted by a fluorine, chlorine or bromine atom, by a C₁₋₃-alkyl group, by a trifluoromethyl, C₁₋₃-alkoxy, trifluoromethoxy, phenyl, amino, C₁₋₃-alkylamino, acetylamino, C₁₋₃-alkoxy-carbonyl or cyano group,

the alkyl and alkoxy groups mentioned in the definition of the above groups or in the alkyl moieties contained in the groups of formula I defined above with more than two carbon atoms may be straight-chain or branched, unless otherwise specified,

the carboxy groups mentioned in the definition of the abovementioned groups may be replaced by a group which can be converted into a carboxy group in vivo or by a group which is negatively charged under physiological conditions, and/or

the amino and imino groups mentioned in the definition of the abovementioned groups may be substituted by a group which can be cleaved in vivo,

their tautomers, their diastereomers, their enantiomers, the mixtures and the salts thereof,

but particularly those compounds of formula I wherein

X₁ denotes the group CR¹,

X₂ denotes the group CR²,

X₃ denotes the group CR³ and

X₄ denotes the group CR⁴,

while R¹, R², R³ and R⁴ in each case denote a hydrogen atom or

one of the groups R¹ to R⁴ denotes a fluorine, chlorine or bromine atom, a C₁₋₃-alkyl group or a trifluoromethyl group and the remainder of the groups R¹ to R⁴ in each case denote a hydrogen atom,

A^{a} denotes a bond, an oxygen atom, a —CH₂—, —(CH₂)₂—, —NH—, or —N(C₁₋₃-alkyl)-group,

while a nitrogen atom of the group A^{a} is not linked to a nitrogen atom of a 5-membered heteroaryl group of the group R^{a},

R^{a} denotes a phenyl, 2-pyridinyl, 3-pyridinyl or 4-pyridinyl group,

a 1-pyrrolyl, 2-pyrrolyl, 3-pyrrolyl, 2-thienyl or 3-thienyl group,

while the nitrogen atom of the pyrrolyl group may be substituted by a C₁₋₃-alkyl group and the phenyl group and the abovementioned heteroaromatic groups in the carbon skeleton may be substituted by a fluorine, chlorine or bromine atom, by a C₁₋₃-alkyl or trifluoromethyl group,

a pyrrolidino, piperidino or morpholino group

R⁵ denotes a hydrogen atom,

Het denotes a 2,4-linked pyrrolylene or imidazolylene group which are bound in each case via the 2 position to the adjacent carbonyl group of formula I and

are substituted by a C₁₋₃-alkyl group at a nitrogen atom and may be substituted in the carbon skeleton by a C₁₋₃-alkyl group or a trifluoromethyl group,

R⁶ denotes a hydrogen atom or a C₁₋₃-alkyl group,

R⁷ denotes the group R^{d}—CH₂— or R^{d}—CH₂—CH₂—, wherein a hydrogen atom of the methylene group may be replaced in the 1 position by a C₁₋₃-alkyl group or a cyclopropyl group and wherein

R^{d} denotes a phenyl, 1-naphthyl, 2-naphthyl, 2-pyridinyl, 3-pyridinyl, 4-pyridinyl, 2-pyrimidinyl or 5-pyrimidinyl group,

while the phenyl group and the abovementioned heteroaromatic groups in the carbon skeleton may be substituted by a fluorine, chlorine or bromine atom, by a C₁₋₄-alkyl, trifluoromethyl, C₁₋₃-alkoxy or fluoromethoxy group,

a phenyl-C≡C—CH₂— group wherein a hydrogen atom of the methylene group in the 1 position may be replaced by a methyl group and independently thereof the phenyl moiety may be substituted by a fluorine, chlorine or bromine atom, by a C₁₋₄-alkyl, trifluoromethyl or phenyl group,

the group R^{b}-A^{b}-E^{b}-CH₂, wherein a hydrogen atom of the methylene group may be replaced in the 1 position by a methyl group and wherein

R^{b} denotes a phenyl group optionally substituted by a fluorine, chlorine or bromine atom, by a C₁₋₃-alkyl, trifluoromethyl, hydroxy, methoxy, carboxy or methoxycarbonyl group,

a pyrrolyl, pyrazolyl, imidazolyl, oxazolyl, isoxazolyl, thiazolyl, isothiazolyl, oxadiazole or thiadiazolyl group bound via a carbon atom or, if A^{b} denotes a bond, also bound via a nitrogen atom, wherein a hydrogen atom bound to a nitrogen atom may be replaced by a C₁₋₃-alkyl group,

a 2-pyridyl, 3-pyridyl, 4-pyridyl, pyrazinyl, 2-pyrimidinyl, 4-pyrimidinyl, 5-pyrimidinyl, 3-pyridazinyl or 4-pyridazinyl group,

while the abovementioned 5- and 6-membered heteroaryl groups in the carbon skeleton may be monosubstituted by a fluorine, chlorine or bromine atom, by a C₁₋₃-alkyl, trifluoromethyl, phenyl, amino, C₁₋₃-alkylamino, di-(C₁₋₃-alkyl)-amino or acetylamino group or, with the exception of 5-membered heteroaryl groups containing more than two heteroatoms, may also be disubstituted by a C₁₋₃-alkyl group and a substituent selected from fluorine, chlorine, bromine, C₁₋₃-alkyl, trifluoromethyl, phenyl,

a C₅₋₆-cycloalkyl group, while

the two hydrogen atoms of the methylene group in the 3-position of the cyclopentyl group or in the 4-position of the cyclohexyl group may be replaced by an n-butylene, n-pentylene or 1,2-ethylenedioxy group,

or a 5- to 6-membered cycloalkyleneimino group wherein

the cycloalkylene moiety may be fused to a phenyl ring optionally substituted by a fluorine, chlorine or bromine atom, by a C₁₋₃-alkyl, trifluoromethyl or C₁₋₃-alkoxy group or

a hydrogen atom may be replaced by a C₁₋₃-alkyl group and/or

the two hydrogen atoms of the methylene group in the 3 position of the 5-membered cycloalkyleneimino group or in the 4 position of the 6-membered cycloalkyleneimino group may be replaced by an n-butylene, n-pentylene or 1,2-ethylenedioxy group,

A^{b} denotes a bond, a —CH₂—, —NH—, —O—CH₂—, —NH—CO— or —CO—NH— group,

wherein a hydrogen atom bound to a nitrogen atom may be replaced in each case by a methyl group,

E^{b} denotes a 1,4-linked phenylene group, optionally substituted by a fluorine, chlorine or bromine atom, by a C₁₋₃-alkyl, trifluoromethyl, C₁₋₃-alkoxy or trifluoromethoxy group, or

the group R^{c}-A^{c}-E^{c}-C₁₋₃-alkyl-, wherein

R^{c} denotes a phenyl group optionally substituted by a fluorine, chlorine or bromine atom, by a C₁₋₃-alkyl, trifluoromethyl, methoxy, carboxy or methoxycarbonyl group,

A^{c} denotes a bond,

E^{c} denotes a pyrrolylene, pyrazolylene, imidazolylene, oxazolylene, isoxazolylene, thiazolylene, isothiazolylene, [1,3,4]-oxadiazolene or [1,3,4]-thiadiazolene group bound via two carbon atoms in the relative positions 1,3, wherein a hydrogen atom bound to a nitrogen atom may be replaced by a C₁₋₃-alkyl group,

or a 1,4-linked pyridinylene, pyridazinylene or pyrimidinylene group,

while the abovementioned 5- and 6-membered heteroarylene groups may be substituted in the carbon skeleton by a fluorine, chlorine or bromine atom, by a C₁₋₃-alkyl, trifluoromethyl or methoxy group,

while the alkyl and alkoxy groups mentioned in the definition of the above groups or in the alkyl moieties contained in the groups of formula I defined above with more than two carbon atoms may be straight-chain or branched, unless otherwise specified,

the carboxy groups mentioned in the definition of the abovementioned groups may be replaced by a group which can be converted into a carboxy group in vivo or by a group which is negatively charged under physiological conditions, and/or

the amino and imino groups mentioned in the definition of the abovementioned groups may be substituted by a group which can be cleaved in vivo,

their tautomers, their diastereomers, their enantiomers, the mixtures and the salts thereof.

The following compounds of general formula I are particularly suitable for the combination according to the invention:

N-(4′-Methylbiphenyl-4-yl)methyl-4-(4′-trifluormethylbiphenyl-2-carbonylamino)-1-methyl-pyrrole-2-carboxylic acid amide,

N-[4-(Pyridin-4-yl)-phenylmethyl]-4-(4′-trifluoromethylbiphenyl-2-carbonylamino)-1-methyl-pyrrole-2-carboxylic acid amide,

N-(4′-Chlorobiphenyl-4-yl)methyl-4-(4′-trifluoromethylbiphenyl-2-carbonylamino)-1-methyl-pyrrole-2-carboxylic acid amide,

N-[3-(4-Methylphenyl )-prop-2-ynyl]-4-(4′-trifluoromethyl-biphenyl-2-carbonylamino)-1-methyl-pyrrole-2-carboxylic acid amide,

N-[3-(4-Isopropylphenyl)-prop-2-ynyl]-4-(4′-trifluoromethyl-biphenyl-2-carbonylamino)-1-methyl-pyrrole-2-carboxylic acid amide,

N-[4-(6-Methylpyridazin-3-yl)-phenylmethyl]-4-(4′-trifluoromethylbiphenyl-2-carbonylamino)-1-methyl-pyrrole-2-carboxylic acid amide,

N-(4′-Methoxycarbonylbiphenyl-4-yl)methyl-4-(4′-trifluoromethyl-biphenyl-2-carbonyl-amino)-1-methyl-pyrrole-2-carboxylic acid amide,

N-[4-(1,4-Dioxa-spiro[4.5]dec-8-yl)-phenylmethyl]-4-(4′-trifluoromethylbiphenyl-2-carbonylamino)-1-methyl-pyrrole-2-carboxylic acid amide,

N-[4-(3,4-Dihydro-2H-quinolin-1-yl)-phenylmethyl]-4-(4′-trifluoromethylbiphenyl-2-carbonylamino)-1-methyl-pyrrole-2-carboxylic acid amide,

N-[4-(Pyridin-3-yl)-phenylmethyl]-4-(4′-trifluoromethylbiphenyl-2-carbonylamino)-1-methyl-pyrrole-2-carboxylic acid amide,

N-(4′-Methylbiphenyl-4-yl)methyl-4-(4′-fluorobiphenyl-2-carbonylamino)-1-methyl-pyrrole-2-carboxylic acid amide,

N-(4′-Methylbiphenyl-4-yl)methyl-4-(4′-methyl biphenyl-2-carbonyl-amino)-1-methyl-pyrrole-2-carboxylic acid amide,

N-(4′-Hydroxycarbonylbiphenyl-4-yl)methyl-4-(4′-trifluoromethyl-biphenyl-2-carbonyl-amino)-1-methyl-pyrrole-2-carboxylic acid amide,

N-(4′-Hydroxybiphenyl-4-yl)methyl-4-(4′-trifluoromethylbiphenyl-2-carbonylamino)-1-methyl-pyrrole-2-carboxylic acid amide,

N-[3-(4-Biphenyl)-prop-2-ynyl]-4-(4′-trifluoromethylbiphenyl-2-carbonylamino)-1-methyl-imidazole-2-carboxylic acid amide,

N-[4-(1,4-Dioxa-spiro[4.5]dec-8-yl)-phenylmethyl]-4-(4′-trifluoromethylbiphenyl-2-carbonylamino)-1-methyl-imidazole-2-carboxylic acid amide,

N-[3-(4-tert.Butylphenyl)-prop-2-ynyl]-4-(4′-trifluoromethyl-biphenyl-2-carbonylamino)-1-methyl-imidazole-2-carboxylic acid amide,

N-[4-(5-Dimethylaminopyridin-2-yl)-phenylmethyl]-4-(4′-trifluoromethylbiphenyl-2-carbonylamino)-1-methyl-pyrrole-2-carboxylic acid amide,

N-[3-(Biphenyl-4-yl)-prop-2-ynyl]-4-(4′-trifluoromethylbiphenyl-2-carbonylamino)-1-methyl-pyrrole-2-carboxylic acid amide,

N-[4-(4-Methylpiperidino)-phenylmethyl)-4-(4′-trifluor-methylbiphenyl-2-carbonyl-amino)-1-methyl-pyrrole-2-carboxylic acid amide,

N-[4-(1,4-Dioxa-8-aza-spiro[4.5]dec-8-yl)-phenylmethyl]-4-(4′-trifluoromethylbiphenyl-2-carbonylamino)-1-methyl-pyrrole-2-carboxylic acid amide,

N-[4-(3-Aza-spiro[5. 5]undec-3-yl)-phenylmethyl]-4-(4′-trifluoromethylbiphenyl-2-carbonylamino)-1-methyl-pyrrole-2-carboxylic acid amide,

N-(4-Benzyloxy-benzyl)-4-(4′-trifluoromethylbiphenyl-2-carbonylamino)-1-methyl-pyrrole-2-carboxylic acid amide and

N-[4-(3,4-Dihydro-1H-isoquinolin-2-yl)-phenylmethyl]-4-(4′-trifluoromethylbiphenyl-2-carbonylamino)-1-methyl-pyrrole-2-carboxylic acid amide

and the salts thereof.

The following compounds of general formula I are most particularly suitable for the combination according to the invention:

(a) N-[3-(Biphenyl-4-yl)-prop-2-ynyl]-4-(4′-trifluoromethylbiphenyl-2-carbonylamino)-1-methyl-pyrrole-2-carboxylic acid amide

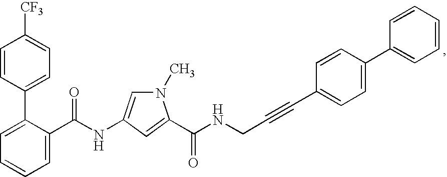

(b) N-[4-(1,4-Dioxa-8-aza-spiro[4.5]dec-8-yl)-phenylmethyl]-4-(4′-trifluoromethylbiphenyl-2-carbonylamino)-1-methyl-pyrrole-2-carboxylic acid amide

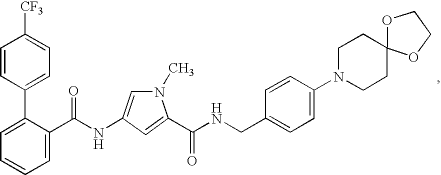

(c) N-[4-(3-Aza-spiro[5.5]undec-3-yl)-phenylmethyl]-4-(4′-trifluoromethylbiphenyl-2-carbonylamino)-1-methyl-pyrrole-2-carboxylic acid amide

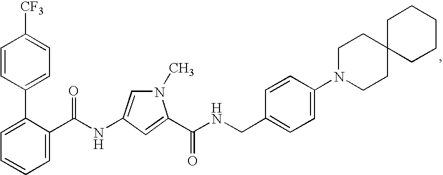

(d) N-[4-(6-Methylpyridazin-3-yl)-phenylmethyl]-4-(4′-trifluoromethylbiphenyl-2-carbonylamino)-1-methyl-pyrrole-2-carboxylic acid amide

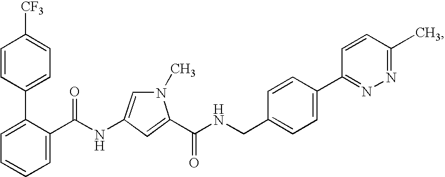

(e) N-(4′-Hydroxybiphenyl-4-yl)methyl-4-(4′-trifluoromethylbiphenyl-2-carbonylamino)-1-methyl-pyrrole-2-carboxylic acid amide

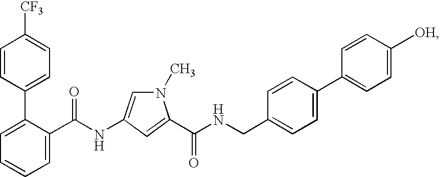

(f) N-[4-(1,4-Dioxa-spiro[4.5]dec-8-yl)-phenylmethyl]-4-(4′-trifluoromethylbiphenyl-2-carbonylamino)-1-methyl-pyrrole-2-carboxylic acid amide

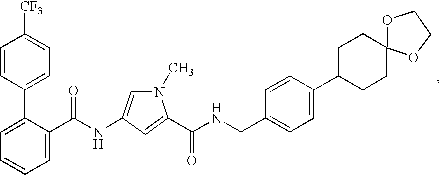

(g) N-(4′-Methylbiphenyl-4-yl)methyl-4-(4′-trifluoromethylbiphenyl-2-carbonylamino)-1-methyl-pyrrole-2-carboxylic acid amide

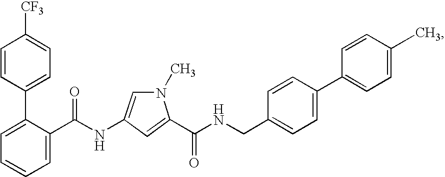

(h) N-[3-(4-Isopropylphenyl)-prop-2-ynyl]-4-(4′-trifluoromethyl-biphenyl-2-carbonylamino)-1-methyl-pyrrole-2-carboxylic acid amide

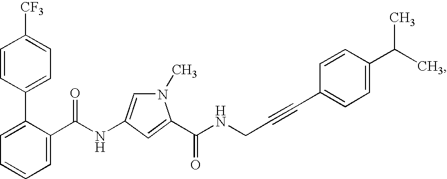

(i) N-[3-(4-Biphenyl)-prop-2-ynyl]-4-(4′-trifluoromethylbiphenyl-2-carbonylamino)-1-methyl-imidazole-2-carboxylic acid amide

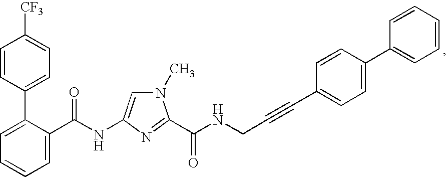

(j) N-[3-(4-Trifluoromethylphenyl)-prop-2-ynyl]-4-(4′-trifluoromethylbiphenyl-2-carbonylamino)-1-methyl-imidazole-2-carboxylic acid amide

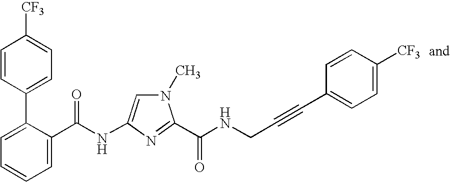

(k) N-[4-(4-Propylpiperidino)-phenylmethyl]-4-(4′-trifluoromethylbiphenyl-2-carbonyl-amino)-1-methyl-pyrrole-2-carboxylic acid amide

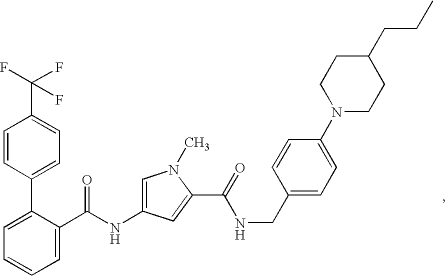

and the salts thereof, but particularly

(a) N-[3-(Biphenyl-4-yl)-prop-2-ynyl]-4-(4′-trifluoromethylbiphenyl-2-carbonylamino)-1-methyl-pyrrole-2-carboxylic acid amide,

(c) N-[4-(3-Aza-spiro[5.5]undec-3-yl)-phenylmethyl]-4-(4′-trifluoromethylbiphenyl-2-carbonylamino)-1-methyl-pyrrole-2-carboxylic acid amide,

(f) N-[4-(1,4-Dioxa-spiro[4.5]dec-8-yl)-phenylmethyl]-4-(4′-trifluoromethylbiphenyl-2-carbonylamino)-1-methyl-pyrrole-2-carboxylic acid amide,

(i) N-[3-(4-Biphenyl)-prop-2-ynyl]-4-(4′-trifluoromethylbiphenyl-2-carbonylamino)-1-methyl-imidazole-2-carboxylic acid amide and

k) N-[4-(4-Propylpiperidino)-phenylmethyl]-4-(4′-trifluoromethylbiphenyl-2-carbonyl-amino)-1-methyl-pyrrole-2-carboxylic acid amide

and the salts thereof.

According to the invention the new compounds may be obtained by methods known from the literature, for example by the following methods:

a. reacting a compound of general formula

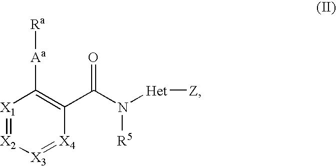

wherein

X₁ to X₄, R^{a}, A^{a}, R⁵ and Het are as hereinbefore defined and Z denotes a carboxy group or a reactive derivative of a carboxy group,

with an amine of general formula

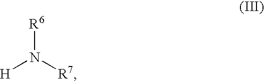

wherein

R⁶ and R⁷ are as hereinbefore defined.

The reaction is expediently carried out with a corresponding halide or anhydride of general formula II in a solvent such as methylene chloride, chloroform, carbon tetrachloride, ether, tetrahydrofuran, dioxane, benzene, toluene, acetonitrile or sulpholane optionally in the presence of an inorganic or organic base at temperatures between −20 and 200° C., but preferably at temperatures between —10 and 160° C. It may, however, also be carried out with the free acid, optionally in the presence of an acid-activating agent, e.g. propanephosphonic acid cycloanhydride or 2-(1H-benzotriazol-1-yl)-1,1,3,3-tetramethyluronium-tetrafluoroborate (TBTU), or a dehydrating agent, e.g. in the presence of isobutyl chloroformate, thionyl chloride, trimethylchlorosilane, hydrogen chloride, sulphuric acid, methanesulphonic acid, p-toluenesulphonic acid, phosphorus trichloride, phosphorus pentoxide, N,N′-dicyclohexylcarbodiimide, N,N′-dicyclohexylcarbodiimide/N-hydroxysuccinimide or 1-hydroxy-benzotriazole, N,N′-carbonyldiimidazole or N,N′-thionyldiimidazole or triphenylphosphine/carbon tetrachloride, at temperatures between −20 and 200° C., but preferably at temperatures between −10 and 160° C.

b. reacting a compound of general formula

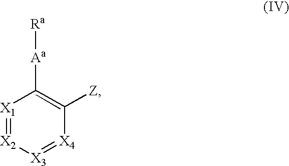

wherein

X₁ to X₄, R^{a} and A^{a} are as hereinbefore defined and Z denotes a carboxy group or a reactive derivative of a carboxy group,

with an amine of general formula

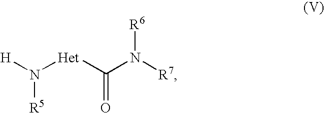

wherein

R⁵ to R⁷ and Het are as hereinbefore defined.

The reaction may be carried out under the conditions specified above for method (a).

If according to the invention a compound of general formula I is obtained which contains an amino, alkylamino or imino group, this may be converted by acylation or sulphonylation into a corresponding acyl or sulphonyl compound of general formula I or

if a compound of general formula I is obtained which contains an amino, alkylamino or imino group, this may be converted by alkylation or reductive alkylation into a corresponding alkyl compound of general formula I or

if a compound of general formula I is obtained which contains a carboxy group this may be converted by esterification into a corresponding ester of general formula I or

if a compound of general formula I is obtained which contains a carboxy or ester group, this may be converted by amidation into a corresponding amide of general formula I or

if a compound of general formula I is obtained which contains an olefinic double bond or a C—C-triple bond, this may be converted by catalytic hydrogenation into a corresponding alkyl or alkylene compound of general formula I.

The subsequent acylation or sulphonylation is optionally carried out in a solvent or mixture of solvents such as methylene chloride, dimethylformamide, benzene, toluene, chlorobenzene, tetrahydrofuran, benzene/tetrahydrofuran or dioxane with a corresponding acyl or sulphonyl derivative, optionally in the presence of a tertiary organic base or in the presence of an inorganic base or in the presence of a dehydrating agent, e.g. in the presence of isobutyl chloroformate, thionyl chloride, trimethylchlorosilane, sulphuric acid, methanesulphonic acid, p-toluenesulphonic acid, phosphorus trichloride, phosphorus pentoxide, N,N′-dicyclohexylcarbodiimide, N,N′-dicyclohexylcarbodiimide/N-hydroxysuccinimide or 1-hydroxy-benzotriazole and optionally additionally in the presence of 4-dimethylaminopyridine, N,N′-carbonyldiimidazole or triphenylphosphine/carbon tetrachloride, conveniently at temperatures between 0 and 150° C., preferably at temperatures between 0 and 80° C.

The subsequent alkylation is optionally carried out in a solvent or mixture of solvents such as methylene chloride, dimethylformamide, benzene, toluene, chlorobenzene, tetrahydrofuran, benzene/tetrahydrofuran or dioxane with an alkylating agent such as a corresponding halide or sulphonic acid ester, e.g. with methyl iodide, ethyl bromide, dimethylsulphate or benzylchloride, optionally in the presence of a tertiary organic base or in the presence of an inorganic base, conveniently at temperatures between 0 and 150° C., preferably at temperatures between 0 and 100° C.

The subsequent reductive alkylation is carried out with a corresponding carbonyl compound such as formaldehyde, acetaldehyde, propionaldehyde, acetone or butyraldehyde in the presence of a complex metal hydride such as sodium borohydride, lithium borohydride or sodium cyanoborohydride conveniently at a pH of 6-7 and at ambient temperature or in the presence of a hydrogenation catalyst, e.g. with hydrogen in the presence of palladium/charcoal, under a hydrogen pressure of 1 to 5 bar. The methylation is however preferably carried out in the presence of formic acid as reducing agent at elevated temperatures, e.g. at temperatures between 60 and 120° C.

The subsequent esterification is optionally carried out in a solvent or mixture of solvents such as methylene chloride, dimethylformamide, benzene, toluene, chlorobenzene, tetrahydrofuran, benzene/tetrahydrofuran or dioxane or particularly advantageously in a corresponding alcohol, optionally in the presence of an acid such as hydrochloric acid or in the presence of a dehydrating agent, e.g. in the presence of isobutyl chloroformate, thionyl chloride, trimethylchlorosilane, sulphuric acid, methanesulphonic acid, p-toluenesulphonic acid, phosphorus trichloride, phosphorus pentoxide, N,N′-dicyclohexylcarbodiimide, N,N′-dicyclohexylcarbodiimide/N-hydroxysuccinimide or 1-hydroxy-benzotriazole and optionally additionally in the presence of 4-dimethylaminopyridine, N,N′-carbonyldiimidazole or triphenyl-phosphine/carbon tetrachloride, conveniently at temperatures between 0 and 150° C., preferably at temperatures between 0 and 80° C.

The subsequent amidation is carried out by reacting a corresponding reactive carboxylic acid derivative with a corresponding amine optionally in a solvent or mixture of solvents such as methylene chloride, dimethylformamide, benzene, toluene, chlorobenzene, tetrahydrofuran, benzene/tetrahydrofuran or dioxane, while the amine used may simultaneously serve as solvent, optionally in the presence of a tertiary organic base or in the presence of an inorganic base or with a corresponding carboxylic acid in the presence of a dehydrating agent, e.g. in the presence of isobutyl chloroformate, thionyl chloride, trimethylchlorosilane, sulphuric acid, methanesulphonic acid, p-toluenesulphonic acid, phosphorus trichloride, phosphorus pentoxide, O-(benzotriazol-1-yl)-N,N,N′,N′-tetramethyluronium-tetrafluoroborate, N,N′-dicyclohexylcarbodiimide, N,N′-dicyclohexylcarbodiimide/N-hydroxysuccinimide or 1-hydroxy-benzotriazole and optionally additionally in the presence of 4-dimethylamino-pyridine, N,N′-carbonyldiimidazole or triphenylphosphine/carbon tetrachloride, conveniently at temperatures between 0 and 150° C., preferably at temperatures between 0 and 80° C.

The subsequent catalytic hydrogenation is carried out with hydrogen in the presence of a catalyst such as palladium/charcoal or platinum in a solvent such as methanol, ethanol, ethyl acetate, dimethylformamide, dimethylformamide/acetone or glacial acetic acid, optionally with the addition of an acid such as hydrochloric acid at temperatures between 0 and 50° C., but preferably at ambient temperature, and under a hydrogen pressure of 1 to 7 bar, but preferably from 3 to 5 bar.

In the reactions described hereinbefore, any reactive groups present such as hydroxy, carboxy, amino, alkylamino or imino groups may be protected during the reaction by conventional protecting groups which are cleaved again after the reaction.

For example, a protecting group for a hydroxy group may be a trimethylsilyl, tert.butyl-dimethylsilyl, acetyl, benzoyl, methyl, ethyl, tert.butyl, trityl, benzyl or tetrahydropyranyl group,

a protecting group for a carboxyl group may be a trimethylsilyl, methyl, ethyl, tert.butyl, benzyl or tetrahydropyranyl group and

protecting groups for an amino, alkylamino or imino group may be a formyl, acetyl, trifluoroacetyl, ethoxycarbonyl, tert.butoxycarbonyl, benzyloxycarbonyl, benzyl, methoxybenzyl or 2,4-dimethoxybenzyl group and additionally, for the amino group, a phthalyl group.

Any protecting group used is optionally subsequently cleaved for example by hydrolysis in an aqueous solvent, e.g. in water, isopropanol/water, acetic acid/water, tetrahydrofuran/water or dioxane/water, in the presence of an acid such as trifluoroacetic acid, hydrochloric acid or sulphuric acid or in the presence of an alkali metal base such as sodium hydroxide or potassium hydroxide or aprotically, e.g. in the presence of iodotrimethylsilane, at temperatures between 0 and 120° C., preferably at temperatures between 10 and 100° C. However, a silyl group may also be cleaved using tetrabutylammonium fluoride as described hereinbefore.

However, a benzyl, methoxybenzyl or benzyloxycarbonyl group is cleaved for example hydrogenolytically, e.g. with hydrogen in the presence of a catalyst such as palladium/charcoal in a suitable solvent such as methanol, ethanol, ethyl acetate or glacial acetic acid, optionally with the addition of an acid such as hydrochloric acid at temperatures between 0 and 100° C., but preferably at temperatures between 20 and 60° C., and at a hydrogen pressure of 1 to 7 bar, but preferably 3 to 5 bar. A 2,4-dimethoxybenzyl group, however, is preferably cleaved in trifluoroacetic acid in the presence of anisole.

A tert.butyl or tert.butyloxycarbonyl group is preferably cleaved by treating with an acid such as trifluoroacetic acid or hydrochloric acid or by treating with iodotrimethylsilane, optionally using a solvent such as methylene chloride, dioxane, methanol or diethyl ether.

A trifluoroacetyl group is preferably cleaved by treating with an acid such as hydrochloric acid, optionally in the presence of a solvent such as acetic acid at temperatures between 50 and 120° C. or by treating with sodium hydroxide solution, optionally in the presence of a solvent such as tetrahydrofuran at temperatures between 0 and 50° C.

A phthalyl group is preferably cleaved in the presence of hydrazine or a primary amine such as methylamine, ethylamine or n-butylamine in a solvent such as methanol, ethanol, isopropanol, toluene/water or dioxane at temperatures between 20 and 50° C.

Moreover, the compounds of general formula I obtained may be resolved into their enantiomers and/or diastereomers, as mentioned hereinbefore. Thus, for example, cis/trans mixtures may be resolved into their cis and trans isomers, and compounds with at least one optically active carbon atom may be separated into their enantiomers.

Thus, for example, the cis/trans mixtures may be resolved by chromatography into the cis and trans isomers thereof, the compounds of general formula I obtained which occur as racemates may be separated by methods known per se (cf. Allinger N. L. and Eliel E. L. in “Topics in Stereochemistry”, Vol. 6, Wiley Interscience, 1971) into their optical antipodes and compounds of general formula I with at least 2 asymmetric carbon atoms may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

The enantiomers are preferably separated by column separation on chiral phases or by recrystallisation from an optically active solvent or by reacting with an optically active substance which forms salts or derivatives such as e.g. esters or amides with the racemic compound, particularly acids and the activated derivatives or alcohols thereof, and separating the diastereomeric mixture of salts or derivatives thus obtained, e.g. on the basis of their differences in solubility, whilst the free antipodes may be released from the pure diastereomeric salts or derivatives by the action of suitable agents. Optically active acids in common use are e.g. the D- and L-forms of tartaric acid or dibenzoyltartaric acid, di-o-tolyltartaric acid, malic acid, mandelic acid, camphorsulphonic acid, glutamic acid, aspartic acid or quinic acid. An optically active alcohol may be for example (+) or (−)-menthol and an optically active acyl group in amides, for example, may be a (+)-or (−)-menthyloxycarbonyl.

Furthermore, the compounds of formula I obtained may be converted into the salts thereof, particularly for pharmaceutical use into the physiologically acceptable salts with inorganic or organic acids. Acids which may be used for this purpose include for example hydrochloric acid, hydrobromic acid, sulphuric acid, phosphoric acid, fumaric acid, succinic acid, lactic acid, citric acid, tartaric acid or maleic acid.

Moreover, if the new compounds of formula I thus obtained contain an acidic group such as a carboxy group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, arginine, cyclohexylamine, ethanolamine, diethanolamine and triethanolamine.

The compounds of general formulae II to V used as starting materials are known from the literature in some cases or may be obtained by methods known from the literature or are described in the Examples.

A compound of general formula II is obtained, for example, by reacting a compound of general formula

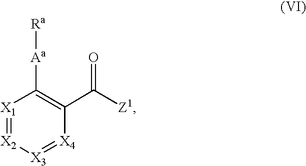

wherein X₁ to X₄, A^{a} and R^{a} are as hereinbefore defined and Z¹ denotes a carboxy group or a reactive derivative of a carboxy group, with an amine of general formula

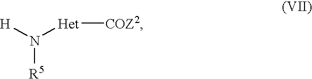

wherein R⁵ and Het are as hereinbefore defined and Z² denotes a protecting group for a carboxy group, and subsequently cleaving the protecting group.

The amines of general formula III are known from the literature or may be obtained by methods known from the literature.

The aromatic or heteroaromatic carboxylic acids according to general formula IV are known from the literature or may be obtained by methods known from the literature from corresponding aryl or heteroaryl educts.

The amino-heteroarylcarboxylic acid amides according to general formula V are also known from the literature or may easily be obtained from optionally substituted amino-heteroarylcarboxylic acids by reacting with the corresponding amines or from nitro-heteroarylcarboxylic acids by reacting with the corresponding amines and subsequently reducing the nitro group.

Starting compounds of formula V′, wherein Het denotes a 5-membered heteroarylene group which contains an imino group substituted by the group R⁹, while R⁹ together with R⁶ denotes a —(CH₂)ₚ— bridge, are obtained for example by the following synthesis plan:

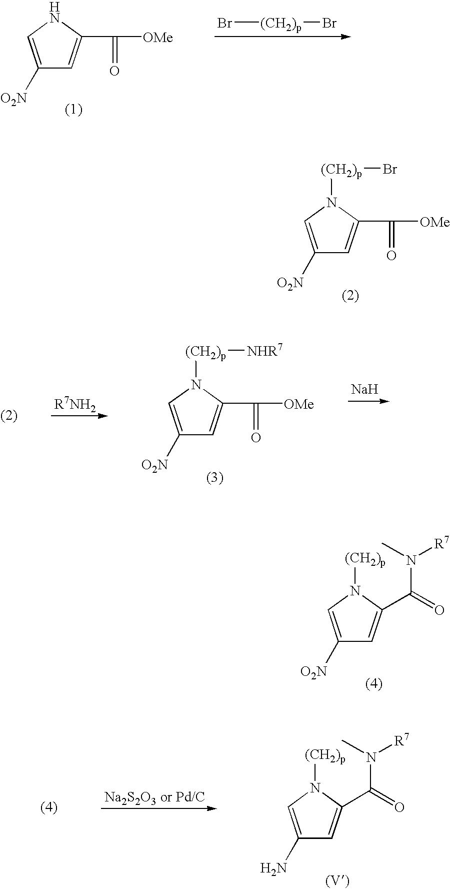

As already mentioned hereinbefore, the compounds of general formula I and the physiologically acceptable salts thereof have valuable pharmacological properties. In particular, they are valuable inhibitors of the microsomal triglyceride-transfer protein (MTP) and are therefore suitable for lowering the plasma levels of the atherogenic lipoproteins.

For example, the compounds according to the invention were investigated for their biological effects as follows:

Inhibitors of MTP were identified by a cell-free MTP activity kit. Solubilised liver microsomes from various species (e.g. rat, pig) could be used as the MTP source. To prepare donor and acceptor vesicles, lipids dissolved in organic solvents were mixed in suitable proportions and applied in a thin layer to the wall of a glass container by blowing the solvent in a nitrogen current. The solution used to prepare donor vesicles contained 400 μM phosphatidylcholine, 75 μM cardiolipin and 10 μM [¹⁴C]-triolein (68.8 μCi/mg). To prepare acceptor vesicles, a solution of 1.2 mM phosphatidylcholine, 5 μM triolein and 15 μM [³H]-dipalmitoylphosphatidylcholine (108 mCi/mg) was used. Vesicles are formed by wetting the dried lipids with test buffer and then subjecting to ultrasound. Vesicle populations of uniform size were obtained by gel filtration of the ultrasonicated lipids. The MTP activity test contains donor vesicles, acceptor vesicles and the MTP source in test buffer. Substances were added from concentrated DMSO-containing stock solutions; the final concentration of DMSO in the test was 0.1%. The reaction was started by the addition of MTP. After a suitable incubation period the transfer process was stopped by the addition of 500 μl of a SOURCE 30Q anion exchanger suspension (Pharmacia Biotech). The mixture was shaken for 5 minutes and the donor vesicles bound to the anion exchanger material were separated off by centrifuging. The radioactivity of [3H] and [14C] found in the supernatant was determined by liquid scintillation measurement and from this the recovery of the acceptor vesicles and the triglyceride transfer rate were calculated.

A second embodiment according to the invention relates to combinations containing MTP inhibitors of general formula VIII

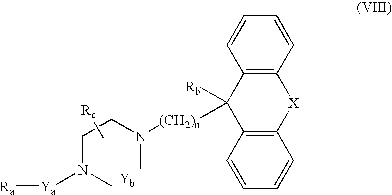

which are already known from WO 01/47899, as well as the isomers and the salts thereof. Reference is made to the entire contents of WO 01/47899 in this regard.

In general formula VIII

n denotes the number 2, 3, 4 or 5,

X denotes a carbon-carbon bond, an oxygen atom, a methylene, ethylene, imino or N—(C₁₋₃-alkyl)-imino group,

Yₐ denotes a carbonyl or sulphonyl group,

Y_{b} denotes the group —(CH₂)ₘ—, where m is the number 2 or 3 and wherein a hydrogen atom may be replaced by a C₁₋₃-alkyl group or a methylene group linked to a nitrogen atom may be replaced by a carbonyl group,

Rₐ denotes a C₁₋₆-alkoxy, phenyl-C₁₋₃-alkoxy or amino group, while the amino group may be mono- or disubstituted by C₁₋₃-alkyl, phenyl-C₁₋₄-alkyl or phenyl groups and the substituents may be identical or different,

a phenyl, naphthyl, tetrahydronaphthyl, phenoxy or heteroaryl group, a C₁₋₉-alkyl group optionally substituted by a hydroxy, C₁₋₃-alkoxy, C₁₋₄-alkoxycarbonyl or C₁₋₄-alkylcarbonyloxy group which may be substituted in the alkyl moiety by a C₁₋₃-alkyl group, by one or two phenyl groups, by a naphthyl, fluorenyl, phenoxy, heteroaryl or C₃₋₇-cycloalkyl group, or a C₃₋₇-cycloalkyl group substituted by a phenyl group,

a phenylcarbonyl, naphthylcarbonyl, tetrahydronaphthylcarbonyl, phenoxy-carbonyl or heteroarylcarbonyl group, a C₁₋₉-alkylcarbonyl group which may be substituted in the alkyl moiety by one or two phenyl groups, by a naphthyl, fluorenyl, phenoxy, heteroaryl or C₃₋₇-cycloalkyl group, or a C₃₋₇-cycloalkyl group substituted by a phenyl group,

while all the phenyl, naphthyl and heteroaryl moieties mentioned under Rₐ hereinbefore may be substituted by the groups R₁ and R₂, wherein

R₁ denotes a hydrogen, fluorine, chlorine or bromine atom, a cyano, C₁₋₃-alkyl, C₂₋₄-alkenyl, phenyl, hydroxy, C₁₋₄-alkoxy, phenyl-C₁₋₃-alkoxy, carboxy, C₁₋₃-alkoxycarbonyl, aminocarbonyl, C₁₋₃-alkylaminocarbonyl, N,N-di-(C₁₋₃-alkyl)-aminocarbonyl, nitro, amino, C₁₋₃-alkylamino, di-(C₁₋₃-alkyl)-amino, phenyl-C₁₋₃-alkylamino, N—(C₁₋₃-alkyl)-phenyl-C₁₋₃-alkylamino, C₁₋₃-alkylcarbonylamino, N—(C₁₋₃-alkyl)-C₁₋₃-alkylcarbonylamino, C₁₋₃-alkylsulphonylamino or N—(C₁₋₃-alkyl)-C₁₋₃-alkylsulphonylamino group and

R₂ denotes a hydrogen, fluorine, chlorine or bromine atom, a C₁₋₃-alkyl, hydroxy or C₁₋₄-alkoxy group, while in the abovementioned alkyl and alkoxy moieties of the groups R₁ and R₂ the hydrogen atoms may be wholly or partly replaced by fluorine atoms, or

R₁ and R₂ together denote a methylenedioxy group,

or while all the phenyl moieties mentioned under Rₐ hereinbefore may each be substituted by three chlorine or bromine atoms or by three to five fluorine atoms,

R_{b} denotes a carboxy, C₁₋₆-alkoxycarbonyl, C₁₋₆-alkoxycarbonyl-C₁₋₃-alkylcarbonyl, C₃₋₇-cycloalkoxycarbonyl or phenyl-C₁₋₃-alkoxycarbonyl group or an R₃NR₄—CO— group wherein

R₃ and R₄, which may be identical or different, denote hydrogen atoms, C₁₋₆-alkyl groups wherein the hydrogen atoms may be wholly or partly replaced by fluorine atoms and the C₁₋₃-alkyl moiety of a C₁₋₃-alkylamino group may be substituted by a carboxy or C₁₋₃-alkoxycarbonyl group or in the 2 or 3 position by an amino, C₁₋₃-alkylamino or di-(C₁₋₃-alkyl)-amino group, C₃₋₇-cycloalkyl, pyridyl, pyridinyl-C₁₋₃-alkyl, phenyl, naphthyl or phenyl-C₁₋₃-alkyl groups, while the abovementioned phenyl groups may each be substituted by a fluorine, chlorine or bromine atom, by a C₁₋₃-alkyl group wherein the hydrogen atoms may be wholly or partly replaced by fluorine atoms, by a hydroxy, C₁₋₃-alkoxy, carboxy, C₁₋₃-alkoxycarbonyl, aminocarbonyl, C₁₋₃-alkylaminocarbonyl, N,N-di-(C₁₋₃-alkyl)-aminocarbonyl or N,N-di-(C₁₋₃-alkyl)-amino group, or

R₃ and R₄ together with the nitrogen atom between them denote a 3- to 5-membered cycloalkyleneimino group, while the methylene group in the 4 position in a 6- or 7-membered cycloalkyleneimino group may additionally be replaced by an oxygen or sulphur atom, by a sulphinyl, sulphonyl, imino or N—(C₁₋₃-alkyl)-imino group,

and R_{c} denotes a hydrogen atom or a C₁₋₃-alkyl group,

while the tricyclic group in the abovementioned general formula I may additionally be mono- or disubstituted by fluorine or chlorine atoms or by methyl or methoxy groups and the substituents may be identical or different,

by the abovementioned heteroaryl groups are meant a 6-membered heteroaryl group containing one, two or three nitrogen atoms, or

a 5-membered heteroaryl group containing an imino group optionally substituted by a C₁₋₃-alkyl group, an oxygen or sulphur atom or

an imino group optionally substituted by a C₁₋₃-alkyl group and one or two nitrogen atoms or an oxygen or sulphur atom and a nitrogen atom,

while in each case a phenyl ring may be fused to the abovementioned heteroaryl groups via a vinylene group,

and the carboxy group mentioned in the definition of the abovementioned groups may also be replaced by a group which can be converted into a carboxy group in vivo or by a group which is negatively charged under physiological conditions,

and all the abovementioned saturated alkyl and alkoxy moieties that contain more than 2 carbon atoms may be straight-chained or branched, unless otherwise stated.

The following compounds of general formula VIII are particularly valuable when combined with fibrates, particularly fenofibrate, and are therefore preferred according to the invention:

9-{4-[4-(4-trifluoromethyl-phenylacetyl)-piperazino]-butyl}-9H-fluorene-9-carboxylic acid-(2,2,2-trifluoro-ethyl)-amide

9-[4-(4-phenylacetyl-piperazino)-butyl]-9H-fluorene-9-carboxylic acid-(2,2,2-trifluoro-ethyl)-amide

9-(4-{4-[2-phenyl-butyryl]-piperazino}-butyl)-9H-fluorene-9-carboxylic acid-(2,2,2-trifluoro-ethyl)-amide

9-(4-{4-(3-phenylpropionyl)-piperazino}-butyl)-9H-fluorene-9-carboxylic acid-(2,2,2-trifluoro-ethyl)-amide

9-{4-[4-(4-phenyl-butyryl)-piperazino]-butyl}-9H-fluorene-9-carboxylic acid-(2,2,2-trifluoro-ethyl)-amide

9-(4-{4-(4-(pyridin-2-yl-acetyl)-piperazino}-butyl)-9H-fluorene-9-carboxylic acid-(2,2,2-trifluoro-ethyl)-amide

9-(4-{4-[2-oxo-2-phenyl-acetyl]-piperazino}-butyl)-9H-fluorene-9-carboxylic acid-(2,2,2-trifluoro-ethyl)-amide

9-(4-{4-[(2,4-dichlorophenyl)-acetyl]-piperazino}-butyl)-9H-fluorene-9-carboxylic acid-(2,2,2-trifluoro-ethyl)-amide.

A particularly preferred embodiment relates to combinations of one of the following MTP inhibitors with fibrates, particularly fenofibrate:

(a) N-[3-(Biphenyl-4-yl)-prop-2-ynyl]-4-(4′-trifluoromethylbiphenyl-2-carbonylamino)-1-methyl-pyrrole-2-carboxylic acid amide,

(c) N-[4-(3-Aza-spiro[5.5]undec-3-yl)-phenylmethyl]-4-(4′-trifluoromethylbiphenyl-2-carbonylamino)-1-methyl-pyrrole-2-carboxylic acid amide,

(f) N-[4-(1,4-Dioxa-spiro[4.5]dec-8-yl)-phenylmethyl]-4-(4′-trifluoromethylbiphenyl-2-carbonylamino)-1-methyl-pyrrole-2-carboxylic acid amide,

(i) N-[3-(4-Biphenyl)-prop-2-ynyl]-4-(4′-trifluoromethylbiphenyl-2-carbonylamino)-1-methyl-imidazole-2-carboxylic acid amide or

k) N-[4-(4-Propylpiperidino)-phenylmethyl]-4-(4′-trifluoromethylbiphenyl-2-carbonyl-amino)-1-methyl-pyrrole-2-carboxylic acid amide and the salts thereof.

In addition, the following MTP inhibitors, for example, may be used according to the invention:

9-[4-[4-[2-(4-trifluoromethylphenyl)benzoylamino]piperidin-1-yl]butyl]-N-(2,2,2-trifluoro-ethyl)-9H-fluorene-9-carboxamide (BMS-201038; compound 9 from Wetterau J R et al., Science 282, 751-754 (1998); compound 1 from Robl J A et al., J Med Chem 44, 851-856 (2001))

9-[4-[2,5-dimethyl-4-[[[4′-(trifluoromethyl)[1,1′-biphenyl]-2-yl]carbonyl]amino]-1H-benzimidazol-1-yl]butyl]-N-(2,2,2-trifluoro-ethyl)-9H-fluorene-9-carboxamide (BMS-212122; compound 3g from Robl J A et al., J Med Chem 44, 851-856 (2001))

2(S)-cyclopentyl-2-[4-(2,4-dimethyl-9H-pyrido[2,3-b]indol-9-ylmethyl)phenyl]-N-(2-hydroxy-1(R)-phenylethyl)acetamide (Implitapide, BAY-13-9952; Sorbera L A et al., Drugs of the Future 25 (11): 1138-1144 (2000))

2-cyclopentyl-2-{4-[(2,4-dimethyl-9H-pyrido[2,3-b]indol-9-yl)methyl]phenyl}-2′-phenylacetohydrazide (WO 00/71502)

2-{4-[(2,4-dimethylpyrimido[1,2-a]indol-10-yl)methyl]phenyl}-3-methyl-2′-phenyl-butane hydrazide (WO 01/74817)

(−)-[2S-[2α,4α(S*)]]-4-[4-[4-[4-[[2-(4-chlorophenyl)-2-[[(4-methyl-4H-1,2,4-triazol-3-yl)thio]methyl]-1,3-dioxolan-4-yl]methoxy]phenyl]-1-piperazinyl]phenyl]-2,4-dihydro-2-(1-methylpropyl)-3H-1,2,4-triazol-3-one (R-103757; compound 40 from WO 96/13499) and the sulphoxides thereof such as e.g. (−)-[2S-[2α,4α(S*)]]-4-[4-[4-[4-[[2-(4-chlorophenyl)-2-[[(4-methyl-4H-1,2,4-triazol-3-yl)sulphonyl]methyl]-1,3-dioxolan-4-yl]methoxy]phenyl]-1-piperazinyl]phenyl]-2,4-dihydro-2-(1-methylpropyl)-3H-1,2,4-triazol-3-one etc. (WO 00/37463)

Compounds from WO 00/05201:

(S)-6-methyl-4′-trifluoromethylbiphenyl-2-carboxylic acid-(2-methylsulphonylamino-indan-5-yl)-amide (Example 13b) (R)-6-methyl-4′-trifluoromethylbiphenyl-2-carboxylic acid-(2-methoxycarbonylamino-indan-5-yl)-amide (Example 13i) and (S)-6-methyl-4′-trifluoromethylbiphenyl-2-carboxylic acid-(2-methoxycarbonylamino-indan-5-yl)-amide

(R)-4-fluoro-4′-trifluoromethylbiphenyl-2-carboxylic acid-(2-methylsulphonylamino-indan-5-yl)-amide (Example 13al) and (S)-4-fluoro-4′-trifluoromethylbiphenyl-2-carboxylic acid-(2-methylsulphonylamino-indan-5-yl)-amide 6-methyl-4′-trifluoromethylbiphenyl-2-carboxylic acid-(2-dimethylaminocarbonylamino-indan-5-yl)-amide (Example 2ey)

4′-trifluoromethyl-biphenyl-2-carboxylic acid-[2-(2H-[1,2,4]triazol-3-ylmethyl)-1,2,3,4-tetrahydro-isoquinolin-6-yl]-amide (CP-346086; WO 97/41111 and WO 96/40640)

4′-trifluoromethyl-biphenyl-2-carboxylic acid-[2-(2-acetylamino-ethyl)-1,2,3,4-tetrahydro-isoquinolin-6-yl]-amide (CP-395919; WO 98/23593 and EP 0 887 345)

The following compounds, for example, may be used as fibrates according to the invention (international generic names):

bezafibrate

ciprofibrate

clofibrate

fenofibrate

gemfibrozil

The substances generally and specifically mentioned in the invention are administered systemically, e.g. by oral or parenteral route. They are preferably given orally. They may be incorporated in systemic formulations such as tablets. capsules, powders, solutions, suspensions, injectable formulations or the like. Suitable pharmaceutically acceptable carriers which can be used together with the substances of this invention include, for example, inert solid fillers or diluents as well as sterile aqueous or organic solutions. If necessary, other substances may be added to the pharmaceutical compositions, such as, for example, antioxidants, lubricants, buffers, scents and sweeteners.

MTP inhibitors and fibrates may be added either in separate systemic formulations or in a combined formulation.

The dosage in which a substance according to this invention is administered to warm-blooded animals, including humans, may vary depending on their physical conditions. This includes allowances for age, weight, sex, breed and general state of health. The dosage is also determined by the method of administration.

Generally, the daily dose of MTP inhibitor will be between about 0.5 mg and about 500 mg, preferably between 1 mg and 200 mg. The range between 1 mg and 50 mg is particularly preferred. This quantity may be given in a single dose or divided up into several doses.

Generally, the daily dose of fibrate is between about 50 mg and about 5000 mg, preferably between 50 mg and 1000 mg. The range from 50 to 600 mg is particularly preferred. This quantity may be given in a single dose or divided up into several doses.

### Description of Test

The effectiveness of the combination of an MTP inhibitor with a fibrate and the effect on hepatic steatosis and liver toxicity can be tested in vivo as follows. Hyperlipaemic rats (e.g. of the rat strain fa/fa) are given the active substances as a suspension in 0.45% NaCl and 5% polyethyleneglycol 400 by oral route using an oesophageal tube (5 ml/kg of body weight). The substances may be given once or several times a day for a period of 4 days, or alternatively over a longer period. The day after the last dose, blood samples are taken by puncturing the retroorbital venous plexus and plasma is prepared. The concentrations of cholesterol and triglycerides in the plasma and the activities of the liver enzymes (e.g. ALT, AST, GLDH) are determined by well-known methods of clinical chemistry. These substrates and enzymes in the plasma may be measured for example with a HITACHI 917 Automatic Analyzer using reagents supplied by Roche Diagnostics (Mannheim) in accordance with the following procedures laid down by Roche Diagnostics:

| | |
|---|---|
| ALT: | BM/HITACHI 917/Keysys No. 1876805 |
| AST: | BM/HITACHI 917/Keysys No. 1876848 |
| GLDH: | Glutamate-Dehydrogenase, No. 1929992 |

cholesterol: BM/HITACHI 917, Boehringer Mannheim System No. 1 491 458

triglycerides: BM/HITACHI 917, Boehringer Mannheim System No. 1 730 711.

In addition, the liver may be removed in order to determine the hepatic steatosis by measuring the lipid content (triglycerides, free fatty acids, cholesterol) in this organ. To do this, 200 mg of liver are homogenised after the addition of 2 ml of isopropanol and extracted for 10 min with shaking. The extract is centrifuged for 10 min at 4° C. and 4000 rpm and one aliquot of the supernatant is used to determine the lipid parameters. The lipids in the liver are measured using commercially available test kits following the manufacturer's instructions (for triglycerides: Triglycerid-Duo S made by BIOMED Labordiagnostik GmbH, Oberschleiβheim; for cholesterol: Cholesterin-Duo S made by BIOMED Labordiagnostik GmbH, Oberschleiβheim; for free fatty acids: NEFA C made by Wako Chemicals GmbH, Neuss).

### BRIEF DESCRIPTION OF THE DRAWINGS

FIGS. 1a and 1b show the findings of the first pharmacological Example (Example A) in graph form. FIG. 1a shows the cholesterol content in the plasma after the administration of an MTP inhibitor on its own (M), after the administration of a fibrate on its own (F) and after the administration of a combination of MTP inhibitor and fibrate (M+F) as well as the corresponding content in an untreated control group. FIG. 1b shows the content of triglycerides in the plasma after the administration of an MTP inhibitor on its own (M), after the administration of a fibrate on its own (F) and after the administration of a combination of MTP inhibitor and fibrate (M+F) as well as the corresponding content in an untreated control group. The numbers above the bars in the diagram indicate the percentage changes compared with the control group.

FIGS. 2a and 2b also refer to the first pharmacological Example (Example A) and show, by means of the activities of alanine-aminotransferase (ALT, FIG. 2a) and glutamate dehydrogenase (GLDH, FIG. 2b), respectively, in the blood plasma, which are characteristic indications of damage to liver cells, the side effects of the administration of an MTP inhibitor on its own (M), a fibrate on its own (F) and a combination of MTP inhibitor and fibrate (M+F) compared with a control group. The numbers above the bars in the diagram indicate the increase compared with the control group.

FIGS. 3a and 3b indicate the content of triglycerides or of free fatty acids in the liver obtained after the administration of an MTP inhibitor on its own (M), a fibrate on its own (F) or a combination of MTP inhibitor and fibrate (M+F) according to pharmacological Example B compared with a control group.

### EXAMPLE A

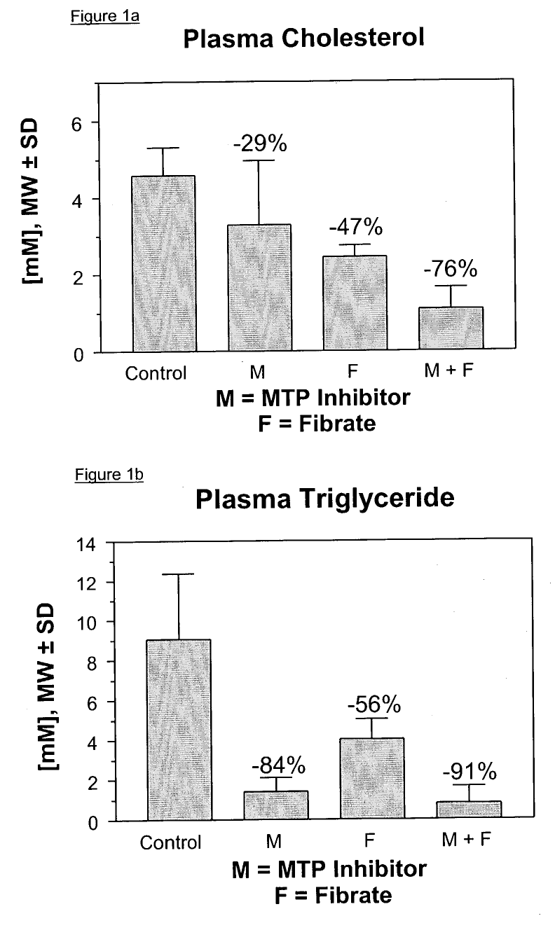

Female fa/fa rats 33 weeks old were either treated four times with an MTP inhibitor (given orally once a day at 7 a.m.) or treated eight times with a fibrate (given twice a day by oral route at 7 a.m. and 4 p.m.) A third group were given both the MTP inhibitor and the fibrate. The MTP inhibitor was 9-[4-[4-[2-(4-trifluoromethylphenyl)benzoylamino]piperidin-1-yl]butyl]-N-(2,2,2-trifluoro-ethyl)-9H-fluorene-9-carboxamide in a dosage of 1 mg/kg. The fibrate was bezafibrate in a dosage of 100 mg/kg. 24 hours after the last dose of the MTP inhibitor or 15 hours after the last dose of the fibrate blood samples were taken from the animals and the cholesterol, triglycerides and liver enzymes in the plasma were measured. Compared with the control group, which had been treated with carrier twice a day, the MTP inhibitor lowered the triglycerides in the plasma by 84%, the fibrate lowered them by 56% and the combination of the two lowered them by 91%. Cholesterol in the plasma was lowered by 29% by the MTP inhibitor, by 47% by the fibrate and by 76% by the combination. This shows that the effects of MTP inhibitor and fibrate on the lipid levels in the plasma are complementary (FIGS. 1a and 1b). The numbers above the bars in the diagram indicate the percentage change compared with the control group.

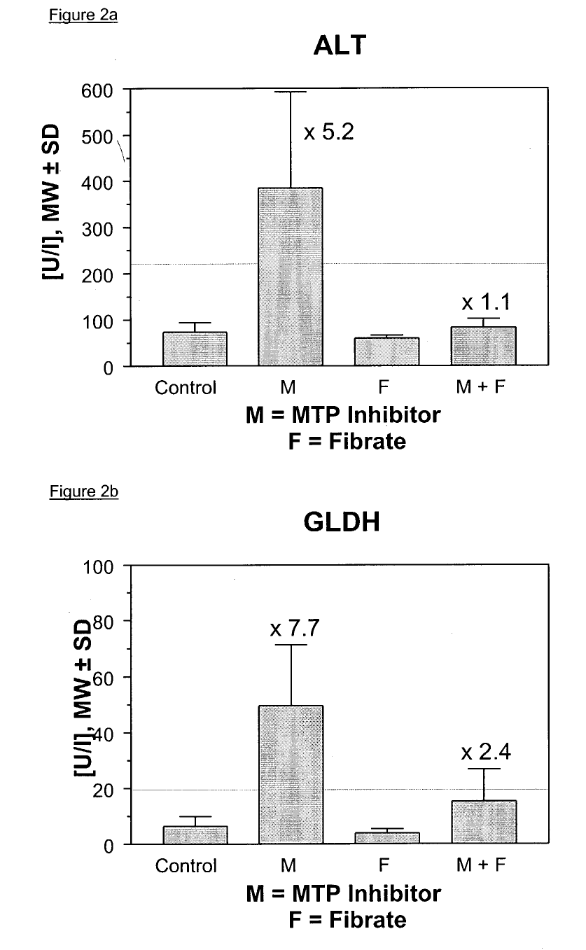

The side effects of the MTP inhibitor on the liver are clearly demonstrated by a 5.2-fold increase in the ALT activity and a 7.7-fold increase in the GLDH activity in the blood plasma compared with the control group. By the combination with the fibrate these increases are either returned to normal levels (ALT) or significantly reduced (GLDH) (FIGS. 2a and 2b). The horizontal line in the diagram indicates three times the level in the control group and is interpreted as the threshold value for a clearly toxic side effect.). The numbers above the bars in the diagram indicate the increase compared with the control group.

### Example B

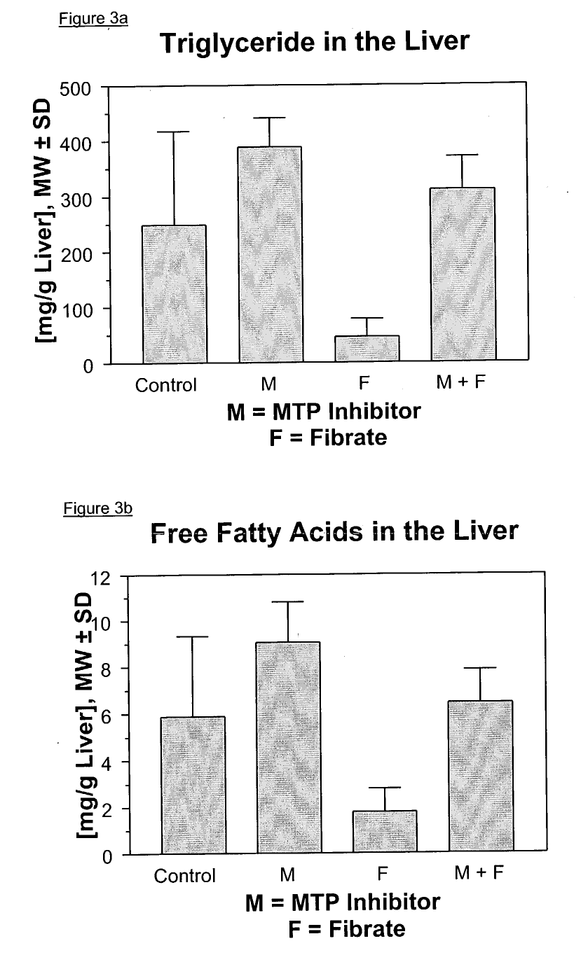

Female fa/fa rats 38 weeks old were either treated four times with an MTP inhibitor (given orally once a day at 7 a.m.) or treated eight times with a fibrate (given twice a day by oral route at 7 a.m. and 4 p.m.) A third group were given both the MTP inhibitor and the fibrate. The MTP inhibitor was 9-[4-[4-[2-(4-trifluoromethylphenyl)benzoylamino]piperidin-1-yl]butyl]-N-(2,2,2-trifluoro-ethyl)-9H-fluorene-9-carboxamide in a dosage of 0.3 mg/kg. The fibrate was bezafibrate in a dosage of 100 mg/kg. 24 hours after the last dose of the MTP inhibitor or 15 hours after the last dose of the fibrate, the animals' livers were removed and the content of triglycerides and free fatty acids in the liver was determined (FIGS. 3a and 3b). The MTP inhibitor leads to an increase in the triglycerides and the free fatty acids in the liver (FIGS. 3a and 3b). By combining it with the fibrate, the lipid accumulation caused by the MTP inhibitor is lowered by about 50% (triglycerides in the liver) or by about 80% (free fatty acids in the liver).

### Example C

Male fa/fa rats 32 weeks old were either treated four times with an MTP inhibitor (given orally once a day between about 7 and 8 a.m.) or treated eight times with a fibrate (given twice a day by oral route between about 7 and 8 a.m. and at 4 p.m.) Another group were given both the MTP inhibitor and the fibrate. The MTP inhibitor was N-[4-(3-aza-spiro[5,5]-undec-3-yl )-phenylmethyl]-4-(4′-trifluoromethylbiphenyl-2-carbonylamino)-1-methyl-oyrrole-2-carboxylic acid amide (compound (c)) in a dosage of 10 mg/kg. The fibrate was fenofibrate in a dosage of 100 mg/kg. 24 hours after the last dose of the MTP inhibitor or 15 hours after the last dose of fenofibrate, blood was taken from the animals and the levels of cholesterol, triglycerides and liver enzymes in the plasma were measured.

The effects of the treatment on the lipid levels in the plasma are shown in the following Table:

| | plasma | plasma |
|---|---|---|
| | cholesterol [mM], | triglycerides [mM], |
| Treatment | MW ± SEM | MW ± SEM |
| Control | 12.0 ± 1.9 | 15.3 ± 5.6 |
| Compound (c) 10 mg/kg | 5.0 ± 0.7 | 0.9 ± 0.1 |
| Fenofibrate 100 mg/kg | 10.2 ± 1.7 | 12.2 ± 2.7 |
| Compound (c) 10 mg/kg | 3.8 ± 0.2 | 2.0 ± 0.6 |
| plus | | |
| Fenofibrate 100 mg/kg | | |

Compared with the control group, which had been treated twice a day with carrier, the MTP inhibitor reduced cholesterol in the plasma by 58%, fenofibrate by 15% and the combination of both by 68%. Triglycerides in the plasma were reduced by 94% by the MTP inhibitor, by 20% by the fenofibrate and by 87% by the combination of both. As in Example A these data show that the effects of MTP inhibitor and fibrate on the lipid levels in the plasma may be complementary.

The effects of the treatment on the activity of liver enzymes in the plasma are shown in the following Table:

| | AST [U/I], | ALT [U/I], |
|---|---|---|
| Treatment | MW ± SEM | MW ± SEM |
| Control | 51.8 ± 5.1 | 64.0 ± 6.4 |
| Compound (c) 10 mg/kg | 232.7 ± 34.2 | 232.9 ± 40.6 |
| Fenofibrate 100 mg/kg | 30.3 ± 2.7 | 47.0 ± 4.9 |
| Compound (c) 10 mg/kg | 40.5 ± 2.8 | 52.9 ± 5.7 |
| plus | | |
| Fenofibrate 100 mg/kg | | |

The side effects of the MTP inhibitor on the liver are clearly demonstrated by a 4.5-fold (AST) or 3.6-fold increase in the activity of these transaminases in the plasma. This increase is completely normalised by combining with fenofibrate.

### Example D

Male fa/fa rats 33 weeks old were either treated four times with an MTP inhibitor (given orally once a day between about 7 and 8 a.m.) or treated eight times with a fibrate (given twice a day by oral route between about 7 and 8 a.m. and at 4 p.m.) Another group were given both the MTP inhibitor and the fibrate. The MTP inhibitor was N-[3-(biphenyl-4-yl)-prop-2-ynyl]-4-(4′-trifluoromethylbiphenyl-2-carbonylamino)-1-methyl-pyrrole-2-carboxylic acid amide (compound (a)) in a dosage of 3 mg/kg. The fibrate was fenofibrate in a dosage of 100 mg/kg. 24 hours after the last dose of the MTP inhibitor or 15 hours after the last dose of fenofibrate, blood was taken from the animals and the levels of cholesterol, triglycerides and liver enzymes in the plasma were measured.

The effects of the treatment on the lipid levels in the plasma are shown in the following Table:

| | plasma | plasma |
|---|---|---|
| | cholesterol [mM], | triglycerides [mM], |
| Treatment | MW ± SEM | MW ± SEM |
| Control | 9.4 ± 1.4 | 9.5 ± 1.4 |
| Compound (a) 3 mg/kg | 7.0 ± 0.9 | 2.3 ± 0.4 |
| Fenofibrate 100 mg/kg | 10.3 ± 1.1 | 13.1 ± 2.8 |
| Compound (a) 3 mg/kg | 4.2 ± 0.9 | 2.6 ± 1.1 |
| plus | | |
| Fenofibrate 100 mg/kg | | |

Compared with the control group, which had been treated twice a day with carrier, the MTP inhibitor reduced cholesterol in the plasma by 26%, fenofibrate led to an increase of 10% and the combination of both led to a reduction of 55%. Triglycerides in the plasma were reduced by 76% by the MTP inhibitor, increased by 38% by the fenofibrate and reduced by 73% by the combination of both. As in Examples A and C, these data show that the effects of MTP inhibitor and fibrate on the lipid levels in the plasma may be complementary.

The effects of the treatment on the activity of liver enzymes in the plasma are shown in the following Table:

| | AST [U/I], | ALT [U/I], |
|---|---|---|
| Treatment | MW ± SEM | MW ± SEM |
| Control | 32.8 ± 8.5 | 45.7 ± 12.1 |
| Compound (a) 3 mg/kg | 218.4 ± 57.1 | 232.8 ± 67.8 |
| Fenofibrate 100 mg/kg | 33.5 ± 3.7 | 44.7 ± 5.3 |
| Compound (a) 3 mg/kg | 31.8 ± 2.1 | 48.8 ± 2.9 |
| plus | | |
| Fenofibrate 100 mg/kg | | |

The side effects of the MTP inhibitor on the liver are clearly demonstrated by a 6.7-fold (AST) or 5.1-fold increase in the activity of these transaminases in the plasma. This increase is completely normalised by combining with fenofibrate. The following are four specific examples of tablets and capsules which contain one or two active substances according to this invention.

1. Tablet Containing 5 mg Active Substance

| | | per batch |
|---|---|---|
| | per tablet | (10,000 tablets) |
| active substance (MTP inhibitor) | 5.0 mg | 50.0 g |
| lactose monohydrate (TLC quality) | 70.8 mg | 708.0 g |
| microcrystalline cellulose | 40.0 mg | 400.0 g |
| carboxymethylcellulose-sodium, | 3.0 mg | 30.0 g |
| indissolubly crosslinked | | |
| magnesium stearate | 1.2 mg | 12.0 g |

The active substance is mixed for 15 minutes with lactose monohydrate, microcrystalline cellulose and carboxymethylcellulose-sodium in a suitable diffusion mixer. Magnesium stearate is added and mixed with the other ingredients for another 3 minutes.

The finished mixture is compressed in a tablet press into round, flat, faceted tablets.

Diameter of the tablet: 7 mm; weight of a tablet: 120 mg

2. Capsules Containing 50 mg Active Substance

| | | per batch |
|---|---|---|
| | per capsule | (10,000 capsule) |
| active substance (MTP inhibitor) | 50.0 mg | 500.0 g |
| lactose monohydrate | 130.0 mg | 1300.0 g |
| maize starch | 65.0 mg | 650.0 g |
| highly dispersed silicon dioxide | 2.5 mg | 25.0 g |
| magnesium stearate | 2.5 mg | 25.0 g |

A starch paste is prepared by swelling some of the maize starch with a suitable amount of hot water. The paste is then left to cool to ambient temperature.

The active substance is premixed in a suitable mixer with lactose monohydrate and maize starch for 15 minutes. The starch paste is added and sufficient water is added to the mixture to produce a homogeneous moist mass. The moist mass is passed through a screen with a mesh size of 1.6 mm. The screened granules are dried on racks at about 55° C. for 12 hours.

The dried granules are then screened through meshes of 1.2 and 0.8 mm. Highly dispersed silicon dioxide is mixed with the granules in a suitable mixer for 3 minutes. Then magnesium stearate is added and mixing is continued for a further 3 minutes.

The finished mixture is packed into empty size 1 gelatine capsule shells using a capsule filling machine.

3. Tablet Containing 200 mg of Active Substances

| | | per batch |
|---|---|---|
| | per tablet | (10,000 tablets) |
| total active substances, e.g. | 200.0 mg | 2000.0 g |
| a) MTP inhibitor, or | 200.0 mg | |
| b) fibrate, or | 200.0 mg | |
| c) MTP inhibitor | 50.0 mg | |
| plus fibrate | 150.0 mg | |
| lactose monohydrate | 167.0 mg | 1670.0 g |
| microcrystalline cellulose | 80.0 mg | 800.0 g |
| HPMC Type 2910 (5 mPa*s quality) | 10.0 mg | 100.0 g |
| poly-1-vinyl-2-pyrrolidone, indissolubly | 20.0 mg | 200.0 g |
| crosslinked | | |
| magnesium stearate | 3.0 mg | 30.0 g |

HPMC (hydroxypropylmethylcellulose) is dispersed in hot water. After cooling, the mixture produces a clear solution.

The active substances are pre-mixed in a suitable mixer for 5 minutes with lactose monohydrate and microcrystalline cellulose. The HPMC solution is added and mixing is continued until a homogeneous moist mass is obtained. The moist mass is passed through a screen with a mesh size of 1.6 mm. The screened granules are dried on racks at about 55° C. for 12 hours.

The dried granules are then passed through screens with mesh sizes of 1.2 and 0.8 mm. Poly-1-vinyl-2-pyrrolidone is mixed with the granules in a suitable mixer for 3 minutes. Then magnesium stearate is added and the ingredients are mixed for an additional 3 minutes.

The finished mixture is compressed in a tablet press to form oblong tablets (16.2×7.9 mm).

Weight of a tablet: 480mg

4. Tablet Containing 500 mg of Active Substances

| | | per batch |
|---|---|---|
| | per tablet | (10,000 tablets) |
| total active substances, e.g. | 500.0 mg | 5000.0 g |
| a) fibrate, or | 500.0 mg | |
| b) MTP inhibitor | 100.0 mg | |
| plus fibrate | 400.0 mg | |
| microcrystalline cellulose | 80.0 mg | 800.0 g |
| HPMC type 2910 (5 mPa*s quality) | 10.0 mg | 100.0 g |
| Poly-1-vinyl-2-pyrrolidone, indissolubly | 20.0 mg | 200.0 g |
| crosslinked | | |
| magnesium stearate | 5.0 mg | 50.0 g |

HPMC is dispersed in hot water. After cooling, the mixture yields a clear solution.

The active substances are pre-mixed in a suitable mixer for 5 minutes with lactose monohydrate and microcrystalline cellulose. The HPMC solution is added and mixing is continued until a homogeneous moist mass is obtained. The moist mass is passed through a screen with a mesh size of 1.6 mm. The screened granules are dried on racks at about 55° C. for 12 hours.

The dried granules are then passed through screens with mesh sizes of 1.2 and 0.8 mm. Poly-1-vinyl-2-pyrrolidone is mixed with the granules in a suitable mixer for 15 minutes. Then magnesium stearate is added and the ingredients are mixed for a further 3 minutes.

The finished mixture is compressed in a tablet press to form oblong tablets (16.5×8.5 mm).

Weight of a tablet: 615 mg

### Additional Examples

### Example 1

### N-[4-(3-methyl-5-phenyl-pyrazol-1-yl)-phenylmethyl]-2-(biphenyl-2-carbonylamino)-thiazole-4-carboxylic acid amide

### a. 4-(3-methyl-5-phenyl-pyrazol-1-yl)-benzonitrile

A solution of 20.0 g (0.118 mol) of 4-cyanophenylhydrazine and 19.1 g (0.118 mol) of benzoylacetone in 600 ml of methanol is combined with 16.7 mg triethylamine and stirred for two days. The solvent is distilled off, the residue taken up in dichloromethane, washed with water and dried with sodium sulphate. Then the mixture is chromatographed on a silica gel column, eluting with dichloromethane.

Yield: 22.2 g (73% of theory),

R_{f} value: 0.9 (silica gel; dichloromethane/methanol=19:1)

C₁₇H₁₃N₃ (259.31)

Mass spectrum: (M+H)⁺=260

### b. 4-(3-methyl-5-phenyl-pyrazol-1-yl)-phenylmethylamine

22.2 g (0.086 mol) of 4-(3-methyl-5-phenyl-pyrazol-1-yl)-benzonitrile are dissolved in 660 ml of methanolic ammonia and after the addition of Raney nickel hydrogenated at ambient temperature with hydrogen (3 bar). The catalyst is filtered off and the solution is concentrated by evaporation. The residue is chromatographed on silica gel, eluting with dichloromethane/methanol=4:1.

Yield: 22 g (97% of theory),

R_{f} value: 0.2 (silica gel; dichloromethane/methanol=9:1)

C₁₇H₁₇N₃ (263.35)

Mass spectrum: (M+H)⁺=264

M⁺=263

### c. ethyl 2-amino-thiazole-4-carboxylate

7.2 g (0.094 mol) of thiourea are dissolved in 100 ml of ethanol, at ambient temperature combined with 12.0 g (0.086 mol) of ethyl bromopyroracemate and then refluxed for 1.5 hours. After cooling the mixture is diluted with 50 ml of water, made alkaline with conc. ammonia and the precipitate is suction filtered.

Yield: 12.5 g (84% of theory),

R_{f} value: 0.5 (silica gel; dichloromethane/ethanol=19:1)

C₆H₈N₂O₂S (172.21)

Mass spectrum: (M−H)⁻=171

(M+H)⁺=173

(M+Na)⁺=195

### d. ethyl 2-(biphenyl-2-carbonylamino)-thiazole-4-carboxylate

1.0 g (5.0 mmol) of 2-biphenylcarboxylic acid are placed in 15 ml of dimethylformamide and after the addition of 0.9 g (5.45 mmol) of ethyl 2-amino-thiazole-4-carboxylate, 1.8 g (5.60 mmol) of O-(benzotriazol-1-yl)-N,N,N′,N′-tetramethyluronium tetrafluoroborate (TBTU) and 2.9 ml (15.4.mmol) of N-ethyl-diisopropyl-amine the mixture is stirred for 12 hours. The solution is concentrated by evaporation and chromatographed on silica gel, eluting with petroleum ether/ethyl acetate (10-30%).

Yield: 0.5 g (28% of theory),

R_{f} value: 0.3 (silica gel; petroleum ether/ethyl acetate=7:3)

C₁₉H₁₆N₂O₃S (352.41)

Mass spectrum: (M+H)⁻=351

(M+Na)⁺=375

### e. 2-(Biphenyl-2-carbonylamino)-thiazole-4-carboxylic acid

0.5 g (1.4 mmol) of ethyl 2-(biphenyl-2-carbonylamino)-thiazole-4-carboxylate are stirred in 30 ml of ethanol and 1.6 ml of 2 molar sodium hydroxide solution for 18 hours at ambient temperature. The solvent is distilled off, the residue is combined with water and acidified with 2 molar hydrochloric acid. The product precipitated is suction filtered.

Yield: 0.3 g (72% of theory),

R_{f} value: 0.4 (silica gel; dichloromethane/ethanol=4:1)

C₁₇H₁₂N₂O₁₃S (324.36)

Mass spectrum: (M−H)⁻=323

### f. N-[4-(3-methyl-5-phenyl-pyrazol-1-yl)-phenylmethyl]-2-(biphenyl-2-carbonylamino)-thiazole-4-carboxylic acid amide

Prepared analogously to Example 1d from 2-(biphenyl-2-carbonylamino)-thiazole-4-carboxylic acid, 4-(3-methyl-5-phenyl-pyrazol-1-yl)-benzylamine, TBTU and N-ethyldiisopropylamine in dimethylformamide.

Yield: 23% of theory,

R_{f} value: 0.60 (silica gel; dichloromethane/ethanol=9:1)

C₃₄H₂₇N₅O₂S (569.69)

Mass spectrum: (M−H)⁻=568

(M+Na)⁺=592

### Example 2

### N-(biphenyl-4-yl)methyl-2-(biphenyl-2-carbonylamino)-thiazole-4-carboxylic acid amide

Prepared analogously to Example 1d from 2-(biphenyl-2-carbonylamino)-thiazole-4-carboxylic acid, 4-phenylbenzylamine, TBTU and N-ethyldiisopropylamine in dimethylformamide.

Yield: 86% of theory,

R_{f} value: 0.40 (silica gel; dichloromethane/ethanol=19:1)

C₃₀H₂₃N₃O₂S (489.60)

Mass spectrum: (M−H)⁻=488

### Example 3

### N-(4-benzoylamino-phenylmethyl)-2-(biphenyl-2-carbonylamino)-thiazole-4-carboxylic acid amide

Prepared analogously to Example 1d from 2-(biphenyl-2-carbonylamino)-thiazole-4-carboxylic acid, 4-benzoylaminobenzylamine, TBTU and N-ethyldiisopropylamine in dimethylformamide.

Yield: 25% of theory,

R_{f} value: 0.60 (silica gel; dichloromethane/ethanol=9:1)

C₃₁H₂₄N₄O₃S (532.62)

Mass spectrum: (M−H)⁻=531

(M+H)⁺=533

(M+Na)⁺=555

### Example 4

### N-(biphenyl-4-yl)methyl-5-(4′-trifluoromethylbiphenyl-2-carbonylamino)-thiophene-2-carboxylic acid amide

### a. N-(biphenyl-4-yl)methyl-5-nitro-thiophene-2-carboxylic acid amide

A mixture of 766 mg (4.0 mmol) of 5-nitrothiophene-2-carboxylic acid chloride, 733 mg (4.0 mmol) of 4-phenylbenzylamine and 1 ml of triethylamine are stirred in 45 ml of tetrahydrofuran for 18 hours. The solvent is distilled off and chromatographed on silica gel, eluting with dichloromethane.

Yield: 540 mg (40% of theory),

R_{f} value: 0.30 (silica gel; dichloromethane)

C₁₈H₁₄N₂O₃S (338.39)

Mass spectrum: (M−H)⁻=337

### b. N-(biphenyl-4-yl)methyl-5-aminothiophene-2-carboxylic acid amide

500 mg (1.47 mmol) of N-(biphenyl-4-yl)methyl-5-nitrothiophene-2-carboxylic acid amide are dissolved in 35 ml of methanol and 15 ml of dichloromethane and after the addition of 300 mg Raney nickel hydrogenated at ambient temperature with hydrogen (3 bar). The catalyst is filtered off and the solution concentrated by evaporation.

Yield: 400 mg (88% of theory),

R_{f} value: 0.30 (silica gel; dichloromethane/ethanol=50:1)

### c. N-(biphenyl-4-yl)methyl-5-(4′-trifluoromethylbiphenyl-2-carbonylamino)-thiophene-2-carboxylic acid amide

Prepared analogously to Example 4a from N-(biphenyl-4-yl)methyl-5-aminothiophene-2-carboxylic acid amide, 4′-trifluoromethylbiphenyl-2-carboxylic acid chloride and triethylamine in tetrahydrofuran.

Yield: 43% of theory

R_{f} value: 0.50 (silica gel; dichloromethane/ethanol=19:1)

C₃₂H₂₃F₃N₂O₂S (556.61)

Mass spectrum: (M−H)⁻=555

### Example 5

### N-[4-(3,4-dihydro-2H-quinolin-1-yl)-phenylmethyl]-6-(4′-trifluoromethylbiphenyl-2-carbonylamino)-pyrimidine-4-carboxylic acid amide

### a. 4-(3,4-dihydro-2H-quinolin-1-yl)-benzonitrile

5.3 g (0.04 mol) of 1,2,3,4-tetrahydroquinoline are dissolved in 60 ml of dimethylsulphoxide, 7.1 g (0.064 mol) of potassium tert. butoxide are added and the mixture is stirred for 20 minutes. Then 7.7 g (0.064 mol) of 4-fluorobenzonitrile in dimethylsulphoxide are added dropwise and the mixture is stirred for three days at 90° C. The reaction mixture is poured onto saturated sodium chloride solution and extracted with ethyl acetate. The combined organic extracts are chromatographed on aluminium oxide, eluting with petroleum ether/dichloromethane 1:1.

Yield: 4.5 g (48% of theory),

R_{f} value: 0.30 (silica gel; dichloromethane/petroleum ether=1:1)

C₁₆H₁₄N₂ (234.30)

Mass spectrum: (M−H)⁻=233

### b. 4-(3,4-dihydro-2H-quinolin-1-yl)-benzylamine

Prepared analogously to Example 1b from 4-(3,4-dihydro-2H-quinolin-1-yl)-benzonitrile, Raney nickel and methanolic ammonia with the addition of hydrogen.

Yield: 88% of theory

R_{f} value: 0.20 (silica gel; dichloromethane/ethanol=19:1)

C₁₆H₁₈N₂ (238.34)

Mass spectrum: (M+H)⁺=239

### c. N-[4-(3,4-dihydro-2H-quinolin-1-yl)-phenylmethyl]-6-chloro-pyrimidine-4-carboxylic acid amide

Prepared analogously to Example 4a from 4-(3,4-dihydro-2H-quinolin-1-yl)-benzylamine, 6-chloropyrimidine-4-carboxylic acid chloride and triethylamine in tetrahydrofuran.

Yield: 69% of theory

R_{f} value: 0.70 (silica gel; dichloromethane/ethanol=50:1)

C₁₂H₁₉ClN₄O (378.86)

Mass spectrum: (M−H)⁻=377/79 (chlorine isotope)

### d. N-[4-(3,4-dihydro-2H-quinolin-1-yl)-phenylmethyl]-6-(2,3-dimethoxy-phenylmethylamino)-pyrimidine-4-carboxylic acid amide

300 mg (0.79 mmol) of N-[4-(3,4-dihydro-2H-quinolin-1-yl)-phenylmethyl]-6-chloro-pyrimidine-4-carboxylic acid amide and 500 mg (3.0 mmol) of 2,4-dimethoxybenzylamine are stirred for two hours at 160° C. After cooling the mixture is chromatographed on silica gel, eluting with dichloromethane.

Yield: 380 mg (94% of theory),

R_{f} value: 0.80 (silica gel; dichloromethane/ethanol=19:1)

C₃₀H₃₁N₅O₃ (509.61)

Mass spectrum: (M−H)⁻=508

(M+Na)⁺=532

### e. N-[4-(3,4-dihydro-2H-quinolin-1-yl)-phenylmethyl]-6-amino-pyrimidine-4-carboxylic acid amide

350 mg (0.68 mmol) of N-[4-(3,4-dihydro-2H-quinolin-1-yl)-phenylmethyl]-6-(2,3-dimethoxy-benzylamino)-pyrimidine-4-carboxylic acid amide are dissolved in 30 ml of dichloromethane and after the addition of 7 ml trifluoroacetic acid stirred for two days. The solvent is distilled off, the mixture is made alkaline with methanolic ammonia and chromatographed on silica gel, eluting with dichloromethane/ethanol=99:1.

Yield: 130 mg (53% of theory),

R_{f} value: 0.70 (silica gel; dichloromethane/ethanol=19:1)

C₂₁H₂₁N₅O (359.43)

Mass spectrum: (M−H)⁻=358

### f. N-[4-(3,4-dihydro-2H-quinolin-1-yl)-phenylmethyl]-6-(4′-trifluoromethylbiphenyl-2-carbonylamino)-pyrimidine-4-carboxylic acid amide

Prepared analogously to Example 4a from N-[4-(3,4-dihydro-2H-quinolin-1-yl)-phenylmethyl]-6-amino-pyrimidine-4-carboxylic acid amide, 4′-trifluoromethylbiphenyl-2-carboxylic acid chloride and triethylamine in tetrahydrofuran.

Yield: 17% of theory

R_{f} value: 0.40 (silica gel; petroleum ether/ethyl acetate=2:1)

C₃₅H₂₈F₃N₅O₂ (607.63)

Mass spectrum: M⁺=607

(M+Na)⁺=630

### Example 6

### N-[4-(3,4-dihydro-1H-isoquinolin-2-yl)-phenylmethyl]-4-(4′-trifluoromethylbiphenyl-2-carbonylamino)-1-methyl-pyrrole-2-carboxylic acid amide

Prepared analogously to Example 1d from 4-(4′-trifluoromethyl-biphenyl-2-carbonylamino)-1-methyl-pyrrole-2-carboxylic acid, 4-(3,4-dihydro-1H-isoquinolin-2-yl)-benzylamine, TBTU and triethylamine in tetrahydrofuran.

Yield: 100% of theory

R_{f} value: 0.60 (silica gel; dichloromethane/ethanol=9:1)

C₃₆H₃₁F₃N₄O₂ (608.67)

Mass spectrum: (M−H)⁺=609

(M−H)⁻=607

(M−HCOO)⁻=653

### Example 7

### N-(4′-methylbiphenyl-4-yl)methyl-5-(4′-trifluoromethylbiphenyl-2-carbonylamino)-nicotinic acid amide

Prepared analogously to Example 1d from 5-(4′-trifluoromethyl-biphenyl-2-carbonylamino)-nicotinic acid, 4′-methylbiphenyl-4-methylamine, TBTU and N-ethyldiisopropylamine in dimethyl-formamide.

Yield: 26% of theory

R_{f} value: 0.49 (silica gel; dichloromethane/ethanol=9:1)

C₃₄H₂₆F₃N₃O₂ (565.60)

Mass spectrum: (M−H)⁻=564

(M+Na)⁺=588

### Example 8

### N-(4-phenylaminocarbonyl-phenylmethyl)-5-(4′-trifluoromethylbiphenyl-2-carbonylamino)-nicotinic acid amide

Prepared analogously to Example 1d from 4-phenylaminocarbonyl-benzylamine, 5-(4′-trifluoromethylbiphenyl-2-carbonyl-amino)-nicotinic acid, TBTU and N-ethyldiisopropylamine in dimethylformamide.

Yield: 21% of theory

R_{f} value: 0.41 (silica gel; dichloromethane/ethanol=9:1)

C₃₄H₂₅F₃N₄O₃ (594.59)

Mass spectrum: M⁺=594

### Example 9

### N-[4-(3-methyl-5-phenyl-pyrazol-1-yl)-phenylmethyl]-5-(4′-trifluoromethylbiphenyl-2-carbonylamino)-nicotinic acid amide

Prepared analogously to Example 1d from 5-(4′-trifluoromethyl-biphenyl-2-carbonylamino)-nicotinic acid, 4-(3-methyl-5-phenyl-pyrazol-1-yl)-benzylamine, TBTU and N-ethyldiisopropylamine in dimethylformamide.

Yield: 32% of theory

R_{f} value: 0.48 (silica gel; dichloromethane/ethanol=9:1)

C₃₇H₂₈F₃N₅O₂ (631.66)

Mass spectrum: (M+Na)⁺=654

### Example 10

### N-(4′-methylbiphenyl-4-yl)methyl-4-(4′-trifluoromethylbiphenyl-2-carbonylamino)-1-methyl-imidazol-2-carboxylic acid amide

Prepared analogously to Example 1d from 4-(4′-trifluoromethyl-biphenyl-2-carbonylamino)-1-methyl-imidazol-2-carboxylic acid, 4′-methylbiphenyl-4-methylamine, TBTU and N-ethyldiisopropylamine in dimethylformamide.

Yield: 10% of theory

R_{f} value: 0.95 (silica gel; dichloromethane/ethanol=4:1)

C₃₃H₂₇F₃N₄O₂ (568.60)

Mass spectrum: (M−H)⁻=567

(M+Na)⁺=591

### Example 11

### N-(biphenyl-4-yl)methyl-4-(4′-trifluoromethylbiphenyl-2-carbonylamino)-1-methyl-imidazol-2-carboxylic acid amide

A solution of 100 mg (0.25 mmol) of 4-(4′-trifluoromethyl-biphenyl-2-carbonylamino)-1-methyl-imidazol-2-carboxylic acid, 48 mg (0.25 mmol) of 4-phenylbenzylamine and 0.2 ml (1.5 mmol) of N-methylmorpholine in 6 ml of dichloromethane is combined with 0.3 ml (0.5 mmol) of propanephosphonic acid cycloanhydride (50 wt. % in ethyl acetate) at −10° C. and stirred for 2 hours with cooling. Then it is washed with 2 molar hydrochloric acid and 2 molar sodium hydroxide solution, the combined organic extracts are dried and concentrated by evaporation.

Yield: 0.12 g (84% of theory),

R_{f} value: 0.59 (silica gel; dichloromethane/ethanol=9:1)

C₃₂H₂₅F₃N₄O₂ (554.57)

Mass spectrum: (M−H)⁻=553

(M+H)⁺=555

(M+Na)⁺=577

### Example 12

### N-[4-(piperidino)-phenylmethyl]-4-(4′-trifluoromethyl-biphenyl-2-carbonylamino)-1-methyl-imidazol-2-carboxylic acid amide

Prepared analogously to Example 11 from 4-(piperidino)-benzylamine and 4-(4′-trifluoromethylbiphenyl-2-carbonylamino)-1-methylimidazole-2-carboxylic acid in dichloromethane with the addition of propanephosphonic acid cycloanhydride and N-methylmorpholine.

Yield: 88% of theory

R_{f} value: 0.53 (silica gel; dichloromethane/ethanol=9:1)

C₃₁H₃₀F₃N₅O₂ (561.61)

Mass spectrum: (M−H)⁻=560

### Example 13

### N-[4-(3,4-dihydro-2H-quinolin-1-yl)-phenylmethyl]-4-(4′-trifluoromethylbiphenyl-2-carbonylamino)-1-methyl-imidazol-2-carboxylic acid amide

Prepared analogously to Example 11 from 4-(3,4-dihydro-2H-quinolin-1-yl)-benzylamine and 4-(4′-trifluoromethylbiphenyl-2-carbonyl-amino)-1-methyl-imidazole-2-carboxylic acid in dichloromethane with the addition of propanephosphonic acid cycloanhydride and N-methyl-morpholine.

Yield: 85% of theory

R_{f} value: 0.71 (silica gel; dichloromethane/ethanol=9:1)

C₃₅H₃₀F₃N₅O₂ (609.65)

Mass spectrum: (M^{-H)}⁻=608

### Example 14

### N-(4′-trifluoromethybiphenyl-4-yl)methyl-4-(4′-trifluoromethyl-biphenyl-2-carbonyl-amino)-1-methyl-imidazol-2-carboxylic acid amide

Prepared analogously to Example 11 from 4′-trifluoromethylbiphenyl-4-methylamine and 4-(4′-trifluoromethylbiphenyl-2-carbonylamino)-1-methyl-imidazol-2-carboxylic acid in dichloromethane with the addition of propanephosphonic acid cycloanhydride and N-methylmorpholine.

Yield: 83% of theory

R_{f} value: 0.52 (silica gel; dichloromethane/ethanol=9:1)

C₃₃H₂₄F₆N₄O₂ (622.57)

Mass spectrum: (M−H)⁻=621

### Example 15

### N-(4′-Chlorobiphenyl-4-yl)methyl-4-(4′-trifluoromethylbiphenyl-2-carbonylamino)-1-methyl-imidazol-2-carboxylic acid amide

Prepared analogously to Example 11 from 4′-chlorobiphenyl-4-methyl-amine and 4-(4′-trifluoromethylbiphenyl-2-carbonylamino)-1-methyl-imidazole-2-carboxylic acid in dichloromethane with the addition of propanephosphonic acid cycloanhydride and N-methylmorpholine.

Yield: 88% of theory

R^{f} value: 0.54 (silica gel; dichloromethane/ethanol=9:1)

C₃₂H₂₄ClF₃N₄O₂ (589.02)

Mass spectrum: (M−H)⁻=587/89 (chlorine isotope)

### Example 16

### N-[4-(pyridin-4-yl)-phenylmethyl]-4-(4′-trifluoromethylbiphenyl-2-carbonylamino)-1-methyl-imidazole-2-carboxylic acid amide

Prepared analogously to Example 11 from 4-(pyridin-4-yl)-benzylamine and 4-(4′-trifluoromethylbiphenyl-2-carbonylamino)-1-methyl-imidazole-2-carboxylic acid in dichloromethane with the addition of propanephosphonic acid cycloanhydride and N-methylmorpholine.

Yield: 94% of theory

R_{f} value: 0.41 (silica gel; dichloromethane/ethanol=9:1)

C₃₁H₂₄F₃N₅O₂ (555.56)

Mass spectrum: (M−H)⁻=554

### Example 17

### N-[4-([1,2,3]-thiadiazol-4-yl)-phenylmethyl]-4-(4′-trifluoro-methylbiphenyl-2-carbonylamino)-1-methyl-imidazole-2-carboxylic acid amide

Prepared analogously to Example 11 from 4-([1,2,3]-thiadiazol-4-yl)-benzylamine and 4-(4′-trifluoromethylbiphenyl-2-carbonyl-amino)-1-methyl-imidazole-2-carboxylic acid in dichloromethane with the addition of propanephosphonic acid cycloanhydride and N-methylmorpholine.

Yield: 88% of theory

R_{f} value: 0.52 (silica gel; dichloromethane/ethanol=9:1)

C₂₈H₂₁F₃N₆O₂S (562.57)

Mass spectrum: (M−H)⁻=561

### Example 18

### N-[4-(6-methyl-pyridazin-3-yl)-phenylmethyl]-4-(4′-trifluoro-methylbiphenyl-2-carbonylamino)-1-methyl-imidazole-2-carboxylic acid amide

### a. 4-(6-methyl-pyridazin-3-yl)-benzonitrile

875 mg (6.8 mmol) of 3-chloro-6-methylpyridazine and 237 mg (0.2 mmol) of tetrakis-triphenylphosphine-palladium(0) are placed in 40 ml of toluene, a solution of 1.0 g (6.8 mmol) of 4-cyano-phenylboric acid in 20 ml of methanol and 1.4 g (13.6 mmol) of sodium carbonate in 20 ml of water are added and the mixture is refluxed for 7 hours. The reaction mixture is stirred for two days at ambient temperature and concentrated by evaporation. The residue is chromatographed on silica gel, eluting with dichloromethane/ethanol=9:1.

Yield: 340 mg (26% of theory),

R_{f} value: 0.53 (silica gel; dichloromethane/ethanol=9:1)

C₁₂H₉N₃ (195.23)

Mass spectrum: (M+H)⁺=196

### b. 4-(6-methyl-pyridazin-3-yl)-benzylamine

Prepared analogously to Example 1b from 4-(6-methyl-pyridazin-3-yl)-benzonitrile and Raney nickel in methanolic ammonia with the addition of hydrogen (3 bar).

Yield: 73% of theory,

R_{f} value: 0.13 (silica gel; dichloromethane/ethanol=75:25)

C₁₂H₁₃N₃ (199.26)

Mass spectrum: (M+H)⁺=200

### c. N-[4-(6-methyl-pyridazin-3-yl)-phenylmethyl]-4-(4′-trifluoro-methylbiphenyl-2-carbonylamino)-1-methyl-imidazole-2-carboxylic acid amide

Prepared analogously to Example 11 from 4-(6-methyl-pyridazin-3-yl)-benzylamine and 4-(4′-trifluoromethylbiphenyl-2-carbonyl-amino)-1-methyl-imidazole-2-carboxylic acid in dichloromethane with the addition of propanephosphonic acid cycloanhydride and N-methylmorpholine.

Yield: 96% of theory

R_{f} value: 0.51 (silica gel; dichloromethane/ethanol=9:1)

C₃₁H₂₅F₃N₆O₂ (570.57)

Mass spectrum: (M−H)⁻=569

(M+H)⁺=571

(M+Na)⁺=593

### Example 19

### N-[3-(4-biphenyl)-prop-2-ynyl]-4-(4′-trifluoromethylbiphenyl-2-carbonylamino)-1-methyl-imidazole-2-carboxylic acid amide

### a. N-tert.-butoxycarbonyl-prop-2-ynylamine

6.9 g (0.12 mol) of propargylamine is placed in 50 ml of dichloromethane, at 0° C. a solution of 27.3 g (0.12 mol) of di-tert.butyldicarbonate in 50 ml of dichloromethane is added dropwise and the mixture is stirred for three hours at ambient temperature. Then it is cooled to −20° C. and the product precipitated is suction filtered.

Yield: 18.2 g (94% of theory),

### b. N-tert.-butoxycarbonyl-3-(4-biphenyl)prop-2-ynylamine

A mixture of 1.3 g (5.3 mmol) of 4-bromobiphenyl, 0.1 g (0.53 mmol) of copper-(I)-iodide, 0.6 g (0.53 mmol) of tetrakis-triphenylphosphine-palladium(0) and 2.2 ml (16.1 mmol) of triethylamine are refluxed in 30 ml of tetrahydrofuran for 10 minutes, then the mixture is combined with 1.0 g (6.4 mmol) of N-tert.-butoxycarbonyl-prop-2-ynylamine and refluxed for a further 10 hours. The precipitate is filtered off and the filtrate is concentrated by evaporation. The residue is chromatographed on silica gel, eluting with petroleum ether/ethyl acetate 96:4.

Yield: 370 mg (22% of theory),

R_{f} value: 0.62 (silica gel; petroleum ether/ethyl acetate=7:3)

C₂₀H₂₁NO₂ (307.4)

Mass spectrum: (M+Na)⁺=330

### c. 3-(4-biphenyl)-prop-2-ynylamine-trifluoroacetate

365 mg (1.1 mmol) of N-tert.-butoxycarbonyl-3-(4-biphenyl)prop-2-ynylamine are stirred for 2 hours in 20 ml of dichloromethane and 2 ml of trifluoroacetic acid. Then it is concentrated by evaporation and the residue is reacted further directly.

Yield: 381 mg (quantitative),

R_{f} value: 0.22 (silica gel; dichloromethane/ethanol=9:1)

### d. N-[3-(4-biphenyl)-prop-2-ynyl]-4-(4′-trifluoromethylbiphenyl-2-carbonylamino)-1-methyl-imidazole-2-carboxylic acid amide

Prepared analogously to Example 11 from 3-biphenyl-4-yl-prop-2-ynylamine-trifluoroacetate and 4-(4′-trifluoromethyl-biphenyl-2-carbonylamino)-1-methyl-imidazole-2-carboxylic acid in dichloromethane with the addition of propanephosphonic acid cycloanhydride and N-methylmorpholine.

Yield: 58% of theory

R_{f} value: 0.59 (silica gel; dichloromethane/ethanol=9:1)

C₃₄H₂₅F₃N₄O₂ (578.59)

Mass spectrum: (M−H)⁻=577

(M+H)⁺=579

(M+Na)⁺=601

### Example 20

### N-(4′-hydroxybiphenyl-4-yl)methyl-4-(4′-trifluoromethylbiphenyl-2-carbonylamino)-1-methyl-imidazole-2-carboxylic acid amide

Prepared analogously to Example 11 from 4′-hydroxybiphenyl-4-methylamine and 4-(4′-trifluoromethyl-biphenyl-2-carbonylamino)-1-methyl-imidazole-2-carboxylic acid in dichloromethane with the addition of propanephosphonic acid cycloanhydride and N-methylmorpholine.

Yield: 30% of theory

R_{f} value: 0.45 (silica gel; dichloromethane/ethanol=9:1)

C₃₂H₂₅F₃N₄O₃ (570.57)

Mass spectrum: (M−H)⁻=569

### Example 21

### N-[3-(4-trifluoromethylphenyl)-prop-2-ynyl]-4-(4′-trifluoro-methylbiphenyl-2-carbonyl-amino)-1-methyl-imidazole-2-carboxylic acid amide

Prepared analogously to Example 11 from 3-(4-trifluoromethylphenyl)-prop-2-ynylamine and 4-(4′-trifluoromethyl-biphenyl-2-carbonyl-amino)-1-methyl-imidazole-2-carboxylic acid in dichloromethane with the addition of propanephosphonic acid cycloanhydride and N-methyl-morpholine.

Yield: 71% of theory

R_{f} value: 0.49 (silica gel; dichloromethane/ethanol=9:1)

C₂₉H₂₀F₆N₄O₂ (570.49)

Mass spectrum: (M−H)⁻=569

(M+Na)⁺=593

### Example 22

### N-[4-(1,4-dioxa-spiro[4.5]dec-8-yl)-phenylmethyl]-4-(4′-trifluoromethylbiphenyl-2-carbonylamino)-1-methyl-imidazole-2-carboxylic acid amide

Prepared analogously to Example 11 from 4-(1,4-dioxa-spiro[4.5]dec-8-yl)-benzylamine and 4-(4′-trifluoromethyl-biphenyl-2-carbonylamino)-1-methyl-imidazole-2-carboxylic acid in dichloromethane with the addition of propanephosphonic acid cycloanhydride and N-methylmorpholine.

Yield: 67% of theory

R_{f} value: 0.62 (silica gel; dichloromethane/ethanol=9:1)

C₃₄H₃₃F₃N₄O₄ (618.66)

Mass spectrum: (M−H)⁻=617

### Example 23

### N-[3-(4-tert.butylphenyl)-prop-2-ynyl]-4-(4′-trifluoromethyl-biphenyl-2-carbonylamino)-1-methyl-imidazole-2-carboxylic acid amide

Prepared analogously to Example 11 from 3-(4-tert.butylphenyl)-prop-2-ynylamine and 4-(4′-trifluoromethyl-biphenyl-2-carbonylamino)-1-methyl-imidazole-2-carboxylic acid in dichloromethane with the addition of propanephosphonic acid cycloanhydride and N-methylmorpholine.

Yield: 33% of theory

R_{f} value: 0.52 (silica gel; dichloromethane/ethanol=9:1)

C₃₂H₂₉F₃N₄O₂ (558.60)

Mass spectrum: (M−H)⁻=557

(M+Na)⁺=581

### Example 24

### N-(4′-methylbiphenyl-4-yl)methyl-4-(4′-trifluoromethylbiphenyl-2-carbonylamino)-1-methyl-pyrrole-2-carboxylic acid amide

Prepared analogously to Example 1d from 4′-methylbiphenyl-4-methyl-amine, 4-(4′-trifluoromethylbiphenyl-2-carbonylamino)-1-methyl-pyrrole-2-carboxylic acid, TBTU and N-ethyldiisopropylamine in dimethylformamide.

Yield: quantitative

R_{f} value: 0.40 (silica gel; dichloromethane/ethanol=19:1)

C₃₄H₂₈F₃N₃O₂ (567.61)

Mass spectrum: (M−H)⁻=566

(M+Na)⁺=590

### Example 25

### N-(4-phenylcarbonylamino-phenylmethyl)-4-(4′-trifluoromethyl-biphenyl-2-carbonylamino)-1-methyl-pyrrole-2-carboxylic acid amide

Prepared analogously to Example 1d from 4-phenylcarbonylamino-benzylamine, 4-(4′-trifluoromethylbiphenyl-2-carbonyl-amino)-1-methyl-pyrrole-2-carboxylic acid, TBTU and N-ethyldiisopropylamine in dimethylformamide.

Yield: 62% of theory

R_{f} value: 0.20 (silica gel; dichloromethane/ethanol=19:1)

C₃₄H₂₇F₃N₄O₃ (596.61)

Mass spectrum: (M−H)⁻=595

(M+Na)⁺=619

### Example 26

### N-[4-(3-methyl-5-phenyl-pyrazol-1-yl)-phenylmethyl]-4-(4′-trifluoromethylbiphenyl-2-carbonylamino)-1-methyl-pyrrole-2-carboxylic acid amide

Prepared analogously to Example 1d from 4-(3-methyl-5-phenyl-pyrazol-1-yl)-benzylamine, 4-(4′-trifluoromethylbiphenyl-2-carbonylamino)-1-methyl-pyrrole-2-carboxylic acid, TBTU and N-ethyl-diisopropylamine in dimethylformamide.

Yield: quantitative

R_{f} value: 0.25 (silica gel; dichloromethane/ethanol=19:1)

C₃₇H₃₀F₃N₅O₂ (633.67)

Mass spectrum: (M−H)⁻=632

(M+Na)⁺=656

### Example 27

### N-(4′-methylbiphenyl-4-yl)methyl-4-(biphenyl-2-carbonylamino)-1-methyl-pyrrole-2-carboxylic acid amide

Prepared analogously to Example 1d from 4′-methylbiphenyl-4-methyl-amine, 4-(biphenyl-2-carbonylamino)-1-methyl-pyrrole-2-carboxylic acid, TBTU and N-ethyldiisopropylamine in dimethylformamide.

Yield: 99% of theory

R_{f} value: 0.40 (silica gel; dichloromethane/ethanol=19:1)

C₃₃H₂₉N₃O₂ (499.61)

Mass spectrum: M⁺=499

### Example 28

### N-benzyl-4-(4′-trifluoromethylbiphenyl-2-carbonylamino)-1-methyl-pyrrole-2-carboxylic acid amide

Prepared analogously to Example 1d from benzylamine, 4-(4′-trifluoromethylbiphenyl-2-carbonylamino)-1-methyl-pyrrole-2-carboxylic acid, TBTU and N-ethyl-diisopropylamine in dimethylformamide.

Yield: quantitative

R_{f} value: 0.60 (silica gel; dichloromethane/ethanol=9:1)

C₂₇H₂₂F₃N₃O₂ (477.49)

Mass spectrum: (M−H)⁻=476

(M+Na)⁺=490

### Example 29

### N-pyridin-2-ylmethyl-4-(4′-trifluoromethylbiphenyl-2-carbonyl-amino)-1-methyl-pyrrole-2-carboxylic acid amide

Prepared analogously to Example 1d from 2-(aminomethyl)-pyridine, 4-(4′-trifluoromethylbiphenyl-2-carbonylamino)-1-methyl-pyrrole-2-carboxylic acid, TBTU and N-ethyl-diisopropylamine in dimethylformamide.

Yield: quantitative

R_{f} value: 0.50 (silica gel; dichloromethane/ethanol=9:1)

C₂₆H₂₁F₃N₄O₂ (478.47)

Mass spectrum: (M−H)⁻=477

### Example 30

### N-pyridin-3-ylmethyl-4-(4′-trifluoromethylbiphenyl-2-carbonyl-amino)-1-methyl-pyrrole-2-carboxylic acid amide

Prepared analogously to Example 1d from 3-(aminomethyl)-pyridine, 4-(4′-trifluoromethylbiphenyl-2-carbonylamino)-1-methyl-pyrrole-2-carboxylic acid, TBTU and N-ethyldiisopropylamine in dimethylformamide.

Yield: quantitative

R_{f} value: 0.40 (silica gel; dichloromethane/ethanol=9:1)

C₂₆H₂₁F₃N₄O₂ (478.47)

Mass spectrum: (M−H)⁻=477

(M+Na)⁺=501

### Example 31

### N-pyridin-4-ylmethyl-4-(4′-trifluoromethylbiphenyl-2-carbonyl-amino)-1-methyl-pyrrole-2-carboxylic acid amide

Prepared analogously to Example 1d from 4-(aminomethyl)-pyridine, 4-(4′-trifluoromethylbiphenyl-2-carbonylamino)-1-methyl-pyrrole-2-carboxylic acid, TBTU and N-ethyldiisopropylamine in dimethylformamide.

Yield: quantitative

R_{f} value: 0.35 (silica gel; dichloromethane/ethanol=9:1)

C₂₆H₂₁F₃N₄O₂ (478.47)

Mass spectrum: (M−H)⁻=477

(M+Na)⁺=501

### Example 32

### N-methoxycarbonylmethyl-4-(4′-trifluoromethylbiphenyl-2-carbonylamino)-1-methyl-pyrrole-2-carboxylic acid amide

Prepared analogously to Example 1d from glycine methyl ester-hydrochloride, 4-(4′-trifluoromethylbiphenyl-2-carbonylamino)-1-methyl-pyrrole-2-carboxylic acid, TBTU and N-ethyldiisopropylamine in dimethylformamide.

Yield: quantitative

R_{f} value: 0.70 (silica gel; dichloromethane/ethanol=9:1)

C₂₃H₂₀F₃N₃O₄ (459.42)

Mass spectrum: (M−H)⁻=458

(M+Na)⁺=482

### Example 33

### N-(2-methoxycarbonylethyl)-4-(4′-trifluoromethylbiphenyl-2-carbonylamino)-1-methyl-pyrrole-2-carboxylic acid amide

Prepared analogously to Example 1d from β-alaninemethylester-hydrochloride, 4-(4′-trifluoromethylbiphenyl-2-carbonylamino)-1-methyl-pyrrole-2-carboxylic acid, TBTU and N-ethyldiisopropylamine in dimethylformamide.

Yield: quantitative

R_{f} value: 0.70 (silica gel; dichloromethane/ethanol=9:1)

C₂₄H₂₂F₃N₃O₄ (473.45)

Mass spectrum: (M−H)⁻=472

(M+Na)⁺=496

### Example 34

### N-(4-[1,2,3]-thiadiazol-4-yl-phenylmethyl)-4-(4′-trifluoro-methylbiphenyl-2-carbonyl-amino)-1-methyl-pyrrole-2-carboxylic acid-amide

Prepared analogously to Example 1d from 4-[1,2,3]-thiadiazol-4-yl-benzylamine, 4-(4′-trifluoromethylbiphenyl-2-carbonyl-amino)-1-methyl-pyrrole-2-carboxylic acid, TBTU and N-ethyldiisopropylamine in dimethylformamide.

Yield: quantitative

R_{f} value: 0.70 (silica gel; dichloromethane/ethanol=9:1)

C₂₉H₂₂F₃N₅O₂S (561.59)

Mass spectrum: (M−H)⁻=560

### Example 35

### N-[2-(4-methylphenyl)pyridine-5-ylmethyl]-4-(4′-trifluoro-methylbiphenyl-2-carbonyl-amino)-1-methyl-pyrrole-2-carboxylic acid amide

Prepared analogously to Example 1d from (2-(4-methylphenyl)pyridin-5-yl)-methylamine, 4-(4′-trifluoromethylbiphenyl-2-carbonyl-amino)-1-methyl-pyrrole-2-carboxylic acid, TBTU and N-ethyl-diisopropylamine in dimethylformamide.

Yield: quantitative

R_{f} value: 0.55 (silica gel; dichloromethane/ethanol=9:1)

C₃₃H₂₇F₃N₄O₂ (568.60)

Mass spectrum: (M−H)⁻=567

(M+Na)⁺=591

### Example 36

### N-[4-(pyridin-4-yl)-phenylmethyl]-4-(4′-trifluoromethylbiphenyl-2-carbonylamino)-1-methyl-pyrrole-2-carboxylic acid amide

Prepared analogously to Example 1d from 4-(pyridin-4-yl)-benzylamine, 4-(4′-trifluoromethylbiphenyl-2-carbonylamino)-1-methyl-pyrrole-2-carboxylic acid, TBTU and N-ethyldiisopropylamine in dimethylformamide.

Yield: quantitative

R_{f} value: 0.45 (silica gel; dichloromethane/ethanol=9:1)

C₃₂H₂₅F₃N₄O₂ (554.57)

Mass spectrum: (M−H)⁻=553

### Example 37

### N-[4-(N-methyl-N-cyclohexylaminocarbonyl)-phenylmethyl]-4-(4′-trifluoromethyl-biphenyl-2-carbonylamino)-1-methyl-pyrrole-2-carboxylic acid amide

Prepared analogously to Example 1d from 4-(N-methyl-N-cyclohexyl-aminocarbonyl)-benzylamine, 4-(4′-trifluoromethylbiphenyl-2-carbonyl-amino)-1-methyl-pyrrole-2-carboxylic acid, TBTU and N-ethyldiisopropylamine in dimethylformamide.

Yield: 98% of theory

R_{f} value: 0.7 (silica gel; dichloromethane/ethanol=9:1)

C₃₅H₃₅F₃N₄O₃ (616.68)

Mass spectrum: (M−H)⁻=615

### Example 38

### N-(4-bromophenylmethyl)-4-(4′-trifluoromethylbiphenyl-2-carbonylamino)-1-methyl-pyrrole-2-carboxylic acid amide

Prepared analogously to Example 1d from 4-bromobenzylamine-hydrochloride, 4-(4′-trifluoromethylbiphenyl-2-carbonyl-amino)-1-methyl-pyrrole-2-carboxylic acid, TBTU and N-ethyldiisopropylamine in dimethylformamide.

Yield: quantitative

R_{f} value: 0.7 (silica gel; dichloromethane/ethanol=9:1)

C₂₇H₂₁BrF₃N₃O₂ (556.38)

Mass spectrum: (M−H)⁻=554/56 (bromine isotope)

### Example 39

### N-(4′-trifluoromethylbiphenyl-4-yl)methyl-4-(4′-trifluoromethyl-biphenyl-2-carbonyl-amino)-1-methyl-pyrrole-2-carboxylic acid amide

Prepared analogously to Example 1d from 4′-trifluoromethylbiphenyl-4-methylamine, 4-(4′-trifluoromethylbiphenyl-2-carbonyl-amino)-1-methyl-pyrrole-2-carboxylic acid, TBTU and N-ethyldiisopropylamine in dimethylformamide.

Yield: quantitative

R_{f} value: 0.7 (silica gel; dichloromethane/ethanol=9:1)

C₃₄H₂₅F₆N₃O₂ (621.58)

Mass spectrum: (M−H)⁻=620

### Example 40

### N-(4′-chlorobiphenyl-4-yl)methyl-4-(4′-trifluoromethylbiphenyl-2-carbonylamino)-1-methyl-pyrrole-2-carboxylic acid amide

Prepared analogously to Example 1d from 4′-chlorobiphenyl-4-methyl-amine, 4-(4′-trifluoromethylbiphenyl-2-carbonyl-amino)-1-methyl-pyrrole-2-carboxylic acid, TBTU and N-ethyldiisopropylamine in dimethylformamide.

Yield: quantitative

R_{f} value: 0.7 (silica gel; dichloromethane/ethanol=9:1)

C₃₃H₂₅ClF₃N₃O₂ (588.03)

Mass spectrum: (M−H)⁻=586/88 (chlorine isotope)

### Example 41

### N-[3-(4-methylphenyl)-prop-2-ynyl]-4-(4′-trifluoromethyl-biphenyl-2-carbonylamino)-1-methyl-pyrrole-2-carboxylic acid amide

Prepared analogously to Example 1d from 3-(4-methyl-phenyl)-prop-2-ynylamine, 4-(4′-trifluoromethylbiphenyl-2-carbonyl-amino)-1-methyl-pyrrole-2-carboxylic acid, TBTU and N-ethyldiisopropylamine in dimethylformamide.

Yield: 57% of theory

R_{f} value: 0.6 (silica gel; dichloromethane/ethanol=9:1)

C₃₀H₂₄F₃N₃O₂ (515.54)

Mass spectrum: (M−H)⁻=514

### Example 42

### N-[3-(4-isopropylphenyl)-prop-2-ynyl]-4-(4′-trifluoromethyl-biphenyl-2-carbonylamino)-1-methyl-pyrrole-2-carboxylic acid amide

Prepared analogously to Example 1d from 3-(4-isopropylphenyl)-prop-2-ynylamine, 4-(4′-trifluoromethylbiphenyl-2-carbonylamino)-1-methyl-pyrrole-2-carboxylic acid, TBTU and N-ethyldiisopropylamine in dimethylformamide.

Yield: 82% of theory

R_{f} value: 0.7 (silica gel; dichloromethane/ethanol=9:1)

C₃₂H₂₈F₃N₃O₂ (543.59)

Mass spectrum: (M−H)⁻=542

### Example 43

### N-hydroxycarbonylmethyl-4-(4′-trifluoromethylbiphenyl-2-carbonylamino)-1-methyl-pyrrole-2-carboxylic acid amide

Prepared analogously to Example 1e from N-methoxycarbonylmethyl-4-(4′-trifluoromethylbiphenyl-2-carbonylamino)-1-methyl-pyrrole-2-carboxylic acid amide and 2 molar sodium hydroxide solution in methanol.

Yield: 77% of theory

R_{f} value: 0.3 (silica gel; dichloromethane/ethanol=4:1)

C₂₂H₁₈F₃N₃O₄ (445.40)

Mass spectrum: (M−H)⁻=444

(M+Na)⁺=468

### Example 44

### N-(2-hydroxycarbonylethyl)-4-(4′-trifluoromethylbiphenyl-2-carbonylamino)-1-methyl-pyrrole-2-carboxylic acid amide

Prepared analogously to Example 1e from N-(2-methoxycarbonylethyl)-4-(4′-trifluoromethylbiphenyl-2-carbonylamino)-1-methyl-pyrrole-2-carboxylic acid amide and 2 molar sodium hydroxide solution in methanol.

Yield: 67% of theory

R_{f} value: 0.3 (silica gel; dichloromethane/ethanol=4:1)

C₂₃H₂₀F₃N₃O₄ (459.42)

Mass spectrum: (M−H)⁻=458

### Example 45

### N-(biphenyl-3-methyl)-4-(4′-trifluoromethylbiphenyl-2-carbonylamino)-1-methyl-pyrrole-2-carboxylic acid amide

Prepared analogously to Example 1d from 3-phenylbenzylamine, 4-(4′-trifluoromethylbiphenyl-2-carbonylamino)-1-methyl-pyrrole-2-carboxylic acid, TBTU and N-ethyldiisopropylamine in dimethylformamide.

Yield: quantitative

R_{f} value: 0.8 (silica gel; dichloromethane/ethanol=9:1)

C₃₃H₂₆F₃N₃O₂ (553.58)

Mass spectrum: (M−H)⁻=552

### Example 46

### N-(2′-methylbiphenyl-4-yl)methyl-4-(4′-trifluoromethylbiphenyl-2-carbonylamino)-1-methyl-pyrrole-2-carboxylic acid amide

Prepared analogously to Example 1d from 2′-methylbiphenyl-4-methylamine, 4-(4′-trifluoromethylbiphenyl-2-carbonylamino)-1-methyl-pyrrole-2-carboxylic acid, TBTU and N-ethyldiisopropylamine in dimethylformamide.

Yield: quantitative

R_{f} value: 0.75 (silica gel; dichloromethane/ethanol=9:1)

C₃₄H₂₈F₃N₃O₂ (567.61)

Mass spectrum: (M−H)⁻=566

### Example 47

### N-(4′-methoxycarbonylbiphenyl-4-yl)methyl-4-(4′-trifluoromethyl-biphenyl-2-carbonylamino)-1-methyl-pyrrole-2-carboxylic acid amide

Prepared analogously to Example 1d from 4′-methoxycarbonylbiphenyl-4-methylamine, 4-(4′-trifluoromethylbiphenyl-2-carbonylamino)-1-methyl-pyrrole-2-carboxylic acid, TBTU and N-ethyldiisopropylamine in dimethylformamide.

Yield: quantitative

R_{f} value: 0.75 (silica gel; dichloromethane/ethanol=9:1)

C₃₅H₂₈F₃N₃O₄ (611.62)

Mass spectrum: (M−H)⁻=610

### Example 48

### N-[4-(piperidino)-phenylmethyl)-4-(4′-trifluoromethyl-biphenyl-2-carbonylamino)-1-methyl-pyrrole-2-carboxylic acid amide

Prepared analogously to Example 1d from 4-(piperidino)-benzylamine, 4-(4′-trifluoromethylbiphenyl-2-carbonylamino)-1-methyl-pyrrole-2-carboxylic acid, TBTU and N-ethyldiisopropylamine in dimethylformamide.

Yield: quantitative

R_{f} value: 0.70 (silica gel; dichloromethane/ethanol=9:1)

C₃₂H₃₁F₃N₄O₂ (560.62)

Mass spectrum: (M−H)⁻=559

### Example 49

### N-[4-(1,4-dioxa-spiro[4.5]dec-8-yl)-phenylmethyl]-4-(4′-trifluoromethylbiphenyl-2-carbonylamino)-1-methyl-pyrrole-2-carboxylic acid amide

Prepared analogously to Example 1d from 4-(1,4-dioxa-spiro[4.5]dec-8-yl)-benzylamine, 4-(4′-trifluoromethylbiphenyl-2-carbonyl-amino)-1-methyl-pyrrole-2-carboxylic acid, TBTU and N-ethyldiisopropylamine in dimethylformamide.

Yield: quantitative

R_{f} value: 0.70 (silica gel; dichloromethane/ethanol=9:1)

C₃₅H₃₄F₃N₃O₄ (617.67)

Mass spectrum: (M+Na)⁺=640

### Example 50

### N-(4-tert.butylphenylmethyl)-4-(4′-trifluoromethylbiphenyl-2-carbonylamino)-1-methyl-pyrrole-2-carboxylic acid amide

Prepared analogously to Example 1d from 4-tert.butylbenzylamine, 4-(4′-trifluoromethylbiphenyl-2-carbonylamino)-1-methyl-pyrrole-2-carboxylic acid, TBTU and N-ethyldiisopropylamine in dimethylformamide.

Yield: quantitative

R_{f} value: 0.70 (silica gel; dichloromethane/ethanol=9:1)

C₃₁H₃₀F₃N₃O₂ (533.59)

### Example 51

### N-(4-chlorophenylmethyl)-4-(4′-trifluoromethylbiphenyl-2-carbonylamino)-1-methyl-pyrrole-2-carboxylic acid amide

Prepared analogously to Example 1d from 4-chlorobenzylamine, 4-(4′-trifluoromethylbiphenyl-2-carbonylamino)-1-methyl-pyrrole-2-carboxylic acid, TBTU and N-ethyldiisopropylamine in dimethylformamide.

Yield: quantitative

R_{f} value: 0.70 (silica gel; dichloromethane/ethanol=9:1)

C₂₇H₂₁ClF₃N₃O₂ (511.93)

Mass spectrum: (M−H)⁻=510/12 (chlorine isotope)

### Example 52

### N-(2-phenylthiazol-4-ylmethyl)-4-(4′-trifluoromethylbiphenyl-2-carbonylamino)-1-methyl-pyrrole-2-carboxylic acid amide

Prepared analogously to Example 1d from (2-phenylthiazol-4-yl)-methylamine, 4-(4′-trifluoromethylbiphenyl-2-carbonylamino)-1-methyl-pyrrole-2-carboxylic acid, TBTU and N-ethyldiisopropylamine in dimethylformamide.

Yield: quantitative

R_{f} value: 0.70 (silica gel; dichloromethane/ethanol=9:1)

C₃₀H₂₃F₃N₄O₂S (560.60)

Mass spectrum: (M−H)⁻=559

### Example 53

### N-(3-chloro-5-trifluoromethylpyridin-2-yl-methyl)-4-(4′-trifluoromethylbiphenyl-2-carbonylamino)-1-methyl-pyrrole-2-carboxylic acid amide

Prepared analogously to Example 1d from 3-chloro-5-trifluoromethyl-pyridin-2-yl-methylamine, 4-(4′-trifluoromethylbiphenyl-2-carbonylamino)-1-methyl-pyrrole-2-carboxylic acid, TBTU and N-ethyl-diisopropylamine in dimethylformamide.

Yield: quantitative

R_{f} value: 0.80 (silica gel; dichloromethane/ethanol=9:1)

C₂₇H₁₉ClF₆N₄O₂ (580.92)

Mass spectrum: (M−H)⁻=579/81 (chlorine isotope)

### Example 54

### N-(5-phenyl-[1,3,4]oxadiazol-2-yl-methyl)-4-(4′-trifluoromethyl-biphenyl-2-carbonyl-amino)-1-methyl-pyrrole-2-carboxylic acid amide

Prepared analogously to Example 1d from (5-phenyl-[1,3,4]oxadiazol-2-yl)-methylamine, 4-(4′-trifluoromethylbiphenyl-2-carbonyl-amino)-1-methyl-pyrrole-2-carboxylic acid, TBTU and N-ethyldiisopropylamine in dimethylformamide.

Yield: 76% of theory

R_{f} value: 0.70 (silica gel; dichloromethane/ethanol=9:1)

C₂₉H₂₂F₃N₅O₃ (545.52)

Mass spectrum: (M−H)⁻=544

### Example 55

### N-[4-(pyrimidin-4-yl-carbonylamino)-phenylmethyl]-4-(4′-trifluoromethylbiphenyl-2-carbonylamino)-1-methyl-pyrrole-2-carboxylic acid amide

Prepared analogously to Example 1d from 4-(pyrimidin-4-yl-carbonylamino)-benzylamine, 4-(4′-trifluoromethylbiphenyl-2-carbonylamino)-1-methyl-pyrrole-2-carboxylic acid, TBTU and N-ethyl-diisopropylamine in dimethylformamide.

Yield: 99% of theory

R_{f} value: 0.70 (silica gel; dichloromethane/ethanol=9:1)

C₃₂H₂₅F₃N₆O₃ (598.58)

Mass spectrum: (M−H)⁻=597

### Example 56

### N-(biphenyl-4-yl)methyl-N-methyl-4-(4′-trifluoromethylbiphenyl-2-carbonylamino)-1-methyl-pyrrole-2-carboxylic acid amide

Prepared analogously to Example 1d from N-methyl-4-phenylbenzylamine, 4-(4′-trifluoromethylbiphenyl-2-carbonylamino)-1-methyl-pyrrole-2-carboxylic acid, TBTU and N-ethyldiisopropylamine in dimethylformamide.

Yield: 77% of theory

R_{f} value: 0.80 (silica gel; dichloromethane/ethanol=9:1)

C₃₄H₂₈F₃N₃O₂ (567.61)

Mass spectrum: (M−H)⁻=566

### Example 57

### N-[4-(3,4-dihydro-2H-quinolin-1-yl)-phenylmethyl]-4-(4′-trifluoromethylbiphenyl-2-carbonylamino)-1-methyl-pyrrole-2-carboxylic acid amide

Prepared analogously to Example 1d from 4-(3,4-dihydro-2H-quinolin-1-yl)-benzylamine, 4-(4′-trifluoromethylbiphenyl-2-carbonyl-amino)-1-methyl-pyrrole-2-carboxylic acid, TBTU and N-ethyldiisopropylamine in dimethylformamide.

Yield: quantitative

R_{f} value: 0.65 (silica gel; dichloromethane/ethanol=9:1)

C₃₆H₃₁F₃N₄O₂ (608.66)

Mass spectrum: (M−H)⁻=607

### Example 58

### N-[4-(pyridin-3-yl)-phenylmethyl]-4-(4′-trifluoromethylbiphenyl-2-carbonylamino)-1-methyl-pyrrole-2-carboxylic acid amide

Prepared analogously to Example 1d from 4-(pyridin-3-yl)-benzylamine, 4-(4′-trifluoromethylbiphenyl-2-carbonylamino)-1-methyl-pyrrole-2-carboxylic acid, TBTU and N-ethyldiisopropylamine in dimethylformamide.

Yield: 37% of theory

R_{f} value: 0.65 (silica gel; dichloromethane/ethanol=9:1)

C₃₂H₂₅F₃N₄O₂ (554.57)

Mass spectrum: (M−H)⁻=553

### Example 59

### N-(4′-methylbiphenyl-4-yl)methyl-4-(4′-fluorobiphenyl-2-carbonylamino)-1-methyl-pyrrole-2-carboxylic acid amide

Prepared analogously to Example 1d from 4′-methylbiphenyl-4-methyl-amine, 4-(4′-fluorobiphenyl-2-carbonylamino)-1-methyl-pyrrole-2-carboxylic acid, TBTU and N-ethyldiisopropylamine in dimethylformamide.

Yield: 82% of theory

R_{f} value: 0.80 (silica gel; dichloromethane/ethanol=9:1)

C₃₃H₂₈FN₃O₂ (517.60)

### Example 60

### N-(4′-methylbiphenyl-4-yl)methyl-4-(4′-methylbiphenyl-2-carbonyl-amino)-1-methyl-pyrrole-2-carboxylic acid amide

Prepared analogously to Example 1d from 4′-methylbiphenyl-4-methyl-amine, 4-(4′-methylbiphenyl-2-carbonylamino)-1-methyl-pyrrole-2-carboxylic acid, TBTU and N-ethyldiisopropylamine in dimethylformamide.

Yield: quantitative

R_{f} value: 0.70 (silica gel; dichloromethane/ethanol=9:1)

C₃₄H₃₁N₃O₂ (513.64)

Mass spectrum: (M−H)⁻=512

### Example 61

### N-(4′-hydroxycarbonylbiphenyl-4-yl)methyl-4-(4′-trifluoromethyl-biphenyl-2-carbonyl-amino)-1-methyl-pyrrole-2-carboxylic acid amide

Prepared analogously to Example 1e from N-(4′-methoxycarbonyl-biphenyl-4-yl)methyl-4-(4′-trifluoromethyl-biphenyl-2-carbonyl-amino)-1-methyl-pyrrole-2-carboxylic acid amide and 2 molar sodium hydroxide solution in ethanol.

Yield: quantitative

R_{f} value: 0.40 (silica gel; dichloromethane/ethanol=9:1)

C₃₄H₂₆F₃N₃O₄ (597.59)

Mass spectrum: (M−H)⁻=596

### Example 62

### N-(4′-hydroxybiphenyl-4-yl)methyl-4-(4′-trifluoromethylbiphenyl-2-carbonylamino)-1-methyl-pyrrole-2-carboxylic acid amide

Prepared analogously to Example 1d from 4-(4-hydroxyphenyl)-benzylamine, 4-(4′-trifluoromethylbiphenyl-2-carbonylamino)-1-methyl-pyrrole-2-carboxylic acid, TBTU and N-ethyldiisopropylamine in dimethylformamide.

Yield: 58% of theory

R_{f} value: 0.50 (silica gel; dichloromethane/ethanol=9:1)

C₃₃H₂₆F₃N₃O₃ (569.58)

Mass spectrum: (M−H)⁻=568

### Example 63

### N-(4-methoxycarbonyl-4-phenyl-hexyl)-4-(4′-trifluoromethyl-biphenyl-2-carbonylamino)-1-methyl-pyrrole-2-carboxylic acid amide

Prepared analogously to Example 1d from methyl 5-amino-2-ethyl-2-phenyl-pentanoate, 4-(4′-trifluoromethylbiphenyl-2-carbonylamino)-1-methyl-pyrrole-2-carboxylic acid, TBTU and N-ethyl-diisopropylamine in dimethylformamide.

Yield: 21% of theory

R_{f} value: 0.40 (silica gel; petroleum ether/ethyl acetate=2:3)

C₃₄H₃₄F₃N₃O₄ (605.66)

Mass spectrum: (M−H)⁻=604

### Example 64

### N-(4′-methylbiphenyl-4-yl)methyl-4-(4′-trifluoromethylbiphenyl-2-carbonylamino)-1H-pyrrole-2-carboxylic acid amide

Prepared analogously to Example 11 from 4′-methylbiphenyl-4-methylamine and 4-(4′-trifluoromethylbiphenyl-2-carbonylamino)-1H-pyrrole-2-carboxylic acid in dichloromethane with the addition of propanephosphonic acid cycloanhydride and N-methylmorpholine.

Yield: 17% of theory

R_{f} value: 0.58 (silica gel; dichloromethane/ethanol=9:1)

C₃₃H₂₆F₃N₃O₂ (553.58)

Mass spectrum: (M−H)⁻=552

### Example 65

### N-(4′-methylbiphenyl-4-yl)methyl-4-(4′-trifluoromethylbiphenyl-2-carbonylamino)-1-ethyl-pyrrole-2-carboxylic acid amide

Prepared analogously to Example 1d from 4′-methylbiphenyl-4-methylamine, 4-(4′-trifluoromethylbiphenyl-2-carbonylamino)-1-ethyl-pyrrole-2-carboxylic acid, TBTU and N-ethyldiisopropylamine in dimethylformamide.

Yield: 78% of theory

R_{f} value: 0.80 (silica gel; dichloromethane/ethanol=9:1)

C₃₅H₃₀F₃N₃O₂ (581.64)

Mass spectrum: (M−H)⁻=580

### Example 66

### N-[4-(6-methylpyridazin-3-yl)-phenylmethyl]-4-(4′-trifluoromethylbiphenyl-2-carbonylamino)-1-methyl-pyrrole-2-carboxylic acid amide

Prepared analogously to Example 1d from 4-(6-methylpyridazin-3-yl)-benzylamine, 4-(4′-trifluoromethylbiphenyl-2-carbonylamino)-1-methyl-pyrrole-2-carboxylic acid, TBTU and N-ethyldiisopropylamine in dimethylformamide.

Yield: 28% of theory

R_{f} value: 0.49 (silica gel; dichloromethane/ethanol=9:1)

C₃₂H₂₆F₃N₅O₂ (569.59)

Mass spectrum: (M−H)⁻=568

(M+H)⁺=570

(M+Na)⁺=592

### Example 67

### N-[4-(pyridin-2-yl)-phenylmethyl]-4-(4′-trifluoromethylbiphenyl-2-carbonylamino)-1-methyl-pyrrole-2-carboxylic acid amide

Prepared analogously to Example 1d from 4-(pyridin-2-yl)-benzylamine, 4-(4′-trifluoromethylbiphenyl-2-carbonylamino)-1-methyl-pyrrole-2-carboxylic acid, TBTU and N-ethyldiisopropylamine in dimethylformamide.

Yield: quantitative

R_{f} value: 0.55 (silica gel; dichloromethane/ethanol=9:1)

C₃₂H₂₅F₃N₄O₂ (554.57)

Mass spectrum: (M−H)⁻=553

(M+Na)⁺=577

### Example 68

### N-[3-(4-methylphenyl)-propyl]-4-(4′-trifluoromethylbiphenyl-2-carbonylamino)-1-methyl-pyrrole-2-carboxylic acid amide

50 mg (0.097 mmol) of N-[3-(4-methyl-phenyl)-prop-2-ynyl]-4-(4′-trifluoromethyl-biphenyl-2-carbonylamino)-1-methyl-pyrrole-2-carboxylic acid amide are dissolved in 10 ml of ethanol and after the addition of 20 mg palladium on activated charcoal (10%) hydrogenated with hydrogen. The catalyst is filtered off and the solution is concentrated by evaporation.

Yield: 40 mg (79% of theory),

R_{f} value: 0.35 (silica gel; petroleum ether/ethyl acetate=1:1)

C₃₀H₂₈F₃N₃O₂ (519.57)

Mass spectrum: (M−H)⁻=518

### Example 69

### N-(4′-methylbiphenyl-4-yl)methyl-4-[2-(morpholin-4-yl)-phenyl-carbonylamino]-1-methyl-pyrrole-2-carboxylic acid amide

### a. ethyl 2-(morpholin-4-yl)-benzoate

A mixture of 1.7 ml (10.6 mmol) of ethyl 2-bromobenzoate, 1.0 ml (11.0 mmol) of morpholine, 5.4 g (16.5 mmol) of caesium carbonate, 75 mg (0.33 mmol) of palladium-II-acetate and 270 mg (0.43 mmol) of 2,2′-bis-(diphenylphosphino)-1,1′-binaphthyl are stirred in 30 ml xylene for 12 hours at 100° C. The solvent is distilled off and the residue is chromatographed on silica gel, eluting with dichloromethane/ethanol 9:1.

Yield: 0.6 g (25% of theory),

R_{f} value: 0.80 (silica gel; dichloromethane/ethanol=19:1)

C₁₃H₁₇NO₃ (235.29)

Mass spectrum: (M+H)⁺=236

(M+Na)⁺=258

### b. 2-(morpholin-4-yl)-benzoic acid

Prepared analogously to Example 1e from ethyl 2-(morpholin-4-yl)-benzoate and 2 molar sodium hydroxide solution in methanol.

Yield: 90% of theory,

R_{f} value: 0.75 (silica gel; dichloromethane/ethanol/ammonia=8:4:0.2)

C₁₁H₁₃NO₃ (207.23)

Mass spectrum: (M−H)⁻=206

(M+H)⁺=208

### c. methyl 1-methyl-4-[2-(morpholin-4-yl)-phenylcarbonylamino]-pyrrole-2-carboxylate

0.2 g (0.89 mmol) of 2-(morpholin-4-yl)-benzoic acid are stirred in 1.0 ml (13.7 mmol) of thionyl chloride with the addition of 2 drops of dimethylformamide for 90 minutes. The solution is concentrated by evaporation, 0.2 g (0.89 mmol) of methyl 1-methyl-4-amino-pyrrole-2-carboxylate, 0.4 ml (2.7 mmol) of triethylamine and 20 ml of tetrahydrofuran are added and the mixture is stirred for 17 hours. The solvent is distilled off, the residue dissolved in dichloromethane and washed with water. The combined organic extracts are dried and concentrated by evaporation.

Yield: 0.3 g (100% of theory),

R_{f} value: 0.35 (silica gel; dichloromethane/ethanol=19:1)

C₁₈H₂₁N₃O₄ (343.39)

Mass spectrum: (M−H)⁻=342

(M+Na)⁺=366

### d. 1-methyl-4-[2-(morpholin-4-yl)-phenylcarbonylamino]-pyrrole-2-carboxylic acid

Prepared analogously to Example 1e from methyl 1-methyl-4-[2-(morpholin-4-yl)-phenylcarbonylamino]-pyrrole-2-carboxylate and 2 molar sodium hydroxide solution in methanol.

Yield: 75% of theory

R_{f} value: 0.60 (silica gel; dichloromethane/ethanol=9:1)

C₁₇H₁₉N₃O₄ (329.36)

Mass spectrum: (M−H)⁻=328

(M+Na)⁺=352

### e. N-(4′-methylbiphenyl-4-yl)methyl-4-[2-(morpholin-4-yl)-phenyl-carbonylamino]-1-methyl-pyrrole-2-carboxylic acid amide

Prepared analogously to Example 1e from 1-methyl-4-[2-(morpholin-4-yl)-phenylcarbonylamino]-pyrrole-2-carboxylic acid, 4′-methylbiphenyl-4-methylamine, TBTU and N-ethyldiisopropylamine in dimethylformamide.

Yield: 94% of theory

R_{f} value: 0.55 (silica gel; dichloromethane/ethanol=9:1)

C₃₁H₃₂N₄O₃ (508.62)

Mass spectrum: (M−H)⁻=507

### Example 70

### N-(4′-methylbiphenyl-4-yl)methyl-4-(4′-trifluoromethylbiphenyl-2-carbonylamino)-1-(3-tert.butoxycarbonylaminopropyl)-pyrrole-2-carboxylic acid amide

Prepared analogously to Example 1d from 4′-trifluoromethylbiphenyl-2-carboxylic acid and N-(4′-methylbiphenyl-4-yl)methyl-4-amino-1-(3-tert.butoxycarbonylaminopropyl)-pyrrole-2-carboxylic acid amide, TBTU and N-ethyldiisopropylamine in dimethylformamide.

Yield: quantitative

R_{f} value: 0.60 (silica gel; dichloromethane/ethanol=9:1)

C₄₁H₄₁F₃N₄O₄ (710.80)

Mass spectrum: (M−H)⁻=709

(M+Na)⁺=733

### Example 71

### N-(4-benzyloxy-benzyl)-N-methyl-4-(4′-trifluoromethylbiphenyl-2-carbonylamino)-1-methyl-pyrrole-2-carboxylic acid amide

Prepared analogously to Example 1d from 4-(4′-trifluoromethyl-biphenyl-2-carbonylamino)-1-methyl-pyrrole-2-carboxylic acid, N-(4-benzyloxy-benzyl)-methylamine, TBTU and N-ethyldiisopropylamine in dimethylformamide.

Yield: 79% of theory

R_{f} value: 0.54 (silica gel; petroleum ether/ethyl acetate=1:2)

C₃₅H₃₀F₃N₃O₃ (597.64)

Mass spectrum: (M−H)⁻=596

(M+H)⁺=598

### Example 72

### N-[4-(2-methoxycarbonyl-ethyl)-phenylmethyl]-4-(4′-trifluoromethyl-biphenyl-2-carbonylamino)-1-methyl-pyrrole-2-carboxylic acid amide

Prepared analogously to Example 1d from 4-(4′-trifluoromethyl-biphenyl-2-carbonylamino)-1-methyl-pyrrole-2-carboxylic acid, 4-(2-methoxycarbonyl-ethyl)-benzylamine, TBTU and triethylamine in tetrahydrofuran.

Yield: 85% of theory

R_{f} value: 0.78 (silica gel; dichloromethane/ethanol=9:1)

C₃₁H₂₈F₃N₃O₄ (563.58)

Mass spectrum: (M−H)⁻=562

(M+H)⁺=564

### Example 73

### N-methyl-N-benzyl-4-(4′-trifluoromethylbiphenyl-2-carbonylamino)-1-methyl-pyrrole-2-carboxylic acid amide

Prepared analogously to Example 1d from 4-(4′-trifluoromethyl-biphenyl-2-carbonylamino)-1-methyl-pyrrole-2-carboxylic acid, N-methyl-benzylamine, TBTU and triethylamine in tetrahydrofuran.

Yield: 79% of theory

R_{f} value: 0.77 (silica gel; dichloromethane/ethanol=9:1)

C₂₈H₂₄F₃N₃O₂ (491.52)

Mass spectrum: (M−H)⁻=490

(M+H)⁺=492

### Example 74

### N-(2-difluoromethoxy-phenylmethyl)-4-(4′-trifluoromethyl-biphenyl-2-carbonylamino)-1-methyl-pyrrole-2-carboxylic acid amide

Prepared analogously to Example 1d from 4-(4′-trifluoromethyl-biphenyl-2-carbonylamino)-1-methyl-pyrrole-2-carboxylic acid, 2-difluoromethoxy-benzylamine, TBTU and triethylamine in tetrahydrofuran.

Yield: 69% of theory

R_{f} value: 0.75 (silica gel; dichloromethane/ethanol=9:1)

C₂₈H₂₂F₅N₃O₃ (543.49)

Mass spectrum: (M−H)⁻=542

(M+H)⁺=544

(M+Na)⁺=566

### Example 75

### N-(2-methyl-phenylmethyl)-4-(4′-trifluoromethylbiphenyl-2-carbonylamino)-1-methyl-pyrrole-2-carboxylic acid amide

Prepared analogously to Example 1d from 4-(4′-trifluoromethyl-biphenyl-2-carbonylamino)-1-methyl-pyrrole-2-carboxylic acid, 2-methyl-benzylamine, TBTU and triethylamine in tetrahydrofuran.

Yield: 66% of theory

R_{f} value: 0.76 (silica gel; dichloromethane/ethanol=9:1)

C₂₈H₂₄F₃N₃O₂ (491.52)

Mass spectrum: (M−H)⁻=490

(M+H)⁺=492

### Example 76

### N-[2-(biphenyl-4-yl)-ethyl]-4-(4′-trifluoromethylbiphenyl-2-carbonylamino)-1-methyl-pyrrole-2-carboxylic acid amide

Prepared analogously to Example 1d from 4-(4′-trifluoromethyl-biphenyl-2-carbonylamino)-1-methyl-pyrrole-2-carboxylic acid, 2-(biphenyl-4-yl)-ethylamine, TBTU and triethylamine in tetrahydrofuran.

Yield: 88% of theory

R_{f} value: 0.76 (silica gel; dichloromethane/ethanol=9:1)

C₃₄H₂₈F₃N₃O₂ (567.61)

Mass spectrum: (M−H)⁻=566

(M+H)⁺=568

(M+Na)⁺=590

### Example 77

### N-[4-(4-methylpiperidino)-phenylmethyl)-4-(4′-trifluoro-methylbiphenyl-2-carbonylamino)-1-methyl-pyrrole-2-carboxylic acid-amide

Prepared analogously to Example 1d from 4-(4′-trifluoromethyl-biphenyl-2-carbonylamino)-1-methyl-pyrrole-2-carboxylic acid, 4-(4-methylpiperidino)-benzylamine, TBTU and triethylamine in tetrahydrofuran.

Yield: 48% of theory

R_{f} value: 0.25 (silica gel; petroleum ether/ethyl acetate=3:2)

C₃₃H₃₃F₃N₄O₂ (574.65)

Mass spectrum: (M−H)⁻=573

(M+H)⁺=575

### Example 78

### N-[4-(1,4-dioxa-8-aza-spiro[4.5]dec-8-yl)-phenylmethyl]-4-(4′-trifluoromethylbiphenyl-2-carbonylamino)-1-methyl-pyrrole-2-carboxylic acid amide

Prepared analogously to Example 1d from 4-(4′-trifluoromethyl-biphenyl-2-carbonylamino)-1-methyl-pyrrole-2-carboxylic acid, 4-(1,4-dioxa-8-aza-spiro[4.5]dec-8-yl)-benzylamine, TBTU and triethylamine in tetrahydrofuran.

Yield: 90% of theory

R_{f} value: 0.65 (silica gel; petroleum ether/ethyl acetate=3:2)

C₃₄H₃₃F₃N₄O₄ (618.66)

Mass spectrum: (M−H)⁻=617

(M+H)⁺=619

### Example 79

### N-[4-(3-aza-spiro[5.5]undec-3-yl)-phenylmethyl]-4-(4′-trifluoromethylbiphenyl-2-carbonylamino)-1-methyl-pyrrole-2-carboxylic acid amide

Prepared analogously to Example 1d from 4-(4′-trifluoromethyl-biphenyl-2-carbonylamino)-1-methyl-pyrrole-2-carboxylic acid, 4-(3-aza-spiro[5.5]undec-3-yl)-benzylamine, TBTU and triethylamine in tetrahydrofuran.

Yield: 65% of theory

R_{f} value: 0.21 (silica gel; petroleum ether/ethyl acetate=3:2)

C₃₇H₃₉F₃N₄O₂ (628.74)

Mass spectrum: (M+H)⁺=629

### Example 80

### N-[1-(4-chlorophenyl)-ethyl]-4-(4′-trifluoromethylbiphenyl-2-carbonylamino)-1-methyl-pyrrole-2-carboxylic acid amide

Prepared analogously to Example 1d from 4-(4′-trifluoromethyl-biphenyl-2-carbonylamino)-1-methyl-pyrrole-2-carboxylic acid, 1-(4-chlorophenyl)-ethylamine, TBTU and triethylamine in tetrahydrofuran.

Yield: 100% of theory

R_{f} value: 0.82 (silica gel; dichloromethane/ethanol=9:1)

C₂₈H₂₃ClF₃N₃O₂ (525.96)

Mass spectrum: (M−H)⁻=524/26 (chlorine isotope)

(M+H)⁺=526/28 (chlorine isotope)

### Example 81

### N-[4-(3-methyl-[1,2,4]oxadiazol-5-yl)methyl-phenylmethyl]-4-(4′-trifluoromethylbiphenyl-2-carbonylamino)-1-methyl-pyrrole-2-carboxylic acid amide

Prepared analogously to Example 1d from 4-(4′-trifluoromethyl-biphenyl-2-carbonylamino)-1-methyl-pyrrole-2-carboxylic acid, 4-(3-methyl-[1,2,4]oxadiazol-5-yl)methyl-benzylamine, TBTU and triethylamine in tetrahydrofuran.

Yield: 84% of theory

R_{f} value: 0.70 (silica gel; dichloromethane/ethanol=9:1)

C₃₁H₂₆F₃N₅O₃ (573.58)

Mass spectrum: (M−H)⁻=572

(M+H)⁺=574

(M+Na)⁺=596

### Example 82

### N-(4-methoxycarbonyl-cyclohexylmethyl)-4-(4′-trifluoromethyl-biphenyl-2-carbonylamino)-1-methyl-pyrrole-2-carboxylic acid amide

Prepared analogously to Example 1d from 4-(4′-trifluoromethyl-biphenyl-2-carbonylamino)-1-methyl-pyrrole-2-carboxylic acid, methyl 4-aminomethyl-cyclohexanecarboxylate, TBTU and triethylamine in tetrahydrofuran.

Yield: 62% of theory

R_{f} value: 0.72 (silica gel; dichloromethane/ethanol=9:1)

C₂₉H₃₀F₃N₃O₄ (541.57)

Mass spectrum: (M−H)⁻=540

(M+H)⁺=542

### Example 83

### N-(4-benzyloxy-benzyl)-4-(4′-trifluoromethylbiphenyl-2-carbonylamino)-1-methyl-pyrrole-2-carboxylic acid amide

Prepared analogously to Example 1d from 4-(4′-trifluoromethyl-biphenyl-2-carbonylamino)-1-methyl-pyrrole-2-carboxylic acid, 4-benzyloxy-benzylamine, TBTU and triethylamine in tetrahydrofuran.

Yield: 83% of theory

R_{f} value: 0.73 (silica gel; dichloromethane/ethanol=9:1)

C₃₄H₂₈F₃N₃O₃ (583.61)

Mass spectrum: (M+H)⁺=584

(M+Na)⁺=606

(M−H)⁻=582

(M+HCOO)⁻=628

### Example 84

### N-[4-(3-methylpiperidino)-phenylmethyl)-4-(4′-trifluoro-methylbiphenyl-2-carbonylamino)-1-methyl-pyrrole-2-carboxylic acid-amide

Prepared analogously to Example 1d from 4-(4′-trifluoromethyl-biphenyl-2-carbonylamino)-1-methyl-pyrrole-2-carboxylic acid, 4-(3-methylpiperidino)-benzylamine, TBTU and triethylamine in tetrahydrofuran.

Yield: 16% of theory

R_{f} value: 0.81 (silica gel; dichloromethane/ethanol=9:1)

C₃₃H₃₃F₃N₄O₂ (574.65)

Mass spectrum: (M+H)⁺=575

(M+HCOO)⁻=619

### Example 85

### N-[cyclopropyl-(4-methoxy-phenyl)-methyl]-4-(4′-trifluoromethyl-biphenyl-2-carbonylamino)-1-methyl-pyrrole-2-carboxylic acid-amide and

### N-[1-(4-methoxy-phenyl)-butyl]-4-(4′-trifluoromethyl-biphenyl-2-carbonylamino)-1-methyl-pyrrole-2-carboxylic acid amide in the ratio 1:1

Prepared analogously to Example 1d from 4-(4′-trifluoromethyl-biphenyl-2-carbonylamino)-1-methyl-pyrrole-2-carboxylic acid, a 1:1 mixture of 1-(4-methoxy-phenyl)-butylamine and C-cyclopropyl-C-(4-methoxy-phenyl)-methylamine, TBTU and triethylamine in tetrahydrofuran.

Yield: 100% of theory

R_{f} value: 0.74 (silica gel; dichloromethane/ethanol=9:1)

N-[cyclopropyl-(4-methoxy-phenyl)-methyl]-4-(4′-trifluoro-methylbiphenyl-2-carbonylamino)-1-methyl-pyrrole-2-carboxylic acid-amide

C₃₁H₂₈F₃N₃O₃ (547.58)

Mass spectrum: (M)⁺=547

(M+H)⁺=548

(M+Na)⁺=570

(M−H)⁻=546

N-[1-(4-methoxy-phenyl)-butyl]-4-(4′-trifluoromethyl-biphenyl-2-carbonylamino)-1-methyl-pyrrole-2-carboxylic acid amide

C₃₁H₃₀F₃N₃O₃ (549.59)

Mass spectrum: (M)⁺=549

(M+H)⁺=550

(M+Na)⁺=572

(M−H)⁻=548

### Example 86

### N-[5-(4-cyano-4-phenyl-piperidino-carbonyl)-1-methyl-pyrrol-3-yl]-4′-trifluoro-methyl-biphenyl-2-carboxylic acid amide

Prepared analogously to Example 1d from 4-(4′-trifluoromethyl-biphenyl-2-carbonylamino)-1-methyl-pyrrole-2-carboxylic acid, 4-cyano-4-phenyl-piperidine, TBTU and triethylamine in tetrahydrofuran.

Yield: 67% of theory

R_{f} value: 0.83 (silica gel; dichloromethane/ethanol=9:1)

C₃₂H₂₇F₃N₄O₂ (556.59)

Mass spectrum: (M−H)⁻=555

(M+H)⁺=557

### Example 87

### N-[4-(9-ethylaminocarbonyl-fluoren-9-yl)-butyl]-4-(4′-trifluoromethylbiphenyl-2-carbonylamino)-1-methyl-pyrrole-2-carboxylic acid amide

Prepared analogously to Example 1d from 4-(4′-trifluoromethyl-biphenyl-2-carbonylamino)-1-methyl-pyrrole-2-carboxylic acid, 4-(9-ethylaminocarbonyl-fluoren-9-yl)-butylamine, TBTU and N-ethyldiisopropylamine in dimethylformamide.

Yield: quantitative

R_{f} value: 0.60 (silica gel; dichloromethane/ethanol=9:1)

C₄₀H₃₇F₃N₄O₃ (678.76)

Mass spectrum: (M−H)⁻=677

(M+Na)⁺=701

### Example 88

### N-(4′-methylbiphenyl-4-yl)methyl-4-(4′-trifluoromethylbiphenyl-2-carbonylamino)-1-(3-aminopropyl)-pyrrole-2-carboxylic acid amide

Prepared analogously to Example 19c from N-(4′-methylbiphenyl-4-yl)methyl-4-(4′-trifluoromethylbiphenyl-2-carbonylamino)-1-(3-tert.butoxycarbonylaminopropyl)-pyrrole-2-carboxylic acid amide and trifluoroacetic acid in dichloromethane.

Yield: quantitative

R_{f} value: 0.35 (silica gel; dichloromethane/ethanol/ammonia=50:45:5)

C₃₆H₃₃F₃N₄O₂ (610.68)

Mass spectrum: (M−H)⁻=609

(M+H)⁺=611

### Example 89

### N-[4-(5-dimethylaminopyridin-2-yl)-phenylmethyl]-4-(4′-trifluoromethylbiphenyl-2-carbonylamino)-1-methyl-pyrrole-2-carboxylic acid amide

Prepared analogously to Example 1d from 4-(4′-trifluoromethyl-biphenyl-2-carbonylamino)-1-methyl-pyrrole-2-carboxylic acid, 4-(5-dimethylamino-pyridin-2-yl)-benzylamine, TBTU and N-ethyl-diisopropylamine in dimethylformamide.

Yield: 57% of theory

R_{f} value: 0.55 (silica gel; dichloromethane/ethanol=19:1)

C₃₄H₃₀F₃N₅O₂ (597.64)

Mass spectrum: (M−H)⁻=596

(M+H)⁺=598

(M+Na)⁺=620

### Example 90

### N-[3-(biphenyl-4-yl)-prop-2-ynyl]-4-(4′-trifluoromethylbiphenyl-2-carbonylamino)-1-methyl-pyrrole-2-carboxylic acid amide

Prepared analogously to Example 1d from 4-(4′-trifluoromethyl-biphenyl-2-carbonylamino)-1-methyl-pyrrole-2-carboxylic acid, 3-(biphenyl-4-yl)-prop-2-ynylamine-trifluoroacetate, TBTU and N-ethyl-diisopropylamine in dimethylformamide.

Yield: 22% of theory

R_{f} value: 0.70 (silica gel; dichloromethane/ethanol=9:1)

C₃₅H₂₆F₃N₃O₂ (577.60)

Mass spectrum: (M−H)⁻=576

(M+H)⁺=578

### Example 91

### N-[3-(4-isopropylphenyl)-prop-2-ynyl]-4-(4′-trifluoromethyl-biphenyl-2-carbonylamino)-1-methyl-imidazole-2-carboxylic acid amide

Prepared analogously to Example 11 from 3-(4-isopropylphenyl)-prop-2-ynylamine and 4-(4′-trifluoromethylbiphenyl-2-carbonyl-amino)-1-methyl-imidazole-2-carboxylic acid in dichloromethane with the addition of propanephosphonic acid cycloanhydride and N-methylmorpholine.

Yield: 24% of theory

R_{f} value: 0.49 (silica gel; dichloromethane/ethanol=9:1)

C₃₁H₂₇F₃N₄O₂ (544.58)

Mass spectrum: (M−H)⁻=543

(M+Na)⁺=567

### Example 92

### N-(4′-methylbiphenyl-4-yl)methyl-4-[2-(pyrrolidin-1-yl)phenyl-carbonylamino]-1-methyl-pyrrole-2-carboxylic acid amide

Prepared analogously to Example 1d from 4-[2-(pyrrolidin-1-yl)-phenylcarbonylamino)-1-methyl-pyrrole-2-carboxylic acid, 4′-methyl-biphenyl-4-methylamine, TBTU and N-ethyldiisopropylamine in dimethylformamide.

Yield: 82% of theory

R_{f} value: 0.60 (silica gel; dichloromethane/ethanol=9:1)

C₃₁H₃₂N₄O₂ (492.62)

Mass spectrum: (M−H)⁻=491

(M+Na)⁺=515

### Example 93

### N-[5-(1,2,3,4-tetrahydroisoquinolin-2-yl-carbonyl)-1-methyl-pyrrol-3-yl]-4′-trifluoro-methylbiphenyl-2-carboxylic acid amide

Prepared analogously to Example 1d from 4-(4′-trifluoromethyl-biphenyl-2-carbonylamino)-1-methyl-pyrrole-2-carboxylic acid, 1,2,3,4-tetrahydroisoquinoline, TBTU and N-ethyldiisopropylamine in dimethylformamide.

Yield: 70% of theory

R_{f} value: 0.72 (silica gel; dichloromethane/ethanol=9:1)

C₂₉H₂₄F₃N₃O₂ (503.52)

Mass spectrum: (M−H)⁻=502

(M+H)⁺=504

### Example 94

### N-[5-(1,3-dihydro-isoindol-2-yl-carbonyl)-1-methyl-pyrrol-3-yl]-4′-trifluoromethyl-biphenyl-2-carboxylic acid amide

Prepared analogously to Example 1d from 4-(4′-trifluoromethyl-biphenyl-2-carbonylamino)-1-methyl-pyrrole-2-carboxylic acid, 2,3-dihydro-1H-isoindole, TBTU and N-ethyldiisopropylamine in dimethylformamide.

Yield: 79% of theory

R_{f} value: 0.64 (silica gel; dichloromethane/ethanol=9:1)

C₂₈H₂₂F₃N₃O₂ (489.50)

Mass spectrum: (M−H)⁻=488

(M+H)⁺=490

(M+Na)⁺=512

### Example 95

### N-(4′-methylbiphenyl-4-yl)methyl-4-[1-oxo-7-(4-trifluoromethylphenyl)-1,3-dihydro-isoindol-2-yl]-1-methyl-pyrrole-2-carboxylic acid amide

### a. methyl 3-methyl-4′-trifluoromethylbiphenyl-2-carboxylate

A mixture of 1.1 g (4.58 mmol) of methyl 2-bromo-6-methyl-benzoate, 0.9 g (4.7 mmol) of 4-(trifluoromethyl)-benzeneboric acid, 0.3 g (0.26 mmol) of tetrakis-triphenyl-phosphine-palladium(O) and 0.2 g (0.24 mmol) of 2,2′-bis-(diphenyl-phosphino)-1,1′-binaphthyl are placed in 150 ml of dimethoxyethane, after 10 minutes combined with 7 ml (7 mmol) of 1 molar sodium carbonate solution and then refluxed for 5 hours. The solvent is distilled off, the residue is distributed in water/dichloromethane, the combined organic extracts are dried and chromatographed on silica gel, eluting with ethyl acetate/petroleum ether 95:5.

Yield: 1.1 g (83% of theory),

R_{f} value: 0.8 (silica gel; dichloromethane/ethanol=99:1)

C₁₆H₁₃F₃O₂ (294.28)

Mass spectrum: (M+Na)⁺=317

### b. methyl 3-bromomethyl-4′-trifluoromethylbiphenyl-2-carboxylate

0.5 g (1.7 mmol) of methyl 3-methyl-4′-trifluoromethylbiphenyl-2-carboxylate are dissolved in 10 ml of carbon tetrachloride and after the addition of 0.45 g (2.57 mmol) of N-bromosuccinimide and 10 mg (0.06 mmol) of 2,2-azoisobutyronitrile refluxed for 7 hours. The succinimide precipitated is suction filtered and the filtrate is concentrated by evaporation. The residue is chromatographed on silica gel, eluting with petroleum ether/dichloromethane 8:2.

Yield: 0.4 g (62% of theory),

R_{f} value: 0.45 (silica gel; petroleum ether/ethyl acetate=9:1)

C₁₆H₁₂BrF₃O₂ (373.17)

Mass spectrum: M⁺=372/74 (bromine isotope)

### c. methyl 4-[1-oxo-7-(4-trifluoromethylphenyl)-1,3-dihydro-isoindol-2-yl]-1-methyl-pyrrole-2-carboxylate

0.4 g (1.0 mmol) of methyl 3-bromomethyl-4′-trifluoromethylbiphenyl-2-carboxylate are dissolved in 15 ml acetonitrile and after the addition of 0.2 g (1.0 mmol) of methyl 4-amino-1-methyl-pyrrole-2-carboxylate stirred for 3.5 hours at 80° C. The solvent is distilled off and the residue is chromatographed on silica gel, eluting with petroleum ether/ethyl acetate 85:15 and 75:25.

Yield: 0.2 g (43% of theory),

R_{f} value: 0.25 (silica gel; dichloromethane/ethanol=99:1)

C₂₂H₁₇F₃N₂O₃ (414.39)

Mass spectrum: (M−H)⁻=413

(M+H)⁺=415

(M+Na)⁺=437

### d. 4-[1-oxo-7-(4-trifluoromethylphenyl)-1,3-dihydro-isoindol-2-yl]-1-methyl-pyrrole-2-carboxylic acid

Prepared analogously to Example 1e from methyl 4-[1-oxo-7-(4-trifluoromethylphenyl)-1,3-dihydro-isoindol-2-yl]-1-methyl-pyrrole-2-carboxylate and sodium hydroxide solution in methanol.

Yield: 85% of theory

R_{f} value: 0.35 (silica gel; dichloromethane/ethanol=19:1)

C₂₁H₁₅F₃N₂O₃ (400.36)

Mass spectrum: (M−H)⁻=399

(M+H)⁺=401

(M+Na)⁺=423

### e. N-(4′-methylbiphenyl-4-yl)methyl-4-[1-oxo-7-(4-trifluoromethylphenyl)-1,3-dihydro-isoindol-2-yl]-1-methyl-pyrrole-2-carboxylic acid amide

Prepared analogously to Example 1d from 4-[1-oxo-7-(4-trifluoromethylphenyl)-1,3-dihydro-isoindol-2-yl]-1-methyl-pyrrole-2-carboxylic acid, C-(4′-methyl-biphenyl-4-yl)methylamine, TBTU and N-ethyl-diisopropylamine in dimethylformamide.

Yield: 96% of theory

R_{f} value: 0.80 (silica gel; dichloromethane/ethanol=9:1)

C₃₅H₂₈F₃N₃O₂ (579.62)

Mass spectrum: (M+H)⁺=580

(M+Na)⁺=602

### Example 96

### N-(4-dimethylaminobutyl)-4-(4′-trifluoromethylbiphenyl-2-carbonylamino)-1-methyl-pyrrole-2-carboxylic acid amide

Prepared analogously to Example 1d from 1-amino-4-(dimethylamino)-butane, 4-(4′-trifluoromethylbiphenyl-2-carbonylamino)-1-methyl-pyrrole-2-carboxylic acid, TBTU and triethylamine in dimethylformamide.

Yield: 99% of theory

R_{f} value: 0.17 (silica gel; ethyl acetate/ethanol/ammonia=50:45:5)

C₂₆H₂₉F₃N₄O₂ (486.54)

Mass spectrum: (M−H)⁻=485

(M+H)⁺=487

### Example 97

### N-(4′-methylbiphenyl-4-yl)methyl-4-(4′-trifluoromethylbiphenyl-2-carbonylamino)-1-(2-methoxycarbonyl-ethyl)-pyrrole-2-carboxylic acid amide

Prepared analogously to Example 4a from 4′-trifluoromethylbiphenyl-2-carboxylic acid chloride, N-(4′-methylbiphenyl-4-yl)methyl-4-amino-1-(2-methoxycarbonyl-ethyl)-pyrrole-2-carboxylic acid and triethylamine in tetrahydrofuran.

Yield: 80% of theory

R_{f} value: 0.60 (silica gel; dichloromethane/ethanol=9:1)

C₃₇H₃₂F₃N₃O₄ (639.68)

Mass spectrum: (M+H)+=640

### Example 98

### N-(4-hydroxycarbonylcyclohexylmethyl)-4-(4′-trifluoromethylbiphenyl-2-carbonylamino)-1-methyl-pyrrole-2-carboxylic acid amide

Prepared analogously to Example 1a from N-(4-methoxycarbonylcyclohexylmethyl)-4-(4′-trifluoromethylbiphenyl-2-carbonylamino)-1-methyl-pyrrole-2-carboxylic acid amide and sodium hydroxide solution in methanol.

Yield: 88% of theory

R_{f} value: 0.91 (silica gel; dichloromethane/ethanol=9:1)

C₂₈H₂₈F₃N₃O₄ (527.54)

Mass spectrum: (M−H)⁻=526

(M+H)⁺=528

### Example 99

### N-(4′-methylbiphenyl-4-yl)methyl-4-(4′-trifluoromethylbiphenyl-2-carbonylamino)-1-(2-hydroxycarbonylethyl)-pyrrole-2-carboxylic acid amide

Prepared analogously to Example 1e from N-(4′-methylbiphenyl-4-yl)methyl-4-(4′-trifluoromethylbiphenyl-2-carbonylamino)-1-(2-methoxycarbonylethyl)-pyrrole-2-carboxylic acid amide and sodium hydroxide solution in methanol.

Yield: 62% of theory

R_{f} value: 0.30 (silica gel; dichloromethane/ethanol=9:1)

C₃₆H₃₀F₃N₃O₄ (625.65)

Mass spectrum: (M−H)−=624

(M+H)⁺=626

(M+Na)⁺=648

### Example 100

### 1-methyl-2-[4-(piperidin-1-yl)methyl-piperidinocarbonyl]-4-(4′-trifluoromethylbiphenyl-2-carbonylamino)-pyrrole

Prepared analogously to Example 1d from 4-(piperidin-1-yl)methyl-piperidine, 4-(4′-trifluoromethylbiphenyl-2-carbonylamino)-1-methyl-pyrrole-2-carboxylic acid, TBTU and triethylamine in dimethylformamide.

Yield: 96% of theory

R_{f} value: 0.29 (silica gel; dichloromethane/ethanol=4:1)

C₃₁H₃₅F₃N₄O₂ (552.64)

Mass spectrum: (M−H)⁻=551

(M+H)⁺=553

### Example 101

### 2-[4-(N-acetyl-N-methyl-aminomethyl)piperidinocarbonyl]-1-methyl-4-(4′-trifluoromethylbiphenyl-2-carbonylamino)-pyrrole

Prepared analogously to Example 1d from N-methyl-N-(piperidin-4-yl)methyl-acetamide, 4-(4′-trifluoromethylbiphenyl-2-carbonylamino)-1-methyl-pyrrole-2-carboxylic acid, TBTU and triethylamine in dimethylformamide.

Yield: quantitative

C₂₉H₃₁F₃N₄O₃ (540.59)

Mass spectrum: (M−H)⁻=539

(M+H)⁺=541

### Example 102

### 2-[7-(4-cyano-phenoxy)-1,2,3,4-tetrahydroisoquinolin-2-ylcarbonyl]-1-methyl-4-(4′-trifluoromethylbiphenyl-2-carbonylamino)-pyrrole

Prepared analogously to Example 1d from 7-(4-cyanophenoxy)-1,2,3,4-tetrahydroisoquinoline, 4-(4′-trifluoromethylbiphenyl-2-carbonylamino)-1-methyl-pyrrole-2-carboxylic acid, TBTU and triethylamine in dimethylformamide.

Yield: 96% of theory

R_{f} value: 0.85 (silica gel; dichloromethane/ethanol=9:1)

C₃₆H₂₇F₃N₄O₃ (620.63)

Mass spectrum: (M−H)⁻=619

(M+H)+=621

### Example 103

### N-(4′-methylbiphenyl-4-yl)methyl-4-(4′-trifluoromethylbiphenyl-2-carbonylamino)-1-isopropyl-pyrrole-2-carboxylic acid amide

### a. ethyl 1-isopropyl-4-nitro-pyrrole-2-carboxylate

0.5 g (2.7 mmol) of ethyl 4-nitropyrrole-2-carboxylate are dissolved in 8 ml of dimethylformamide and after batchwise addition of 73 mg (3 mmol) of sodium hydride stirred for another 45 minutes. Then 0.29 ml (2.9 mmol) of isopropyl iodide are added and the mixture is stirred for 12 hours. The reaction mixture is diluted with water and extracted with dichloromethane. The combined organic extracts are dried and concentrated by evaporation. The residue is chromatographed on silica gel, eluting with dichloromethane.

Yield: 0.32 g (49% of theory)

R_{f} value: 0.7 (silica gel; dichloromethane/ethanol=99:1)

### b. ethyl 4-amino-1-isopropyl-pyrrole-2-carboxylate

0.32 g (1.4 mmol) of ethyl 1-isopropyl-4-nitro-pyrrole-2-carboxylate are dissolved in 30 ml of ethanol and, after the addition of 0.15 g of 10% palladium on activated charcoal, hydrogenated with hydrogen at ambient temperature. The catalyst is filtered off and the solution is concentrated by evaporation.

Yield: 0.26 g (94% of theory)

R_{f} value: 0.15 (silica gel; dichloromethane/ethanol=99:1)

### c. ethyl 4-(4′-trifluoromethylbiphenyl-2-carbonylamino)-1-isopropyl-pyrrole-2-carboxylate

Prepared analogously to Example 4a from 4′-trifluoromethylbiphenyl-2-carboxylic acid chloride, ethyl 4-amino-1-isopropyl-pyrrole-2-carboxylate and triethylamine in tetrahydrofuran.

Yield: 65% of theory

R_{f} value: 0.75 (silica gel; dichloromethane/ethanol=19:1)

### d. 4-(4′-trifluoromethylbiphenyl-2-carbonylamino)-1-isopropyl-pyrrole-2-carboxylic acid

Prepared analogously to Example 1e from ethyl 4-(4′-trifluoromethylbiphenyl-2-carbonylamino)-1-isopropyl-pyrrole-2-carboxylate and sodium hydroxide solution in methanol.

Yield: 80% of theory

R_{f} value: 0.4 (silica gel; dichloromethane/ethanol=19:1)

### e. N-(4′-methylbiphenyl-4-yl)methyl-4-(4′-trifluoromethylbiphenyl-2-carbonylamino)-1-isopropyl-pyrrole-2-carboxylic acid amide

Prepared analogously to Example 1d from (4′-methylbiphenyl-4-yl)-methylamine, 4-(4′-trifluoromethylbiphenyl-2-carbonylamino)-1-isopropyl-pyrrole-2-carboxylic acid, TBTU and N-ethyl-diisopropylamine in dimethylformamide.

Yield: 94% of theory

R_{f} value: 0.75 (silica gel; dichloromethane/ethanol=9:1)

C₃₆H₃₂F₃N₃O₂ (595.67)

Mass spectrum: (M−H)⁻=594

(M+H)+=596

### Example 104

### N-[3-(biphenyl-4-yl)-propyl]-4-(4′-trifluoromethylbiphenyl-2-carbonylamino)-1-methyl-imidazole-2-carboxylic acid amide

Prepared analogously to Example 103b from N-[3-(4-biphenyl)-prop-2-ynyl]-4-(4′-trifluoromethylbiphenyl-2-carbonylamino)-1-methyl-imidazole-2-carboxylic acid amide and 10% palladium on activated charcoal in ethanol.

Yield: 99% of theory

R_{f} value: 0.5 (silica gel; petroleum ether/ethyl acetate=1:1)

C₃₄H₂₉F₃N₄O₂ (582.63)

Mass spectrum: (M−H)⁻=581

(M+H)+=583

### Example 105

### N-(cyclohexylmethyl)-4-(4′-trifluoromethylbiphenyl-2-carbonylamino)-1-methyl-pyrrole-2-carboxylic acid amide

Prepared analogously to Example 1d from (aminomethyl)-cyclohexane, 4-(4′-trifluoromethylbiphenyl-2-carbonylamino)-1-methyl-pyrrole-2-carboxylic acid, TBTU and triethylamine in dimethylformamide.

Yield: 99% of theory

R_{f} value: 0.7 (silica gel; dichloromethane/ethanol=9:1)

C₂₇H₂₈F₃N₃O₂ (483.53)

Mass spectrum: (M−H)⁻=482

(M+H)+=484

### Example 106

### N-(4′-methylbiphenyl-4-yl)methyl-4-(2-phenoxyphenyl-carbonylamino)-1-methyl-pyrrole-2-carboxylic acid amide

Prepared analogously to Example 1d from 2-phenoxybenzoic acid, N-(4′-methylbiphenyl-4-yl)methyl-4-amino-1-methyl-pyrrole-2-carboxylic acid amide, TBTU and N-ethyl-diisopropylamine in dimethylformamide.

Yield: quantitative

R_{f} value: 0.4 (silica gel; dichloromethane/ethanol=19:1)

C₃₃H₂₉N₃O₃ (515.61)

Mass spectrum: (M+H)⁺=516

(M+HCOO)−=560

### Example 107

### N-(4′-methylbiphenyl-4-yl)methyl-4-[2-(2-phenylethyl)phenyl-carbonylamino]-1-methyl-pyrrole-2-carboxylic acid amide

Prepared analogously to Example 1d from 2-(2-phenylethyl)benzoic acid, N-(4′-methylbiphenyl-4-yl)methyl-4-amino-1-methyl-pyrrole-2-carboxylic acid amide, TBTU and N-ethyl-diisopropylamine in dimethylformamide.

Yield: quantitative

R_{f} value: 0.5 (silica gel; dichloromethane/ethanol=19:1)

C₃₅H₃₃N₃O₂ (527.67)

Mass spectrum: (M−H)−=526

(M+H)+=528

### Example 108

### N-[4-(tert.butoxycarbonylaminomethyl)-phenylmethyl]-4-(4′-trifluoromethyl-biphenyl-2-carbonylamino)-1-methyl-pyrrole-2-carboxylic acid amide

Prepared analogously to Example 1d from 4-tert.butoxycarbonylaminomethyl-benzylamine, 4-(4′-trifluoromethylbiphenyl-2-carbonylamino)-1-methyl-pyrrole-2-carboxylic acid, TBTU and triethylamine in dimethylformamide.

Yield: 96% of theory

R_{f} value: 0.67 (silica gel; dichloromethane/ethanol=9:1)

C₃₃H₃₃F₃N₄O₄ (606.65)

Mass spectrum: (M−H)−=605

(M+Na)+=629

### Example 109

### N-(4-aminomethyl)phenylmethyl-4-(4′-trifluoromethyl-biphenyl-2-carbonylamino)-1-methyl-pyrrole-2-carboxylic acid amide

Prepared analogously to Example 19c from N-[4-(tert.butoxycarbonylaminomethyl)-phenylmethyl]-4-(4′-trifluoromethyl-biphenyl-2-carbonylamino)-1-methyl-pyrrole-2-carboxylic acid amide and trifluoroacetic acid in dichloromethane.

Yield: quantitative

R_{f} value: 0.7 (silica gel; dichloromethane/ethanol=4:1)

C₂₈H₂₅F₃N₄O₂ (506.53)

Mass spectrum: (M−H)−=505

(M+H)+=507

### Example 110

### N-(4′-methylbiphenyl-4-yl)methyl-4-[3-methyl-2-(piperidin-1-yl)-phenyl-carbonylamino]-1-methyl-pyrrole-2-carboxylic acid amide

Prepared analogously to Example 1d from 3-methyl-2-(piperidin-1-yl)-benzoic acid, N-(4′-methylbiphenyl-4-yl)methyl-4-amino-1-methyl-pyrrole-2-carboxylic acid amide, TBTU and triethylamine in dimethylformamide.

Yield: 66% of theory

R_{f} value: 0.4 (silica gel; dichloromethane/ethanol=4:1)

C₃₃H₃₆N₄O₂ (520.68)

Mass spectrum: (M+H)+=521

### Example 111

### N-(4′-methylbiphenyl-4-yl)methyl-4-[2-(benzylamino)-phenyl-carbonylamino]-1-methyl-pyrrole-2-carboxylic acid amide

Prepared analogously to Example 1d from N-benzylanthranilic acid, N-(4′-methylbiphenyl-4-yl)methyl-4-amino-1-methyl-pyrrole-2-carboxylic acid amide, TBTU and triethylamine in dimethylformamide.

Yield: 74% of theory

R_{f} value: 0.44 (silica gel; dichloromethane/ethanol=9:1)

C₃₄H₃₂N₄O₂ (528.65)

Mass spectrum: (M−H)−=527

(M+H)+=529

### Example 112

### N-(4′-methylbiphenyl-4-yl)methyl-4-[2-(4-methyl-phenylsulphonylamino)-phenylcarbonylamino]-1-methyl-pyrrole-2-carboxylic acid amide

Prepared analogously to Example 1d from 2-(4-methyl-phenylsulphonylamino)-benzoic acid, N-(4′-methylbiphenyl-4-yl)methyl-4-amino-1-methyl-pyrrole-2-carboxylic acid amide, TBTU and triethylamine in dimethylformamide.

Yield: 5% of theory

R_{f} value: 0.65 (silica gel; dichloromethane/ethanol=9:1)

C₃₄H₃₂N₄O₄S (592.72)

Mass spectrum: (M−H)−=591

### Example 113

### N-[4-(4-propylpiperidino)-phenylmethyl]-4-(4′-trifluoromethylbiphenyl-2-carbonylamino)-1-methyl-pyrrole-2-carboxylic acid-amide

Prepared analogously to Example 1d from 4-(4′-trifluoromethyl-biphenyl-2-carbonylamino)-1-methyl-pyrrole-2-carboxylic acid, 4-(4-propylpiperidino)-benzylamine, TBTU and triethylamine in tetrahydrofuran.

Yield:100% of theory

R_{f} value: 0.80 (silica gel; dichloromethane/ethanol=9:1)

C₃₅H₃₇F₃N₄O₂ (602.71)

Mass spectrum: (M+H)⁺=603

### Example 114

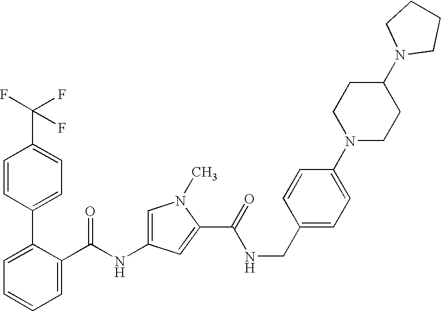

### N-{4-[4-(pyrrolidin-1-yl)-piperidino]-phenylmethyl}-4-(4′-trifluoromethylbiphenyl-2-carbonyl-amino)-1-methyl-pyrrole-2-carboxylic acid-amide

Can be prepared analogously to Example 1d from 4-(4′-trifluoromethyl-biphenyl-2-carbonylamino)-1-methyl-pyrrole-2-carboxylic acid, 4-[4-(pyrrolidin-1-yl)-piperidino]-benzylamine, TBTU and triethylamine in tetrahydrofuran.

### Example 115

### N-[4-(4-phenylpiperidino)-phenylmethyl]-4-(4′-trifluoromethylbiphenyl-2-carbonylamino)-1-methyl-pyrrole-2-carboxylic acid-amide

Can be prepared analogously to Example 1d from 4-(4′-trifluoromethyl-biphenyl-2-carbonylamino)-1-methyl-pyrrole-2-carboxylic acid, 4-(4-phenylpiperidino)-benzylamine, TBTU and triethylamine in tetrahydrofuran.

### Example 116

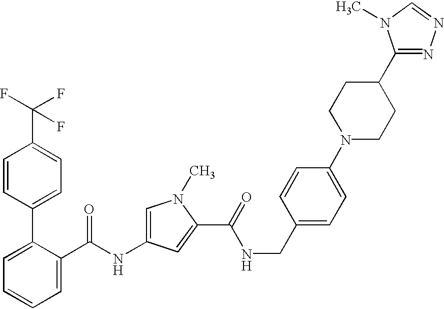

### N-{4-[4-(4-methyl-4-H-[1,2,4]triazol-3-yl)-piperidino]-phenylmethyl}-4-(4′-trifluoromethylbiphenyl-2-carbonyl-amino)-1-methyl-pyrrole-2-carboxylic acid-amide

Can be prepared analogously to Example 1d from 4-(4′-trifluoromethyl-biphenyl-2-carbonylamino)-1-methyl-pyrrole-2-carboxylic acid, 4-[4-(4-methyl-4-H-[1,2,4]triazol-3-yl)-piperidino]-benzylamine, TBTU and triethylamine in tetrahydrofuran.

### Example 117

### N-[4-(4,4-dimethylpiperidino)-phenylmethyl]-4-(4′-trifluoromethylbiphenyl-2-carbonylamino)-1-methyl-pyrrole-2-carboxylic acid-amide

Can be prepared analogously to Example 1d from 4-(4′-trifluoromethyl-biphenyl-2-carbonylamino)-1-methyl-pyrrole-2-carboxylic acid, 4-(4,4-dimethylpiperidino)-benzylamine, TBTU and triethylamine in tetrahydrofuran.

### Example 118

### N-{4-[4-(4-methylphenyl)piperidino]-phenylmethyl}-4-(4′-trifluoromethylbiphenyl-2-carbonyl-amino)-1-methyl-pyrrole-2-carboxylic acid-amide

Can be prepared analogously to Example 1d from 4-(4′-trifluoromethyl-biphenyl-2-carbonylamino)-1-methyl-pyrrole-2-carboxylic acid, 4-[4-(4-methylphenyl)piperidino]-benzylamine, TBTU and triethylamine in tetrahydrofuran.

### Example 119

### (S)-N-[1-(naphth-2-yl)-ethyl]-4-(4′-trifluoromethylbiphenyl-2-carbonylamino)-1-methyl-pyrrole-2-carboxylic acid amide

Can be prepared analogously to Example 1d from 4-(4′-trifluoromethyl-biphenyl-2-carbonylamino)-1-methyl-pyrrole-2-carboxylic acid, (S)-1-(naphth-2-yl)-ethylamine, TBTU and triethylamine in tetrahydrofuran.

### Example 120

### (R)-N-[1-(naphth-2-yl)-ethyl]-4-(4′-trifluoromethylbiphenyl-2-carbonylamino)-1-methyl-pyrrole-2-carboxylic acid amide

Prepared analogously to Example 1d from 4-(4′-trifluoromethyl-biphenyl-2-carbonylamino)-1-methyl-pyrrole-2-carboxylic acid, (R)-1-(naphth-2-yl)-ethylamine, TBTU and triethylamine in tetrahydrofuran.

Yield: 98% of theory

R_{f} value: 0.79 (silica gel; dichloromethane/ethanol=9:1)

C₃₂H₂₆ClF₃N₃O₂ (541.58)

Mass spectrum: (M−H)⁻=540

(M+H)⁺=542

(M+HCOO)=586

### Example 121 (Corresponds to Enantiomerically Pure Example 80)

### (S)-N-[1-(4-chlorophenyl)-ethyl]-4-(4′-trifluoromethylbiphenyl-2-carbonylamino)-1-methyl-pyrrole-2-carboxylic acid amide

Prepared analogously to Example 1d from 4-(4′-trifluoromethyl-biphenyl-2-carbonylamino)-1-methyl-pyrrole-2-carboxylic acid, (R)-1-(4-chlorophenyl)-ethylamine, TBTU and triethylamine in tetrahydrofuran.

Yield: 77% of theory

R_{f} value: 0.83 (silica gel; dichloromethane/ethanol=9:1)

C₂₈H₂₃ClF₃N₃O₂ (525.96)

Mass spectrum: (M−H)⁻=524/26 (chlorine isotope)

(M+H)⁺=526/28 (chlorine isotope)

### Example 122 (Corresponds to Enantiomerically Pure Example 80)

### (R)-N-[1-(4-chlorophenyl)-ethyl]-4-(4′-trifluoromethylbiphenyl-2-carbonylamino)-1-methyl-pyrrole-2-carboxylic acid amide

Prepared analogously to Example 1d from 4-(4′-trifluoromethyl-biphenyl-2-carbonylamino)-1-methyl-pyrrole-2-carboxylic acid, (S)-1-(4-chlorophenyl)-ethylamine, TBTU and triethylamine in tetrahydrofuran.

Yield: 56% of theory

R_{f} value: 0.82 (silica gel; dichloromethane/ethanol=9:1)

C₂₈H₂₃ClF₃N₃O₂ (525.96)

Mass spectrum: (M−H)⁻=524/26 (chlorine isotope)

(M+H)⁺=526/28 (chlorine isotope)

### Example 123

Tablets containing 5 mg of active substance per tablet

| Composition: | |
|---|---|
| active substance | 5.0 mg |
| lactose monohydrate | 70.8 mg |
| microcrystalline cellulose | 40.0 mg |
| sodium carboxymethylcellulose, | 3.0 mg |
| insolubly crosslinked | |
| magnesium stearate | 1.2 mg |

### Preparation

The active substance is mixed for 15 minutes with lactose monohydrate, microcrystalline cellulose and sodium carboxymethylcellulose in a suitable diffusion mixer. Magnesium stearate is added and mixed with the other substances for another 3 minutes.

The finished mixture is compressed in a tablet press to form facetted flat round tablets.

Diameter of the tablet: 7 mm

Weight of a tablet: 120 mg

### Example 124

### Capsules Containing 50 mg of Active Substance Per Capsule

| Composition: | |
|---|---|
| active substance | 50.0 mg |
| lactose monohydrate | 130.0 mg |
| corn starch | 65.0 mg |
| highly dispersed silicon dioxide | 2.5 mg |
| magnesium stearate | 2.5 mg |

### Preparation

A starch paste is prepared by swelling some of the corn starch in a suitable amount of hot water. The paste is then left to cool to room temperature.

The active substance is premixed for 15 minutes in a suitable mixer with lactose monohydrate and corn starch. The starch paste is added and the mixture is mixed with sufficient water to produce a moist homogeneous mass. The moist mass is passed through a screen with a mesh size of 1.6 mm. The screened granules are dried on racks at about 55° C. for 12 hours.

The dried granules are then passed through screens with mesh sizes of 1.2 and 0.8 mm. Highly dispersed silica is mixed with the granules in a suitable mixer for 3 minutes. Then magnesium stearate is added and mixing is continued for another 3 minutes.

The finished mixture is packed into empty size 1 hard gelatine capsule shells using a capsule filling machine.

### Example 125

### Tablets Containing 200 mg of Active Substance Per Tablet

| Composition: | |
|---|---|
| active substance | 200.0 mg |
| lactose-monohydrate | 167.0 mg |
| microcrystalline cellulose | 80.0 mg |
| hydroxypropyl-methylcellulose, type 2910 | 10.0 mg |
| poly-1-vinyl-2-pyrrolidone, insolubly crosslinked | 20.0 mg |
| magnesium stearate | 3.0 mg |

### Preparation

HPMC is dispersed in hot water. After cooling, the mixture yields a clear solution.

The active substance is premixed in a suitable mixer for 5 minutes with lactose monohydrate and microcrystalline cellulose. The HPMC solution is added and the mixing is continued until a homogeneous moist composition is obtained. The moist composition is passed through a screen with a mesh size of 1.6 mm. The screened granules are dried on racks at about 55° C. for 12 hours.

The dried granules are then passed through screens with mesh sizes of 1.2 and 0.8 mm. Poly-1-vinyl-2-pyrrolidone is mixed with the granules in a suitable mixer for 3 minutes. Then magnesium stearate is added and mixing is continued for another 3 minutes.

The finished mixture is compressed in a tablet press to form oblong tablets (16.2×7.9 mm).

Weight of a tablet: 480 mg

We claim:

1. A method for lowering the liver toxicity of MTP inhibitors comprising the step of administering to a mammal a therapeutically effective amount of a fibrate.

2. The method according to claim 1, wherein the fibrate is selected from the list consisting of:
bezafibrate,
ciprofibrate,
clofibrate,
fenofibrate and
gemfibrozil.

3. The method according to claim 1, wherein the MTP inhibitor is a compound of general formula I 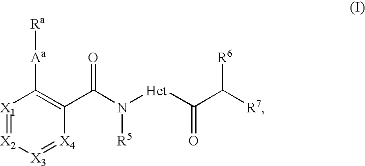
wherein
X₁ denotes the group CR¹,
X₂ denotes the group CR²,
X₃ denotes the group CR³ and
X₄ denotes the group CR⁴ or
one or two of the groups X₁ to X₄ in each case denote a nitrogen atom and the remainder of the groups X₁ to X₄ denote three or two of the groups CR¹ to CR⁴,
while R¹, R², R³ and R⁴ in each case denote a hydrogen atom or
one or two of the groups R¹ to R⁴ independently of one another in each case denote a fluorine, chlorine or bromine atom, a C₁₋₃-alkyl group, a trifluoromethyl, hydroxy, C₁₋₃-alkoxy, trifluoromethoxy, amino, C₁₋₃-alkylamino or di-(C₁₋₃-alkyl)-amino group and the remainder of the groups R¹ to R⁴ in each case represent a hydrogen atom,
while R⁴ additionally together with R⁵ may assume the meaning of a —(CH₂)ₙ— bridge wherein n denotes the number 1, 2 or 3, and
A^{a} denotes a bond, an oxygen or sulphur atom, an —NH, —N(C₁₋₃-alkyl), sulphinyl, sulphonyl or carbonyl group,
one of the groups —CH₂—, —(CH₂)₂—, —CH═CH—, —C≡C—, —OCH₂—, —CH₂O—, —NH—CH₂—, —CH₂—NH—, —NH—CO—, —CO—NH—, —NH—SO₂— or —SO₂—NH—,
wherein a hydrogen atom bound to a carbon atom and/or a hydrogen atom bound to a nitrogen atom may be replaced in each case by a C₁₋₃-alkyl group and wherein a heteroatom of the group A^{a} is not linked to a nitrogen atom of a 5-membered heteroaryl group of the group R^{a},
R^{a} denotes a phenyl, 1-naphthyl or 2-naphthyl group,
a 5-membered heteroaryl group bound via a carbon or nitrogen atom, which contains
an imino group optionally substituted by a C₁₋₄-alkyl or C₁₋₄-alkylcarbonyl group, an oxygen or sulphur atom,
an imino group optionally substituted by a C₁₋₄-alkyl group or an oxygen or sulphur atom and additionally a nitrogen atom or
an imino group optionally substituted by a C₁₋₄-alkyl group and two nitrogen atoms or
an oxygen or sulphur atom and two nitrogen atoms,
a 6-membered heteroaryl group which contains one or two nitrogen atoms,
while a phenyl ring may be fused to the abovementioned 5- or 6-membered heteroaryl groups via two adjacent carbon atoms and the bicyclic heteroaryl groups thus formed may be bound via the heteroaromatic or carbocyclic moiety and
wherein the abovementioned phenyl and naphthyl groups as well as the mono- and bicyclic heteroaryl groups in the carbon skeleton may be monosubstituted by a fluorine, chlorine or bromine atom, by a C₁₋₄-alkyl group, by a C₃₋₇-cycloalkyl, trifluoromethyl, phenyl, hydroxy, C₁₋₃-alkoxy, trifluoromethoxy, amino, C₁₋₃-alkylamino, di-(C₁₋₃-alkyl)amino, acetylamino, N—(C₁₋₃-alkyl)-acetylamino, propionylamino, N—(C₁₋₃-alkyl)-propionylamino, acetyl, propionyl, C₁₋₃-alkoxy-carbonyl, aminocarbonyl, C₁₋₃-alkylamino-carbonyl, di-(C₁₋₃-alkyl)amino-carbonyl or cyano group or, with the exception of 5-membered heteroaryl groups containing more than two heteroatoms, may also be disubstituted by the abovementioned substituents, while the substituents may be identical or different,
a C₃₋₇-cycloalkyl group, wherein
in each case the methylene group in the 4 position of a 6- or 7-membered cycloalkyl group may be replaced by an oxygen or sulphur atom, by a sulphinyl or sulphonyl group or by an imino group optionally substituted by a C₁₋₅-alkyl, phenyl, C₁₋₄-alkyl-carbonyl, C₁₋₄-alkoxy-carbonyl, C₁₋₃-alkyl-aminocarbonyl or di-(C₁₋₃-alkyl)-aminocarbonyl group,
a 4- to 7-membered cycloalkyleneimino group wherein
the cycloalkylene moiety may be fused to a phenyl ring or
one or two hydrogen atoms may be replaced in each case by a C₁₋₃-alkyl group and/or
in each case the methylene group in the 4 position of a 6- or 7-membered cycloalkyleneimino group may be substituted by a hydroxycarbonyl, C₁₋₃-alkoxycarbonyl, amino, C₁₋₃-alkylamino, di-(C₁₋₃-alkyl)amino, aminocarbonyl, C₁₋₃-alkylamino-carbonyl, di-(C₁₋₃-alkyl)-aminocarbonyl or phenyl-C₁₋₃-alkylamino group or
may be replaced by an oxygen or sulphur atom, by a sulphinyl or sulphonyl group or by an imino group optionally substituted by a C₁₋₅-alkyl, phenyl, C₁₋₄-alkyl-carbonyl, C₁₋₄-alkoxy-carbonyl, C₁₋₃-alkyl-aminocarbonyl or di-(C₁₋₃-alkyl)-aminocarbonyl group or
in a 5-, 6- or 7-membered cycloalkyleneimino group a —CH₂— group linked to the imino nitrogen atom may be replaced by a carbonyl group or a —(CH₂)₂— group linked to the imino nitrogen atom may be replaced by a —CO—NR⁸— group or a —(CH₂)₃— group linked to the imino nitrogen atom may be replaced by a —CO—NR⁸—CO— group,
while R⁸ denotes a hydrogen atom or a C₁₋₃-alkyl group,
R⁵ denotes a hydrogen atom or a C₁₋₅-alkyl group,
Het denotes a 5-membered heteroarylene group bound via two carbon atoms or, if Het denotes a double-bonded pyrrole group, it may also be bound via a carbon atom and the imino-nitrogen atom, the latter being linked to the adjacent carbonyl group in formula (I), which contains
an imino group substituted by the group R⁹, an oxygen or sulphur atom or
an imino group substituted by the group R⁹ or an oxygen or sulphur atom and additionally a nitrogen atom,
while R⁹ denotes a hydrogen atom, a C₁₋₅-alkyl group, a C₂₋₃-alkyl group terminally substituted by an amino, C₁₋₃-alkylamino, di-(C₁₋₃-alkyl)-amino or C₁₋₅-alkoxy-carbonyl-amino group, a carboxy-C₁₋₃-alkyl, C₁₋₃-alkoxy-carbonyl-C₁₋₃-alkyl, phenyl, phenyl-C₁₋₃-alkyl, C₁₋₅-alkylcarbonyl or phenylcarbonyl group or R⁹ together with R⁶ denotes a —(CH₂)ₚ— bridge, wherein p denotes the number 2 or 3,
or an imino group optionally substituted by a C₁₋₃-alkyl group and two nitrogen atoms or
an oxygen or sulphur atom and two nitrogen atoms,
or a 6-membered heteroarylene group which contains one or two nitrogen atoms,
while the abovementioned heteroarylene groups in the carbon skeleton may be monosubstituted by a fluorine, chlorine or bromine atom, by a C₁₋₅-alkyl group, by a C₃₋₇-cycloalkyl, trifluoromethyl, hydroxy, C₁₋₃-alkoxy, trifluoromethoxy, amino, C₁₋₃-alkylamino, di-(C₁₋₃-alkyl)amino, acetylamino, N—(C₁₋₃-alkyl)-acetylamino, propionylamino, N—(C₁₋₃-alkyl)-propionylamino, acetyl, propionyl, benzoyl, C₁₋₃-alkoxy-carbonyl, aminocarbonyl, C₁₋₃-alkylamino-carbonyl-di-(C₁₋₃-alkyl)-amino-carbonyl or cyano group or, with the exception of 5-membered monocyclic heteroaryl groups containing more than one heteroatom, may also be disubstituted by the abovementioned substituents, while the substituents may be identical or different,
R⁶ denotes a hydrogen atom or a C₁₋₆-alkyl group,
R⁷ denotes a C₁₋₉-alkyl group,
a straight-chain or branched, mono-, di- or triunsaturated C₃₋₉-alkenyl or C₃₋₉-alkynyl group, while the multiple bonds are isolated from the nitrogen-carbon bond,
a straight-chain C₂₋₆-alkyl group which is terminally substituted by an amino, C₁₋₃-alkylamino or di-(C₁₋₃-alkyl)-amino group,
a C₁₋₆-alkyl group substituted by a C₃₋₇-cycloalkyl group , while
a hydrogen atom in the 3 position of the cyclopentyl group and in the 4 position of a 6- or 7-membered cycloalkyl group may be replaced in each case by a hydroxy, hydroxy-C₁₋₃-alkyl, C₁₋₅-alkoxy, C₁₋₅-alkoxy-C₁₋₃-alkyl, phenyl-C₁₋₃-alkoxy-C₁₋₃-alkyl, amino, C₁₋₅-alkylamino, di-(C₁₋₅-alkyl)amino, phenyl-C₁₋₃-alkylamino, C₁₋₅-alkyl-carbonylamino, benzoylamino, amino-C₁₋₃-alkyl, C₁₋₃-alkylamino-C₁₋₃-alkyl, di-(C₁₋₃-alkyl)amino-C₁₋₃-alkyl, phenyl-C₁₋₃-alkylamino-C₁₋₃-alkyl, C₁₋₃-alkyl-carbonylamino-C₁₋₃-alkyl, benzoylamino-C₁₋₃-alkyl, phenylamino-carbonyl, phenyl-C₁₋₃-alkylamino-carbonyl, carboxy or C₁₋₃-alkoxy-carbonyl group or
in each case the methylene group in the 4 position of a 6- or 7-membered cycloalkyl group may be replaced by an oxygen or sulphur atom or by an imino group optionally substituted by a C₁₋₆-alkyl, phenyl, C₁₋₆-alkyl-carbonyl, benzoyl,
phenyl-(C₁₋₃-alkyl)-carbonyl, C₁₋₆-alkyl-aminocarbonyl,
di-(C₁₋₅-alkyl)-aminocarbonyl, phenylaminocarbonyl,
N—(C₁₋₃-alkyl)-phenylaminocarbonyl, phenyl-C₁₋₃-alkylamino-carbonyl or
N—(C₁₋₃-alkyl)-phenyl-C₁₋₃-alkylamino-carbonyl group or
in a 5- or 6-membered cycloalkyl group one or two single bonds separated from each other by at least one bond and separated from position 1 may in each case be fused to a phenyl group, while in a bi-or tricyclic ring system thus formed the hydrogen atom bound to the saturated carbon atom in position 1 may be replaced by a C₁₋₅-alkylamino-carbonyl, di-(C₁₋₅-alkyl)amino-carbonyl, phenyl-C₁₋₃-alkylamino-carbonyl or C₁₋₅-alkoxy-carbonyl group, wherein terminal methyl groups in each case may be wholly or partially fluorinated,
a C₁₋₆-alkyl group optionally substituted by a C₃₋₇-cycloalkyl group, which is substituted
by a carboxy or C₁₋₃-alkoxycarbonyl group,
by a phenyl, 1-naphthyl or 2-naphthyl group,
by a 5-membered heteroaryl group bound via a carbon or nitrogen atom, which contains
an imino group optionally substituted by a C₁₋₃-alkyl, trifluoromethyl, phenyl, phenyl-C₁₋₃-alkyl, C₁₋₃-alkylcarbonyl, phenylcarbonyl or phenyl-C₁₋₃-alkylcarbonyl group, an oxygen or sulphur atom,
an imino group optionally substituted by a C₁₋₃-alkyl group or an oxygen or sulphur atom and additionally a nitrogen atom or
an imino group optionally substituted by a C₁₋₃-alkyl group and two nitrogen atoms or
an oxygen or sulphur atom and two nitrogen atoms,
by a 6-membered heteroaryl group, which contains one or two nitrogen atoms,
while a phenyl ring may be fused to the abovementioned 5- or 6-membered heteroaryl groups via two adjacent carbon atoms and the bicyclic heteroaryl groups thus formed may be bound via the heteroaromatic or carbocyclic moiety,
while the abovementioned phenyl and naphthyl groups as well as the mono- and bicyclic heteroaryl groups in the carbon skeleton may be monosubstituted by a fluorine, chlorine or bromine atom, by a C₁₋₅-alkyl, trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, amino-C₁₋₃-alkyl, C₁₋₃-alkylamino-C₁₋₃-alkyl, di-(C₁₋₃-alkyl)amino-C₁₋₃-alkyl, C₁₋₅-alkoxy-carbonylamino-C₁₋₃-alkyl, acetylamino, propionylamino, N—(C₁₋₃-alkyl)-benzoylamino, acetyl, propionyl, carboxy, C₁₋₃-alkoxy-carbonyl, C₁₋₃-alkoxy-carbonyl-C₁₋₃-alkyl, aminocarbonyl, C₁₋₃-alkylamino-carbonyl, di-(C₁₋₃-alkyl)amino-carbonyl, or cyano group or, with the exception of 5-membered heteroaryl groups containing more than two heteroatoms, may also be disubstituted by the abovementioned substituents, while the substituents may be identical or different,
a C₁₋₆-alkyl group substituted by a phenyl group and a carboxy, C₁₋₃-alkoxy-carbonyl, aminocarbonyl, C₁₋₃-alkyl-aminocarbonyl or di-(C₁₋₃-alkyl)-aminocarbonyl group,
a phenyl-C₂₋₅-alkenylene-CH₂, phenyl-C₂₋₅-alkynylene-CH₂, heteroaryl-C₂₋₅-alkenylene-CH₂ or heteroaryl-C₂₋₅-alkynylene-CH₂ group, wherein a hydrogen atom of the methylene group in position 1 may be replaced by a C₁₋₃-alkyl group and independently thereof the phenyl moiety as well as the heteroaryl moiety may be mono- or disubstituted by fluorine, chlorine or bromine atoms, by C₁₋₆-alkyl, C₃₋₇-cycloalkyl, trifluoromethyl, C₁₋₃-alkoxy, phenyl, heteroaryl or cyano groups, while the substituents may be identical or different and disubstitution by two aromatic groups is excluded,
while heteroaryl denotes a 5-membered heteroaryl group bound via a carbon or nitrogen atom, which contains
an imino group substituted optionally by a C₁₋₃-alkyl group, an oxygen or sulphur atom,
an imino group substituted optionally by a C₁₋₃-alkyl group or an oxygen or sulphur atom and additionally a nitrogen atom or
an imino group substituted optionally by a C₁₋₃-alkyl group and two nitrogen atoms or
an oxygen or sulphur atom and two nitrogen atoms,
or a 6-membered heteroaryl group, which contains one or two nitrogen atoms,
while a phenyl ring may be fused to the abovementioned 5- or 6-membered heteroaryl groups via two adjacent carbon atoms and the bicyclic heteroaryl groups thus formed may be bound via the heteroaromatic or carbocyclic moiety,
the group R^{b}-A^{b}-E^{b}-C₁₋₃-alkyl optionally substituted in the C₁₋₃-alkyl moiety by a C₁₋₄-alkyl or C₃₋₅-cycloalkyl group, wherein
R^{b} denotes a phenyl group optionally mono- or disubstituted by fluorine, chlorine, bromine or iodine atoms, by C₁₋₄-alkyl, C₂₋₄-alkenyl, C₂₋₄-alkynyl, C₃₋₇-cycloalkyl, trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, amino-C₁₋₃-alkyl, C₁₋₃-alkylamino-C₁₋₃-alkyl, di-(C₁₋₃-alkyl)amino-C₁₋₃-alkyl, acetylamino, propionylamino, acetyl, propionyl, carboxy, C₁₋₃-alkoxy-carbonyl, C₁₋₃-alkoxy-carbonyl-C₁₋₃-alkyl, aminocarbonyl, C₁₋₃-alkylamino-carbonyl, di-(C₁₋₃-alkyl)amino-carbonyl or cyano groups, while the substituents may be identical or different,
a 5-membered heteroaryl group which
may be bound via a carbon atom or, if A^{b} denotes a bond, a —CH₂, —(CH₂)₂, sulphonyl or carbonyl group, may also be bound via a nitrogen atom and which contains
an imino group optionally substituted by a C₁₋₃-alkyl group, an oxygen or sulphur atom,
an imino group optionally substituted by a C₁₋₃-alkyl group or an oxygen or sulphur atom and additionally a nitrogen atom or
an imino group optionally substituted by a C₁₋₃-alkyl group and two nitrogen atoms or
an oxygen or sulphur atom and two nitrogen atoms,
a 6-membered heteroaryl group, which contains one or two nitrogen atoms,
while a phenyl ring may be fused to the abovementioned 5- or 6-membered heteroaryl groups via two adjacent carbon atoms and the bicyclic heteroaryl groups thus formed may be bound via the heteroaromatic or carbocyclic moiety, while the abovementioned mono- and bicyclic heteroaryl groups may be monosubstituted in the carbon skeleton by a fluorine, chlorine or bromine atom, by a C₁₋₄-alkyl, C₂₋₄-alkenyl, C₂₋₄-alkynyl, C₃₋₇-cycloalkyl, trifluoromethyl, phenyl, hydroxy, C₁₋₃-alkoxy, trifluoromethoxy, amino, C₁₋₃-alkylamino, di-(C₁₋₃-alkyl)-amino, acetylamino, propionylamino, acetyl, propionyl, C₁₋₃-alkoxy-carbonyl, aminocarbonyl, C₁₋₃-alkylamino-carbonyl, di-(C₁₋₃-alkyl)amino-carbonyl or cyano group or, with the exception of 5-membered heteroaryl groups containing more than two heteroatoms, may also be disubstituted by the abovementioned substituents, while the substituents may be identical or different,
a C₃₋₇-cycloalkyl group wherein
one or two hydrogen atoms in each case may be replaced by a C₁₋₃-alkyl group and/or
in each case the methylene group in the 4 position of a 6- or 7-membered cycloalkyl group may be replaced by an oxygen or sulphur atom, by a sulphinyl, sulphonyl or by an imino group optionally substituted by a C₁₋₃-alkyl, C₁₋₃-alkyl-carbonyl, C₁₋₃-alkoxy-carbonyl, C₁₋₃-alkyl-aminocarbonyl or di-(C₁₋₃-alkyl)-aminocarbonyl group or
the two hydrogen atoms of the methylene group in the 3-position of a cyclopentyl group or in 3- or 4-position of a cyclohexyl or cycloheptyl group may be replaced by an n-butylene, n-pentylene, n-hexylene, 1,2-ethylenedioxy or 1,3-propylenedioxy group and in the rings thus formed one or two hydrogen atoms may be replaced by C₁₋₃-alkyl groups,
a 4- to 7-membered cycloalkyleneimino group wherein
the cycloalkylene moiety may be fused to a phenyl ring or
one or two hydrogen atoms in each case may be replaced by a C₁₋₃-alkyl group and/or
in each case the carbon atom in the 4 position of a 6- or 7-membered cycloalkyleneimino group may be substituted by a hydroxy-C₁₋₃-alkyl, C₁₋₆-alkoxy-C₁₋₃-alkyl, hydroxycarbonyl, C₁₋₆-alkoxycarbonyl, aminocarbonyl, C₁₋₃-alkylamino-carbonyl, di-(C₁₋₃-alkyl)-aminocarbonyl-, 4- to 7-membered cycloalkyleneimino, phenyl, 4-(C₁₋₃-alkyl)-1,2,4-triazol-3-yl, phenyl-C₁₋₃-alkylamino or N—(C₁₋₃-alkyl)-phenyl-C₁₋₃-alkylamino group or
may be replaced by an oxygen or sulphur atom, by a sulphinyl or sulphonyl group or by an imino group optionally substituted by a C₁₋₃-alkyl, phenyl, C₁₋₃-alkyl-carbonyl, benzoyl, phenyl-C₁₋₃-alkyl-carbonyl, C₁₋₃-alkyl-aminocarbonyl, di-(C₁₋₃-alkyl)-aminocarbonyl, phenylaminocarbonyl or N—(C₁₋₃-alkyl)-phenylaminocarbonyl group or
the two hydrogen atoms of the methylene group in the 3 position of a 5-membered cycloalkyleneimino group or in the 3 or 4 position of a 6- or 7-membered cycloalkyleneimino group may be replaced by an n-butylene, n-pentylene, n-hexylene, 1,2-ethylenedioxy or 1,3-propylenedioxy group and in the rings thus formed one or two hydrogen atoms may be replaced by C₁₋₃-alkyl groups or
in a 5-, 6- or 7-membered cycloalkyleneimino group a —CH₂— group linked to the imino nitrogen atom may be replaced by a carbonyl group or a —(CH₂)₂— group linked to the imino nitrogen atom may be replaced by a —CO—NR— group or a —(CH₂)₃— group linked to the imino nitrogen atom may be replaced by a —CO—NR⁸—CO— group,
while R⁸ denotes a hydrogen atom or a C₁₋₃-alkyl group,
A^{b} denotes a bond, an oxygen or sulphur atom, an —NH, —N(C₁₋₃-alkyl), sulphinyl, sulphonyl or a carbonyl group,
one of the groups —CH₂—, —(CH₂)₂—, —O—CH₂—, —CH₂—O—, NH—CH₂—, —CH₂—NH—, —NH—CO—, —CO—NH—, —NH—SO—₂, —SO₂—NH—, —CH═CH— or —C≡C—
wherein a hydrogen atom bound to a carbon atom and/or a hydrogen atom bound to a nitrogen atom may be replaced by a C₁₋₃-alkyl group in each case and a heteroatom of the group A^{b} is not linked to a nitrogen atom of a 5-membered heteroaryl group of the group R^{b},
E^{b} denotes a phenylene group optionally substituted by a fluorine, chlorine or bromine atom, by a C₁₋₄-alkyl group, by a trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, amino-C₁₋₃-alkyl, C₁₋₃-alkylamino-C₁₋₃-alkyl, di-(C₁₋₃-alkyl)amino-C₁₋₃-alkyl, acetylamino, propionylamino, acetyl, propionyl, carboxy, C₁₋₃-alkoxy-carbonyl, C₁₋₃-alkoxy-carbonyl-C₁₋₃-alkyl, aminocarbonyl, C₁₋₃-alkylamino-carbonyl, di-(C₁₋₃-alkyl)amino-carbonyl or cyano group,
the group R^{c}-A^{c}-E^{c}-C₁₋₃-alkyl optionally substituted in the C₁₋₃-alkyl moiety by a C₁₋₄-alkyl or C₃₋₅-cycloalkyl group wherein
R^{c} assumes the meanings given for R^{b} hereinbefore, while any reference to A^{b} must be replaced by a reference to A^{c},
A^{c} assumes the meanings given for A^{b} hereinbefore, while any reference to R^{b} must be replaced by a reference to R^{c},
E^{c} denotes a 5-membered heteroarylene group bound via two carbon atoms or via a carbon atom and an imino-nitrogen atom, while the imino-nitrogen atom of
the heteroarylene group is not linked to a heteroatom of the group A^{c} and the heteroarylene group contains
an imino group optionally substituted by a C₁₋₃-alkyl group, an oxygen or sulphur atom,
an imino group optionally substituted by a C₁₋₃-alkyl group or an oxygen or sulphur atom and additionally a nitrogen atom or
an imino group optionally substituted by a C₁₋₃-alkyl group and two nitrogen atoms or
an oxygen or sulphur atom and two nitrogen atoms,
or a 6-membered heteroarylene group, which contains one or two nitrogen atoms,
while a phenyl ring may be fused to the abovementioned 5-membered heteroarylene groups containing one or two heteroatoms as well as to the abovementioned 6-membered heteroarylene groups via two adjacent carbon atoms and the bicyclic heteroarylene groups thus formed may be bound via the heteroaromatic and/or carbocyclic moiety,
and while the abovementioned mono- and bicyclic heteroarylene groups in the carbon skeleton may be substituted by a fluorine, chlorine or bromine atom, by a C₁₋₄-alkyl group, by a C₃₋₇-cycloalkyl, trifluoromethyl, hydroxy, C₁₋₃-alkoxy, trifluoromethoxy, amino, C₁₋₃-alkylamino, acetylamino, propionylamino, acetyl, propionyl, C₁₋₃-alkoxy-carbonyl, aminocarbonyl, C₁₋₃-alkylamino-carbonyl or cyano group,
or R⁶ and R⁷ together denote an n-alkylene bridge with 3 to 6 carbon atoms, wherein
one or two hydrogen atoms in each case may be replaced by a C₁₋₃-alkyl group and/or
a —CH₂—CH₂— group may be replaced by a 1,2-linked phenylene group which may be mono- or disubstituted by fluorine, chlorine or bromine atoms, by C₁₋₃-alkyl, trifluoromethyl, hydroxy, C₁₋₃-alkoxy, trifluoromethoxy, amino, C₁₋₃-alkylamino, di-(C₁₋₃-alkyl)amino, acetylamino, propionylamino, acetyl, propionyl, C₁₋₃-alkoxy-carbonyl, aminocarbonyl, C₁₋₃-alkylamino-carbonyl, cyano, phenyloxy or phenyl-C₁₋₃-alkyl groups, while disubstitution with the last-named group is excluded,
while the abovementioned phenyloxy- and phenyl-C₁₋₃-alkyl group in the phenyl moiety may in turn be substituted by a fluorine, chlorine or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino or cyano group,
or in each case the carbon atom in the 3 position of a n-pentylene or n-hexylene group may be monosubstituted by a C₁₋₃-alkyl group terminally substituted by a phenyl, cyano, hydroxy, C₁₋₃-alkoxy, amino, C₁₋₃-alkylamino, di-(C₁₋₃-alkyl)-amino or a 5- to 7-membered cycloalkyleneimino group, by a carboxy, C₁₋₃-alkoxycarbonyl, amino-C₁₋₃-alkyl, C₁₋₃-alkylamino-C₁₋₃-alkyl, N—C₁₋₃-alkyl-N—(C₁₋₃-alkyl-carbonyl)-amino-C₁₋₃-alkyl, di-(C₁₋₃-alkyl)-amino-C₁₋₃-alkyl, aminocarbonyl, C₁₋₃-alkylamino-carbonyl or di-(C₁₋₃-alkyl)-aminocarbonyl group or may be disubstituted by a phenyl group and a cyano, hydroxy or C₁₋₃-alkoxy group or
the methylene group in the 3 position of a n-pentylene or n-hexylene group may be replaced by an oxygen or sulphur atom, by a sulphinyl or sulphonyl group or by an imino group optionally substituted by a C₁₋₃-alkyl, phenyl-C₁₋₃-alkyl,
C₁₋₃-alkyl-carbonyl, benzoyl, C₁₋₃-alkyl-aminocarbonyl, di-(C₁₋₃-alkyl)-aminocarbonyl, phenylaminocarbonyl or N—(C₁₋₃-alkyl)-phenylaminocarbonyl group or
a methylene group in position 1 of an n-butylene, n-pentylene or n-hexylene group may be replaced by a carbonyl group,
while the phenyl groups mentioned as being unsubstituted or monosubstituted in the definition of the abovementioned groups as well as aromatic or heteroaromatic parts of molecules may, unless otherwise stated, optionally additionally be substituted in the carbon skeleton by fluorine, chlorine or bromine atoms, by C₁₋₃-alkyl groups, by trifluoromethyl, hydroxy, C₁₋₃-alkoxy, trifluoromethoxy, amino, C₁₋₃-alkylamino, acetylamino, acetyl, C₁₋₃-alkoxy-carbonyl, aminocarbonyl, C₁₋₃-alkylamino-carbonyl or cyano groups, while the substituents may be identical or different and the resulting aromatic groups and parts of molecules may be at most disubstituted,
the hydrogen atoms in the C₁₋₃-alkyl and alkoxy groups mentioned in the definition of the above groups may be wholly or partially replaced by fluorine atoms and
the alkyl and alkoxy groups mentioned in the definition of the above groups or in the alkyl moieties contained in the groups of formula I defined above with more than two carbon atoms may be straight-chain or branched, unless otherwise specified,
the carboxy groups mentioned in the definition of the abovementioned groups may be replaced by a group which can be converted into a carboxy group in vivo or by a group which is negatively charged under physiological conditions, and/or
the amino and imino groups mentioned in the definition of the abovementioned groups may be substituted by a group which can be cleaved in vivo, the tautomers, the diastereomers, the enantiomers, the mixtures and the salts thereof.

4. The method of claim 3, wherein the MTP inhibitor is a compound of general formula I; and
X₁ to X₄ are defined as in claim 3,
A^{a} denotes a bond, an oxygen atom, a —NH—, —N(C₁₋₃-alkyl)-, sulphonyl or carbonyl group,
one of the groups —CH₂—, —(CH₂)₂—, —NH—CH₂—, —CH₂—NH—, —NH—CO—, —CO—NH—, —NH—SO₂— or —SO₂—NH—,
wherein a hydrogen atom bound to a carbon atom and/or a hydrogen atom bound to a nitrogen atom may be replaced in each case by a C₁₋₃-alkyl group and a heteroatom of group A^{a} is not linked to a nitrogen atom of a 5-membered heteroaryl group of the group R^{a},
R^{a} denotes a phenyl group,
a 5-membered heteroaryl group bound via a carbon or nitrogen atom which contains
an imino group optionally substituted by a C₁₋₄-alkyl or C₁₋₄-alkylcarbonyl group, an oxygen or sulphur atom or
an imino group optionally substituted by a C₁₋₄-alkyl group or an oxygen or sulphur atom and additionally a nitrogen atom,
a 6-membered heteroaryl group, which contains one or two nitrogen atoms,
while the abovementioned phenyl and heteroaryl groups may be substituted in the carbon skeleton by a fluorine, chlorine or bromine atom, by a C₁₋₄-alkyl group, by a C₃₋₇-cycloalkyl, trifluoromethyl, phenyl, hydroxy, C₁₋₃-alkoxy, trifluoromethoxy, amino, C₁₋₃-alkylamino, di-(C₁₋₃-alkyl)amino, acetylamino, N—(C₁₋₃-alkyl)-acetylamino, acetyl or cyano group,
a C₃₋₇-cycloalkyl group, wherein
the methylene group in the 4 position of a 6-membered cycloalkyl group may be replaced by an oxygen or sulphur atom, by a sulphonyl group or by an imino group optionally substituted by a C₁₋₃-alkyl, phenyl, C₁₋₄-alkyl-carbonyl or C₁₋₄-alkoxy-carbonyl group,
a 4- to 7-membered cycloalkyleneimino group wherein
one or two hydrogen atoms in each case may be replaced by a C₁₋₃-alkyl group and/or
in each case the methylene group in the 4 position of a 6- or 7-membered cycloalkyleneimino group may be replaced by an oxygen or sulphur atom, by a sulphonyl group or by an imino group optionally substituted by a C₁₋₅-alkyl, phenyl, C₁₋₄-alkyl-carbonyl, C₁₋₄-alkoxy-carbonyl, C₁₋₃-alkyl-aminocarbonyl or di-(C₁₋₃-alkyl)-aminocarbonyl group or
in a 5-, 6- or 7-membered cycloalkyleneimino group a —CH₂— group linked to the imino nitrogen atom may be replaced by a carbonyl group or a —(CH₂)₂— group linked to the imino nitrogen atom may be replaced by a —CO—NR— group or a —(CH₂)₃— group linked to the imino nitrogen atom may be replaced by a —CO—NR⁸—CO— group,
while R⁸ denotes a hydrogen atom or a C₁₋₃-alkyl group,
R⁵ denotes a hydrogen atom or a C₁₋₃-alkyl group,
Het denotes a 5-membered heteroarylene group bound via two carbon atoms which contains
an imino group substituted by the group R⁹, an oxygen or sulphur atom or
an imino group substituted by the group R⁹ or an oxygen or sulphur atom and additionally a nitrogen atom,
while R⁹ denotes a hydrogen atom, a C₁₋₅-alkyl group, a —C₂₋₃-alkyl group terminally substituted by an amino, C₁₋₃-alkylamino, di-(C₁₋₃-alkyl)-amino or C₁₋₅-alkoxy-carbonyl-amino group, a carboxy-C₁₋₃-alkyl, C₁₋₃-alkoxy-carbonyl-C₁₋₃-alkyl, phenyl, phenyl-C₁₋₃-alkyl, C₁₋₅-alkylcarbonyl or phenylcarbonyl group or R⁹ together with R⁶ denotes a —(CH₂)ₚ— bridge wherein p denotes the number 2 or 3,
or an imino group optionally substituted by a C₁₋₃-alkyl group and two nitrogen atoms or
an oxygen or sulphur atom and two nitrogen atoms,
or a 6-membered heteroarylene group, which contains one or two nitrogen atoms,
while the abovementioned heteroarylene groups in the carbon skeleton may be substituted by a fluorine, chlorine or bromine atom, by a C₁₋₃-alkyl group, by a cyclopropyl, trifluoromethyl, C₁₋₃-alkoxy, trifluoromethoxy, C₁₋₃-alkylamino, di-(C₁₋₃-alkyl)amino, acetylamino, N—(C₁₋₃-alkyl)-acetylamino, acetyl, C₁₋₃-alkylamino-carbonyl or di-(C₁₋₃-alkyl)amino-carbonyl group,
R⁶ denotes a hydrogen atom or a C₁₋₄-alkyl group,
R⁷ denotes a C₁₋₆-alkyl group,
a straight-chain C₂₋₆-alkyl group which is terminally substituted by an amino, C₁₋₃-alkylamino or di-(C₁₋₃-alkyl)-amino group,
a C₁₋₆-alkyl group substituted by an C₃₋₇-cycloalkyl group, while
a hydrogen atom in the 3 position of the cyclopentyl group and in the 4 position of a 6- or 7-membered cycloalkyl group may be replaced in each case by a C₁₋₅-alkoxy, phenyl-C₁₋₃-alkoxy-C₁₋₃-alkyl, phenyl-C₁₋₃-alkylamino, C₁₋₅-alkyl-carbonylamino, benzoylamino, phenyl-C₁₋₃-alkylamino-C₁₋₃-alkyl, benzoylamino-C₁₋₃-alkyl, phenylamino-carbonyl, phenyl-C₁₋₃-alkylamino-carbonyl, carboxy or C₁₋₃-alkoxy-carbonyl group or
in each case the methylene group in the 4 position of a 6- or 7-membered cycloalkyl group may be replaced by an imino group optionally substituted by a phenyl, C₁₋₆-alkyl-carbonyl, benzoyl, phenyl-(C₁₋₃-alkyl)-carbonyl, phenylaminocarbonyl, N—(C₁₋₃-alkyl)-phenylaminocarbonyl, phenyl-C₁₋₃-alkylamino-carbonyl or N—(C₁₋₃-alkyl)-phenyl-C₁₋₃-alkylamino-carbonyl group or
in a 5- or 6-membered cycloalkyl group one or two single bonds separated by at least one bond from each other and from position 1 may each be fused to a phenyl group, while in a bi-or tricyclic ring system thus formed the hydrogen atom bound to the saturated carbon atom in position 1 may be replaced by a C₁₋₃-alkylamino-carbonyl, di-(C₁₋₃-alkyl)amino-carbonyl or C₁₋₅-alkoxy-carbonyl group, while terminal methyl groups in each case may be wholly or partly fluorinated,
a C₁₋₆-alkyl group optionally substituted by a C₃₋₇-cycloalkyl group which is substituted
by a carboxy or C₁₋₃-alkoxycarbonyl group,
by a phenyl, 1-naphthyl or 2-naphthyl group,
by a 5-membered heteroaryl group bound via a carbon or nitrogen atom which contains
an imino group optionally substituted by a C₁₋₃-alkyl or trifluoromethyl group, an oxygen or sulphur atom or
an imino group optionally substituted by a C₁₋₃-alkyl group or an oxygen or sulphur atom and additionally a nitrogen atom,
by a 6-membered heteroaryl group, which contains one or two nitrogen atoms,
while the abovementioned phenyl groups as well as the heteroaryl groups in the carbon skeleton may be monosubstituted by a fluorine, chlorine or bromine atom, by a C₁₋₃-alkyl, trifluoromethyl, C₁₋₃-alkoxy, fluoromethoxy, difluoromethoxy, trifluoromethoxy, C₁₋₃-alkylamino, di-(C₁₋₃-alkyl)amino, amino-C₁₋₃-alkyl, acetylamino, acetyl, C₁₋₃-alkoxy-carbonyl-C₁₋₃-alkyl, C₁₋₅-alkoxy-carbonylamino-C₁₋₃-alkyl, C₁₋₃-alkylamino-carbonyl or di-(C₁₋₃-alkyl)amino-carbonyl group or may also be disubstituted by the abovementioned substituents, while the substituents may be identical or different,
a C₁₋₆-alkyl group substituted by a phenyl group and a carboxy, C₁₋₃-alkoxy-carbonyl, aminocarbonyl, C₁₋₃-alkyl-aminocarbonyl or di-(C₁₋₃-alkyl)-aminocarbonyl group,
a phenyl-C₂₋₃-alkenylene-CH₂ or phenyl-C₂₋₃-alkynylene-CH₂ group, wherein a hydrogen atom of the methylene group in the 1 position may be replaced by a methyl group and independently thereof the phenyl moiety may be substituted by a fluorine, chlorine or bromine atom, by a C₁₋₄-alkyl, C₃₋₇-cycloalkyl, trifluoromethyl, C₁₋₃-alkoxy, phenyl, pyridyl, pyrimidinyl, pyrazinyl, thienyl, pyrrolyl, pyrazolyl or thiazolyl group,
the group R^{b}-A^{b}-E^{b}-C₁₋₃-alkyl optionally substituted by a methyl group in the C₁₋₃-alkyl moiety, wherein
R^{b} denotes a phenyl group optionally mono- or disubstituted by fluorine, chlorine or bromine atoms, by C₁₋₃-alkyl, cyclopropyl, trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, acetylamino, acetyl, carboxy, C₁₋₃-alkoxy-carbonyl, aminocarbonyl, C₁₋₃-alkylamino-carbonyl, di-(C₁₋₃-alkyl)amino-carbonyl or cyano groups, while the substituents may be identical or different,
a 5-membered heteroaryl group which
may be bound via a carbon atom or, if A^{b} denotes a bond, a —CH₂—, (CH₂)₂—, sulphonyl or carbonyl group, may also be bound via a nitrogen atom and
contains an imino group optionally substituted by a C₁₋₃-alkyl group, an oxygen or sulphur atom,
an imino group optionally substituted by a C₁₋₃-alkyl group or an oxygen or sulphur atom and additionally a nitrogen atom or
an imino group optionally substituted by a C₁₋₃-alkyl group and two nitrogen atoms or
an oxygen or sulphur atom and two nitrogen atoms,
a 6-membered heteroaryl group, which contains one or two nitrogen atoms,
while the abovementioned heteroaryl groups in the carbon skeleton may be monosubstituted by a fluorine, chlorine or bromine atom, by a C₁₋₄-alkyl, C₃₋₇-cycloalkyl, trifluoromethyl, phenyl, hydroxy, C₁₋₃-alkoxy, trifluoromethoxy, amino, C₁₋₃-alkylamino, di-(C₁₋₃-alkyl)-amino, acetylamino, acetyl, C₁₋₃-alkoxy-carbonyl, aminocarbonyl, C₁₋₃-alkylamino-carbonyl or di-(C₁₋₃-alkyl)amino-carbonyl group or, with the exception of 5-membered heteroaryl groups containing more than two heteroatoms, may also be disubstituted by the abovementioned substituents, while the substituents may be identical or different,
a C₃₋₇-cycloalkyl group wherein
one or two hydrogen atoms in each case may be replaced by a C₁₋₃-alkyl group and/or
the methylene group in the 4 position of a cyclohexyl group may be replaced by an oxygen atom, by a sulphonyl group or by an imino group optionally substituted by a C₁₋₃-alkyl, C₁₋₃-alkyl-carbonyl, C₁₋₃-alkoxy-carbonyl, C₁₋₃-alkyl-aminocarbonyl or di-(C₁₋₃-alkyl)-aminocarbonyl group or
the two hydrogen atoms of the methylene group in the 3 position of a cyclopentyl group or in the 3- or 4-position of a cyclohexyl or cycloheptyl group may be replaced by an n-butylene, n-pentylene, n-hexylene, 1,2-ethylenedioxy or 1,3-propylenedioxy group,
a 4- to 7-membered cycloalkyleneimino group wherein
the cycloalkylene moiety may be fused to a phenyl ring or
one or two hydrogen atoms in each case may be replaced by a C₁₋₃-alkyl group and/or
in each case the carbon atom in the 4 position of a 6- or 7-membered cycloalkyleneimino group may be substituted by a 4- to 7-membered cycloalkyleneimino, phenyl or 4-(C₁₋₃-alkyl)-1,2,4-triazol-3-yl group or
may be replaced by an oxygen atom, by a sulphonyl group or by an imino group optionally substituted by a C₁₋₃-alkyl, C₁₋₃-alkyl-carbonyl, C₁₋₃-alkyl-aminocarbonyl or di-(C₁₋₃-alkyl)-aminocarbonyl group or
the two hydrogen atoms of the methylene group in the 3 position of a 5-membered cycloalkyleneimino group or in the 3 or 4 position of a 6- or 7-membered cycloalkyleneimino group may be replaced by an n-butylene, n-pentylene, n-hexylene, 1,2-ethylenedioxy or 1,3-propylenedioxy group or
in a 5-, 6- or 7-membered cycloalkyleneimino group a —CH₂— group linked to the imino nitrogen atom may be replaced by a carbonyl group
A^{b} denotes a bond, an oxygen atom, a —NH—, —N(C₁₋₃-alkyl)-, sulphonyl or a carbonyl group,
one of the groups —CH₂—, —(CH₂)₂—, —C≡C—, —O—CH₂—, —CH₂—O—, —NH—CH₂—, —CH₂—NH—, —NH—CO—, —CO—NH—, —NH—SO₂—, —SO₂—NH—,
wherein a hydrogen atom bound to a carbon atom and/or a hydrogen atom bound to a nitrogen atom may be replaced by a C₁₋₃-alkyl group in each case and a heteroatom of group A^{b} is not linked to a nitrogen atom of a 5-membered heteroaryl group of the group R^{b}, and
E^{b} denotes a phenylene group optionally substituted by a fluorine, chlorine or bromine atom, by a C₁₋₄-alkyl group, by a trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, acetylamino, acetyl, carboxy, C₁₋₃-alkoxy-carbonyl, C₁₋₃-alkoxy-carbonyl-C₁₋₃-alkyl, aminocarbonyl, C₁₋₃-alkylamino-carbonyl, di-(C₁₋₃-alkyl)amino-carbonyl or cyano group, or
the group R^{c}-A^{c}-E^{c}-C₁₋₃-alkyl, wherein
R^{c} has the meanings given for R^{b} hereinbefore, while any reference to A^{b} must be replaced by a reference to A^{c},
A^{c} denotes a bond, an oxygen atom, a —CH₂—, —NH—, —N(C₁₋₃-alkyl)-, —NH—CO—, —CO—NH— or carbonyl group,
while a heteroatom of the group A^{c} is not linked to a nitrogen atom of a 5-membered heteroaryl group of the group R^{c}, and
E^{c} denotes a 5-membered heteroarylene group bound via two carbon atoms or via a carbon atom and an imino-nitrogen atom, while the imino-nitrogen atom of the heteroarylene group is not linked to a heteroatom of the group A^{c} and the heteroarylene group contains
an imino group optionally substituted by a C₁₋₃-alkyl group, an oxygen or sulphur atom,
an imino group optionally substituted by a C₁₋₃-alkyl group or an oxygen or sulphur atom and additionally a nitrogen atom or
an imino group optionally substituted by a C₁₋₃-alkyl group and two nitrogen atoms or
an oxygen or sulphur atom and two nitrogen atoms,
or a 6-membered heteroarylene group, which contains one or two nitrogen atoms,
while the abovementioned 5- and 6-membered heteroarylene groups in the carbon skeleton may be substituted by a fluorine, chlorine or bromine atom, by a C₁₋₄-alkyl group, by a C₃₋₇-cycloalkyl, trifluoromethyl, hydroxy, C₁₋₃-alkoxy, trifluoromethoxy, amino, C₁₋₃-alkylamino, acetylamino, acetyl, C₁₋₃-alkoxy-carbonyl, aminocarbonyl, C₁₋₃-alkylamino-carbonyl or cyano group,
or R⁶ and R⁷ together denote an n-alkylene bridge with 4 or 5 carbon atoms wherein
a hydrogen atom may be replaced by a C₁₋₃-alkyl group and/or a —CH₂—CH₂— group may be replaced by a 1,2-linked phenylene group, which may be substituted by a fluorine, chlorine or bromine atom, by a C₁₋₃-alkyl, trifluoromethyl, hydroxy, C₁₋₃-alkoxy, trifluoromethoxy, amino, C₁₋₃-alkylamino, di-(C₁₋₃-alkyl)amino, acetylamino, acetyl, C₁₋₃-alkoxy-carbonyl, aminocarbonyl, C₁₋₃-alkylamino-carbonyl or cyano group or by a phenyloxy or phenyl-C₁₋₃-alkyl group optionally substituted in the phenyl moiety by a fluorine, chlorine or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino or cyano group,
or the carbon atom in the 3 position of an n-pentylene group may be monosubstituted by a C₁₋₃-alkyl group terminally substituted by an amino, C₁₋₃-alkylamino, di-(C₁₋₃-alkyl)-amino or a 5- to 7-membered cycloalkyleneimino group, by a phenyl, C₁₋₃-alkoxycarbonyl, aminocarbonyl, C₁₋₃-alkylamino-carbonyl or di-(C₁₋₃-alkyl)-aminocarbonyl group or may be disubstituted by a phenyl group and a cyano group or
the methylene group in the 3 position of an n-pentylene group may be replaced by an oxygen atom, by a sulphonyl group or by an imino group optionally substituted by a C₁₋₃-alkyl or C₁₋₃-alkyl-carbonyl group,
while the phenyl groups mentioned as being unsubstituted or monosubstituted in the definition of the abovementioned groups as well as aromatic or heteroaromatic parts of molecules may, unless otherwise stated, optionally additionally be substituted in the carbon skeleton by a fluorine, chlorine or bromine atom, by a C₁₋₃-alkyl group, by a trifluoromethyl, hydroxy, C₁₋₃-alkoxy, trifluoromethoxy, amino, C₁₋₃-alkylamino, acetylamino, acetyl, C₁₋₃-alkoxy-carbonyl, aminocarbonyl, C₁₋₃-alkylamino-carbonyl or cyano group,
the alkyl and alkoxy groups mentioned in the definition of the above groups or in the alkyl moieties contained in the groups of formula I defined above with more than two carbon atoms may be straight-chain or branched, unless otherwise specified,
the carboxy groups mentioned in the definition of the abovementioned groups may be replaced by a group which can be converted into a carboxy group in vivo or by a group which is negatively charged under physiological conditions, and/or the amino and imino groups mentioned in the definition of the abovementioned groups may be substituted by a group which can be cleaved in vivo,
their tautomers, their diastereomers, their enantiomers, the mixtures and the salts thereof.

5. The method of claim 4, wherein the MTP inhibitor is a compound of general formula I; and
X₁ denotes the group CR¹,
X₂ denotes the group CR²,
X₃ denotes the group CR³ and
X₄ denotes the group CR⁴ or
one of the groups X₁ to X₄ denotes a nitrogen atom and the remainder of the groups X₁ to X₄ denote three of the groups CR¹ to CR⁴,
while R¹, R², R³ and R⁴ in each case denote a hydrogen atom or
one or two of the groups R¹ to R⁴ independently of one another in each case denote a fluorine, chlorine or bromine atom, a C₁₋₃-alkyl group, a trifluoromethyl, amino, C₁₋₃-alkylamino or di-(C₁₋₃-alkyl)-amino group and the remainder of the groups R¹ to R⁴ in each case represent a hydrogen atom,
while R⁴ additionally together with R⁵ may assume the meaning of a —(CH₂)ₙ— bridge wherein n denotes the number 1, 2 or 3, and
A^{a} denotes a bond, an oxygen atom, a —CH₂— —(CH₂)₂—, —NH—, —N(C₁₋₃-alkyl)-, sulphonyl or carbonyl group or an —NH—CH₂—, —NH—CO, —NH—SO₂— group linked to the group R^{a} in formula (I) via the carbon or sulphur atom,
while a heteroatom of the group A^{a} is not linked to a nitrogen atom of a 5-membered heteroaryl group of the group R^{a},
R^{a} denotes a phenyl or pyridinyl group,
a pyrrolyl, furanyl, thienyl, pyrazolyl, imidazolyl, oxazolyl, isoxazolyl or thiazolyl group bound via a carbon or nitrogen atom,
while a nitrogen atom of the pyrrolyl, pyrazolyl and imidazolyl group may be substituted by a C₁₋₃-alkyl group and the phenyl group as well as the abovementioned heteroaromatic groups in the carbon skeleton may be substituted by a fluorine, chlorine or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino or cyano group,
a 5- to 7-membered cycloalkyleneimino group wherein
the methylene group in the 4 position of a 6-membered cycloalkyleneimino group may be substituted by a methyl group or replaced by an oxygen or sulphur atom or by an imino group optionally substituted by a C₁₋₃-alkyl group or
in a piperidino group a —CH₂— group linked to the imino nitrogen atom may be replaced by a carbonyl group or a —(CH₂)₂— group linked to the imino nitrogen atom may be replaced by a —CO—NR⁸— group or a —(CH₂)₃— group linked to the imino nitrogen atom may be replaced by a —CO—NR⁸—CO— group,
while R⁸ denotes a hydrogen atom or a C₁₋₃-alkyl group,
R⁵ denotes a hydrogen atom or a C₁₋₃-alkyl group,
Het denotes a 5-membered heteroarylene group bound via two carbon atoms which contains
an imino group substituted by the group R⁹, an oxygen or sulphur atom or
an imino group substituted by the group R⁹ or an oxygen or sulphur atom and additionally contains a nitrogen atom,
while R⁹ denotes a hydrogen atom, a C₁₋₃-alkyl group, a —C₂₋₃-alkyl group terminally substituted by an amino, C₁₋₃-alkylamino, di-(C₁₋₃-alkyl)-amino or C₁₋₄-alkoxy-carbonyl-amino group, a carboxy-C₁₋₃-alkyl, C₁₋₃-alkoxy-carbonyl-C₁₋₃-alkyl or C₁₋₃-alkylcarbonyl group or R⁹ together with R⁶ denotes a —(CH₂)ₚ— bridge wherein p is the number 2 or 3,
or a pyridinylene or pyrimidinylene group,
while the abovementioned heteroarylene groups in the carbon skeleton may be substituted by a fluorine, chlorine or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino or cyano group,
R⁶ denotes a hydrogen atom or a C₁₋₃-alkyl group,
R⁷ denotes a C₁₋₆-alkyl group,
a straight-chain C₂₋₆-alkyl group which is terminally substituted by an amino, C₁₋₃-alkylamino or di-(C₁₋₃-alkyl)-amino group,
a C₁₋₄-alkyl group terminally substituted by a C₃₋₇-cycloalkyl group, while a hydrogen atom in the 4 position of a cyclohexyl group may be replaced by a C₁₋₅-alkoxy, C₁₋₃-alkoxy-C₁₋₃-alkyl, phenyl-C₁₋₃-alkoxy-methyl, phenyl-C₁₋₃-alkylamino, phenyl-C₁₋₂-alkyl-carbonylamino, benzoylamino, phenylaminocarbonyl, phenyl-C₁₋₃-alkyl-aminocarbonyl, carboxy or C₁₋₃-alkoxy-carbonyl group or
in a cyclopentyl group one or two single bonds separated from each other and from position 1 by at least one bond may each be fused to a phenyl group, while in a bi-or tricyclic ring system thus formed the hydrogen atom bound to the saturated carbon atom in the 1 position may be replaced by a C₁₋₃-alkylamino-carbonyl or di-(C₁₋₃-alkyl)amino-carbonyl group, wherein terminal methyl groups in each case may be wholly or partly fluorinated,
a C₁₋₆-alkyl group optionally substituted by a C₃₋₅-cycloalkyl group which is substituted
by a carboxy or C₁₋₃-alkoxycarbonyl group or
by a phenyl, 1-naphthyl, 2-naphthyl, pyridinyl, pyrimidinyl, pyrrolyl, furanyl, thienyl, pyrazolyl, imidazolyl, oxazolyl, isoxazolyl, thiazolyl or isothiazolyl group,
while a nitrogen atom of the pyrrolyl, pyrazolyl and imidazolyl group may be substituted by a C₁₋₃-alkyl or trifluoromethyl group and the phenyl group as well as the abovementioned heteroaromatic groups in the carbon skeleton may be substituted by a fluorine, chlorine or bromine atom, by a C₁₋₄-alkyl, trifluoromethyl, C₁₋₃-alkoxy, fluoromethoxy, difluoromethoxy, trifluoromethoxy, C₁₋₄-alkoxy-carbonylamino-C₁₋₃-alkyl, amino, C₁₋₃-alkylamino, di-(C₁₋₃-alkyl)amino or cyano group,
a C₁₋₆-alkyl group substituted by a phenyl group and a carboxy or C₁₋₃-alkoxy-carbonyl group,
a phenyl-C₂₋₃-alkynylene-CH₂ group wherein a hydrogen atom of the methylene group in the 1 position may be replaced by a methyl group and independently thereof the phenyl moiety may be substituted by a fluorine, chlorine or bromine atom or by a C₁₋₄-alkyl, trifluoromethyl, C₁₋₃-alkoxy, phenyl or cyano group,
the group R^{b}-A^{b}-E^{b}-C₁₋₃-alkyl optionally substituted in the C₁₋₃-alkyl moiety by a methyl group, wherein
R^{b} denotes a phenyl group optionally substituted by a fluorine, chlorine or bromine atom, by a C₁₋₃-alkyl, trifluoromethyl, hydroxy, C₁₋₃-alkoxy, fluoromethoxy, difluoromethoxy, trifluoromethoxy, carboxy or C₁₋₃-alkoxy-carbonyl group,
a 5-membered heteroaryl group which
may be bound via a carbon atom or, if A^{b} denotes a bond, may also be bound via a nitrogen atom and contains
an imino group optionally substituted by a C₁₋₃-alkyl group, an oxygen or sulphur atom,
an imino group optionally substituted by a C₁₋₃-alkyl group or an oxygen or sulphur atom and additionally a nitrogen atom or
an imino group optionally substituted by a C₁₋₃-alkyl group and two nitrogen atoms or
an oxygen or sulphur atom and two nitrogen atoms,
a 6-membered heteroaryl group, which contains one or two nitrogen atoms,
while the abovementioned heteroaryl groups may be monosubstituted in the carbon skeleton by a fluorine, chlorine or bromine atom, by a C₁₋₃-alkyl, trifluoromethyl, phenyl, C₁₋₃-alkoxy, trifluoromethoxy, amino, C₁₋₃-alkylamino, di-(C₁₋₃-alkyl)-amino or acetylamino group or, with the exception of 5-membered heteroaryl groups containing more than two heteroatoms, may also be disubstituted by a C₁₋₄-alkyl group and one substituent selected from fluorine, chlorine, bromine, C₁₋₃-alkyl, trifluoromethyl, phenyl, C₁₋₃-alkoxy and trifluoromethoxy,
a C₃₋₆-cycloalkyl group, wherein
the two hydrogen atoms of the methylene group in the 3 position of a cyclopentyl group or in the 3- or 4-position of a cyclohexyl group may be replaced by an n-butylene, n-pentylene or 1,2-ethylenedioxy group,
a 5- to 7-membered cycloalkyleneimino group wherein
the cycloalkylene moiety may be fused to a phenyl ring or
a hydrogen atom may be replaced by a C₁₋₃-alkyl group and/or
in each case the carbon atom in the 4 position of a 6- or 7-membered cycloalkyleneimino group may be substituted by a 4- to 7-membered cycloalkyleneimino, phenyl or 4-(C₁₋₃-alkyl)-1,2,4-triazol-3-yl group or
the two hydrogen atoms of the methylene group in the 3 position of a 5-membered cycloalkyleneimino group or in the 3 or 4 position of a 6- or 7-membered cycloalkyleneimino group may be replaced by an n-butylene, n-pentylene or 1,2-ethylenedioxy group,
A^{b} denotes a bond, an oxygen atom, a —CH₂—, —NH—, —O—CH₂—, carbonyl, —NH—CO— or —CO—NH— group,
wherein a hydrogen atom bound to a nitrogen atom may be replaced in each case by a C₁₋₃-alkyl group,
E^{b} denotes a phenylene group optionally substituted by a fluorine, chlorine or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino or C₁₋₃-alkoxy-carbonyl group, or
the group R^{c}-A^{c}-E^{c}-C₁₋₃-alkyl, wherein
R^{c} denotes a phenyl group optionally substituted by a fluorine, chlorine or bromine atom, by a C₁₋₃-alkyl, trifluoromethyl, C₁₋₃-alkoxy, trifluoromethoxy, carboxy or C₁₋₃-alkoxy-carbonyl group or
a 5- to 7-membered cycloalkyleneimino group wherein
the cycloalkylene moiety may be fused to a phenyl ring or
a hydrogen atom may be replaced by a C₁₋₃-alkyl group and/or
the two hydrogen atoms of the methylene group in the 3 position of a 5-membered cycloalkyleneimino group or in the 3 or 4 position of a 6- or 7-membered cycloalkyleneimino group may be replaced by an n-butylene, n-pentylene or 1,2-ethylenedioxy group,
A^{c} denotes a bond,
E^{c} denotes a 5-membered heteroarylene group bound via two carbon atoms which contains
an imino group optionally substituted by a C₁₋₃-alkyl group, an oxygen or sulphur atom,
an imino group optionally substituted by a C₁₋₃-alkyl group or an oxygen or sulphur atom and additionally a nitrogen atom or
an imino group optionally substituted by a C₁₋₃-alkyl group and two nitrogen atoms or
an oxygen or sulphur atom and two nitrogen atoms,
or a pyridinylene, pyridazinylene or pyrimidinylene group,
while the abovementioned 5- and 6-membered heteroarylene groups in the carbon skeleton may be substituted by a fluorine, chlorine or bromine atom, by a C₁₋₃-alkyl, trifluoromethyl, C₁₋₃-alkoxy, trifluoromethoxy, amino, C₁₋₃-alkylamino, acetylamino, C₁₋₃-alkoxy-carbonyl or cyano group,
or R⁶ and R⁷ together denote an n-alkylene bridge with 4 or 5 carbon atoms, wherein
a hydrogen atom may be replaced by a C₁₋₃-alkyl group and/or
a —CH₂—CH₂— group may be replaced by a 1,2-linked phenylene group optionally substituted by a phenyloxy or benzyl group, while
the phenyloxy or benzyl group in the aromatic moiety and the phenylene group may be substituted independently of one another by a fluorine, chlorine or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino, C₁₋₃-alkoxy-carbonyl or cyano group,
or the carbon atom in the 3 position of an n-pentylene group may be monosubstituted by a C₁₋₃-alkyl group terminally substituted by an amino, C₁₋₃-alkylamino, di-(C₁₋₃-alkyl)-amino, acetylamino or N-(methyl)-acetylamino group or a 5- to 7-membered cycloalkyleneimino group or may be disubstituted by a phenyl group and a cyano group,
while the phenyl groups mentioned in the definition of the abovementioned groups may, unless otherwise stated, be substituted by a fluorine, chlorine or bromine atom, by a C₁₋₃-alkyl group, by a trifluoromethyl, C₁₋₃-alkoxy, trifluoromethoxy, phenyl, amino, C₁₋₃-alkylamino, acetylamino, C₁₋₃-alkoxy-carbonyl or cyano group,
the alkyl and alkoxy groups mentioned in the definition of the above groups or in the alkyl moieties contained in the groups of formula I defined above with more than two carbon atoms may be straight-chain or branched, unless otherwise specified,
the carboxy groups mentioned in the definition of the abovementioned groups may be replaced by a group which can be converted into a carboxy group in vivo or by a group which is negatively charged under physiological conditions, and/or
the amino and imino groups mentioned in the definition of the abovementioned groups may be substituted by a group which can be cleaved in vivo,
their tautomers, their diastereomers, their enantiomers, the mixtures and the salts thereof.

6. The method of claim 5, wherein the MTP inhibitor is a compound of general formula I; and
X₁ denotes the group CR¹,
X₂ denotes the group CR²,
X₃ denotes the group CR³ and
X₄ denotes the group CR⁴or
one of the groups X₁ to X₄ denotes a nitrogen atom and the remainder of the groups X₁ to X₄ denote three of the groups CR¹ to CR⁴,
while R¹, R², R³ and R⁴ in each case denote a hydrogen atom or
one or two of the groups R¹ to R⁴ independently of one another each denote a fluorine, chlorine or bromine atom, a C₁₋₃-alkyl group, a trifluoromethyl, amino, C₁₋₃-alkylamino or di-(C₁₋₃-alkyl)-amino group and the remainder of the groups R¹ to R⁴ each represent a hydrogen atom,
while R⁴ additionally together with R⁵ may assume the meaning of a —(CH₂)ₙ— bridge wherein n denotes the number 1, 2 or 3, and
A^{a} denotes a bond, an oxygen atom, a —CH₂, —(CH₂)₂, —NH, —N(C₁₋₃-alkyl), sulphonyl or carbonyl group or a —NH—CH₂, —NH—CO, —NH—SO₂ group linked to the group R^{a} in formula (I) via the carbon or sulphur atom,
while a heteroatom of group A^{a} is not linked to a nitrogen atom of a 5-membered heteroaryl group of the group R^{a},
R^{a} denotes a phenyl or pyridinyl group, a pyrrolyl, furanyl, thienyl, pyrazolyl, imidazolyl, oxazolyl, isoxazolyl or thiazolyl group bound via a carbon or nitrogen atom,
while a nitrogen atom of the pyrrolyl, pyrazolyl and imidazolyl group may be substituted by a C₁₋₃-alkyl group and the phenyl group as well as the abovementioned heteroaromatic groups in the carbon skeleton may be substituted by a fluorine, chlorine or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino or cyano group,
a 5- to 7-membered cycloalkyleneimino group wherein
the methylene group in the 4 position of a 6-membered cycloalkyleneimino group may be substituted by a methyl group or may be replaced by an oxygen or sulphur atom or by an imino group optionally substituted by a C₁₋₃-alkyl group or
in a piperidino group a —CH₂— group linked to the imino nitrogen atom may be replaced by a carbonyl group or a —(CH₂)₂— group linked to the imino nitrogen atom may be replaced by a —CO—NR⁸— group or a —(CH₂)₃— group linked to the imino nitrogen atom may be replaced by a —CO—NR⁸—CO— group,
while R⁸ denotes a hydrogen atom or a C₁₋₃-alkyl group,
R⁵ denotes a hydrogen atom or a C₁₋₃-alkyl group,
Het denotes a 2,4-linked pyrrolylene or imidazolylene group which are bound in each case via the 2 position to the adjacent carbonyl group of formula I and
are substituted at a nitrogen atom by a C₁₋₃-alkyl group and in the carbon skeleton may be substituted by a C₁₋₃-alkyl group or a trifluoromethyl group,
R⁶ denotes a hydrogen atom or a C₁₋₃-alkyl group,
R⁷ denotes a C₁₋₄-alkyl group terminally substituted by a C₃₋₇-cycloalkyl group, while
a hydrogen atom in the 4 position of a cyclohexyl group may be replaced by a C₁₋₅-alkoxy, C₁₋₃-alkoxy-C₁₋₃-alkyl, phenyl-C₁₋₃-alkoxy-methyl, phenyl-C₁₋₃-alkylamino, phenyl-C₁₋₂-alkyl-carbonylamino, benzoylamino, phenylaminocarbonyl, phenyl-C₁₋₃-alkyl-aminocarbonyl, carboxy or C₁₋₃-alkoxy-carbonyl group or
in a cyclopentyl group one or two single bonds separated from each other and from position 1 by at least one bond may each be fused to a phenyl group, while in a bi- or tricyclic ring system thus formed the hydrogen atom bound to the saturated carbon atom in the 1 position may be replaced by a C₁₋₃-alkylamino-carbonyl or di-(C₁₋₃-alkyl)amino-carbonyl group, while terminal methyl groups may each be wholly or partly fluorinated,
a C₁₋₆-alkyl group optionally substituted by a C₃₋₅-cycloalkyl group which is substituted
by a phenyl, 1-naphthyl, 2-naphthyl, pyridinyl, pyrimidinyl, pyrrolyl, furanyl, thienyl, pyrazolyl, imidazolyl, oxazolyl, isoxazolyl, thiazolyl or isothiazolyl group, while a nitrogen atom of the pyrrolyl, pyrazolyl and imidazolyl group may be substituted by a C₁₋₃-alkyl or trifluoromethyl group and the phenyl group and the abovementioned heteroaromatic groups in the carbon skeleton may be substituted by a fluorine, chlorine or bromine atom, by a C₁₋₄-alkyl, trifluoromethyl, C₁₋₃-alkoxy, fluoromethoxy, difluoromethoxy, trifluoromethoxy, C₁₋₄-alkoxy-carbonylamino-C₁₋₃-alkyl, amino, C₁₋₃-alkylamino, di-(C₁₋₃-alkyl)amino or cyano group,
a C₁₋₆-alkyl group substituted by a phenyl group and a carboxy or C₁₋₃-alkoxy-carbonyl group,
a phenyl-C₂₋₃-alkynylene-CH₂ group wherein a hydrogen atom of the methylene group may be replaced in the 1 position by a methyl group and independently thereof the phenyl moiety may be substituted by a fluorine, chlorine or bromine atom, by a C₁₋₄-alkyl, trifluoromethyl, C₁₋₃-alkoxy, phenyl or cyano group,
the group R^{b}-A^{b}-E^{b}-C₁₋₃-alkyl optionally substituted in the C₁₋₃-alkyl moiety by a methyl group, wherein
R^{b} denotes a phenyl group optionally substituted by a fluorine, chlorine or bromine atom, by a C₁₋₃-alkyl, trifluoromethyl, hydroxy, C₁₋₃-alkoxy, fluoromethoxy, difluoromethoxy, trifluoromethoxy, carboxy or C₁₋₃-alkoxy-carbonyl group,
a 5-membered heteroaryl group which
may be bound via a carbon atom or, if A^{b} denotes a bond, may also be bound via a nitrogen atom and contains an imino group optionally substituted by a C₁₋₃-alkyl group, an oxygen or sulphur atom, an imino group optionally substituted by a C₁₋₃-alkyl group or an oxygen or sulphur atom and additionally a nitrogen atom or
an imino group optionally substituted by a C₁₋₃-alkyl group and two nitrogen atoms or
an oxygen or sulphur atom and two nitrogen atoms,
a 6-membered heteroaryl group which contains one or two nitrogen atoms,
while the abovementioned heteroaryl groups in the carbon skeleton may be monosubstituted by a fluorine, chlorine or bromine atom, by a C₁₋₃-alkyl, trifluoromethyl, phenyl, C₁₋₃-alkoxy, trifluoromethoxy, amino, C₁₋₃-alkylamino, di-(C₁₋₃-alkyl)-amino or acetylamino group or, with the exception of 5-membered heteroaryl groups containing more than two heteroatoms, may also be disubstituted by a C₁₋₄-alkyl group and a substituent selected from fluorine, chlorine, bromine, C₁₋₃-alkyl, trifluoromethyl, phenyl, C₁₋₃-alkoxy and trifluoromethoxy,
a C₃₋₆-cycloalkyl group, while
the two hydrogen atoms of the methylene group in the 3 position of a cyclopentyl group or in the 3- or 4-position of a cyclohexyl group may be replaced by an n-butylene, n-pentylene or 1,2-ethylenedioxy group,
a 5- to 7-membered cycloalkyleneimino group wherein
the cycloalkylene moiety may be fused to a phenyl ring or
a hydrogen atom may be replaced by a C₁₋₃-alkyl group and/or
in each case the carbon atom in the 4 position of a 6- or 7-membered cycloalkyleneimino group may be substituted by a 4- to 7-membered cycloalkyleneimino, phenyl or 4-(C₁₋₃-alkyl)-1,2,4-triazol-3-yl group or
the two hydrogen atoms of the methylene group in the 3 position of a 5-membered cycloalkyleneimino group or in the 3 or 4 position of a 6- or 7-membered cycloalkyleneimino group may be replaced by an n-butylene, n-pentylene or 1,2-ethylenedioxy group,
A^{b} denotes a bond, an oxygen atom, a —CH₂—, —NH—, —O—CH₂—, carbonyl-, —NH—CO or —CO—NH-group,
wherein a hydrogen atom bound to a nitrogen atom may be replaced in each case by a C₁₋₃-alkyl group,
E^{b} denotes a phenylene group optionally substituted by a fluorine, chlorine or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino or C₁₋₃-alkoxy-carbonyl group, or
the group R^{c}-A^{c}-E^{c}-C₁₋₃-alkyl, wherein
R^{c} denotes a phenyl group optionally substituted by a fluorine, chlorine or bromine atom, by a C₁₋₃-alkyl, trifluoromethyl, C₁₋₃-alkoxy, trifluoromethoxy, carboxy or C₁₋₃-alkoxy-carbonyl group or
a 5- to 7-membered cycloalkyleneimino group wherein
the cycloalkylene moiety may be fused to a phenyl ring or
a hydrogen atom may be replaced by a C₁₋₃-alkyl group and/or
the two hydrogen atoms of the methylene group in the 3 position of a 5-membered cycloalkyleneimino group or in the 3 or 4 position of a 6- or 7-membered cycloalkyleneimino group may be replaced by an n-butylene, n-pentylene or 1,2-ethylenedioxy group,
A^{c} denotes a bond,
E^{c} denotes a 5-membered heteroarylene group bound via two carbon atoms which contains
an imino group optionally substituted by a C₁₋₃-alkyl group, an oxygen or sulphur atom,
an imino group optionally substituted by a C₁₋₃-alkyl group or an oxygen or sulphur atom and additionally a nitrogen atom or
an imino group optionally substituted by a C₁₋₃-alkyl group and two nitrogen atoms or
an oxygen or sulphur atom and two nitrogen atoms,
or a pyridinylene, pyridazinylene or pyrimidinylene group,
while the abovementioned 5- and 6-membered heteroarylene groups in the carbon skeleton may be substituted by a fluorine, chlorine or bromine atom, by a C₁₋₃-alkyl, trifluoromethyl, C₁₋₃-alkoxy, trifluoromethoxy, amino, C₁₋₃-alkylamino, acetylamino, C₁₋₃-alkoxy-carbonyl or cyano group,
or R⁶ and R⁷ together denote an n-alkylene bridge with 4 or 5 carbon atoms wherein
a hydrogen atom may be replaced by a C₁₋₃-alkyl group and/or
a —CH₂—CH₂— group may be replaced by a 1,2-linked phenylene group optionally substituted by a phenyloxy or benzyl group, while
the phenyloxy or benzyl group in the aromatic moiety and the phenylene group may be substituted independently of one another by a fluorine, chlorine or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino, C₁₋₃-alkoxy-carbonyl or cyano group,
or the carbon atom in the 3 position of an n-pentylene group may be monosubstituted by a C₁₋₃-alkyl group terminally substituted by an amino, C₁₋₃-alkylamino, di-(C₁₋₃-alkyl)-amino, acetylamino or N-(methyl)-acetylamino group or a 5- to 7-membered cycloalkyleneimino group or may be disubstituted by a phenyl group and a cyano group,
while the phenyl groups mentioned in the definition of the abovementioned groups may, unless otherwise stated, be substituted by a fluorine, chlorine or bromine atom, by a C₁₋₃-alkyl group, by a trifluoromethyl, C₁₋₃-alkoxy, trifluoromethoxy, phenyl, amino, C₁₋₃-alkylamino, acetylamino, C₁₋₃-alkoxy-carbonyl or cyano group,
the alkyl and alkoxy groups mentioned in the definition of the above groups or in the alkyl moieties contained in the groups of formula I defined above with more than two carbon atoms may be straight-chain or branched, unless otherwise specified,
the carboxy groups mentioned in the definition of the abovementioned groups may be replaced by a group which can be converted into a carboxy group in vivo or by a group which is negatively charged under physiological conditions, and/or
the amino and imino groups mentioned in the definition of the abovementioned groups may be substituted by a group which can be cleaved in vivo,
their tautomers, their diastereomers, their enantiomers, the mixtures and the salts thereof.

7. The method of claim 6, wherein the MTP inhibitor is a compound of general formula I; and
X₁ denotes the group CR¹,
X₂ denotes the group CR²,
X₃ denotes the group CR³ and
X₄ denotes the group CR⁴,
while R¹, R², R³ and R⁴ in each case denote a hydrogen atom or
one of the groups R¹ to R⁴ denotes a fluorine, chlorine or bromine atom, a C₁₋₃-alkyl group or a trifluoromethyl group and the remainder of the groups R¹ to R⁴ in each case denote a hydrogen atom,
A^{a} denotes a bond, an oxygen atom, a —CH₂— —(CH₂)₂— —NH—, or —N(C₁₋₃-alkyl)-group,
while a nitrogen atom of the group A^{a} is not linked to a nitrogen atom of a 5-membered heteroaryl group of the group R^{a},
R^{a} denotes a phenyl, 2-pyridinyl, 3-pyridinyl or 4-pyridinyl group,
a 1-pyrrolyl, 2-pyrrolyl, 3-pyrrolyl, 2-thienyl or 3-thienyl group,
while the nitrogen atom of the pyrrolyl group may be substituted by a C₁₋₃-alkyl group and the phenyl group and the abovementioned heteroaromatic groups in the carbon skeleton may be substituted by a fluorine, chlorine or bromine atom, by a C₁₋₃-alkyl or trifluoromethyl group,
a pyrrolidino, piperidino or morpholino group
R⁵ denotes a hydrogen atom,
Het denotes a 2,4-linked pyrrolylene or imidazolylene group which are bound in each case via the 2 position to the adjacent carbonyl group of formula I and
are substituted by a C₁₋₃-alkyl group at a nitrogen atom and may be substituted in the carbon skeleton by a C₁₋₃-alkyl group or a trifluoromethyl group,
R⁶ denotes a hydrogen atom or a C₁₋₃-alkyl group,
R⁷ denotes the group R^{d}—CH₂— or R^{d}—CH₂—CH₂—, wherein a hydrogen atom of the methylene group may be replaced in the 1 position by a C₁₋₃-alkyl group or a cyclopropyl group and wherein
R^{d} denotes a phenyl, 1-naphthyl, 2-naphthyl, 2-pyridinyl, 3-pyridinyl, 4-pyridinyl, 2-pyrimidinyl or 5-pyrimidinyl group,
while the phenyl group and the abovementioned heteroaromatic groups in the carbon skeleton may be substituted by a fluorine, chlorine or bromine atom, by a C₁₋₄-alkyl, trifluoromethyl, C₁₋₃-alkoxy or fluoromethoxy group,
a phenyl —C≡C—CH₂— group wherein a hydrogen atom of the methylene group in the 1 position may be replaced by a methyl group and independently thereof the phenyl moiety may be substituted by a fluorine, chlorine or bromine atom, by a C₁₋₄-alkyl, trifluoromethyl or phenyl group,
the group R^{b}-A^{b}-E^{b}-CH₂—, wherein a hydrogen atom of the methylene group may be replaced in the 1 position by a methyl group and wherein
R^{b} denotes a phenyl group optionally substituted by a fluorine, chlorine or bromine atom, by a C₁₋₃-alkyl, trifluoromethyl, hydroxy, methoxy, carboxy or methoxycarbonyl group,
a pyrrolyl, pyrazolyl, imidazolyl, oxazolyl, isoxazolyl, thiazolyl, isothiazolyl, oxadiazol or thiadiazolyl group bound via a carbon atom or, if A^{b} denotes a bond, also bound via a nitrogen atom, wherein a hydrogen atom bound to a nitrogen atom may be replaced by a C₁₋₃-alkyl group,
a 2-pyridyl, 3-pyridyl, 4-pyridyl, pyrazinyl, 2-pyrimidinyl, 4-pyrimidinyl, 5-pyrimidinyl, 3-pyridazinyl or 4-pyridazinyl group,
while the abovementioned 5- and 6-membered heteroaryl groups in the carbon skeleton may be monosubstituted by a fluorine, chlorine or bromine atom, by a C₁₋₃-alkyl, trifluoromethyl, phenyl, amino, C₁₋₃-alkylamino, di-(C₁₋₃-alkyl)-amino or acetylamino group or, with the exception of 5-membered heteroaryl groups containing more than two heteroatoms, may also be disubstituted by a C₁₋₃-alkyl group and a substituent selected from fluorine, chlorine, bromine, C₁₋₃-alkyl, trifluoromethyl, phenyl,
a C₅₋₆-cycloalkyl group, while
the two hydrogen atoms of the methylene group in the 3-position of the cyclopentyl group or in the 4-position of the cyclohexyl group may be replaced by an n-butylene, n-pentylene or 1,2-ethylenedioxy group,
or a 5- to 6-membered cycloalkyleneimino group wherein
the cycloalkylene moiety may be fused to a phenyl ring optionally substituted by a fluorine, chlorine or bromine atom, by a C₁₋₃-alkyl, trifluoromethyl or C₁₋₃-alkoxy group or
a hydrogen atom may be replaced by a C₁₋₃-alkyl group and/or
the two hydrogen atoms of the methylene group in the 3 position of the 5-membered cycloalkyleneimino group or in the 4 position of the 6-membered cycloalkyleneimino group may be replaced by an n-butylene, n-pentylene or 1,2-ethylenedioxy group,
A^{b} denotes a bond, a —CH₂—, —NH—, —O—CH₂—, —NH—CO— or —CO—NH— group,
wherein a hydrogen atom bound to a nitrogen atom may be replaced in each case by a methyl group,
E^{b} denotes a 1,4-linked phenylene group, optionally substituted by a fluorine, chlorine or bromine atom, by a C₁₋₃-alkyl, trifluoromethyl, C₁₋₃-alkoxy or trifluoromethoxy group, or
the group R^{c}-A^{c}-E^{c}-C₁₋₃-alkyl, wherein
R^{c} denotes a phenyl group optionally substituted by a fluorine, chlorine or bromine atom, by a C₁₋₃-alkyl, trifluoromethyl, methoxy, carboxy or methoxycarbonyl group,
A^{c} denotes a bond,
E^{c} denotes a pyrrolylene, pyrazolylene, imidazolylene, oxazolylene, isoxazolylene, thiazolylene, isothiazolylene, [1,3,4]-oxadiazolene or [1,3,4]-thiadiazolene group bound via two carbon atoms in the relative positions 1,3, wherein a hydrogen atom bound to a nitrogen atom may be replaced by a C₁₋₃-alkyl group,
or a 1,4-linked pyridinylene, pyridazinylene or pyrimidinylene group,
while the abovementioned 5- and 6-membered heteroarylene groups may be substituted in the carbon skeleton by a fluorine, chlorine or bromine atom, by a C₁₋₃-alkyl, trifluoromethyl or methoxy group,
while the alkyl and alkoxy groups mentioned in the definition of the above groups or in the alkyl moieties contained in the groups of formula I defined above with more than two carbon atoms may be straight-chain or branched, unless otherwise specified,
the carboxy groups mentioned in the definition of the abovementioned groups may be replaced by a group which can be converted into a carboxy group in vivo or by a group which is negatively charged under physiological conditions, and/or
the amino and imino groups mentioned in the definition of the abovementioned groups may be substituted by a group which can be cleaved in vivo,
their tautomers, their diastereomers, their enantiomers, the mixtures and the salts thereof.

8. The method of claim 7, wherein the MTP inhibitor is one of the following compounds of general formula I:
(a) N-[3-(Biphenyl-4-yl)-prop-2-ynyl]-4-(4′-trifluoromethylbiphenyl-2-carbonylamino)-1-methyl-pyrrole-2-carboxylic acid amide,
(b) N-[4-(1,4-Dioxa-8-aza-spiro[4.5]dec-8-yl)-phenylmethyl]-4-(4′-trifluoromethyl-biphenyl-2-carbonylamino)-1-methyl-pyrrole-2-carboxylic acid amide,
(c) N-[4-(3-Aza-spiro[5.5]undec-3-yl)-phenylmethyl]-4-(4′-trifluoromethylbiphenyl-2-carbonylamino)-1-methyl-pyrrole-2-carboxylic acid amide,
(d) N-[4-(6-Methylpyridazin-3-yl)-phenylmethyl]-4-(4′-trifluoromethylbiphenyl-2-carbonylamino)-1-methyl-pyrrole-2-carboxylic acid amide,
(e) N-(4′-Hydroxybiphenyl-4-yl)methyl-4-(4′-trifluoromethylbiphenyl-2-carbonylamino)-1-methyl-pyrrole-2-carboxylic acid amide,
(f) N-[4-(1,4-Dioxa-spiro[4.5]dec-8-yl)-phenylmethyl]-4-(4′-trifluoromethylbiphenyl-2-carbonylamino)-1-methyl-pyrrole-2-carboxylic acid amide,
(g) N-(4′-Methylbiphenyl-4-yl)methyl-4-(4′-trifluoromethylbiphenyl-2-carbonylamino)-1-methyl-pyrrole-2-carboxylic acid amide,
(h) N-[3-(4-Isopropylphenyl)-prop-2-ynyl]-4-(4′-trifluoromethyl-biphenyl-2-carbonylamino)-1-methyl-pyrrole-2-carboxylic acid amide,
(i) N-[3-(4-Biphenyl)-prop-2-ynyl]-4-(4′-trifluoromethylbiphenyl-2-carbonylamino)-1-methyl-imidazole-2-carboxylic acid amide,
(j) N-[3-(4-Trifluoromethylphenyl)-prop-2-ynyl]-4-(4′-trifluoromethylbiphenyl-2-carbonylamino)-1-methyl-imidazole-2-carboxylic acid amide und
(k) N-[4-(4-Propylpiperidino)-phenylmethyl]-4-(4′-trifluoromethylbiphenyl-2-carbonyl-amino)-1-methyl-pyrrol-2-carboxylic acid amide
and the salts thereof.

9. The method of claim 8, wherein the MTP inhibitor is selected from the list consisiting of compounds of general formula I:
(a) N-[3-(Biphenyl-4-yl)-prop-2-ynyl]-4-(4′-trifluoromethylbiphenyl-2-carbonylamino)-1-methyl-pyrrole-2-carboxylic acid amide,
(c) N-[4-(3-Aza-spiro[5.5]undec-3-yl)-phenylmethyl]-4-(4′-trifluoromethylbiphenyl-2-carbonylamino)-1-methyl-pyrrole-2-carboxylic acid amide,
(f) N-[4-(1,4-Dioxa-spiro[4.5]dec-8-yl)-phenylmethyl]-4-(4′-trifluoromethylbiphenyl-2-carbonylamino)-1-methyl-pyrrole-2-carboxylic acid amide,
(i) N-[3-(4-Biphenyl)-prop-2-ynyl]-4-(4′-trifluoromethylbiphenyl-2-carbonylamino)-1-methyl-imidazole-2-carboxylic acid amide und
k) N-[4-(4-Propylpiperidino)-phenylmethyl]-4-(4′-trifluoromethylbiphenyl-2-carbonylamino)-1-methyl-pyrrole-2-carboxylic acid amide
and the salts thereof.

10. The method of claim 1, wherein the MTP inhibitor is selected from the list consisting of:
9-{4-[4-(4-Trifluoromethyl-phenylacetyl)-piperazino]-butyl}-9H-fluoren-9-carboxylic acid-(2,2,2-trifluoroethyl)-amide,
9-[4-(4-Phenylacetyl-piperazino)-butyl]-9H-fluorene-9-carboxylic acid-(2,2,2-trifluoroethyl)-amide,
9-(4-{4-[2-Phenyl-butyryl]-piperazino}-butyl)-9H-fluorene-9-carboxylic acid-(2,2,2-trifluoroethyl)-amide,
9-(4-{4-(3-Phenylpropionyl)-piperazino}-butyl)-9H-fluorene-9-carboxylic acid-(2,2,2-trifluoroethyl)-amide,
9-{4-[4-(4-Phenyl-butyryl)-piperazino]-butyl}-9H-fluorene-9-carboxylic acid-(2,2,2-trifluoroethyl)-amide,
9-(4-{4-(4-(Pyridin-2-yl-acetyl)-piperazino}-butyl)-9H-fluorene-9-carboxylic acid-(2,2,2-trifluoroethyl)-amide,
9-(4-{4-[2-Oxo-2-phenyl-acetyl]-piperazino}-butyl)-9H-fluorene-9-carboxylic acid-(2,2,2-trifluoroethyl)-amide,
9-(4-{4-[(2,4-Dichlorophenyl)-acetyl]-piperazino}-butyl)-9H-fluorene-9-carboxylic acid-(2,2,2-trifluoroethyl)-amide,
9-[4-[4-[2-(4-Trifluoromethylphenyl)benzoylamino]piperidin-1-yl]butyl]-N-(2,2,2-trifluoroethyl)-9H-fluorene-9-carboxamide,
9-[4-[2,5-Dimethyl-4-[[[4′-(trifluoromethyl)[1,1′-biphenyl]-2-yl]carbonyl]amino]-1H-benzimidazol-1-yl]butyl]-N-(2,2,2-trifluoroethyl)-9H-fluorene-9-carboxamide,
2(S)-Cyclopentyl-2-(4-(2,4-dimethyl-9H-pyrido[2,3-b]indol-9-ylmethyl)phenyl)-N-(2-hydroxy-1(R)-phenylethyl)acetamide,
2-Cyclopentyl-2-{4-[(2,4-dimethyl-9H-pyrido[2,3-b]indol-9-yl)methyl]phenyl}-2′-phenylacetohydrazide,
2-{4-[(2,4-Dimethylpyrimido[1,2-a]indol-10-yl)methyl]phenyl}-3-methyl-2′-phenyl-butanehydrazide,
(−)-[2S-[2α,4α(S*)]]-4-[4-[4-[4-[[2-(4-chlorophenyl)-2-[[(4-methyl-4H-1,2,4-triazol-3-yl)thio]methyl]-1,3-dioxolan-4-yl]methoxy]phenyl]-1-piperazinyl]phenyl]-2,4-dihydro-2-(1-methylpropyl)-3H-1,2,4-triazol-3-one,
(−)-[2S-[2α,4α(S*)]]-4-[4-[4-[4-[[2-(4-chlorophenyl)-2-[[(4-methyl-4H-1,2,4-triazol-3-yl)sulphonyl]methyl]-1,3-dioxolan-4-yl]methoxy]phenyl]-1-piperazinyl]phenyl]-2,4-dihydro-2-(1-methylpropyl)-3H-1,2,4-triazol-3-one,
(S)-6-Methyl-4′-trifluoromethylbiphenyl-2-carboxylic acid-(2-methylsulphonylamino-indan-5-yl)-amide,
(R)-6-Methyl-4′-trifluoromethylbiphenyl-2-carboxylic acid-(2-methoxycarbonylamino-indan-5-yl)-amide,
(S)-6-Methyl-4′-trifluoromethylbiphenyl-2-carboxylic acid-(2-methoxycarbonylamino-indan-5-yl)-amide,
(R)-4-Fluoro-4′-trifluoromethylbiphenyl-2-carboxylic acid-(2-methylsulphonylamino-indan-5-yl)-amide,
(S)-4-Fluoro-4′-trifluoromethylbiphenyl-2-carboxylic acid-(2-methylsulphonylamino-indan-5-yl)-amide,
6-Methyl-4′-trifluoromethylbiphenyl-2-carboxylic acid-(2-dimethylaminocarbonylamino-indan-5-yl)-amide,
4′-Trifluoromethyl-biphenyl-2-carboxylic acid-[2-(2H-[1,2,4]triazol-3-ylmethyl)-1,2,3,4-tetrahydro-isoquinolin-6-yl]-amide and
4′-Trifluoromethyl-biphenyl-2-carboxylic acid-[2-(2-acetylamino-ethyl)-1,2,3,4-tetrahydro-isoquinolin-6-yl]-amide
as well as the tautomers, diastereomers, enantiomers, mixtures thereof and the salts thereof.

11. The method according to claim 10, wherein the MTP inhibitor is selected from the list consisting of:
9-[4-[4-[2-(4-Trifluoromethylphenyl)benzoylamino]piperidin-1-yl]butyl]-N-(2,2,2-trifluoroethyl)-9H-fluorene-9-carboxamide,
9-[4-[2,5-Dimethyl-4-[[[4′-(trifluoromethyl)[1,1′-biphenyl]-2-yl]carbonyl]amino]-1H-benzimidazol-1-yl]butyl]-N-(2,2,2-trifluoroethyl)-9H-fluorene-9-carboxamide,
4′-Trifluoromethyl-biphenyl-2-carboxylic acid-[2-(2H-[1,2,4]triazol-3-ylmethyl)-1,2,3,4-tetrahydro-isoquinolin-6-yl]-amide and
4′-Trifluoromethyl-biphenyl-2-carboxylic acid-[2-(2-acetylamino-ethyl)-1,2,3,4-tetrahydro-isoquinolin-6-yl]-amide
as well as the tautomers, diastereomers, enantiomers, mixtures thereof and the salts thereof.

12. A method of claim 11 wherein the MTP inhibitor is 9-[4-[4-[2-(4-trifluoromethylphenyl)benzoylamino]piperidin-1-yl]butyl]-N-(2,2,2-trifluoro-ethyl)-9H-fluorene-9-carboxamide and the fibrate is bezafibrate.

13. Pharmaceutical composition comprised of an MTP inhibitor combined with a fibrate.

14. Pharmaceutical composition according to claim 13 containing at least one MTP inhibitor combined with
a) bezafibrate,
b) ciprofibrate,
c) clofibrate,
d) fenofibrate or
e) gemfibrozil.

15. Pharmaceutical composition according to claim 13 wherein said MTP inhibitor is of the general formula 1 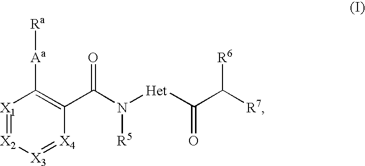
wherein
X₁ denotes the group CR¹,
X₂ denotes the group CR²,
X₃ denotes the group CR³ and
X₄ denotes the group CR⁴ or
one or two of the groups X₁ to X₄ in each case denote a nitrogen atom and the remainder of the groups X₁ to X₄ denote three or two of the groups CR¹ to CR⁴,
while R¹, R², R³ and R⁴ in each case denote a hydrogen atom or
one or two of the groups R¹ to R⁴ independently of one another in each case denote a fluorine, chlorine or bromine atom, a C₁₋₃-alkyl group, a trifluoromethyl, hydroxy, C₁₋₃-alkoxy, trifluoromethoxy, amino, C₁₋₃-alkylamino or di-(C₁₋₃-alkyl)-amino group and the remainder of the groups R¹ to R⁴ in each case represent a hydrogen atom,
while R⁴ additionally together with R⁵ may assume the meaning of a —(CH₂)ₙ— bridge wherein n denotes the number 1, 2 or 3, and
A^{a} denotes a bond, an oxygen or sulphur atom, an —NH, —N(C₁₋₃-alkyl), sulphinyl, sulphonyl or carbonyl group,
one of the groups —CH₂—, —(CH₂)₂—, —CH═CH—, —C≡C—, —OCH₂—, —CH₂O—, —NH—CH₂—, —CH₂—NH—, —NH—CO—, —CO—NH—, —NH—SO₂— or —SO₂—NH—,
wherein a hydrogen atom bound to a carbon atom and/or a hydrogen atom bound to a nitrogen atom may be replaced in each case by a C₁₋₃-alkyl group and wherein a heteroatom of the group A^{a} is not linked to a nitrogen atom of a 5-membered heteroaryl group of the group R^{a},
R^{a} denotes a phenyl, 1-naphthyl or 2-naphthyl group,
a 5-membered heteroaryl group bound via a carbon or nitrogen atom, which contains
an imino group optionally substituted by a C₁₋₄-alkyl or C₁₋₄-alkylcarbonyl group, an oxygen or sulphur atom,
an imino group optionally substituted by a C₁₋₄-alkyl group or an oxygen or sulphur atom and additionally a nitrogen atom or
an imino group optionally substituted by a C₁₋₄-alkyl group and two nitrogen atoms or
an oxygen or sulphur atom and two nitrogen atoms,
a 6-membered heteroaryl group which contains one or two nitrogen atoms,
while a phenyl ring may be fused to the abovementioned 5- or 6-membered heteroaryl groups via two adjacent carbon atoms and the bicyclic heteroaryl groups thus formed may be bound via the heteroaromatic or carbocyclic moiety and
wherein the abovementioned phenyl and naphthyl groups as well as the mono- and bicyclic heteroaryl groups in the carbon skeleton may be monosubstituted by a fluorine, chlorine or bromine atom, by a C₁₋₄-alkyl group, by a C₃₋₇-cycloalkyl, trifluoromethyl, phenyl, hydroxy, C₁₋₃-alkoxy, trifluoromethoxy, amino, C₁₋₃-alkylamino, di-(C₁₋₃-alkyl)amino, acetylamino, N—(C₁₋₃-alkyl)-acetylamino, propionylamino, N—(C₁₋₃-alkyl)-propionylamino, acetyl, propionyl, C₁₋₃-alkoxy-carbonyl, aminocarbonyl, C₁₋₃-alkylamino-carbonyl, di-(C₁₋₃-alkyl)amino-carbonyl or cyano group or, with the exception of 5-membered heteroaryl groups containing more than two heteroatoms, may also be disubstituted by the abovementioned substituents, while the substituents may be identical or different,
a C₃₋₇-cycloalkyl group, wherein
in each case the methylene group in the 4 position of a 6- or 7-membered cycloalkyl group may be replaced by an oxygen or sulphur atom, by a sulphinyl or sulphonyl group or by an imino group optionally substituted by a C₁₋₅-alkyl, phenyl, C₁₋₄-alkyl-carbonyl, C₁₋₄-alkoxy-carbonyl, C₁₋₃-alkyl-aminocarbonyl or di-(C₁₋₃-alkyl)-aminocarbonyl group,
a 4- to 7-membered cycloalkyleneimino group wherein
the cycloalkylene moiety may be fused to a phenyl ring or
one or two hydrogen atoms may be replaced in each case by a C₁₋₃-alkyl group and/or
in each case the methylene group in the 4 position of a 6- or 7-membered cycloalkyleneimino group may be substituted by a hydroxycarbonyl, C₁₋₃-alkoxycarbonyl, amino, C₁₋₃-alkylamino, di-(C₁₋₃-alkyl)amino, aminocarbonyl, C₁₋₃-alkylamino-carbonyl, di-(C₁₋₃-alkyl)-aminocarbonyl or phenyl-C₁₋₃-alkylamino group or
may be replaced by an oxygen or sulphur atom, by a sulphinyl or sulphonyl group or by an imino group optionally substituted by a C₁₋₅-alkyl, phenyl, C₁₋₄-alkyl-carbonyl, C₁₋₄-alkoxy-carbonyl, C₁₋₃-alkyl-aminocarbonyl or di-(C₁₋₃-alkyl)-aminocarbonyl group or
in a 5-, 6- or 7-membered cycloalkyleneimino group a —CH₂— group linked to the imino nitrogen atom may be replaced by a carbonyl group or a —(CH₂)₂— group linked to the imino nitrogen atom may be replaced by a —CO—NR⁸— group or a —(CH₂)₃— group linked to the imino nitrogen atom may be replaced by a —CO—NR⁸—CO— group,
while R⁸ denotes a hydrogen atom or a C₁₋₃-alkyl group,
R⁵ denotes a hydrogen atom or a C₁₋₅-alkyl group,
Het denotes a 5-membered heteroarylene group bound via two carbon atoms or, if Het denotes a double-bonded pyrrole group, it may also be bound via a carbon atom and the imino-nitrogen atom, the latter being linked to the adjacent carbonyl group in formula (I), which contains
an imino group substituted by the group R⁹, an oxygen or sulphur atom or
an imino group substituted by the group R⁹ or an oxygen or sulphur atom and additionally a nitrogen atom,
while R⁹ denotes a hydrogen atom, a C₁₋₅-alkyl group, a C₂₋₃-alkyl group terminally substituted by an amino, C₁₋₃-alkylamino, di-(C₁₋₃-alkyl)-amino or C₁₋₅-alkoxy-carbonyl-amino group, a carboxy-C₁₋₃-alkyl, C₁₋₃-alkoxy-carbonyl-C₁₋₃-alkyl, phenyl, phenyl-C₁₋₃-alkyl, C₁₋₅-alkylcarbonyl or phenylcarbonyl group or R⁹ together with R⁶ denotes a —(CH₂)ₚ— bridge, wherein p denotes the number 2 or 3,
or an imino group optionally substituted by a C₁₋₃-alkyl group and two nitrogen atoms or
an oxygen or sulphur atom and two nitrogen atoms,
or a 6-membered heteroarylene group which contains one or two nitrogen atoms,
while the abovementioned heteroarylene groups in the carbon skeleton may be monosubstituted by a fluorine, chlorine or bromine atom, by a C₁₋₅-alkyl group, by a C₃₋₇-cycloalkyl, trifluoromethyl, hydroxy, C₁₋₃-alkoxy, trifluoromethoxy, amino, C₁₋₃-alkylamino, di-(C₁₋₃-alkyl)amino, acetylamino, N—(C₁₋₃-alkyl)-acetylamino, propionylamino, N—(C₁₋₃-alkyl)-propionylamino, acetyl, propionyl, benzoyl, C₁₋₃-alkoxy-carbonyl, aminocarbonyl, C₁₋₃-alkylamino-carbonyl-di-(C₁₋₃-alkyl)-amino-carbonyl or cyano group or, with the exception of 5-membered monocyclic heteroaryl groups containing more than one heteroatom, may also be disubstituted by the abovementioned substituents, while the substituents may be identical or different,
R⁶ denotes a hydrogen atom or a C₁₋₆-alkyl group,
R⁷ denotes a C₁₋₉-alkyl group,
a straight-chain or branched, mono-, di- or triunsaturated C₃₋₉-alkenyl or C₃₋₉-alkynyl group, while the multiple bonds are isolated from the nitrogen-carbon bond,
a straight-chain C₂₋₆-alkyl group which is terminally substituted by an amino, C₁₋₃-alkylamino or di-(C₁₋₃-alkyl)-amino group,
a C₁₋₆-alkyl group substituted by a C₃₋₇-cycloalkyl group, while
a hydrogen atom in the 3 position of the cyclopentyl group and in the 4 position of a 6- or 7-membered cycloalkyl group may be replaced in each case by a hydroxy, hydroxy-C₁₋₃-alkyl, C₁₋₅-alkoxy, C₁₋₅-alkoxy-C₁₋₃-alkyl, phenyl-C₁₋₃-alkoxy-C₁₋₃-alkyl, amino, C₁₋₅-alkylamino, di-(C₁₋₅-alkyl)amino, phenyl-C₁₋₃-alkylamino, C₁₋₅-alkyl-carbonylamino, benzoylamino, amino-C₁₋₃-alkyl, C₁₋₃-alkylamino-C₁₋₃-alkyl, di-(C₁₋₃-alkyl)amino-C₁₋₃-alkyl, phenyl-C₁₋₃-alkylamino-C₁₋₃-alkyl, C₁₋₃-alkyl-carbonylamino-C₁₋₃-alkyl, benzoylamino-C₁₋₃-alkyl, phenylamino-carbonyl, phenyl-C₁₋₃-alkylamino-carbonyl, carboxy or C₁₋₃-alkoxy-carbonyl group or
in each case the methylene group in the 4 position of a 6- or 7-membered cycloalkyl group may be replaced by an oxygen or sulphur atom or by an imino group optionally substituted by a C₁₋₆-alkyl, phenyl, C₁₋₆-alkyl-carbonyl, benzoyl, phenyl-(C₁₋₃-alkyl)-carbonyl, C₁₋₆-alkyl-aminocarbonyl, di-(C₁₋₅-alkyl)-aminocarbonyl, phenylaminocarbonyl, N—(C₁₋₃-alkyl)-phenylaminocarbonyl, phenyl-C₁₋₃-alkylamino-carbonyl or N—(C₁₋₃-alkyl)-phenyl-C₁₋₃-alkylamino-carbonyl group or
in a 5- or 6-membered cycloalkyl group one or two single bonds separated from each other by at least one bond and separated from position 1 may in each case be fused to a phenyl group, while in a bi-or tricyclic ring system thus formed the hydrogen atom bound to the saturated carbon atom in position 1 may be replaced by a C₁₋₅-alkylamino-carbonyl, di-(C₁₋₅-alkyl)amino-carbonyl, phenyl-C₁₋₃-alkylamino-carbonyl or C₁₋₅-alkoxy-carbonyl group, wherein terminal methyl groups in each case may be wholly or partially fluorinated,
a C₁₋₆-alkyl group optionally substituted by a C₃₋₇-cycloalkyl group, which is substituted by a carboxy or C₁₋₃-alkoxycarbonyl group,
by a phenyl, 1-naphthyl or 2-naphthyl group,
by a 5-membered heteroaryl group bound via a carbon or nitrogen atom, which contains
an imino group optionally substituted by a C₁₋₃-alkyl, trifluoromethyl, phenyl, phenyl-C₁₋₃-alkyl, C₁₋₃-alkylcarbonyl, phenylcarbonyl or phenyl-C₁₋₃-alkylcarbonyl group, an oxygen or sulphur atom,
an imino group optionally substituted by a C₁₋₃-alkyl group or an oxygen or sulphur atom and additionally a nitrogen atom or
an imino group optionally substituted by a C₁₋₃-alkyl group and two nitrogen atoms or
an oxygen or sulphur atom and two nitrogen atoms,
by a 6-membered heteroaryl group, which contains one or two nitrogen atoms,
while a phenyl ring may be fused to the abovementioned 5- or 6-membered heteroaryl groups via two adjacent carbon atoms and the bicyclic heteroaryl groups thus formed may be bound via the heteroaromatic or carbocyclic moiety,
while the abovementioned phenyl and naphthyl groups as well as the mono- and bicyclic heteroaryl groups in the carbon skeleton may be monosubstituted by a fluorine, chlorine or bromine atom, by a C₁₋₅-alkyl, trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, amino-C₁₋₃-alkyl, C₁₋₃-alkylamino-C₁₋₃-alkyl, di-(C₁₋₃-alkyl)amino-C₁₋₃-alkyl, C₁₋₅-alkoxy-carbonylamino-C₁₋₃-alkyl, acetylamino, propionylamino, N—(C₁₋₃-alkyl)-benzoylamino, acetyl, propionyl, carboxy, C₁₋₃-alkoxy-carbonyl, C₁₋₃-alkoxy-carbonyl-C₁₋₃-alkyl, aminocarbonyl, C₁₋₃-alkylamino-carbonyl, di-(C₁₋₃-alkyl)amino-carbonyl, or cyano group or, with the exception of 5-membered heteroaryl groups containing more than two heteroatoms, may also be disubstituted by the abovementioned substituents, while the substituents may be identical or different,
a C₁₋₆-alkyl group substituted by a phenyl group and a carboxy, C₁₋₃-alkoxy-carbonyl, aminocarbonyl, C₁₋₃-alkyl-aminocarbonyl or di-(C₁₋₃-alkyl)-aminocarbonyl group,
a phenyl-C₂₋₅-alkenylene-CH₂, phenyl-C₂₋₅-alkynylene-CH₂, heteroaryl-C₂₋₅-alkenylene-CH₂ or heteroaryl-C₂₋₅-alkynylene-CH₂ group, wherein a hydrogen atom of the methylene group in position 1 may be replaced by a C₁₋₃-alkyl group and independently thereof the phenyl moiety as well as the heteroaryl moiety may be mono- or disubstituted by fluorine, chlorine or bromine atoms, by C₁₋₆-alkyl, C₃₋₇-cycloalkyl, trifluoromethyl, C₁₋₃-alkoxy, phenyl, heteroaryl or cyano groups, while the substituents may be identical or different and disubstitution by two aromatic groups is excluded,
while heteroaryl denotes a 5-membered heteroaryl group bound via a carbon or nitrogen atom, which contains
an imino group substituted optionally by a C₁₋₃-alkyl group, an oxygen or sulphur atom,
an imino group substituted optionally by a C₁₋₃-alkyl group or an oxygen or sulphur atom and additionally a nitrogen atom or
an imino group substituted optionally by a C₁₋₃-alkyl group and two nitrogen atoms or an oxygen or sulphur atom and two nitrogen atoms,
or a 6-membered heteroaryl group, which contains one or two nitrogen atoms,
while a phenyl ring may be fused to the abovementioned 5- or 6-membered heteroaryl groups via two adjacent carbon atoms and the bicyclic heteroaryl groups thus formed may be bound via the heteroaromatic or carbocyclic moiety,
the group R^{b}-A^{b}-E^{b}-C₁₋₃-alkyl optionally substituted in the C₁₋₃-alkyl moiety by a C₁₋₄-alkyl or C₃₋₅-cycloalkyl group, wherein
R^{b} denotes a phenyl group optionally mono- or disubstituted by fluorine, chlorine, bromine or iodine atoms, by C₁₋₄-alkyl, C₂₋₄-alkenyl, C₂₋₄-alkynyl, C₃₋₇-cycloalkyl, trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, amino-C₁₋₃-alkyl, C₁₋₃-alkylamino-C₁₋₃-alkyl, di-(C₁₋₃-alkyl)amino-C₁₋₃-alkyl, acetylamino, propionylamino, acetyl, propionyl, carboxy, C₁₋₃-alkoxy-carbonyl, C₁₋₃-alkoxy-carbonyl-C₁₋₃-alkyl, aminocarbonyl, C₁₋₃-alkylamino-carbonyl, di-(C₁₋₃-alkyl)amino-carbonyl or cyano groups, while the substituents may be identical or different,
a 5-membered heteroaryl group which
may be bound via a carbon atom or, if A^{b} denotes a bond, a —CH₂, —(CH₂)₂, sulphonyl or carbonyl group, may also be bound via a nitrogen atom and which contains
an imino group optionally substituted by a C₁₋₃-alkyl group, an oxygen or sulphur atom, an imino group optionally substituted by a C₁₋₃-alkyl group or an oxygen or sulphur atom and additionally a nitrogen atom or
an imino group optionally substituted by a C₁₋₃-alkyl group and two nitrogen atoms or an oxygen or sulphur atom and two nitrogen atoms,
a 6-membered heteroaryl group, which contains one or two nitrogen atoms,
while a phenyl ring may be fused to the abovementioned 5- or 6-membered heteroaryl groups via two adjacent carbon atoms and the bicyclic heteroaryl groups thus formed may be bound via the heteroaromatic or carbocyclic moiety,
while the abovementioned mono- and bicyclic heteroaryl groups may be monosubstituted in the carbon skeleton by a fluorine, chlorine or bromine atom, by a C₁₋₄-alkyl, C₂₋₄-alkenyl, C₂₋₄-alkynyl, C₃₋₇-cycloalkyl, trifluoromethyl, phenyl, hydroxy, C₁₋₃-alkoxy, trifluoromethoxy, amino, C₁₋₃-alkylamino, di-(C₁₋₃-alkyl)-amino, acetylamino, propionylamino, acetyl, propionyl, C₁₋₃-alkoxy-carbonyl, aminocarbonyl, C₁₋₃-alkylamino-carbonyl, di-(C₁₋₃-alkyl)amino-carbonyl or cyano group or, with the exception of 5-membered heteroaryl groups containing more than two heteroatoms, may also be disubstituted by the abovementioned substituents, while the substituents may be identical or different,
a C₃₋₇-cycloalkyl group wherein
one or two hydrogen atoms in each case may be replaced by a C₁₋₃-alkyl group and/or
in each case the methylene group in the 4 position of a 6- or 7-membered cycloalkyl group may be replaced by an oxygen or sulphur atom, by a sulphinyl, sulphonyl or by an imino group optionally substituted by a C₁₋₃-alkyl, C₁₋₃-alkyl-carbonyl, C₁₋₃-alkoxy-carbonyl, C₁₋₃-alkyl-aminocarbonyl or di-(C₁₋₃-alkyl)-aminocarbonyl group or
the two hydrogen atoms of the methylene group in the 3-position of a cyclopentyl group or in 3- or 4-position of a cyclohexyl or cycloheptyl group may be replaced by an n-butylene, n-pentylene, n-hexylene, 1,2-ethylenedioxy or
1,3-propylenedioxy group and in the rings thus formed one or two hydrogen atoms may be replaced by C₁₋₃-alkyl groups,
a 4- to 7-membered cycloalkyleneimino group wherein
the cycloalkylene moiety may be fused to a phenyl ring or
one or two hydrogen atoms in each case may be replaced by a C₁₋₃-alkyl group and/or
in each case the carbon atom in the 4 position of a 6- or 7-membered cycloalkyleneimino group may be substituted by a hydroxy-C₁₋₃-alkyl, C₁₋₆-alkoxy-C₁₋₃-alkyl, hydroxycarbonyl, C₁₋₆-alkoxycarbonyl, aminocarbonyl, C₁₋₃-alkylamino-carbonyl, di-(C₁₋₃-alkyl)-aminocarbonyl-, 4- to 7-membered cycloalkyleneimino, phenyl, 4-(C₁₋₃-alkyl)-1,2,4-triazol-3-yl, phenyl-C₁₋₃-alkylamino or N—(C₁₋₃-alkyl)-phenyl-C₁₋₃-alkylamino group or
may be replaced by an oxygen or sulphur atom, by a sulphinyl or sulphonyl group or by an imino group optionally substituted by a C₁₋₃-alkyl, phenyl, C₁₋₃-alkyl-carbonyl, benzoyl, phenyl-C₁₋₃-alkyl-carbonyl, C₁₋₃-alkyl-aminocarbonyl, di-(C₁₋₃-alkyl)-aminocarbonyl, phenylaminocarbonyl or N—(C₁₋₃-alkyl)-phenylaminocarbonyl group or
the two hydrogen atoms of the methylene group in the 3 position of a 5-membered cycloalkyleneimino group or in the 3 or 4 position of a 6- or 7-membered cycloalkyleneimino group may be replaced by an n-butylene, n-pentylene, n-hexylene, 1,2-ethylenedioxy or 1,3-propylenedioxy group and in the rings thus formed one or two hydrogen atoms may be replaced by C₁₋₃-alkyl groups or
in a 5-, 6- or 7-membered cycloalkyleneimino group a —CH₂— group linked to the imino nitrogen atom may be replaced by a carbonyl group or a —(CH₂)₂— group linked to the imino nitrogen atom may be replaced by a —CO—NR⁸— group or a —(CH₂)₃— group linked to the imino nitrogen atom may be replaced by a —CO—NR⁸—CO— group,
while R⁸ denotes a hydrogen atom or a C₁₋₃-alkyl group,
A^{b} denotes a bond, an oxygen or sulphur atom, an —NH, —N(C₁₋₃-alkyl), sulphinyl, sulphonyl or a carbonyl group,
one of the groups —CH₂—, —(CH₂)₂—, —O—CH₂—, —CH₂—O—, NH—CH₂—, —CH₂—NH—, —NH—CO—, —CO—NH—, —NH—SO—₂, —SO₂—NH—, —CH═CH— or —C≡C—
wherein a hydrogen atom bound to a carbon atom and/or a hydrogen atom bound to a nitrogen atom may be replaced by a C₁₋₃-alkyl group in each case and a heteroatom of the group A^{b} is not linked to a nitrogen atom of a 5-membered heteroaryl group of the group R^{b},
E^{b} denotes a phenylene group optionally substituted by a fluorine, chlorine or bromine atom, by a C₁₋₄-alkyl group, by a trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, amino-C₁₋₃-alkyl, C₁₋₃-alkylamino-C₁₋₃-alkyl, di-(C₁₋₃-alkyl)amino-C₁₋₃-alkyl, acetylamino, propionylamino, acetyl, propionyl, carboxy, C₁₋₃-alkoxy-carbonyl, C₁₋₃-alkoxy-carbonyl-C₁₋₃-alkyl, aminocarbonyl, C₁₋₃-alkylamino-carbonyl, di-(C₁₋₃-alkyl)amino-carbonyl or cyano group,
the group R^{c}-A^{c}-E^{c}-C₁₋₃-alkyl optionally substituted in the C₁₋₃-alkyl moiety by a C₁₋₄-alkyl or C₃₋₅-cycloalkyl group wherein
R^{c} assumes the meanings given for R^{b} hereinbefore, while any reference to A^{b} must be replaced by a reference to A^{c},
A^{c} assumes the meanings given for A^{b} hereinbefore, while any reference to R^{b} must be replaced by a reference to R^{c},
E^{c} denotes a 5-membered heteroarylene group bound via two carbon atoms or via a carbon atom and an imino-nitrogen atom, while the imino-nitrogen atom of the heteroarylene group is not linked to a heteroatom of the group A^{c} and the heteroarylene group contains
an imino group optionally substituted by a C₁₋₃-alkyl group, an oxygen or sulphur atom,
an imino group optionally substituted by a C₁₋₃-alkyl group or an oxygen or sulphur atom and additionally a nitrogen atom or
an imino group optionally substituted by a C₁₋₃-alkyl group and two nitrogen atoms or
an oxygen or sulphur atom and two nitrogen atoms,
or a 6-membered heteroarylene group, which contains one or two nitrogen atoms,
while a phenyl ring may be fused to the abovementioned 5-membered heteroarylene groups containing one or two heteroatoms as well as to the abovementioned 6-membered heteroarylene groups via two adjacent carbon atoms and the bicyclic heteroarylene groups thus formed may be bound via the heteroaromatic and/or carbocyclic moiety,
and while the abovementioned mono- and bicyclic heteroarylene groups in the carbon skeleton may be substituted by a fluorine, chlorine or bromine atom, by a C₁₋₄-alkyl group, by a C₃₋₇-cycloalkyl, trifluoromethyl, hydroxy, C₁₋₃-alkoxy, trifluoromethoxy, amino, C₁₋₃-alkylamino, acetylamino, propionylamino, acetyl, propionyl, C₁₋₃-alkoxy-carbonyl, aminocarbonyl, C₁₋₃-alkylamino-carbonyl or cyano group,
or R⁶ and R⁷ together denote an n-alkylene bridge with 3 to 6 carbon atoms, wherein
one or two hydrogen atoms in each case may be replaced by a C₁₋₃-alkyl group and/or
a —CH₂—CH₂— group may be replaced by a 1,2-linked phenylene group which may be mono- or disubstituted by fluorine, chlorine or bromine atoms, by C₁₋₃-alkyl, trifluoromethyl, hydroxy, C₁₋₃-alkoxy, trifluoromethoxy, amino, C₁₋₃-alkylamino, di-(C₁₋₃-alkyl)amino, acetylamino, propionylamino, acetyl, propionyl, C₁₋₃-alkoxy-carbonyl, aminocarbonyl, C₁₋₃-alkylamino-carbonyl, cyano, phenyloxy or phenyl-C₁₋₃-alkyl groups, while disubstitution with the last-named group is excluded,
while the abovementioned phenyloxy- and phenyl-C₁₋₃-alkyl group in the phenyl moiety may in turn be substituted by a fluorine, chlorine or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino or cyano group,
or in each case the carbon atom in the 3 position of a n-pentylene or n-hexylene group may be monosubstituted by a C₁₋₃-alkyl group terminally substituted by a phenyl, cyano, hydroxy, C₁₋₃-alkoxy, amino, C₁₋₃-alkylamino, di-(C₁₋₃-alkyl)-amino or a 5- to 7-membered cycloalkyleneimino group, by a carboxy, C₁₋₃-alkoxycarbonyl, amino-C₁₋₃-alkyl, C₁₋₃-alkylamino-C₁₋₃-alkyl, N—C₁₋₃-alkyl-N—(C₁₋₃-alkyl-carbonyl)-amino-C₁₋₃-alkyl, di-(C₁₋₃-alkyl)-amino-C₁₋₃-alkyl, aminocarbonyl, C₁₋₃-alkylamino-carbonyl or di-(C₁₋₃-alkyl)-aminocarbonyl group or may be disubstituted by a phenyl group and a cyano, hydroxy or C₁₋₃-alkoxy group or
the methylene group in the 3 position of a n-pentylene or n-hexylene group may be replaced by an oxygen or sulphur atom, by a sulphinyl or sulphonyl group or by an imino group optionally substituted by a C₁₋₃-alkyl, phenyl-C₁₋₃-alkyl, C₁₋₃-alkyl-carbonyl, benzoyl, C₁₋₃-alkyl-aminocarbonyl, di-(C₁₋₃-alkyl)-aminocarbonyl, phenylaminocarbonyl or N—(C₁₋₃-alkyl)-phenylaminocarbonyl group or
a methylene group in position 1 of an n-butylene, n-pentylene or n-hexylene group may be replaced by a carbonyl group,
while the phenyl groups mentioned as being unsubstituted or monosubstituted in the definition of the abovementioned groups as well as aromatic or heteroaromatic parts of molecules may, unless otherwise stated, optionally additionally be substituted in the carbon skeleton by fluorine, chlorine or bromine atoms, by C₁₋₃-alkyl groups, by trifluoromethyl, hydroxy, C₁₋₃-alkoxy, trifluoromethoxy, amino, C₁₋₃-alkylamino, acetylamino, acetyl, C₁₋₃-alkoxy-carbonyl, aminocarbonyl, C₁₋₃-alkylamino-carbonyl or cyano groups, while the substituents may be identical or different and the resulting aromatic groups and parts of molecules may be at most disubstituted,
the hydrogen atoms in the C₁₋₃-alkyl and alkoxy groups mentioned in the definition of the above groups may be wholly or partially replaced by fluorine atoms and
the alkyl and alkoxy groups mentioned in the definition of the above groups or in the alkyl moieties contained in the groups of formula I defined above with more than two carbon atoms may be straight-chain or branched, unless otherwise specified,
the carboxy groups mentioned in the definition of the abovementioned groups may be replaced by a group which can be converted into a carboxy group in vivo or by a group which is negatively charged under physiological conditions, and/or
the amino and imino groups mentioned in the definition of the abovementioned groups may be substituted by a group which can be cleaved in vivo,
the tautomers, the diastereomers, the enantiomers, the mixtures and the salts thereof.

16. Pharmaceutical composition according to claim 13 wherein said MTP inhibitor is an MTP inhibitor of selected from the list consisiting of:
(a) N-[3-(Biphenyl-4-yl)-prop-2-ynyl]-4-(4′-trifluoromethylbiphenyl-2-carbonylamino)-1-methyl-pyrrole-2-carboxylic acid amide,
(b) N-[4-(1,4-Dioxa-8-aza-spiro[4.5]dec-8-yl)-phenylmethyl]-4-(4′-trifluoromethyl-biphenyl-2-carbonylamino)-1-methyl-pyrrole-2-carboxylic acid amide,
(c) N-[4-(3-Aza-spiro[5.5]undec-3-yl)-phenylmethyl]-4-(4′-trifluoromethylbiphenyl-2-carbonylamino)-1-methyl-pyrrole-2-carboxylic acid amide,
(d) N-[4-(6-Methylpyridazin-3-yl)-phenylmethyl]-4-(4′-trifluoromethylbiphenyl-2-carbonylamino)-1-methyl-pyrrole-2-carboxylic acid amide,
(e) N-(4′-Hydroxybiphenyl-4-yl)methyl-4-(4′-trifluoromethylbiphenyl-2-carbonylamino)-1-methyl-pyrrole-2-carboxylic acid amide,
(f) N-[4-(1,4-Dioxa-spiro[4.5]dec-8-yl)-phenylmethyl]-4-(4′-trifluoromethylbiphenyl-2-carbonylamino)-1-methyl-pyrrole-2-carboxylic acid amide,
(g) N-(4′-Methylbiphenyl-4-yl)methyl-4-(4′-trifluoromethylbiphenyl-2-carbonylamino)-1-methyl-pyrrole-2-carboxylic acid amide,
(h) N-[3-(4-Isopropylphenyl)-prop-2-ynyl]-4-(4′-trifluoromethyl-biphenyl-2-carbonylamino)-1-methyl-pyrrole-2-carboxylic acid amide,
(i) N-[3-(4-Biphenyl)-prop-2-ynyl]-4-(4′-trifluoromethylbiphenyl-2-carbonylamino)-1-methyl-imidazole-2-carboxylic acid amide,
(j) N-[3-(4-Trifluoromethylphenyl)-prop-2-ynyl]-4-(4′-trifluoromethylbiphenyl-2-carbonylamino)-1-methyl-imidazole-2-carboxylic acid amide und
(k) N-[4-(4-Propylpiperidino)-phenylmethyl]-4-(4′-trifluoromethylbiphenyl-2-carbonyl-amino)-1-methyl-pyrrol-2-carboxylic acid amide
and the salts thereof.

17. Pharmaceutical composition according to claim 13 wherein said MTP inhibitor is an MTP inhibitor of is selected from the list consisting of:
9-{4-[4-(4-Trifluoromethyl-phenylacetyl)-piperazino]-butyl}-9H-fluoren-9-carboxylic acid-(2,2,2-trifluoroethyl)-amide,
9-[4-(4-Phenylacetyl-piperazino)-butyl]-9H-fluorene-9-carboxylic acid-(2,2,2-trifluoroethyl)-amide,
9-(4-{4-[2-Phenyl-butyryl]-piperazino}-butyl)-9H-fluorene-9-carboxylic acid-(2,2,2-trifluoroethyl)-amide,
9-(4-{4-(3-Phenylpropionyl)-piperazino}-butyl)-9H-fluorene-9-carboxylic acid-(2,2,2-trifluoroethyl)-amide,
9-{4-[4-(4-Phenyl-butyryl)-piperazino]-butyl}-9H-fluorene-9-carboxylic acid-(2,2,2-trifluoroethyl)-amide,
9-(4-{4-(4-(Pyridin-2-yl-acetyl)-piperazino}-butyl)-9H-fluorene-9-carboxylic acid-(2,2,2-trifluoroethyl)-amide,
9-(4-{4-[2-Oxo-2-phenyl-acetyl]-piperazino}-butyl)-9H-fluorene-9-carboxylic acid-(2,2,2-trifluoroethyl)-amide,
9-(4-{4-[(2,4-Dichlorophenyl)-acetyl]-piperazino}-butyl)-9H-fluorene-9-carboxylic acid-(2,2,2-trifluoroethyl)-amide,
9-[4-[4-[2-(4-Trifluoromethylphenyl)benzoylamino]piperidin-1-yl]butyl]-N-(2,2,2-trifluoroethyl)-9H-fluorene-9-carboxamide,
9-[4-[2,5-Dimethyl-4-[[[4′-(trifluoromethyl)[1,1′-biphenyl]-2-yl]carbonyl]amino]-1H-benzimidazol-1-yl]butyl]-N-(2,2,2-trifluoroethyl)-9H-fluorene-9-carboxamide,
2(S)-Cyclopentyl-2-(4-(2,4-dimethyl-9H-pyrido[2,3-b]indol-9-ylmethyl)phenyl)-N-(2-hydroxy-1(R)-phenylethyl)acetamide,
2-Cyclopentyl-2-{4-[(2,4-dimethyl-9H-pyrido[2,3-b]indol-9-yl)methyl]phenyl}-2′-phenylacetohydrazide,
2-{4-[(2,4-Dimethylpyrimido[1,2-a]indol-10-yl)methyl]phenyl}-3-methyl-2′-phenyl-butanehydrazide,
(−)-[2S-[2α,4α(S*)]]-4-[4-[4-[4-[[2-(4-chlorophenyl)-2-[[(4-methyl-4H-1,2,4-triazol-3-yl)thio]methyl]-1,3-dioxolan-4-yl]methoxy]phenyl]-1-piperazinyl]phenyl]-2,4-dihydro-2-(1-methylpropyl)-3H-1 ,2,4-triazol-3-one,
(−)-[2S-[2α,4α(S*)]-4-[4-[4-[4-[[2-(4-chlorophenyl)-2-[[(4-methyl-4H-1,2,4-triazol-3-yl)sulphonyl]methyl]-1,3-dioxolan-4-yl]methoxy]phenyl]-1-piperazinyl]phenyl]-2,4-dihydro-2-(1-methylpropyl)-3H-1,2,4-triazol-3-one,
(S)-6-Methyl-4′-trifluoromethylbiphenyl-2-carboxylic acid-(2-methylsulphonylamino-indan-5-yl)-amide,
(R)-6-Methyl-4′-trifluoromethylbiphenyl-2-carboxylic acid-(2-methoxycarbonylamino-indan-5-yl)-amide,
(S)-6-Methyl-4′-trifluoromethylbiphenyl-2-carboxylic acid-(2-methoxycarbonylamino-indan-5-yl)-amide,
(R)-4-Fluoro-4′-trifluoromethylbiphenyl-2-carboxylic acid-(2-methylsulphonylamino-indan-5-yl)-amide,
(S)-4-Fluoro-4′-trifluoromethylbiphenyl-2-carboxylic acid-(2-methylsulphonylamino-indan-5-yl)-amide,
6-Methyl-4′-trifluoromethylbiphenyl-2-carboxylic acid-(2-dimethylaminocarbonylamino-indan-5-yl)-amide,
4′-Trifluoromethyl-biphenyl-2-carboxylic acid-[2-(2H-[1,2,4]triazol-3-ylmethyl)-1,2,3,4-tetrahydro-isoquinolin-6-yl]-amide and
4′-Trifluoromethyl-biphenyl-2-carboxylic acid-[2-(2-acetylamino-ethyl)-1,2,3,4-tetrahydro-isoquinolin-6-yl]-amide
as well as the tautomers, diastereomers, enantiomers, mixtures thereof and the salts thereof.

18. The Pharmaceutical composition of claim 17 wherein the MTP inhibitor is 9-[4-[4-[2-(4-trifluoromethylphenyl)benzoylamino]piperidin-1-yl]butyl]-N-(2,2,2-trifluoro-ethyl)-9H-fluorene-9-carboxamide and the fibrate is bezafibrate.

19. A pharmaceutical composition according to claim 13 suitable for oral administration.

20. Products comprised of an MTP inhibitor and a fibrate as a combined preparation to be administered simultaneously, separately or at different times, for lowering lipids.

21. Product according to claim 20, wherein the fibrate is selected from the list consisting of:
a) bezafibrate,
b) ciprofibrate,
c) clofibrate,
d) fenofibrate and
e) gemfibrozil.

22. Products comprised of an MTP inhibitor and a fibrate as a combined preparation to be administered simultaneously, separately or at different times, for lowering lipids wherein said MTP inhibitor is the MTP inhibitor of claim 15.

23. Products comprised of an MTP inhibitor according to claim 22 and wherein the fibrate is selected from the list consisting
a) bezafibrate,
b) ciprofibrate,
c) clofibrate,
d) fenofibrate and
e) gemfibrozil.

24. Products comprised of an MTP inhibitor according to claim 16 and a fibrate as a combined preparation to be administered simultaneously, separately or at different times, for lowering lipids.

25. Products comprised of an MTP inhibitor according to claim 17 and a fibrate as a combined preparation to be administered simultaneously, separately or at different times, for lowering lipids.

26. Product according to claim 20 comprised of 9-[4-[4-[2-(4-trifluoromethylphenyl)benzoylamino]piperidin-1-yl]butyl]-N-(2,2,2-trifluoro-ethyl)-9H-fluorene-9-carboxamide combined with bezafibrate.

27. Product according to claim 20 suitable for oral use.

28. A method for reducing the liver toxicity of an MTP inhibitor by administration of a pharmaceutical composition comprised of a fibrate and one or more MTP inhibitors.

29. The method of claim 28 wherein said fibrate is selected from among
a) bezafibrate,
b) ciprofibrate,
c) clofibrate,
d) fenofibrate and
e) gemfibrozil.

30. A method for reducing the liver toxicity of an MTP inhibitor by administration of a pharmaceutical composition comprised of a fibrate and one or more MTP inhibitors wherein the MTP inhibitor is the MTP inhibitor of claim 15.

31. A method for reducing the liver toxicity of an MTP inhibitor by administration of a pharmaceutical composition comprised of a fibrate and one or more MTP inhibitors wherein the MTP inhibitor is the MTP inhibitor of claim 16.

32. A method for reducing the liver toxicity of an MTP inhibitor by administration of a pharmaceutical composition comprised of a fibrate and one or more MTP inhibitors wherein the MTP inhibitor is the MTP inhibitor of claim 17.

33. A method of claim 30 wherein the MTP inhibitor is 9-[4-[4-[2-(4-trifluoromethylphenyl)benzoylamino]piperidin-1-yl]butyl]-N-(2,2,2-trifluoro-ethyl)-9H-fluorene-9-carboxamide and the firbrate is bezafibrate.

34. The method of claim 30 wherein the pharmaceutical composition is an oral formulation.

34. A method for treating disease by administration of a pharmaceutical composition according to claim 15, wherein the groups X₁, X₂, X₃, X₄, R^{a}, A^{a}, R⁵, Het, R⁶ and R⁷ are defined in claim 15.

35. A method for treating disease by administration of a pharmaceutical composition according to claim 16.

36. The method of claim 34 wherein the disease is selected from the list consisting of hyperlipidaemia, dyslipidaemia, atherosclerosis, diabetes mellitus, obesity or pancreatitis.

37. The method of claim 35 wherein the disease is selected from the list consisiting of hyperlipidaemia, dyslipidaemia, atherosclerosis, diabetes mellitus, obesity or pancreatitis.

38. A process for preparing a pharmaceutical composition according to claim 13 wherein said MTP inhibitor and said fibrate are converted into a suitable formulation using excipients and carriers.
